SEQUENCE LISTING (8 sequences)

SEQ ID NO: 1 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 123 aa, synthetic construct
EVQLVQSGPGLVKPSETLSLTCTVSGGSIDSGYYYWGWIRQPPGKGLEWIGSIYYIGTTYYIPSLKSRVTISVDTSKNRFSLKLTSVTAADTAVYYCARQPITVAGHDAFDIWGQGTMVTVSS

SEQ ID NO: 4 - protein, 107 aa, synthetic construct [sequence omitted]

SEQ ID NO: 5 - protein, 120 aa, synthetic construct
QVQLQQWGPGLVKPSQTLSLTCTVSGGSINSGDYYWSWIRQPPGKGLESIGYIYYSGSTYYNPSLKSRVTISADTSKNQFSLKLTSVTAADTAVYYCARERVYSSSLDYWGQGTLVTVSS

SEQ ID NO: 6 - protein, 107 aa, synthetic construct [sequence omitted]

SEQ ID NO: 7 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2026/0315611 A1
(43) Pub. Date: Oct. 15, 2026

(54) LINKER COMPOUNDS AND LIGAND-DRUG CONJUGATES, PREPARATION METHODS AND USES THEREOF

(71) Applicant: Biocytogen Pharmaceuticals (Beijing) Co., Ltd., Beijing (CN)

(72) Inventors: Yuelei Shen, Beijing (CN); Yi Yang, Beijing (CN); Chengzhang Shang, Beijing (CN); Lei Zhang, Beijing (CN)

(21) Appl. No.: 19/481,573

(22) PCT Filed: May 10, 2024
(86) PCT No.: PCT/CN2024/092145

§ 371 Date: Nov. 5, 2025

(30) Foreign Application Priority Data
May 12, 2023 (WO) PCT/CN2023/093976
Sep. 7, 2023 (WO) PCT/CN2023/117368
Sep. 22, 2023 (WO) PCT/CN2023/120684

(51) Int. Cl. A61K 47/68; A61P 35/00; C07D 491/22; C07K 16/28; C07K 16/30; C07K 16/32

(52) U.S. Cl. CPC A61K 47/68037; A61K 47/6803; A61K 47/68031; A61K 47/6851; A61P 35/00; C07D 491/22; C07K 16/2863; C07K 16/30; C07K 16/32; C07K 2317/31

(57) ABSTRACT

Provided are novel linker compounds and ligand-drug conjugates, and more specifically to linker compounds comprising a short polypeptide and use thereof in preparation of ligand-drug conjugates, ligand-drug conjugates comprising the linker, and preparation and use thereof. The novel linker compounds improve the coupling efficiency of the ligand-drug conjugates as well as the homogeneity and overall stability of the conjugates, further improving the efficacy and safety of the ligand-drug conjugates.

### CLAIM OF PRIORITY

This application claims priority to PCT/CN2023/093976, filed on May 12, 2023, PCT/CN2023/117368, filed on Sep. 7, 2023, and PCT/CN2023/120684, filed on Sep. 22, 2023. The entire contents of the foregoing applications are incorporated herein by reference.

### FIELD OF THE INVENTION

The present invention belongs to the technical field of medicinal chemistry and relates to novel linker compounds and ligand-drug conjugates, and more specifically to linker compounds comprising a short polypeptide and use thereof in preparation of ligand-drug conjugates, ligand-drug conjugates comprising the linker, and preparation and use thereof.

### BACKGROUND OF THE INVENTION

The basic modules of an antibody-drug conjugate (ADC) comprise an antibody, a linker, and a toxin molecule, in which the antibody is used to deliver the toxin molecule to tumor site for enrichment, thereby killing tumor cells. Most of the traditional toxin molecules are highly active tubulin inhibitors or cytotoxic drugs that directly target DNA, which usually have relatively large toxic side effects, which limits the application of ADCs. The advantage of antibody-drug conjugates is to improve targeting, specific antibody and antigen binding, carry the drug to surroundings of the target cells, effectively kill tumor cells by releasing the drug in the vicinity of the target cells and reducing toxic side effects. Camptothecin drugs have promising applications in ADC drugs.

Recently Immunomedics has invented a new ADC drug IMMU-132 using the camptothecin SN38 as the warhead molecule, which has shown good anti-tumor effects, and Daiichi Sankyo has invented another ADC drug DS-8201a using the camptothecin DXd as the warhead molecule, which has also shown good anti-tumor effects. In existing ADC technology, camptothecin compounds are linked to antibodies primarily by modifying existing linker technology. In general, the ideal linker for ADCs must meet the following requirements: firstly, the small molecule must not be detached from the antibody in the plasma, and upon entry into the cell, the linker must break under the right conditions to release the active small molecule rapidly; secondly, the linker must have good physicochemical properties so that it can be linked to the antibody to form a conjugate; and thirdly, the linker must be easy to prepare so as to lay the foundation for the large-scale production of ADCs. IMMU-132 uses a pH-sensitive linker, which is less stable, while DS-8201a uses a tetrapeptide structure containing glycine-glycine-phenylalanine-glycine (GGFG), which has better stability. However, the toxins released by the ADC drugs mentioned above are SN38 and Dxd, both of which are Pgp substrates and still have problems such as tumor multidrug resistance. In addition, the tetrapeptide structure of GGFG has relatively low water solubility and is subject to polymerization during coupling of some antibodies, and immune-related side effects such as interstitial lung disease in vivo. AstraZeneca published a class of novel DNA topoisomerase 1 inhibitor antibody-drug conjugates (Design and Preclinical Evaluation of a Novel B7-H4-Directed Antibody-Drug Conjugate, AZD8205, Alone and in Combination with the PARP1-Selective Inhibitor AZD5305. Clin Cancer Res 2022: OF1-OF16.), in which the linker toxin AZ'0133 showed better in vivo efficacy. However, the toxin may have a low metabolic efficiency, while the linker may improve hydrophilicity only by introducing PEG units, which may improve aggregation during coupling to some extent, while the improvement of the overall stability of the antibody conjugate is very limited by the reassortment method (Nat Biotechnol 2015; 33:733-5.). In addition, Daiichi Sankyo's GGFG-Dxd based ADC drugs have a side effect of interstitial lung disease (ILD), which may be related to non-specific killing of immune-related cells, especially at high drug loading levels. Therefore, a further effective increase in the hydrophilicity of the linker, together with the discovery and availability of camptothecin derivatives with somewhat different metabolic pathways acting as toxins, will hopefully lead to further improvements in efficacy and/or to ADC drugs with a better safety profile.

The technical problem to be solved by the present invention is to explore the discovery of superior antitumor camptothecin compounds. Based on a comprehensive understanding of the ADC drugs, the present inventors have designed a series of active derivatives of antitumor camptothecin. It has been found experimentally that the antitumor small molecule compounds exhibit higher antitumor activity in cellular assays. In addition, the linker modification improves the coupling efficiency of the antibody conjugates as well as the homogeneity and overall stability of the conjugates, further improving the efficacy and safety of the antibody-drug conjugates.

### SUMMARY OF THE INVENTION

In one aspect, the present invention is directed to a compound of Formula (I):

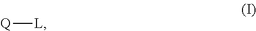

or a pharmaceutically acceptable salt, solvate, stereoisomer, or isotopic variant thereof, wherein
Q, and L are described herein.

In another aspect, the present invention is directed to a compound of Formula (II):

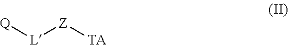

or a pharmaceutically acceptable salt, solvate, stereoisomer, or isotopic variant thereof, wherein
Q, L′, Z, and TA are described herein.

In a further aspect, the present invention is directed to a ligand-drug conjugate of Formula (III):

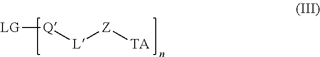

or a pharmaceutically acceptable salt, solvate, stereoisomer, or isotopic variant thereof, wherein
Q′, L′, Z, TA, LG and n are described herein.

In a further aspect, the present invention is directed to a pharmaceutical composition comprising a ligand-drug conjugate of Formula (III) or a pharmaceutically acceptable salt, solvate, stereoisomer, or isotopic variant thereof as provided herein; and a pharmaceutically acceptable diluent, carrier or excipient.

In a further aspect, the present invention is directed to use of a ligand-drug conjugate of Formula (III) or a pharmaceutically acceptable salt, solvate, stereoisomer, or isotopic variant thereof as provided herein in preparation of a medicament for treating a tumor in a subject.

In a further aspect, the present invention is directed to a ligand-drug conjugate of Formula (III) or a pharmaceutically acceptable salt, solvate, stereoisomer, or isotopic variant thereof as provided herein for use in treating a tumor in a subject.

In a further aspect, the present invention is directed to a method for treating a tumor in a subject comprising administrating to the subject an effective amount of a ligand-drug conjugate of Formula (III) or a pharmaceutically acceptable salt, solvate, stereoisomer, or isotopic variant thereof as provided herein.

### DESCRIPTION OF DRAWINGS

FIG. 1 lists amino acid sequences discussed in the disclosure.

FIG. 2 shows the average tumor volume in different groups of B-NDG mice that were injected with gastric cancer patient-derived tumor fragments, and were treated with T-(II′)-1, T-(II′)-2, T-(II′)-2(DAR4) or T-(II′)-4. PBS was used as a control.

FIG. 3 shows the average tumor volume in different groups of B-NDG mice that were injected with Hep-G2 cells, and were treated with hIgG1-(II′)-1, hIgG1-(II′)-2, hIgG1-(II′)-4, T-(II′)-1, T-(II′)-2, T-(II′)-2(DAR4) or T-(II′)-4. PBS was used as a control.

FIGS. 4A-4C show serum levels of total antibody in 0 minute, 15 minutes, 4 h, 24 h, 72 h, 10 day, 14 days, 21 days after administering with hIgG1-GGFG-Dxd (FIG. 4A), T-GGFG-Dxd (FIG. 4A), hIgG1-(II′)-4 (FIG. 4B), T-(II′)-4 (FIG. 4B) or Trastuzumab analog (FIG. 4C).

FIG. 4D shows serum levels of free payload in 0 minute, 15 minutes, 4 h, 24 h, 72 h after administering with hIgG1-GGFG-Dxd, T-GGFG-Dxd, hIgG1-(II′)-4, T-(II′)-4 or Trastuzumab analog.

FIGS. 5A-5C show the ratios of free payload to total payload in plasma of human (FIG. 5A), monkey (Macaca fascicularis) (FIG. 5B), or SD rat (FIG. 5C) in 0 day, 1 day, 2 days, 6 days, 8 days, 11 days and 14 days after the adding of T-(II′)-1, T-(II′)-2, or T-(II′)-4 to the plasma. T-GGFG-Dxd was used as a positive control.

FIGS. 6A-6B show endocytosis activities of anti-TROP2/EGFR bispecific antibody and ADCs in A431 cells (FIG. 6A) or NCI-H292 cells (FIG. 6B). ISO-CPT2(DAR8) was used as an isotype control. Sacituzumab govitecan and Cetuximab were used as positive controls.

FIG. 7 shows the average tumor volume in different groups of B-NDG mice that were injected with patient-derived breast tumor fragments, and were treated with PBS or ADCs.

FIG. 8 shows the average tumor volume in different groups of B-NDG mice that were injected with SKOV-3 cells, and were treated with PBS or ADCs.

FIG. 9 shows the average tumor volume in different groups of B-NDG mice that were injected with A431 cells, and were treated with PBS, antibody, or ADCs.

FIGS. 10A-10F show the average tumor volume in different groups of BALB/c nude mice that were injected with head and neck squamous cell carcinoma patient-derived tumor fragments (FIG. 10A), esophageal cancer patient-derived tumor fragments (FIG. 10B), colorectal cancer patient-derived tumor fragments (FIG. 10C and FIG. 10D), or gastric cancer patient-derived tumor fragments (FIG. 10E and FIG. 10F), and were treated with T-6F7-E-6C4-(II′)-2(DAR8). Saline was used as a control.

FIGS. 11A-11B show the concentration of total antibody (FIG. 11A) and CPT2 (FIG. 11B) in serum after administration of T-6F7-E-6C4-(II′)-2(DAR4) or T-6F7-E-6C4-(II′)-2(DAR8) to B-NDG mice.

FIGS. 11C-11D show the concentration of total antibody (FIG. 11C) and CPT2 (FIG. 11D) in tumor tissue after administration of T-6F7-E-6C4-(II′)-2(DAR4) or T-6F7-E-6C4-(II′)-2 (DAR8) to B-NDG mice.

FIGS. 12A-12B show the ratios of free CPT2 to total ADC in plasma of human, monkey (Macaca fascicularis), or SD rat in 0 day, 1 day, 2 days, 6 days, 8 days, 11 days and 14 days after the adding of T-6F7-E-6C4-(II′)-2(DAR4) (FIG. 12A) or T-6F7-E-6C4-(II′)-2(DAR8) (FIG. 12B) to the plasma. PBS was used as a control.

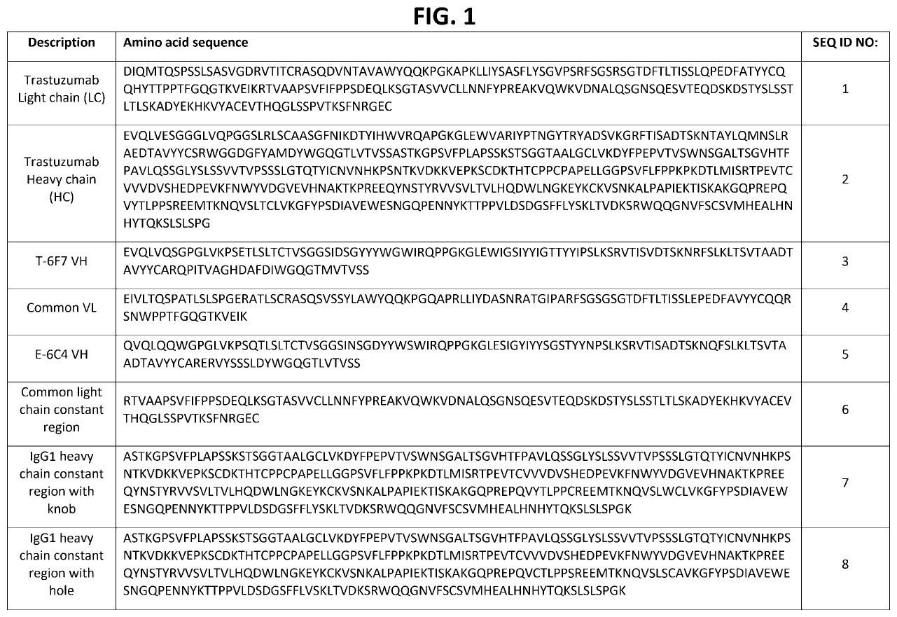

### DETAILED DESCRIPTION OF THE INVENTION

Reference will now be made in detail to certain embodiments, examples of which are illustrated in the accompanying detailed description. While enumerated embodiments will be described, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literatures and similar materials differs from or contradicts this application, including but not limited to defined terms, term usage, described techniques, or the like, this application controls.

It is appreciated that certain features of the present invention, which are, for clarity, described in the context of separate embodiments, can also be provided in combination in a single embodiment. Conversely, various features of the present invention, which are, for brevity, described in the context of a single embodiment, can also be provided separately or in any suitable sub-combination.

### Definitions

The terms used herein but not defined have their ordinary meaning and the meaning of such terms is independent at each occurrence thereof. Nevertheless, unless otherwise stated, the following definitions apply throughout the specification and claims.

As used herein, the singular forms “a”, “an”, and “the” include plural referents unless expressly stated to the contrary.

As used herein, the terms “comprise” and “include” are intended to specify the presence of stated features, integers, components, or steps, but they do not preclude the presence or addition of one or more other features, integers, components, steps, or groups thereof.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the given value by a variance of 20%, typically 10%, more typically 5%, and even more typically 1%. Sometimes, such a range can lie within the experimental error, type of standard methods used for the measurement and/or determination of a given value or range.

Definitions of specific functional groups and chemical terms are described in more detail below. For purpose of this invention, the chemical elements are identified in accordance with the Periodic Table of the Elements, CAS version, Handbook of Chemistry and Physics, 75^{th} Edition, inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

All ranges cited herein are inclusive, unless expressly stated to the contrary.

When a range of values is listed, it is intended to encompass each value and sub-range within the range. For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

When any variable occurs more than one time in any constituent or in Formula (I)-(IV) or in any other formula depicting and describing the compounds of the present invention, its definition at each occurrence is independent of its definition at every other occurrence. Also, combinations of substituents and/or variables are permissible only if such combinations result in stable compounds.

As used herein, the term “alkyl”, whether as part of another term or used independently, refers to an acyclic straight or branched chain saturated hydrocarbon group, which may be optionally substituted (i.e., unsubstituted or substituted) independently with one or more substituents described below. The term “Cᵢ₋ⱼ alkyl” refers to an alkyl having i to j carbon atoms. In certain embodiments, alkyl groups contain 1 to 12 carbon atoms. In certain embodiments, alkyl groups contain 1 to 11 carbon atoms. In certain embodiments, alkyl groups contain 1 to 11 carbon atoms, 1 to 10 carbon atoms, 1 to 9 carbon atoms, 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Non-limiting examples of alkyl groups include methyl; ethyl; n- and iso-propyl; n-, sec-, iso- and tert-butyl; neopentyl, and the like. Alkyl groups may be optionally substituted, as valency permits, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In certain embodiments, alkyl groups may be optionally substituted with halo, amino, hydroxy, methoxy, nitro, cyano, etc. Each of the substituents may itself be unsubstituted or, as valency permits, substituted with unsubstituted substituent(s) defined herein for each respective group.

As used herein, the term “alkylidene”, whether as part of another term or used independently, refers to a divalent substituent that is a monovalent alkyl having one hydrogen atom replaced with a valency. Alkylidene groups may be unsubstituted or substituted. An optionally substituted alkylidene is an alkylidene that is optionally substituted as described herein for alkyl.

As used herein, the term “alkenyl”, whether as part of another term or used independently, refers to linear or branched-chain hydrocarbon radical having at least one carbon-carbon double bond, which may be optionally substituted (i.e., unsubstituted or substituted) independently with one or more substituents described herein, and includes radicals having “cis” and “trans” orientations, or alternatively, “E” and “Z” orientations. In certain embodiments, alkenyl groups contain 2 to 12 carbon atoms. In certain embodiments, alkenyl groups contain 2 to 11 carbon atoms. In certain embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms. In certain embodiments, alkenyl groups contain 2 carbon atoms. Non-limiting examples of alkenyl groups include ethylenyl (or vinyl), propenyl, butenyl, pentenyl, 1-methyl-2-buten-1-yl, 5-hexenyl, and the like. An optionally substituted alkenyl is an alkenyl that is optionally substituted as described herein for alkyl.

As used herein, the term “alkenylidene”, whether as part of another term or used independently, a divalent substituent that is a monovalent alkenyl having one hydrogen atom replaced with a valency. Alkenylidene groups may be unsubstituted or substituted. An optionally substituted alkenylidene is an alkenylidene that is optionally substituted as described herein for alkyl.

As used herein, the term “cycloalkyl”, whether as part of another term or used independently, refers to a non-aromatic, saturated monocyclic and polycyclic ring system, in which all the ring atoms are carbon and which contains at least three ring forming carbon atoms. In certain embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Particularly, cycloalkyl groups may contain 3 to 10 ring forming carbon atoms (i.e., a C₃₋₁₀ cycloalkyl). Particularly, cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be optionally substituted (i.e., unsubstituted or substituted) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

As used herein, the term “cycloalkenyl”, whether as part of another term or used independently, refers to a non-aromatic, unsaturated monocyclic and polycyclic ring system having at least one carbon-carbon double bond, in which all the ring atoms are carbon and which contains at least three ring forming carbon atoms. The cycloalkynyl may be optionally substituted (i.e., unsubstituted or substituted) independently with one or more substituents described herein. In certain embodiments, the cycloalkenyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Particularly, cycloalkenyl groups may contain 5 to 6 ring forming carbon atoms (i.e., a C₅₋₆ cycloalkenyl). Particularly, cycloalkenyl groups may be monocyclic or bicyclic. In certain embodiments, the cycloalkenyl may have one, two, three, four, five, six double bonds, or more. In certain embodiments, the cycloalkenyl may have one, two, or three double bonds. Non-limiting examples of alkenyl groups include cyclopropenyl, cyclobutenyl, cyclopentenyl, cyclopentadienyl, cyclohexenyl, cyclohexadienyl, cycloheptenyl, cycloheptadienyl, and the like. An optionally substituted cycloalkenyl is an cycloalkenyl that is optionally substituted as described herein for alkenyl.

As used herein, the term “aryl”, whether as part of another term or used independently, refers to a mono-, bicyclic, or multicyclic carbocyclic ring system having at least one aromatic rings. Aryl groups may be 6- to 12-membered, for example, 8- to 12-membered, 6- to 10-membered, 6-membered. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be optionally substituted (i.e., unsubstituted or substituted) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

As used herein, the term “alkoxy”, whether as part of another term or used independently, refers to a chemical substituent of formula —OR, where R is an alkyl group, particularly C₁₋₁₂ alkyl, C₁₋₁₀ alkyl, C₁₋₆ alkyl, etc. Alkoxy groups may be unsubstituted or substituted. An optionally substituted alkoxy is an alkoxy group that is optionally substituted as defined herein for alkyl.

As used herein, the term “heteroalkyl”, whether as part of another term or used independently, refers to an alkyl group (e.g., an alkyl group defined herein) interrupted one or more times by one or two heteroatoms each time. Each heteroatom is independently O, N, or S. None of the heteroalkyl groups includes two contiguous oxygen atoms. The heteroalkyl group may be unsubstituted or substituted (e g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. In certain embodiments, carbon atoms are found at the termini of a heteroalkyl group. In certain embodiments, heteroalkyl is PEG.

As used herein, the term “heteroaryl”, whether as part of another term or used independently, refers to a monocyclic ring system, or a fused or bridged bicyclic, tricyclic, or tetracyclic ring system; the ring system contains one, two, three, or four heteroatoms independently selected from the group consisting of nitrogen, oxygen, and sulfur; and at least one of the rings is an aromatic ring. Heteroaryl groups may be 5- to 12-membered, for example, 8- to 12-membered, 5- to 10-membered, 5- to 6-membered. Heteroaryl groups have a carbon count of 1 to 16 carbon atoms unless otherwise specified. Certain heteroaryl groups may have a carbon count up to 9 carbon atoms. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, quinazolinyl, quinolinyl, thiadiazolyl (e.g., 1,3,4-thiadiazole), thiazolyl, thienyl, triazolyl (e.g., 1H-1,2,3-triazolyl), tetrazolyl, dihydroindolyl, tetrahydroquinolyl, tetrahydroisoquinolyl, etc. The term bicyclic, tricyclic, and tetracyclic heteroaryl groups include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Non-limiting examples of fused heteroaryl groups include 1,2,3,5,8,8a-hexahydroindolizine; 2,3-dihydrobenzofuran; 2,3-dihydroindole; and 2,3-dihydrobenzothiophene. Heteroaryl groups may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclylalkyl; heteroaryl; heteroarylalkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

As used herein, the term “heterocyclyl”, whether as part of another term or used independently, refers to a monocyclic, bicyclic, tricyclic, or tetracyclic ring system having fused or bridged 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl groups may be 3- to 12-membered, for example, 3- to 10-membered, 4- to 12-membered, 4- to 10-membered, 5- to 12-membered, 5- to 10-membered, 5- to 8-membered. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Non-limiting examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

As used herein, the term “heterocyclylalkyl”, whether as part of another term or used independently, refers to an alkyl group substituted with a heterocyclyl group. Heterocyclylalkyl groups may be unsubstituted or substituted. The heterocyclyl and alkyl portions of an optionally substituted heterocyclylalkyl are optionally substituted as described for heterocyclyl and alkyl, respectively.

As used herein, the term “halogen” or “halo” refers to fluoride, chloride, bromide and iodide, particularly fluoride and chloride, and more particularly fluoride.

As used herein, the term “heteroatom” refers to nitrogen (N), oxygen (O), and sulfur (S), and may include any oxidized form of nitrogen and sulfur, and any quaternized form of a basic nitrogen, unless otherwise stated.

As used herein, the term “substituted”, when refers to a chemical group, means the chemical group has one or more hydrogen atoms that is/are removed and replaced by substituents. The term “substituent”, as used herein, has the ordinary meaning known in the art and refers to a chemical moiety that is covalently attached to, or if appropriate, fused to, a parent group. It is to be understood that substitution at a given atom is limited by valency. Examples of substituents include, but not limited to, halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic group, and aliphatic group. It is understood that the substituent can be further substituted.

When a moiety is noted as being “optionally substituted” in Formula (I)-(IV) or any embodiment thereof, it means that Formula (I) or the embodiment thereof encompasses both compounds that are substituted with the noted substituent (or substituents) on the moiety and compounds that do not contain the noted substituent (or substituents) on the moiety (i.e., wherein the moiety is unsubstituted).

As used herein, the wavy line,

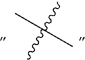

denotes a point of attachment of a moiety to another moiety.

The compounds of Formula (I)-(IV) or any other formula depicting and describing the compounds of the present invention may have one or more chiral (asymmetric) centers. The present invention encompasses all stereoisomeric forms of the compounds of Formula (I)-(IV) or any other formula depicting and describing the compounds of the present invention. Centers of asymmetry that are present in the compounds of Formula (I)-(IV) or any other formula depicting and describing the compounds of the present invention can all independently of one another have (R) or (S) configuration. When bonds to a chiral carbon are depicted as straight lines in the structural Formulas of the invention, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the Formula or by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present invention is intended to include all pharmaceutically acceptable isotopic variants of the compounds of Formula (I)-(IV) or any other formula depicting and describing the compounds of the present invention. Examples of isotopes suitable for inclusion in the compounds of the present invention include, but not limited to, isotopes of hydrogen, such as ²H (i.e., D) and ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopic variants of the compounds of Formula (T)-(IV) or any other formula depicting and describing the compounds of the present invention, for example those incorporating a radioactive isotope, may be useful in drug and/or substrate tissue distribution studies. Particularly, compounds having the depicted structures that differ only in the replacement with heavier isotopes, such as the replacement of hydrogen by deuterium (²H, or D), can afford certain therapeutic advantages, for example, resulting from greater metabolic stability, increased in vivo half-life, or reduced dosage requirements and, hence, may be utilized in some particular circumstances. Isotopic variants of compounds of Formula (I)-(IV) or any other formula depicting and describing the compounds of the present invention can generally be prepared by conventional techniques known to those skilled in the art or by processes analogous to those described in the accompanying examples and synthesis using an appropriate isotopically-labeled reagent in place of the non-labeled reagent previously employed.

As used herein, the term “deuterium substitution” refers to the substitution of one or more hydrogens on a compound or group by deuterium. When a compound or group is deuterated, one, two, three or even more hydrogen atoms on the compound or group can be substituted with deuterium until all the hydrogen atoms on the compound or group are substituted with deuterium, at which point the compound or group can be referred to as “fully deuterated”.

In some embodiments, the deuterium isotopic abundance is greater than the natural deuterium isotopic abundance (0.015%), preferably greater than 50%, more preferably greater than 60%, greater than 70%, greater than 80%, greater than 90%, greater than 95%, greater than 96%, greater than 97%, greater than 98%, greater than 99%, greater than 99.5%, or 100% at the position of the deuterium substituent.

In some cases, such as when the expressions “hydrogen” and “deuterium” appear as parallel alternatives or when the expression “hydrogen” is replaced by “deuterium, the term “hydrogen” stands for the hydrogen isotope “¹H” and the term “deuterium” stands for the hydrogen isotope “²H”; or it is understood that at this position in the compound, a state where hydrogen is present in its natural abundance of the various isotopes at that position is replaced by a state where deuterium is present in an abundance greater than the natural deuterium isotope (e.g. where the deuterium abundance is greater than 50%, greater than 60%, greater than 70%, greater than 80%, greater than 90%, greater than 95%, greater than 96%, greater than 97%, greater than 98%, greater than 99%, greater than 99.5%, or 100%).

The compounds as provided herein are described with reference to both generic formulas and specific compounds. In addition, the compounds of the present invention may exist in a number of different forms or derivatives, all within the scope of the invention. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable salt”, unless otherwise stated, includes salts that retain the biological effectiveness of the free acid/base form of the specified compound and that are not biologically or otherwise undesirable. Pharmaceutically acceptable salts may include salts formed with inorganic bases or acids and organic bases or acids. In cases where the compounds of the present invention contain one or more acidic or basic groups, the invention also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminum salts or as ammonium salts. More non-limiting examples of such salts include lithium salts, sodium salts, potassium salts, calcium salts, magnesium salts, barium salts, or salts with ammonia or organic amines such as ethylamine, ethanolamine, diethanolamine, triethanolamine, piperidine, N-methylglutamine, or amino acids. These salts are readily available, for instance, by reacting the compound having an acidic group with a suitable base, e.g., lithium hydroxide, sodium hydroxide, sodium propoxide, potassium hydroxide, potassium ethoxide, magnesium hydroxide, calcium hydroxide, or barium hydroxide. Other base salts of compounds of the present invention include but are not limited to copper (I), copper (II), iron (II), iron (III), manganese (II), and zinc salts. Compounds of the present invention which contain one or more basic groups, e.g., groups which can be protonated, can be present in salt form, and can be used according to the invention in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid, or aspartic acid, and other acids known to those skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates, and glutamates. The stoichiometry of the salts formed from the compounds of the invention may moreover be an integral or non-integral multiple of one.

Compounds of the present invention which contain basic nitrogen-containing groups can be quaternized using agents such as C₁₋₄alkyl halides, for example, methyl, ethyl, isopropyl, and tert-butyl chloride, bromide, and iodide; diC₁₋₄alkyl sulfates, for example, dimethyl, diethyl, and diamyl sulfate; C₁₀₋₁₈alkyl halides, for example, decyl, dodecyl, lauryl, myristyl, and stearyl chloride, bromide, and iodide; and arylC₁₋₄alkyl halides, for example, benzyl chloride and phenethyl bromide.

If the compounds of the present invention simultaneously contain acidic and basic groups in the molecule, the invention also includes, in addition to the salt forms mentioned, inner salts or betaines (zwitterions). The respective salts can be obtained by customary methods which are known to those skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts. For a review on more suitable salts, see Stahl and Wermuth, Handbook of Pharmaceutical Salts: Properties, Selection, and Use (Wiley-VCH, 2002).

The compound of Formula (I)-(IV) or any other formula depicting and describing the compounds of the present invention and pharmaceutically acceptable salts thereof may exist in unsolvated and solvated forms. As used herein, the term “solvate” refers to a molecular complex comprising the compound of Formula (I), or a pharmaceutically acceptable salt thereof, and one or more pharmaceutically acceptable solvent molecules. For example, the term “hydrate” is employed when the solvent is water.

Pharmaceutically acceptable solvates in accordance with the present invention may include those wherein the solvent of crystallization may be isotopically substituted, e.g., D₂O, d₆-acetone, d₆-DMSO.

One way of carrying out the present invention is to administer a compound of Formula (I)-(IV) or any other formula depicting and describing the compounds of the present invention in the form of a prodrug. Thus, certain derivatives of a compound of Formula (I)-(IV) or any other formula depicting and describing the compounds of the present invention which may have little or no pharmacological activity themselves can, when administered into or onto the body, be converted into a compound of Formula (I)-(IV) or any other formula depicting and describing the compounds of the present invention having the desired activity, for example by hydrolytic cleavage, particularly hydrolytic cleavage promoted by an esterase or peptidase enzyme. Such derivatives are referred to as “prodrugs”. Further information on the use of prodrugs may be found in, e.g., T. Higuchi and W. Stella, “Pro-drugs as Novel Delivery Systems”, Vol. 14, ACS Symposium Series, and E. B. Roche (Ed.), “Bioreversible Carriers in Drug Design”, Pergamon Press, 1987, American Pharmaceutical Association. Reference can also be made to Nature Reviews/Drug Discovery, 2008, 7, 355, and Current Opinion in Drug Discovery and Development, 2007, 10, 550.

Prodrugs in accordance with the present invention can, for example, be produced by replacing appropriate functionalities present in the compounds of Formula (I)-(IV) or any other formula depicting and describing the compounds of the present invention with certain moieties known to those skilled in the art as “pro-moieties” as described, for example, in H. Bundgaard, “Design of Prodrugs”, Elsevier, 1985, and Y. M. Choi-Sledeski and C. G. Wermuth, “Designing Prodrugs and Bioprecursors”, Practice of Medicinal Chemistry, 4^{th} Edition, Chapter 28, 657-696, Elsevier, 2015. Thus, a prodrug in accordance with the present invention may include, but not limited to, (a) an ester or amide derivative of a carboxylic acid in a compound of Formula (I)-(IV) or any other formula depicting and describing the compounds of the present invention, if any; (b) an amide, imine, carbamate or amine derivative of an amino group in a compound of Formula (I); (c) an oxime or imine derivative of a carbonyl group in a compound of Formula (I)-(IV) or any other formula depicting and describing the compounds of the present invention, if any; or (d) a methyl, primary alcohol or aldehyde group that can be metabolically oxidized to a carboxylic acid in a compound of Formula (I)-(IV) or any other formula depicting and describing the compounds of the present invention, if any.

### Linking Agent Compounds

As used herein, the term “linking agent compound” refers to a compound that can connect a ligand and a therapeutic agent together to form a ligand-drug conjugate by reacting with a group of the ligand compound and the therapeutic agent compound respectively by, for example, a coupling reaction.

In a first aspect, the present invention is directed to a compound of Formula (I):

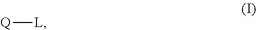

or a pharmaceutically acceptable salt, solvate, stereoisomer, or isotopic variant thereof, wherein
Q denotes to a junction moiety capable of being coupled to a ligand via a bond selected from the group consisting of carbonyl, thioether, amide, disulfide and hydrazone bond;
L denotes to a linker moiety capable of connecting Q to a therapeutic agent, and having a structure of:

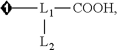

where L₁ is a polypeptide residue consisting of three to eight amino acid residues which comprises at least one amino acid residue with a side chain carboxyl group, for example, glutamic acid residue or aspartic acid residue, where “—COOH” denotes carboxyl group of an amino acid residue at C-terminal of the polypeptide residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O) (CH₂CH₂O)ₘ(CH₂)ₚC(O)NHR^{L2d}, R^{L2d} is H or C₁₋₆ alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4, for example, 1, 2, 3, or 4; and
[symbol] denotes to the N-terminal side of the polypeptide residue covalently attached to the junction moiety Q.

The present invention is also directed to use of the compound of Formula (I) or a pharmaceutically acceptable salt, solvate, stereoisomer, or isotopic variant thereof in preparation of a ligand-drug conjugate, for example, a ligand-drug conjugate as described herein.

The present invention is also directed to the compound of Formula (I) or a pharmaceutically acceptable salt, solvate, stereoisomer, or isotopic variant thereof for use in preparation of a ligand-drug conjugate, for example, a ligand-drug conjugate as described herein.

In some embodiments, the junction moiety Q has a structure of:

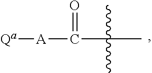

wherein Q^{a} is a function group capable of being coupled to a ligand;
A is selected from optionally substituted C₃₋₈ alkylidene, optionally substituted C₃₋₈ alkenylidene, optionally substituted C₃₋₆ cycloalkenylidene, optionally substituted C₃₋₈ cycloalkylidene, optionally substituted diglycol to octaglycol acyl, where alkylidene, alkenylidene, cycloalkenylidene, cycloalkylidene, diglycol to octaglycol acyl is optionally substituted with 1 to 4 substituents independently selected from the group consisting of halogen, —CN, R^{Qa1}, —OR^{Qa1}, —SR^{Qa1}, and —N(R^{Qa1})₂, where each R^{Qa1} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“ [symbol] ” denotes to the site covalently attached to the linker moiety L.

In some preferred embodiments, the function group Q^{a} is selected from the group consisting of

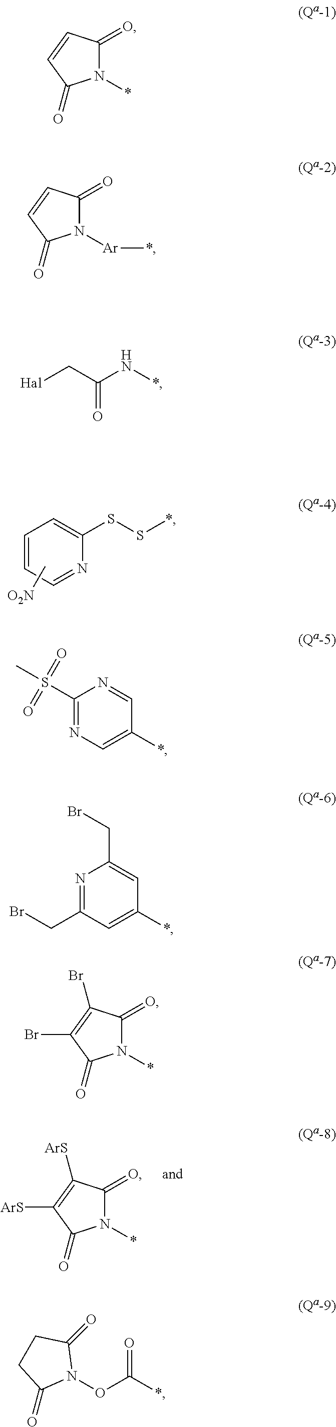

where Hal is a halogen selected from the group consisting of Cl, Br and I;
Ar is selected from the group consisting of optionally substituted C₅₋₆ cycloalkenyl, optionally substituted C₆ aryl and optionally substituted 5- to 6-membered heteroaryl, where cycloalkenyl, aryl and heteroaryl optionally substituted with 1 to 4 substituents independently selected from the group consisting of halogen, —CN, R^{Qa2}, —OR^{Qa2}, —SR^{Qa2}, and —N(R^{Qa2})₂, where each R^{Qa2} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“*” denotes to the site covalently attached to A.

It should be understood that in the cases when the group “Ar” is inside of the molecular structure of a compound, it can also refer to a divalent substituent.

In some preferred embodiments, Ar is selected from the group consisting of optionally substituted cyclopentadienyl, optionally substituted phenyl, and optionally substituted 5- to 6-membered heteroaryl.

In some preferred embodiments, the junction moiety Q is selected from the group consisting of

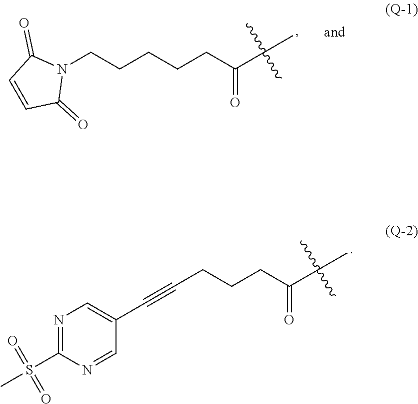 [SMILES: *C(=O)CCCCCN1C(=O)C=CC1=O ; *C(=O)CCCC#Cc1cnc(S(C)(=O)=O)nc1]

In some embodiments, the polypeptide residue L₁ has a sequence as shown below:

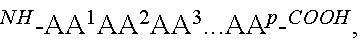

wherein each AA¹, AA², AA³, . . . AA^{p} is independently optionally substituted amino acid residue, and at least one of AA¹, AA², AA³, . . . AA^{p} is an amino acid residue with a side chain carboxyl group, preferably Glu or Asp;
p is an integer from 3 to 8, preferably from 3 to 5, for example, 3, 4, or 5;
“NH—” denotes to N-terminal side of the polypeptide residue;
“—COOH” denotes to C-terminal side of the polypeptide residue.

As used herein, the term “amino acid” refers to an organic compound that contains basic amino and acidic carboxyl groups. Amino acids include naturally occurring and synthetic α, β, γ or δ amino acids, and include amino acids where the amino and side chain reactive groups are appropriately protected. Naturally occurring amino acids include, but are not limited to, those occurring in natural proteins, namely glycine, alanine, valine, leucine, isoleucine, methionine, phenylalanine, tryptophan, proline, serine, threonine, cysteine, tyrosine, asparagine, glutamine, aspartic acid, glutamic acid, lysine, arginine and histidine. Amino acids also include non-natural amino acids such as amino acid variants and derivatives. Examples of synthetic amino acids include: citrulline, norvaline, isoleucine, ortholeucine, beta-alanine, ornithine, α-methylamino acids, D-amino acids, histidine-like amino acids, N-alkyl amino acid, amino acids with excess methylene groups in the side chain, and the like.

The amino acids may be in the form of a single enantiomer or in the form of a racemic or enantiomeric mixture. Preferred amino acids are the naturally occurring amino acids in the L-configuration.

Amino acids may be represented herein by their commonly known three-letter symbols or by the single-letter symbols recommended by the IUPAC.

The term “amino acid residue” refers to the corresponding residue obtained when a hydrogen atom is removed from the N-terminal amine group and/or the C-terminal carboxyl group of an amino acid.

When talking about substituted amino acids or substituted amino acid residues, it means that the reactive groups on the side chain of the amino acid or amino acid residue such as carboxyl, mercapto and amine groups can be further substituted with chemical groups, for example, C₁₋₆ alkyl, C₁₋₆ haloalkyl, C₃₋₆ cycloalkyl, C₃₋₁₀ heterocyclyl, C₆₋₁₀ aryl, and C₅₋₁₀ heteroaryl.

In some preferred embodiments, each AA¹, AA², AA³, . . . AA^{p} independently optionally substituted amino acid residue selected from the group consisting of Glu, Asp, Pro, Nva, Leu, Ile, Met, Tyr, Trp, Ser, Thr, Cys, Asn, Gln, Arg, Phe, Lys, Val, Ala, Cit, Gly, and N-alkyl amino acids, and at least one of AA1, AA2, AA3, . . . AAp is Glu or Asp. In some preferred embodiments, unsubstituted and substituted Lys can be comprised, for example, an amino acid residue represented by “-Lys(NR^{Lys1}R^{Lys2})”, where R^{Lys1} and R^{Lys2} are each independently H or C₁₋₆ alkyl, i.e., when substituted, the hydrogen of the amine group on side chain of lysine residue is substituted with at least one C₁₋₆ alkyl.

In some preferred embodiments, AA¹ is an amino acid residue with a side chain carboxyl group, preferably Glu or Asp; each AA², AA³, . . . AA^{p} is independently optionally substituted amino acid residue selected from the group consisting of Pro, Nva, Leu, Ile, Met, Tyr, Trp, Ser, Thr, Cys, Asn, Gln, Arg, Phe, Lys, Val, Ala, Cit, and Gly. In some preferred embodiments, unsubstituted and substituted Lys can be comprised, for example, an amino acid residue represented by “-Lys(NR^{Lys1}R^{Lys2})”, where R^{Lys1} and R^{Lys2} are each independently H or C₁₋₆ alkyl, i.e., when substituted, the hydrogen of the amine group on side chain of lysine residue is substituted with at least one C₁₋₆ alkyl.

In some preferred embodiments, the polypeptide residue L₁ is selected from the group consisting of ^{NH}-Glu-Phe-Lys(NR^{Lys1}R^{Lys2})—^{COOH}, ^{NH}-Glu-Val-Lys(NR^{Lys1}R^{Lys2})—^{COOH}, ^{NH}-Glu Ala-Ala-Ala-^{COOH}, ^{NH}-Glu-Ala-Ala-^{COOH}, ^{NH}-Glu-Val-Ala-^{COOH}, ^{NH}-Glu-Val-Cit-^{COOH}, ^{NH}-Glu-Gly-Gly-Phe-Gly-^{COOH}, ^{NH}-Asp-Phe-Lys-^{COOH}, ^{NH}-Asp-Ala-Ala-Ala-^{COOH}, ^{NH}-Asp-Val-Ala-^{COOH}, ^{NH}-Asp-Val-Cit-^{COOH}, ^{NH}-Asp-Gly-Gly-Phe-Gly-^{COOH}, and ^{NH}-Asp-Val-Lys(NR^{Lys1}R^{Lys2})—^{COOH} where R^{Lys1} and R^{Lys2} are each independently H or C₁₋₆ alkyl, preferably C₁₋₃ alkyl, more preferably C₃ alkyl, for example, propyl or isopropyl. In some preferred embodiment, R^{Lys1} and R^{Lys2} are the same, for example, C₁₋₃ alkyl, preferably C₃ alkyl, for example, propyl or isopropyl.

In some preferred embodiments, the hydrophilic group L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O)(CH₂CH₂O)ₘ(CH₂)ₚC(O)NHR^{L2d}, and at most two of R^{L2a}, R^{L2b}, and R^{L2c} are simultaneously H.

In some embodiments, in the hydrophilic group L₂, when R^{L2d} is C₁₋₆ alkyl substituted with 1 to 6 hydroxy groups, these hydroxyl groups can be substituted on the same or different carbon atoms where valence bonding allows.

In some preferred embodiments, in the hydrophilic group L₂, R^{L2d} is C₄₋₆ alkyl substituted with 3 to 5 hydroxy groups, where no more than one hydroxyl group is substituted on each carbon atom.

In some preferred embodiment, in the hydrophilic group L₂, R^{L2d} is selected from the group consisting of:

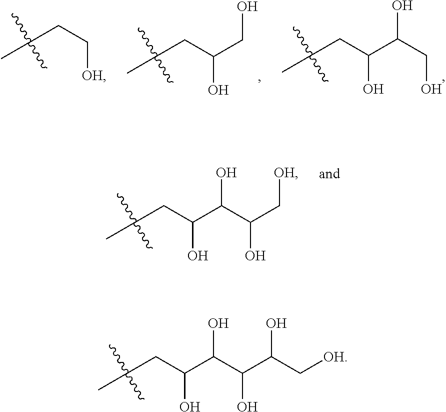

In some preferred embodiments, the hydrophilic group L₂ has a structure of —NHC(R^{L2a})(R^{L2b})(R^{L2c}), where R^{L2a}, R^{L2b}, and R^{L2c} are each independently selected from the group consisting of H, —CH₂O(CH₂)₂C(O)OH, and —CH₂O(CH₂)₂C(O)NHR^{L2d}, at most two of R^{L2a}, R^{L2b}, and R^{L2e} are simultaneously H, and R^{L2d} is C₄₋₆ alkyl substituted with 3 to 5 hydroxy groups.

In some preferred embodiment, the hydrophilic group L₂ is selected from the group consisting of:

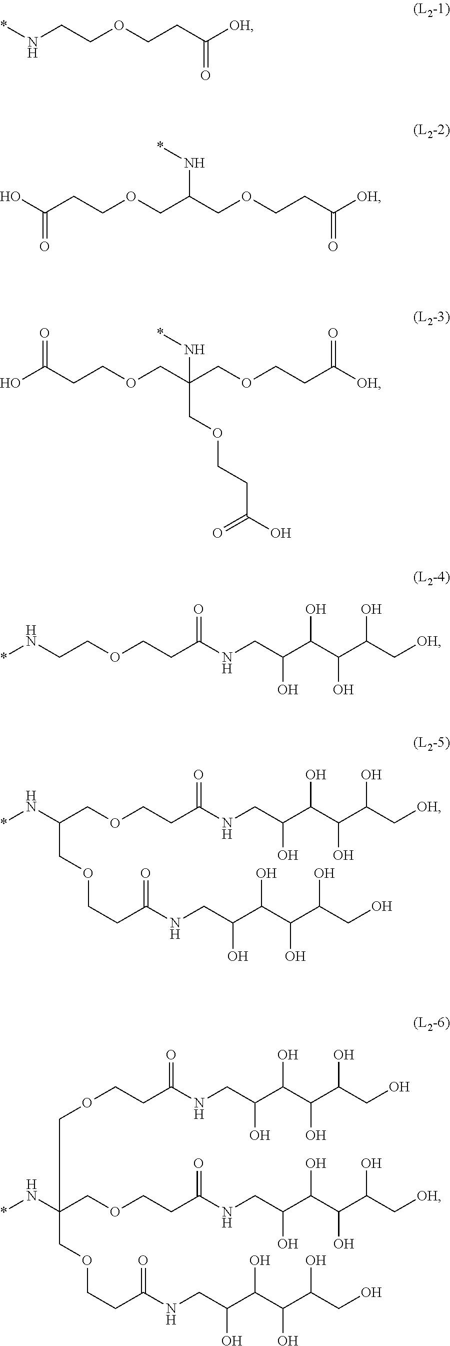 [SMILES: *NCCOCCC(=O)O ; *NC(COCCC(=O)O)COCCC(=O)O ; *NC(COCCC(=O)O)(COCCC(=O)O)COCCC(=O)O ; *NCCOCCC(=O)NCC(O)C(O)C(O)C(O)CO ; *NC(COCCC(=O)NCC(O)C(O)C(O)C(O)CO)COCCC(=O)NCC(O)C(O)C(O)C(O)CO ; *NC(COCCC(=O)NCC(O)C(O)C(O)C(O)CO)(COCCC(=O)NCC(O)C(O)C(O)C(O)CO)COCCC(=O)NCC(O)C(O)C(O)C(O)CO]

“*” denotes the site covalently attached to polypeptide residue L₁.

In some preferred embodiments, the hydrophilic group L₂ may be a group with a specific stereo-configuration, such as, but not limited to

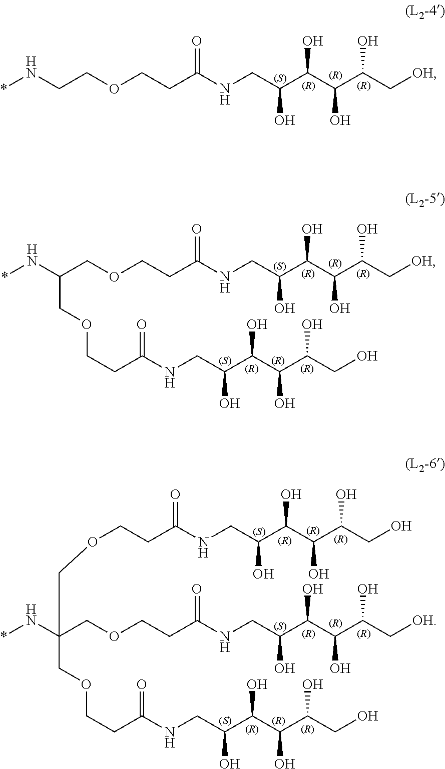

In some preferred embodiments, the compound of Formula (I) is a compound selected from the group consisting of:

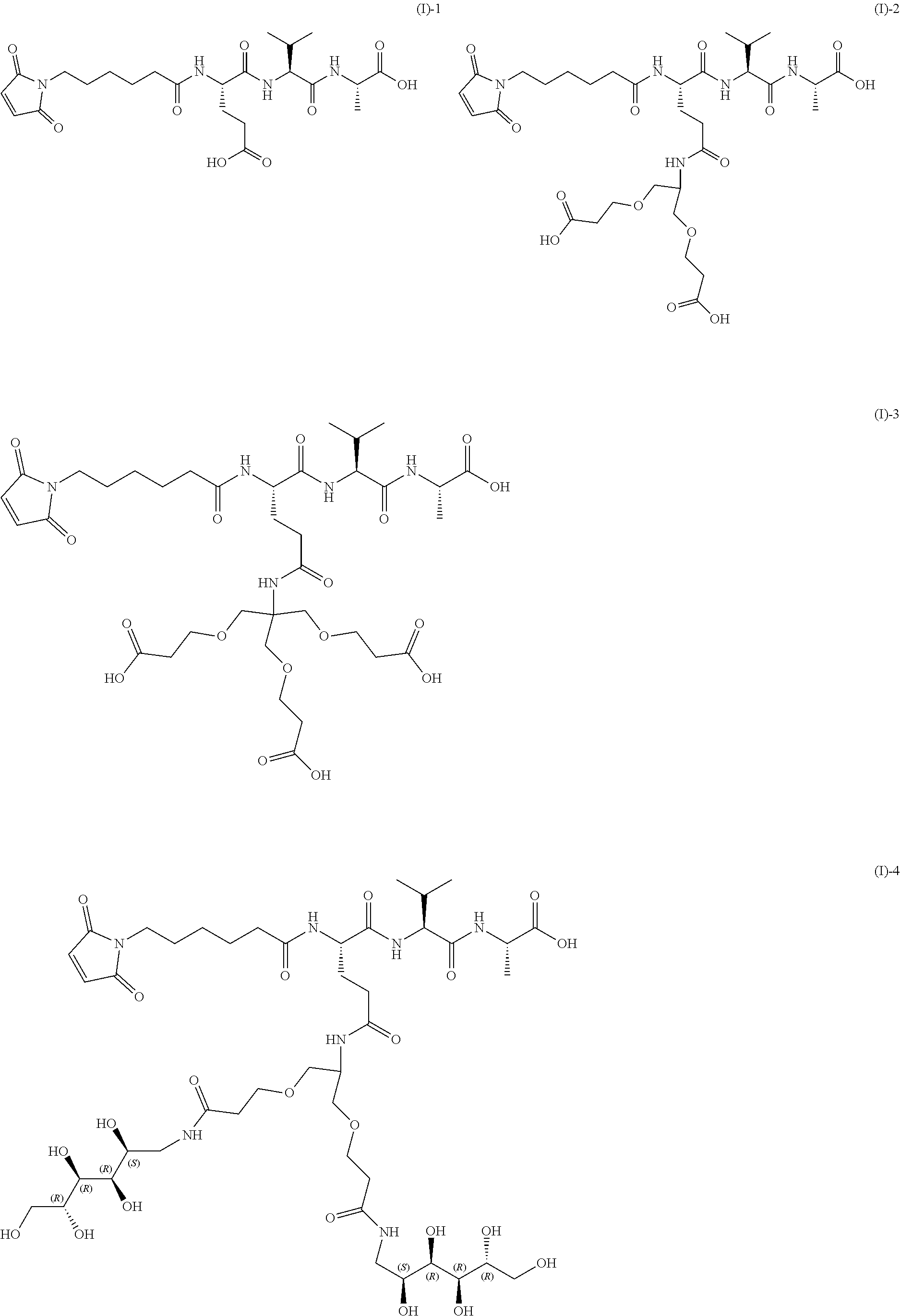

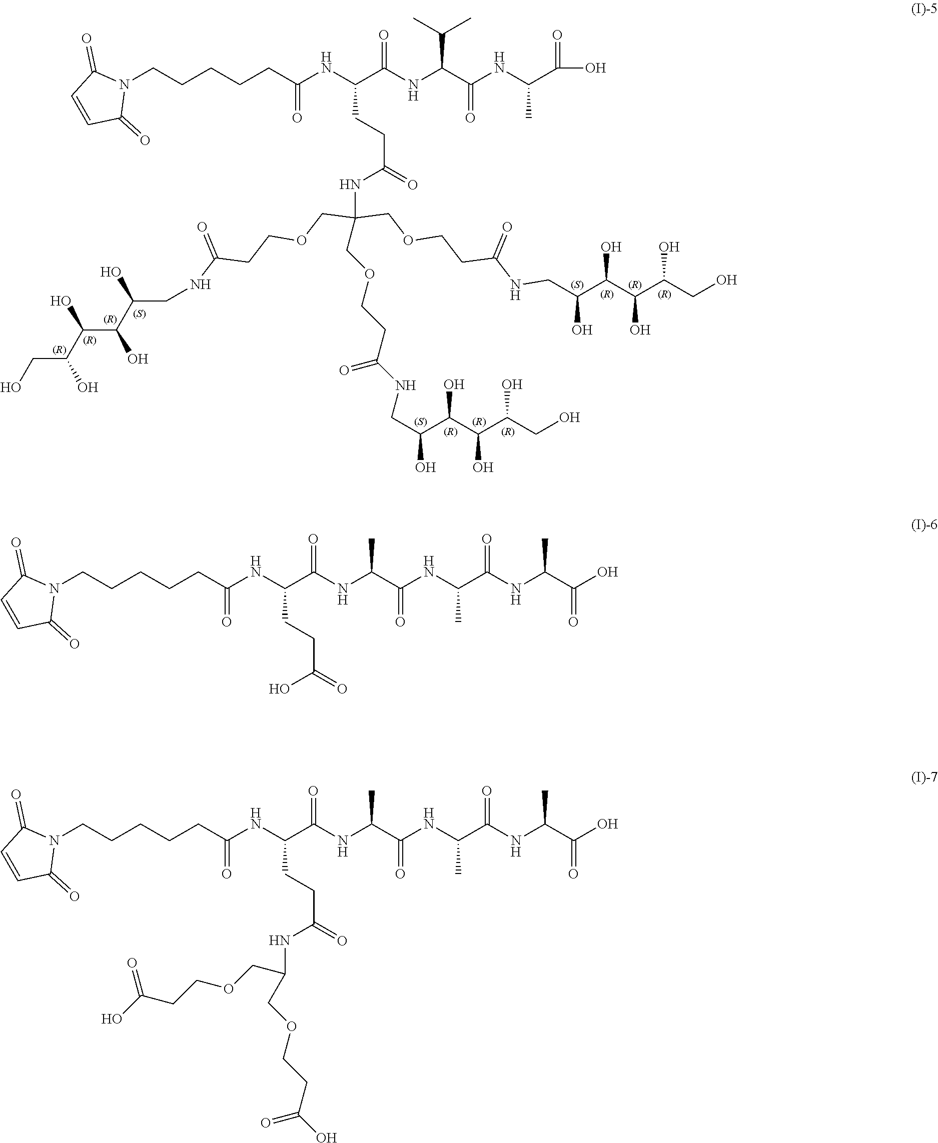

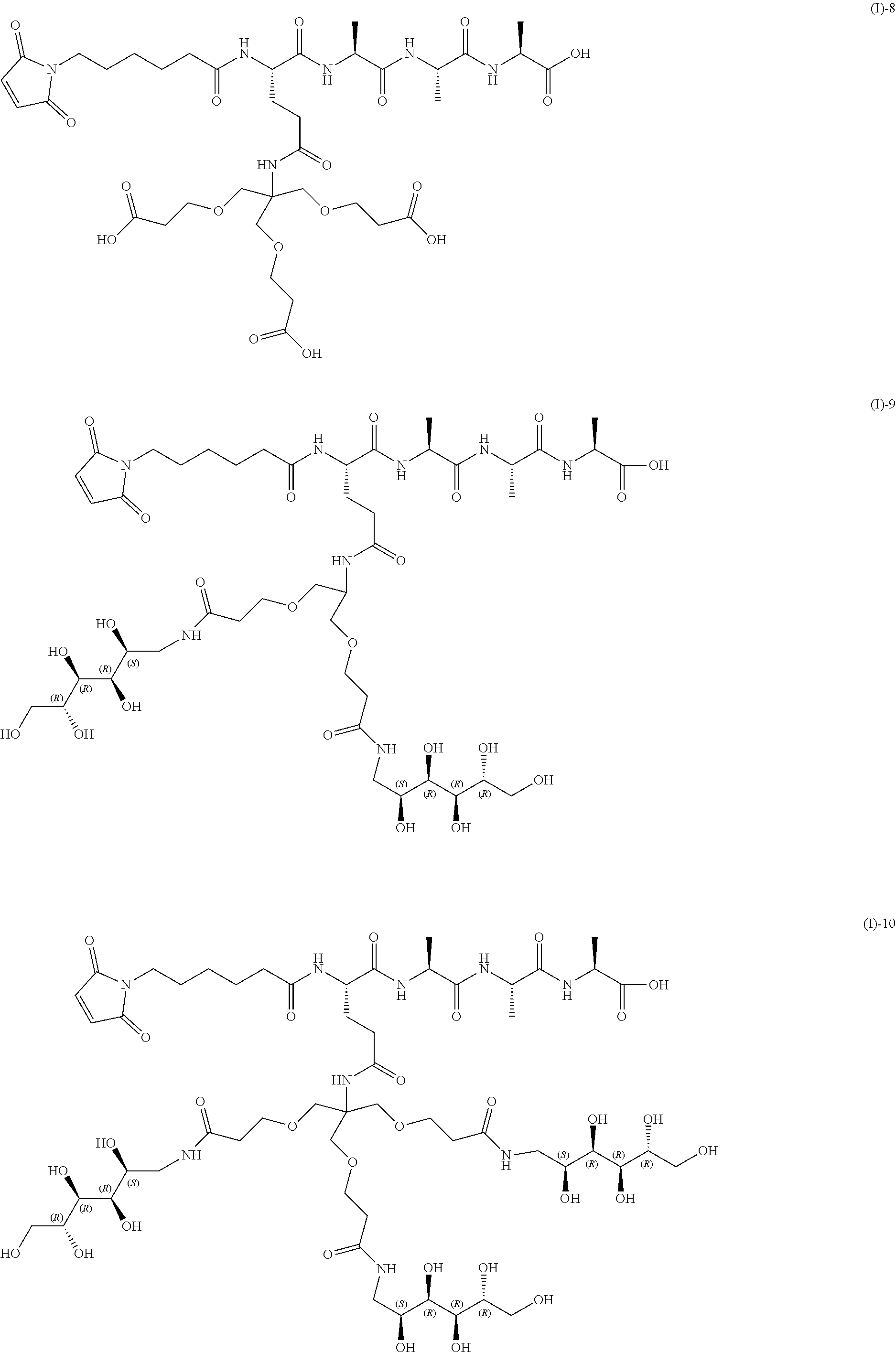

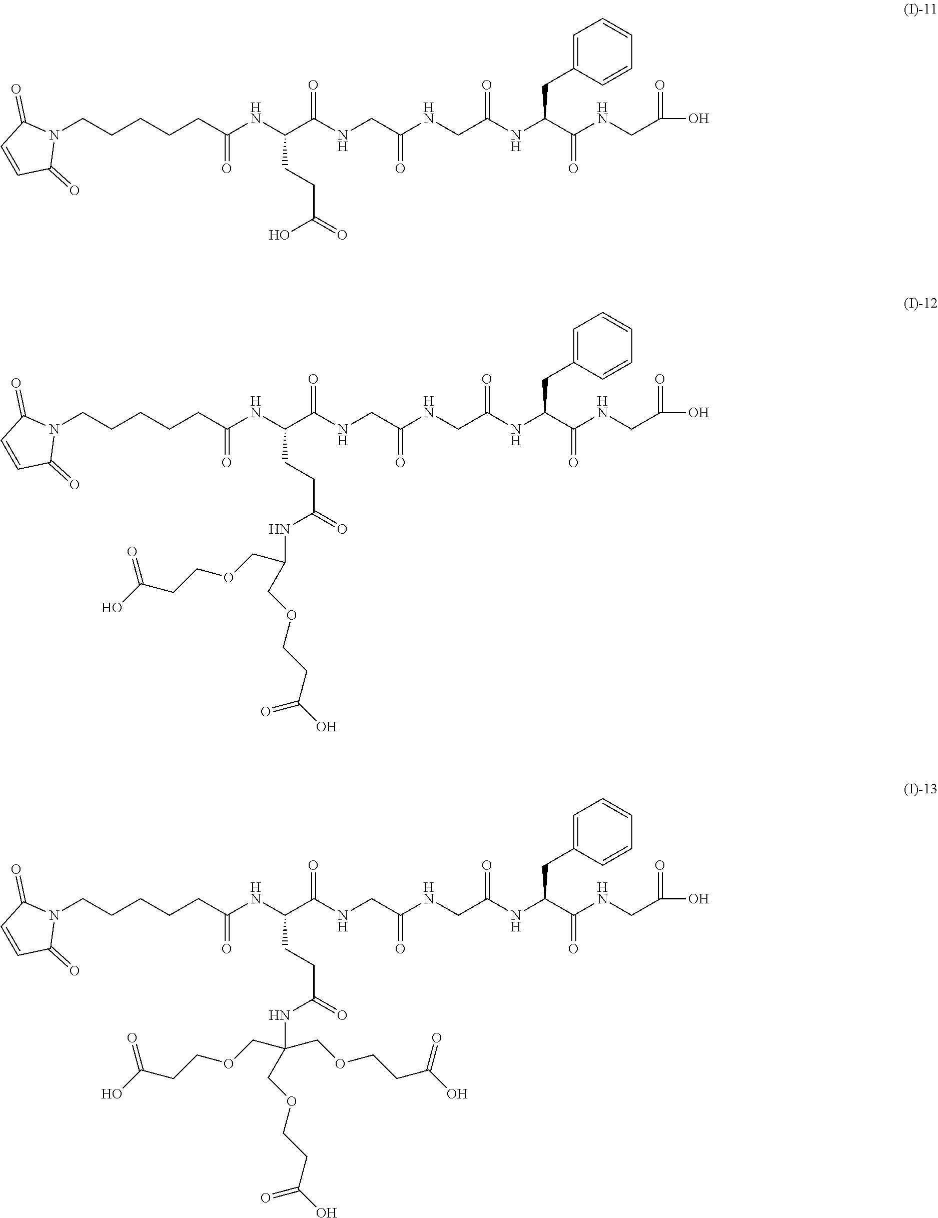 [SMILES: O=C(O)CC[C@H](NC(=O)CCCCCN1C(=O)C=CC1=O)C(=O)NCC(=O)NCC(=O)N[C@@H](Cc1ccccc1)C(=O)NCC(=O)O ; O=C(O)CCOCC(COCCC(=O)O)NC(=O)CC[C@H](NC(=O)CCCCCN1C(=O)C=CC1=O)C(=O)NCC(=O)NCC(=O)N[C@@H](Cc1ccccc1)C(=O)NCC(=O)O ; O=C(O)CCOCC(COCCC(=O)O)(COCCC(=O)O)NC(=O)CC[C@H](NC(=O)CCCCCN1C(=O)C=CC1=O)C(=O)NCC(=O)NCC(=O)N[C@@H](Cc1ccccc1)C(=O)NCC(=O)O]

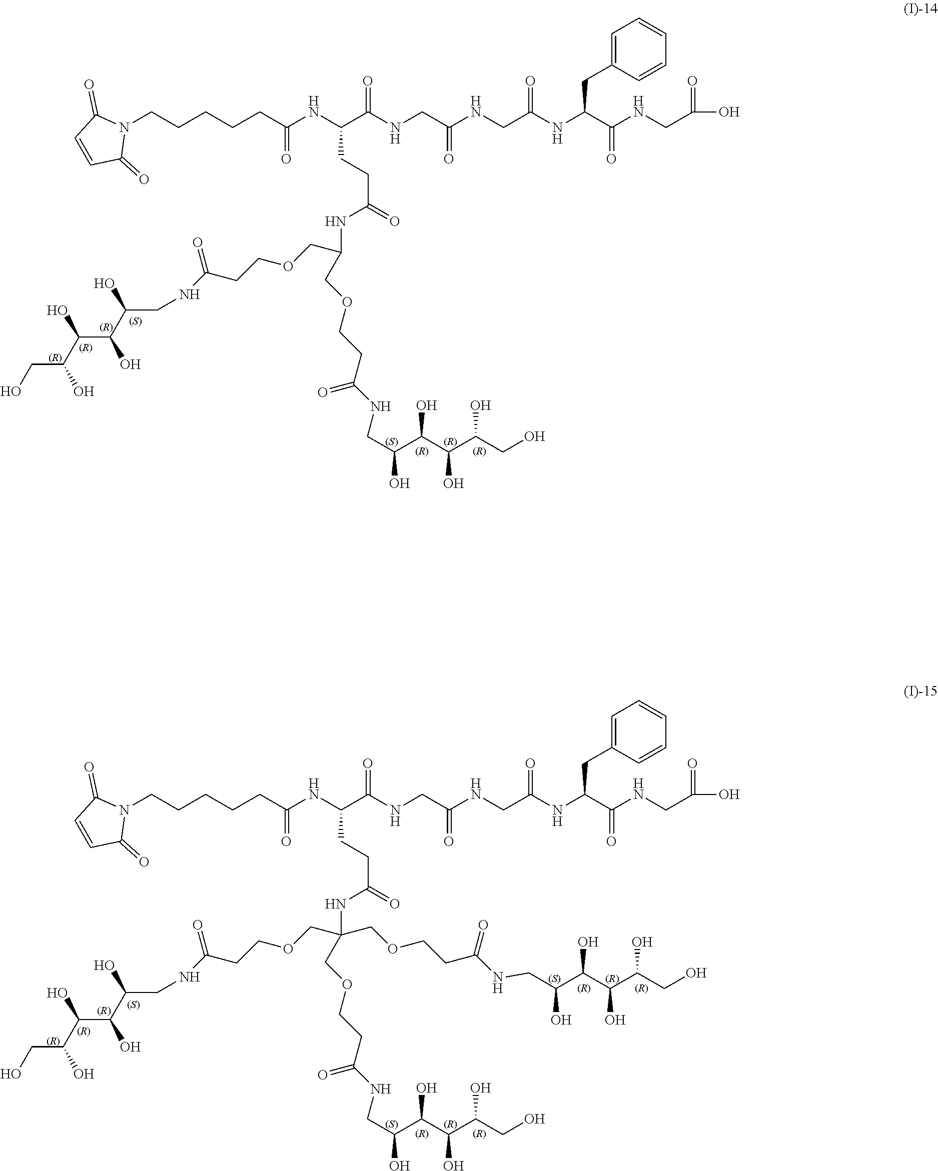

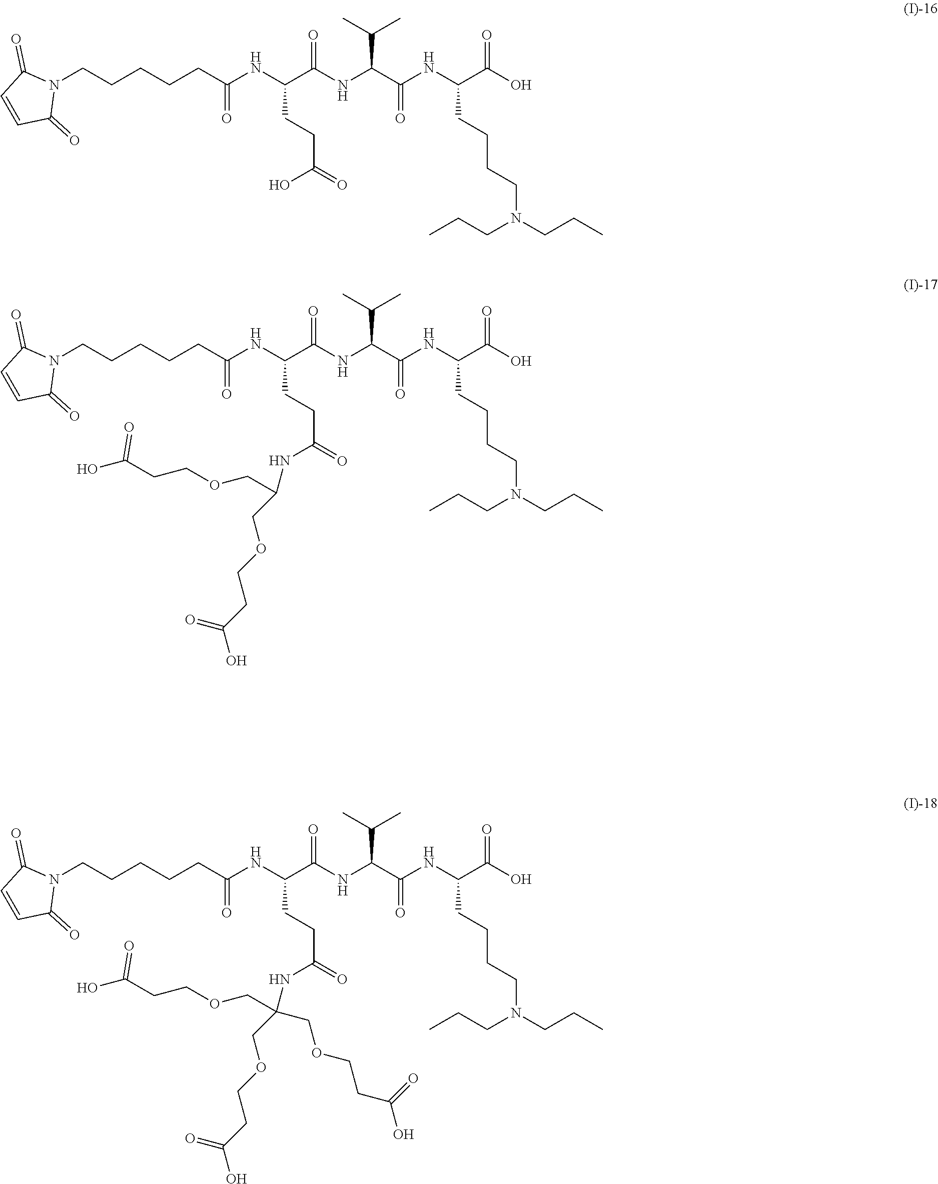 [SMILES: CCCN(CCC)CCCC[C@H](NC(=O)[C@@H](NC(=O)[C@H](CCC(=O)O)NC(=O)CCCCCN1C(=O)C=CC1=O)C(C)C)C(=O)O ; CCCN(CCC)CCCC[C@H](NC(=O)[C@@H](NC(=O)[C@H](CCC(=O)NC(COCCC(=O)O)COCCC(=O)O)NC(=O)CCCCCN1C(=O)C=CC1=O)C(C)C)C(=O)O ; CCCN(CCC)CCCC[C@H](NC(=O)[C@@H](NC(=O)[C@H](CCC(=O)NC(COCCC(=O)O)(COCCC(=O)O)COCCC(=O)O)NC(=O)CCCCCN1C(=O)C=CC1=O)C(C)C)C(=O)O]

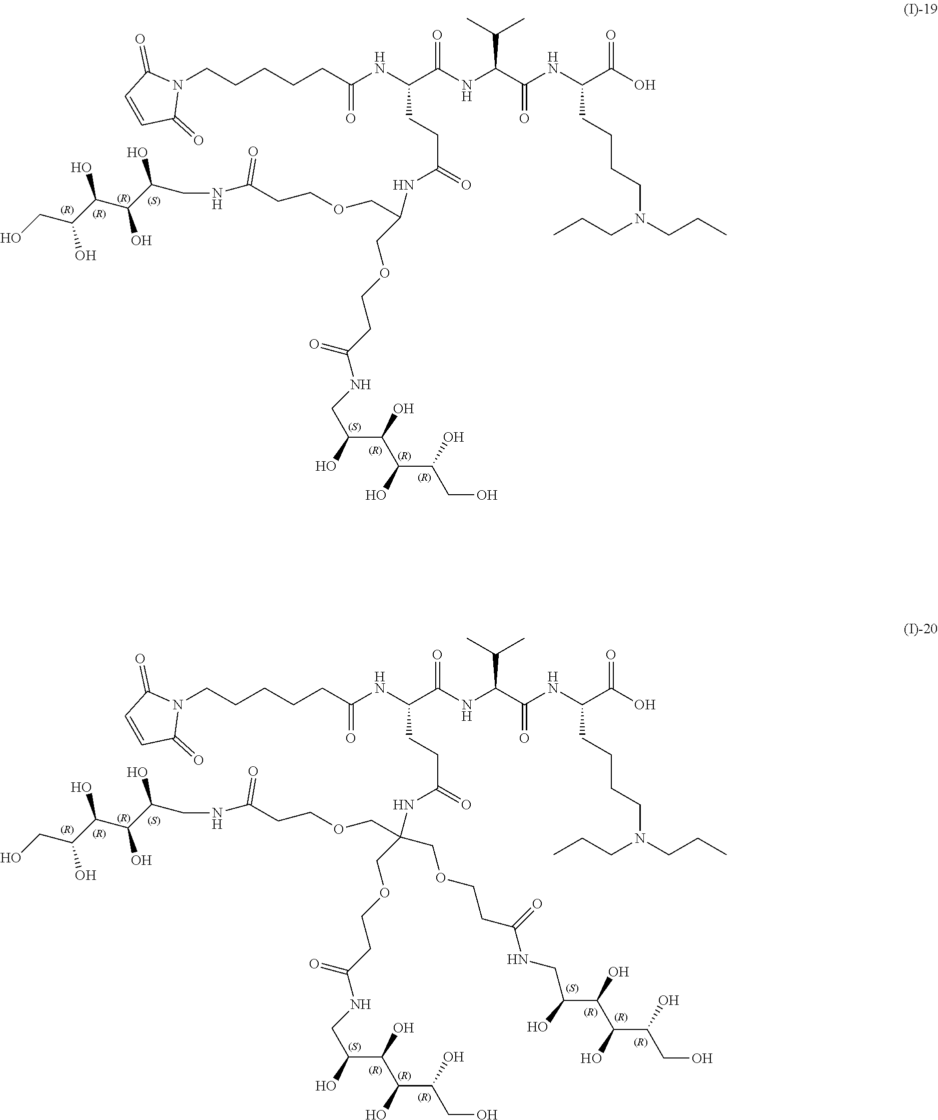

### Linker-Therapeutic Agent Compounds

As used herein, the term “linker-therapeutic agent compound” refers to a compound formed by attaching a linking agent compound of the present invention, i.e., the compound of Formula (I) to a therapeutic agent.

In a second aspect, the present invention is directed to a compound of Formula (II)

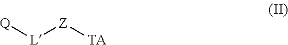

or a pharmaceutically acceptable salt, solvate, stereoisomer, or isotopic variant thereof, wherein
TA denotes to a therapeutic agent;
Z is absent or denotes to an auxiliary moiety connecting L′ to the therapeutic agent TA via a bond selected from the group consisting of disulfide, thioether, thioester, hydrazone, ester, ether, carbamate and amide bond;
Q denotes to a junction moiety capable of being coupled to a ligand via a bond selected from the group consisting of carbonyl, thioether, amide, disulfide and hydrazone bond;
L′ denotes to a linker moiety connecting Q to the therapeutic agent TA, and having a structure of:

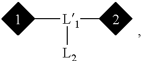

where L′₁ is a polypeptide residue consisting of three to eight amino acid residues which comprises at least one amino acid residue with a side chain carboxyl group, for example, glutamic acid residue or aspartic acid residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)ₘ(CH₂)ₚC(O)OH, and —(CH₂O)ₘ(CH₂)ₚC(O)NHR^{L2d}, R^{L2d} is C₁₋₆ alkyl optionally substituted with 1 to 6 hydroxy groups, each m is independently an integer from 1 to 6, and each p is independent an integer from 1 to 4;
[symbol] denotes to the N-terminal side of the polypeptide residue covalently attached to the junction moiety Q;
[symbol] denotes to the C-terminal side of the polypeptide residue covalently attached to the therapeutic agent TA.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some preferred embodiments, the cytotoxic agent is a camptothecin compound, an analogue or a derivative thereof. In some preferred embodiments, the camptothecin compound is a compound of the following structure:

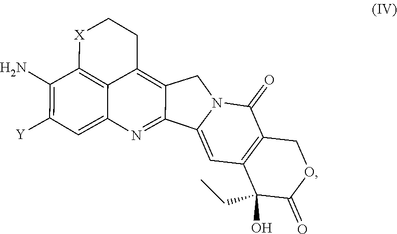

wherein X is selected from the group consisting of —CH₂—, O and S; Y is selected from the group consisting of H, D, and F.

In some embodiments, the auxiliary moiety Z is selected from the group consisting of mercapto, disulfide, amino, carboxyl, aldehyde, maleimide, haloacetyl, hydrazide and hydroxyl groups.

In some embodiments, the junction moiety Q is defined as descried in the first aspect.

In some embodiments, the polypeptide residue L′₁ has a sequence as shown below:

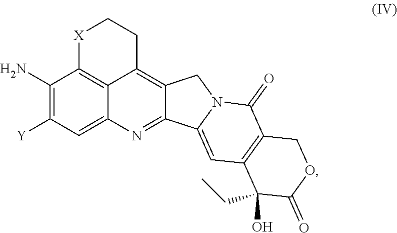

wherein each AA¹, AA², AA³, . . . AA^{p} is independently optionally substituted amino acid residue, and at least one of AA¹, AA², AA³, . . . AA^{p} is an amino acid residue with a side chain carboxyl group, preferably Glu or Asp;
p is an integer from 3 to 8, preferably from 3 to 5, for example, 3, 4, or 5;
“NH—” denotes to N-terminal side of the polypeptide residue;
“—C(═O)” denotes to C-terminal side of the polypeptide residue.

In some embodiments, the polypeptide residue L′₁ is defined as descried in the first aspect, except that the C-terminal of the polypeptide residue L is —COOH (carboxyl group), while the C-terminal of the polypeptide residue L′₁ is —C(O)— (carbonyl group).

In some embodiments, the linker moiety L′ is coupled to the therapeutic agent via a bond selected from the group consisting of carbonyl, amide, and ester bond, preferably an amide bond.

In some embodiments, the linker moiety L′ is first connected to the auxiliary moiety Z, then coupled to the therapeutic agent via a bond selected from the group consisting of disulfide, thioether, thioester, hydrazone, ester, ether, carbamate and amide bond.

In some embodiments, the hydrophilic group L₂ is defined as descried in the first aspect.

In some preferred embodiments, the present invention is directed to a compound of Formula (II′)

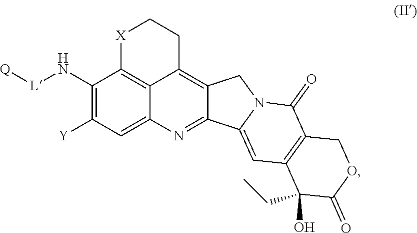

or a pharmaceutically acceptable salt, solvate, stereoisomer, or isotopic variant thereof, wherein
X is selected from the group consisting of —CH₂—, O, and S; Y is selected from the group consisting of H, D, and F;
Q denotes to a junction moiety capable of being coupled to a ligand via a bond selected from the group consisting of carbonyl, thioether, amide, disulfide and hydrazone bond;
L′ denotes to a linker moiety connecting Q to a therapeutic agent, and having a structure of:

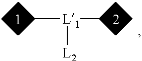

where L₁ is a polypeptide residue consisting of three to eight amino acid residues which comprises at least one amino acid residue with a side chain carboxyl group, for example, glutamic acid residue or aspartic acid residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O)(CH₂CH₂O)ₘ(CH₂)ₚC(O)NHR^{L2d}, R^{L2d} H or is C₁₋₆ alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4 for example, 1, 2, 3, or 4;
[symbol] denotes to the N-terminal side of the polypeptide residue covalently attached to the junction moiety Q;
[symbol] denotes to the C-terminal side of the polypeptide residue covalently attached to the therapeutic agent TA.

In some embodiments, the junction moiety Q is defined as descried for Formula (II).

In some embodiments, the polypeptide residue L′₁ is defined as descried for Formula (II).

In some embodiments, the hydrophilic group L₂ is defined as descried for Formula (II).

In some preferred embodiments, the compound of Formula (II′) of the present invention is selected from the group consisting of:

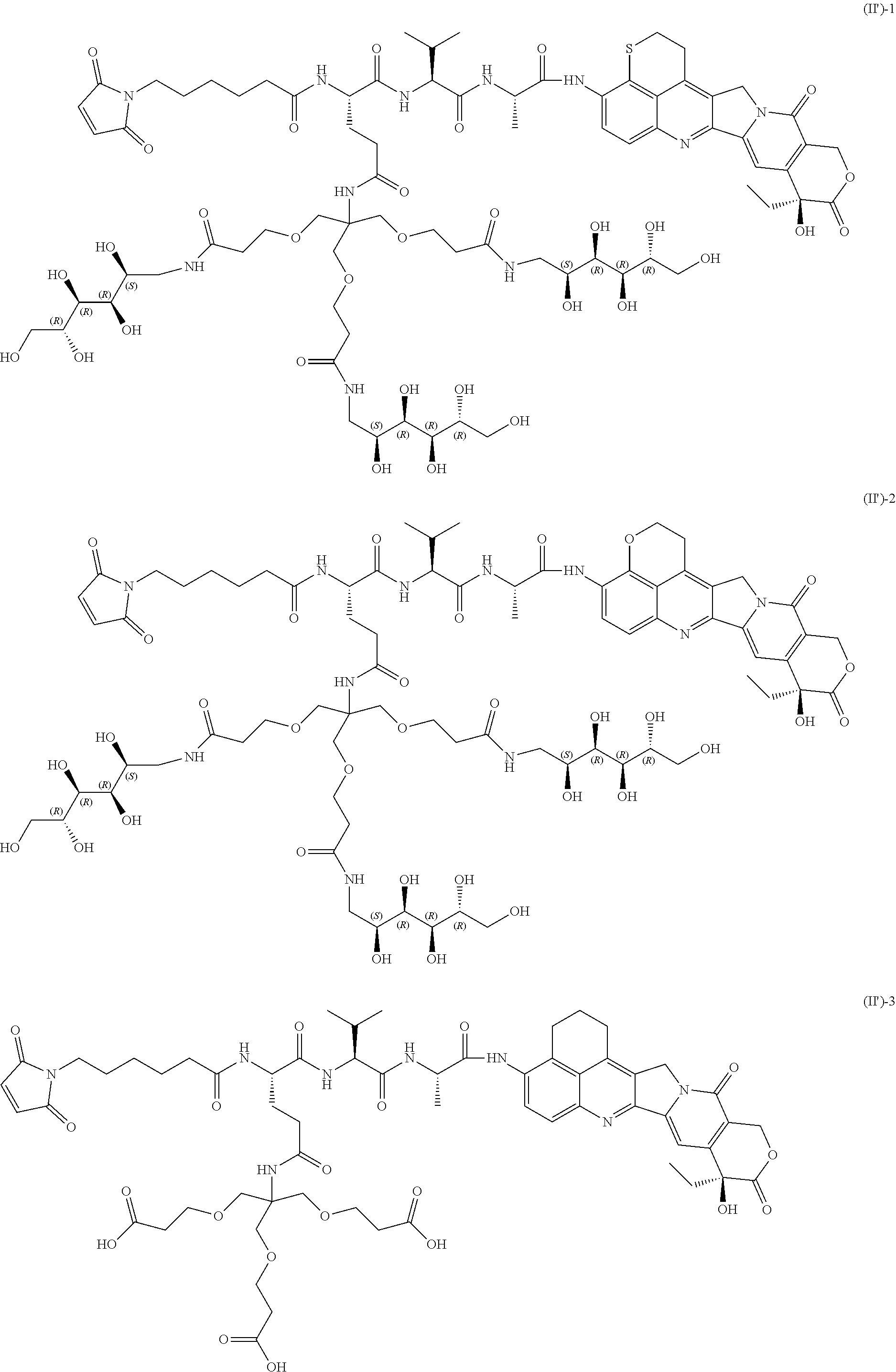

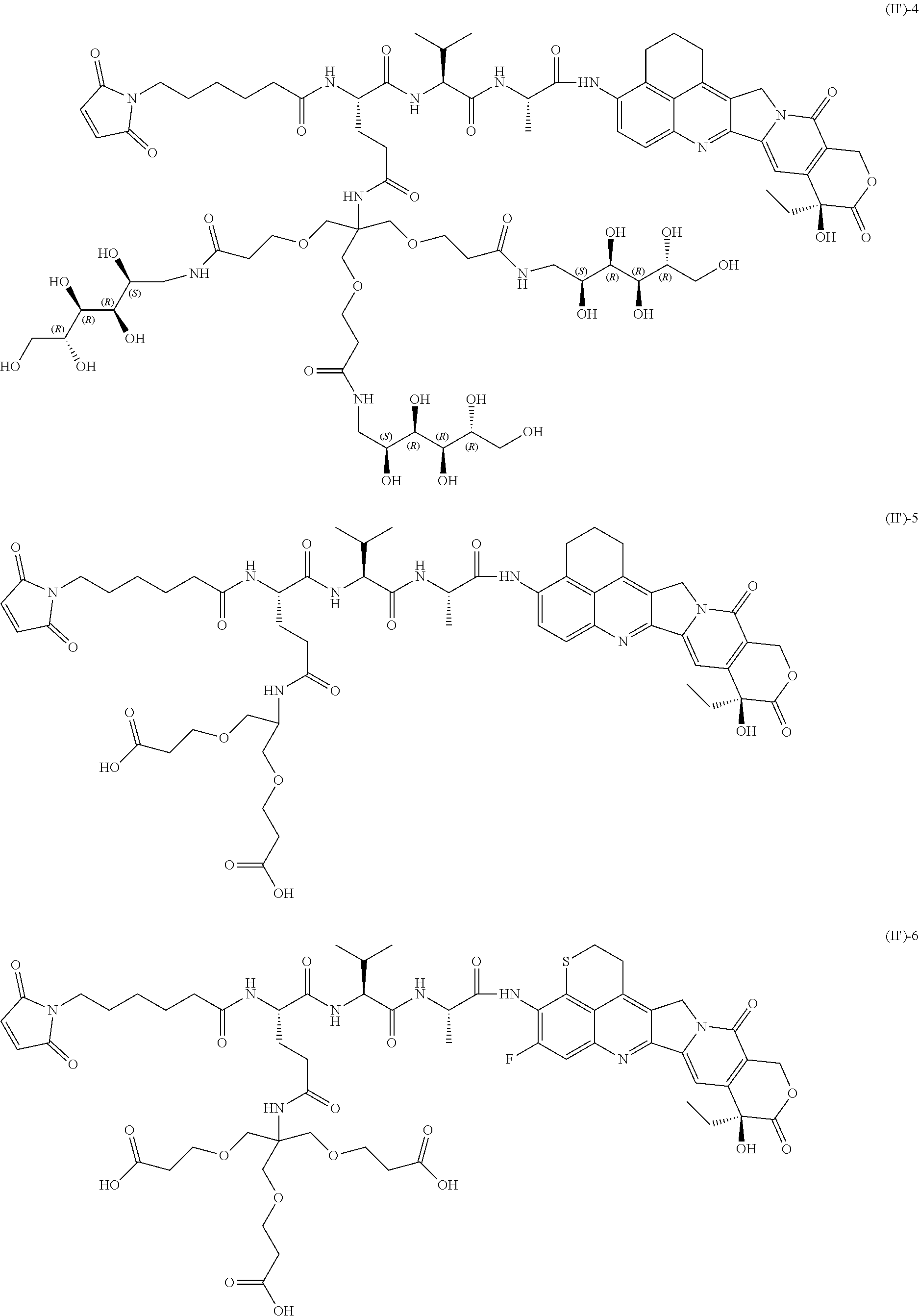

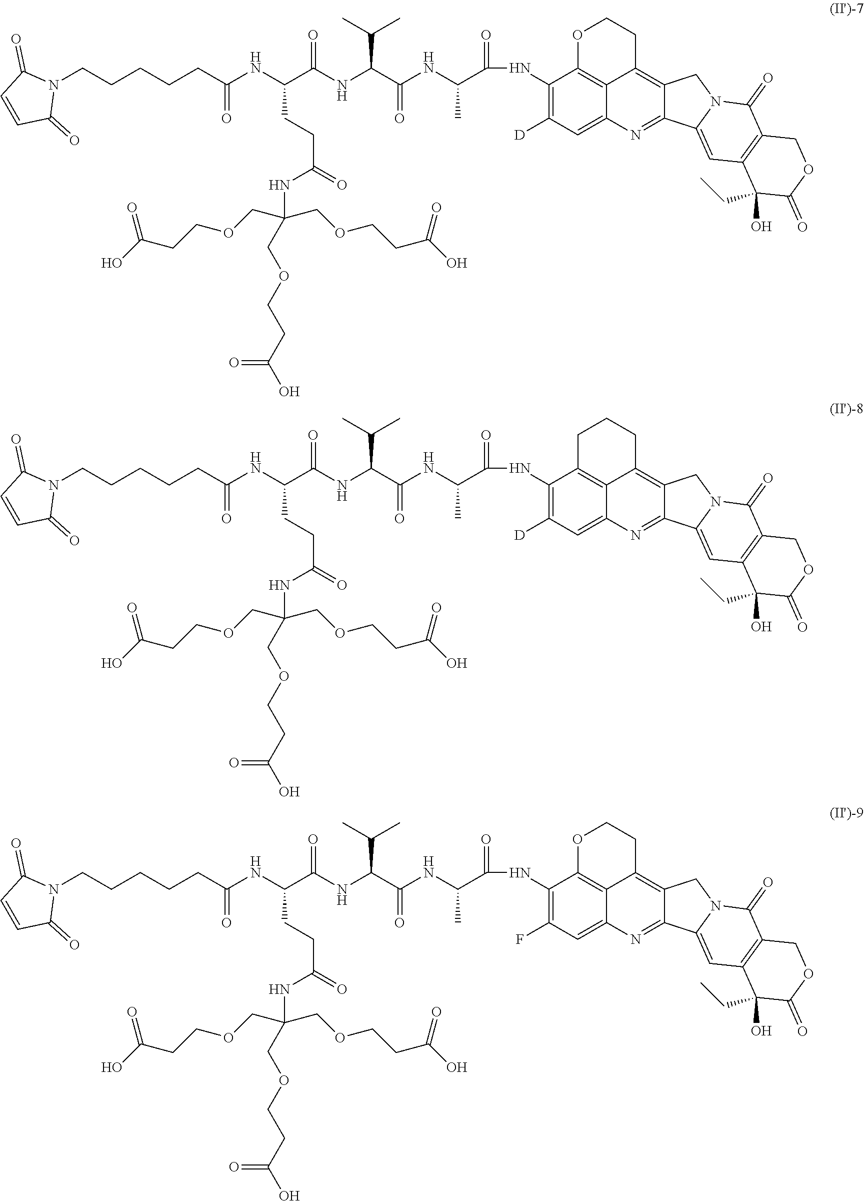

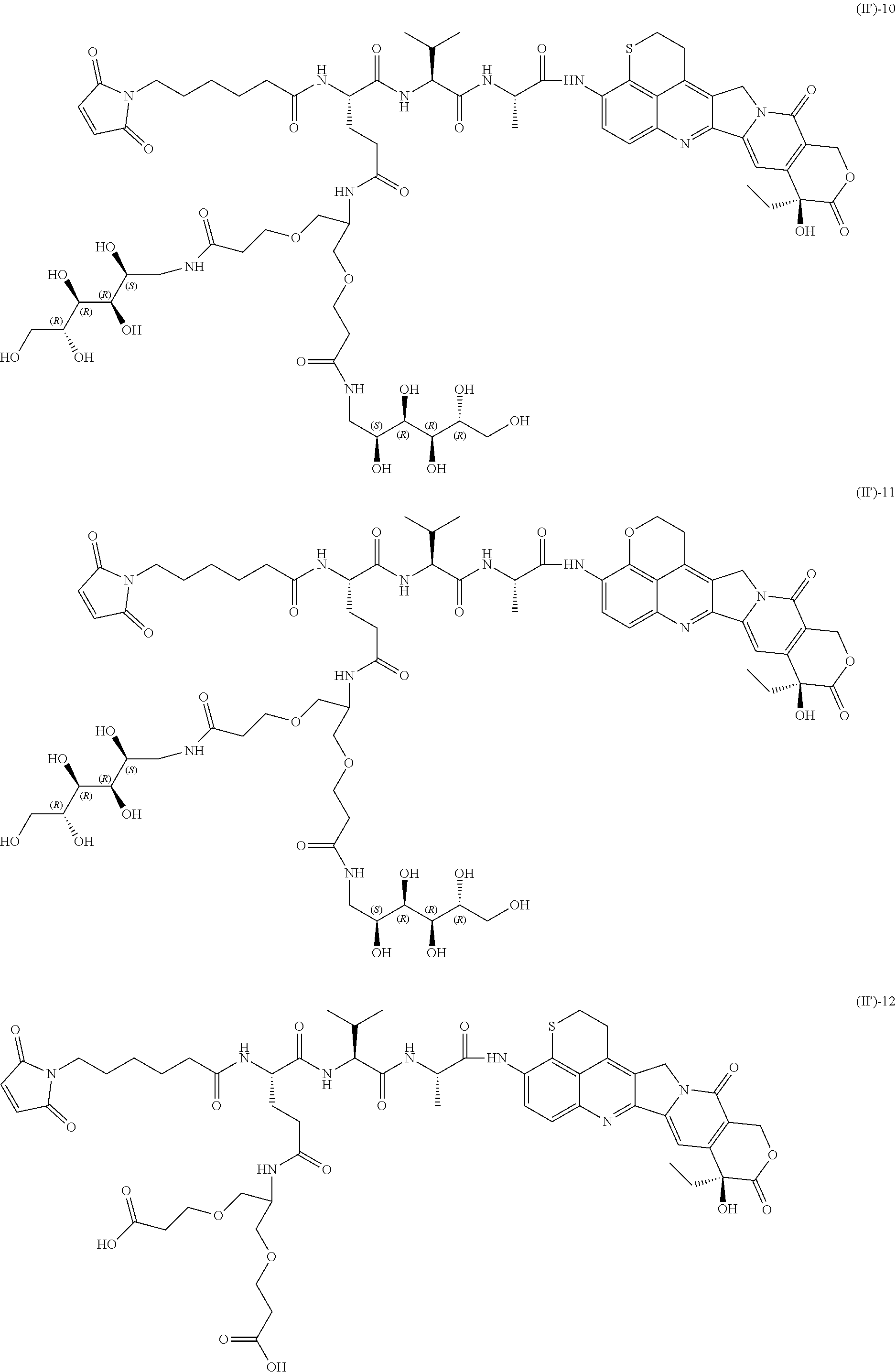

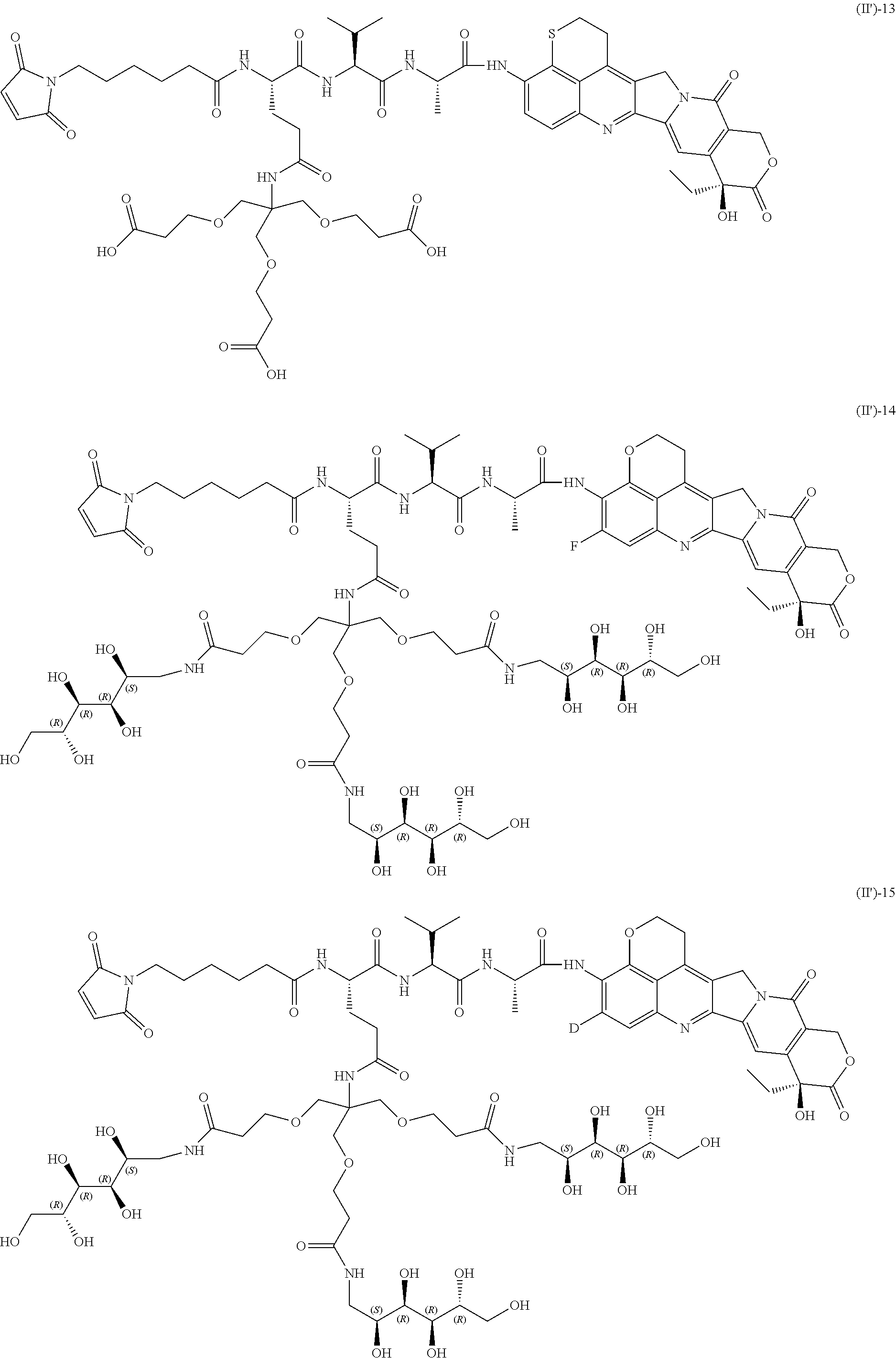

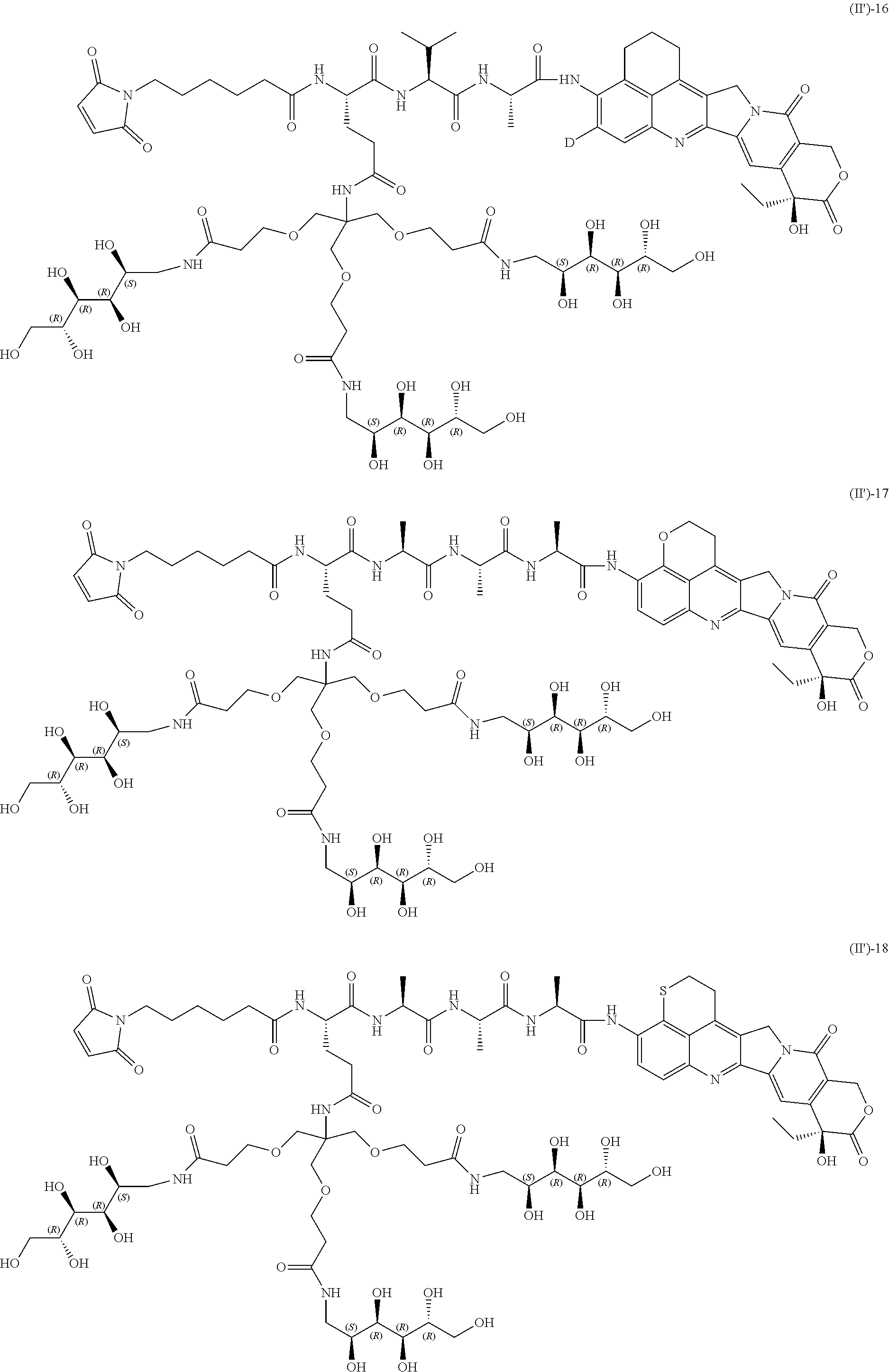

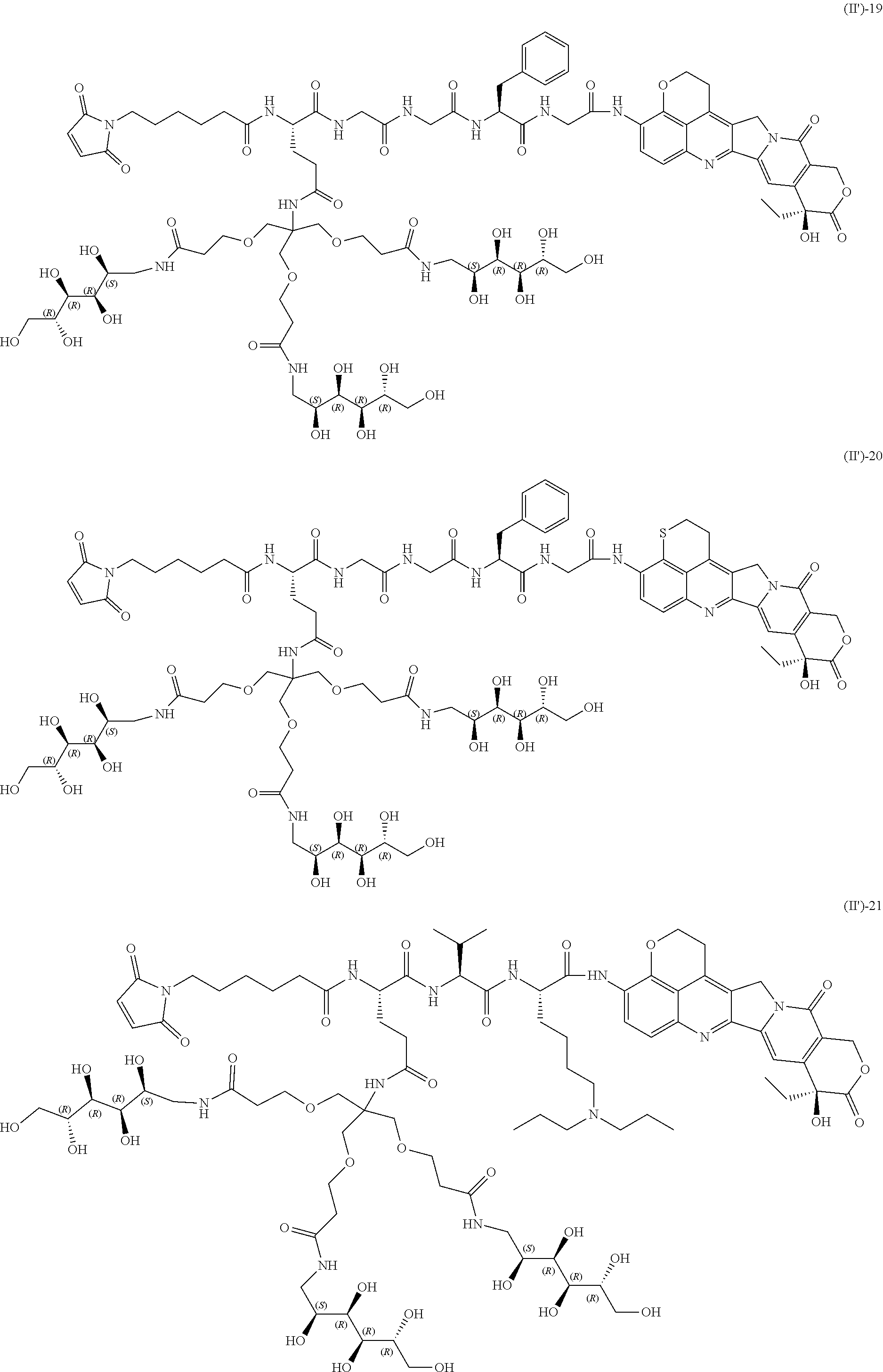

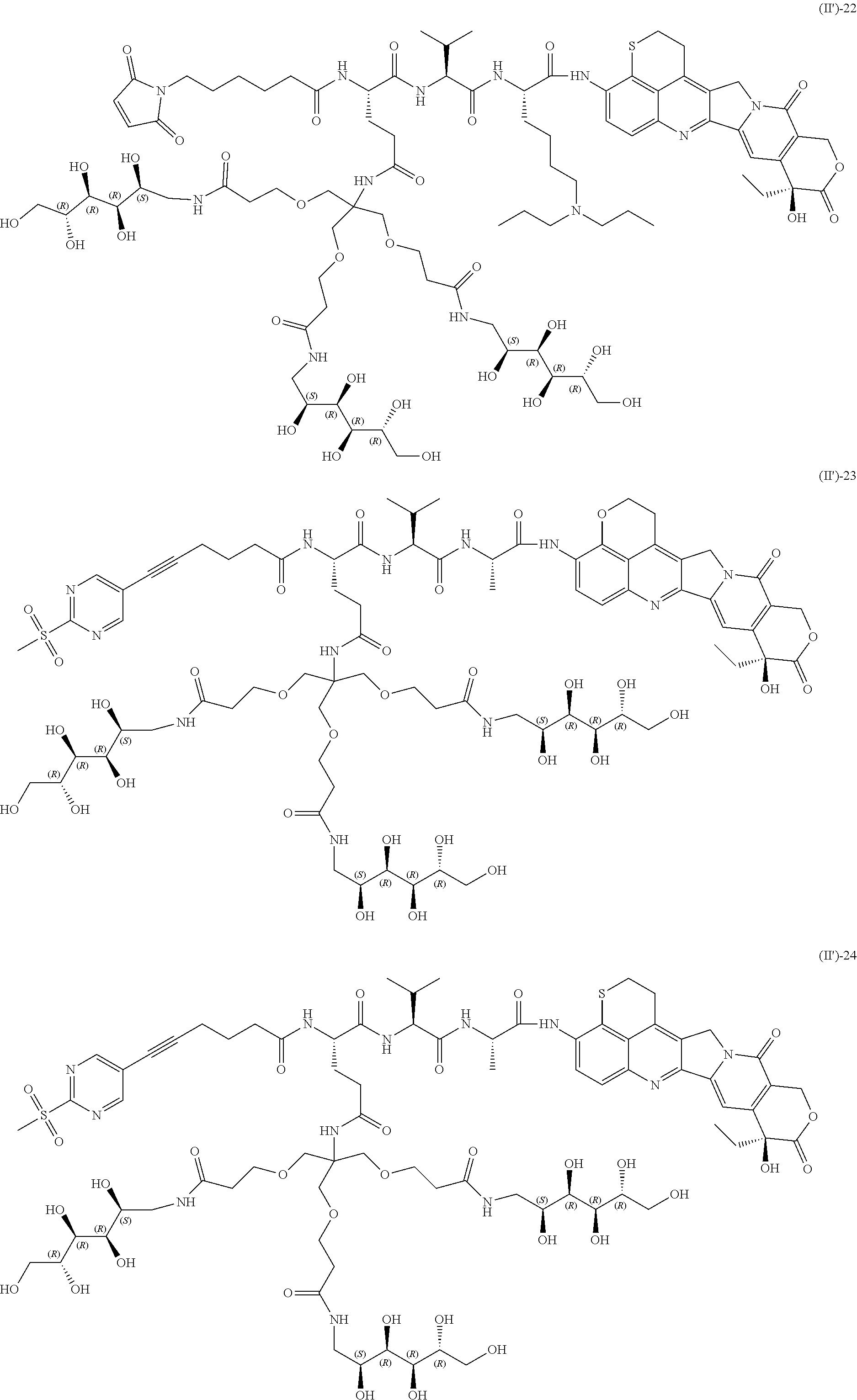

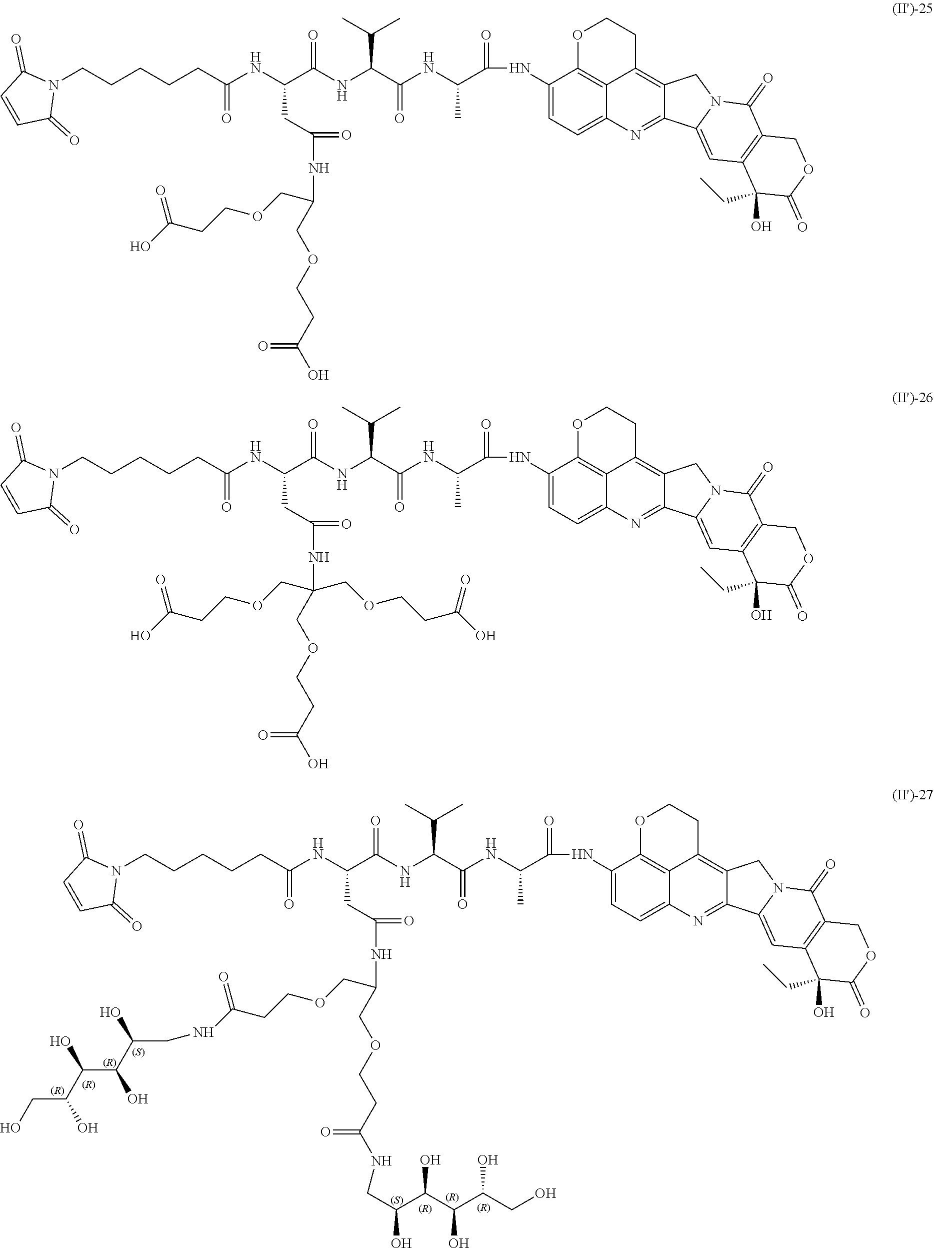

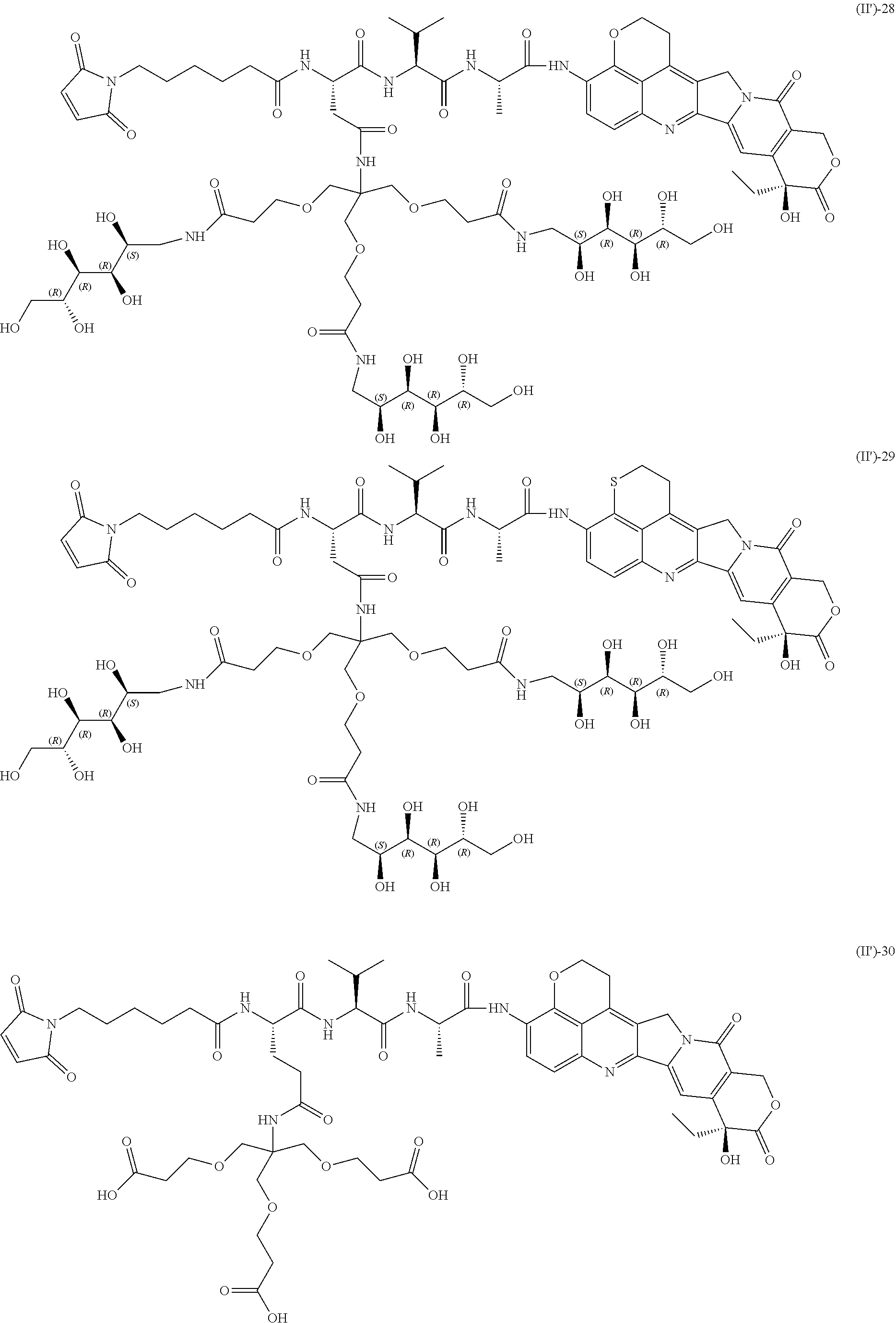

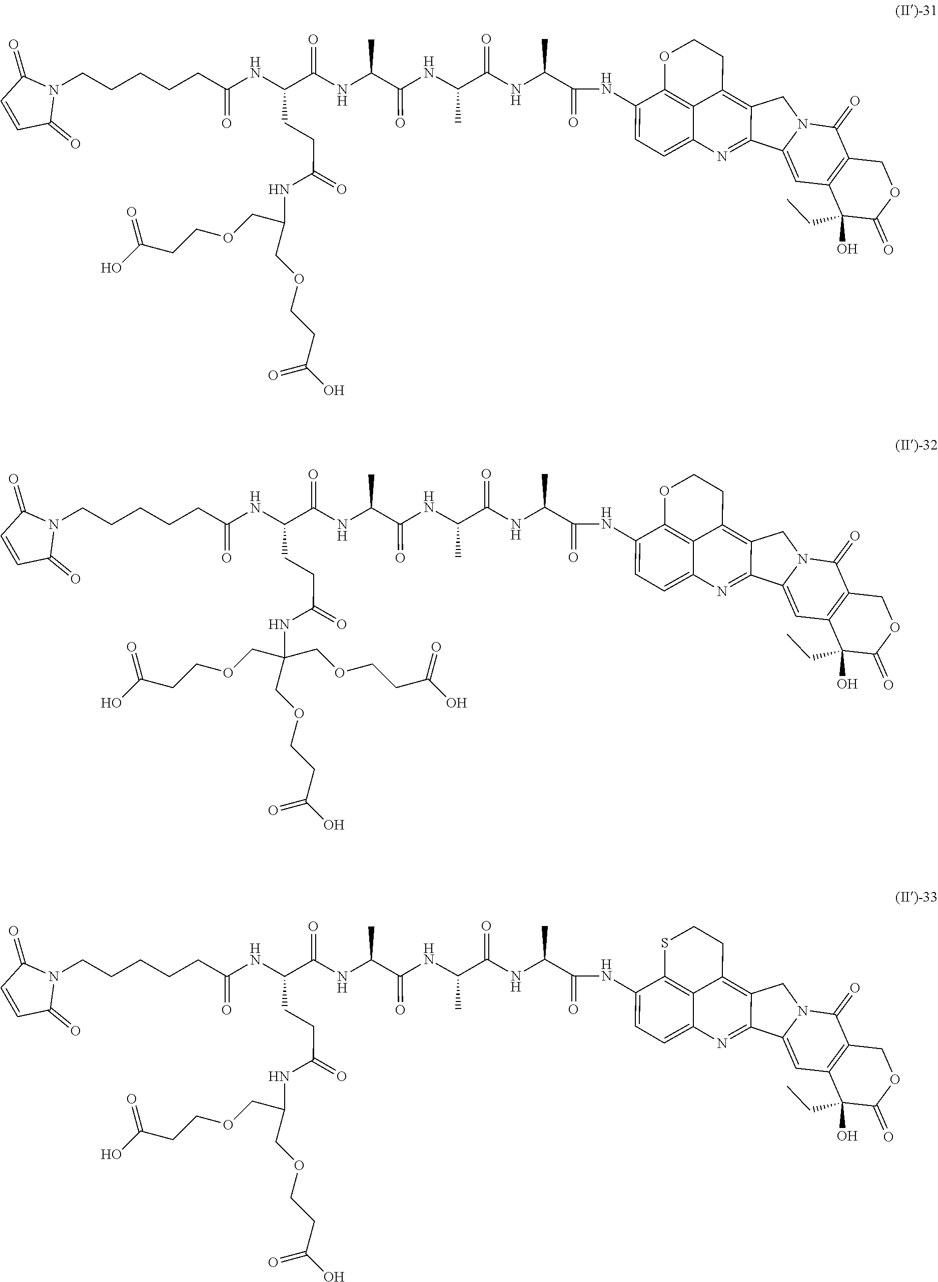

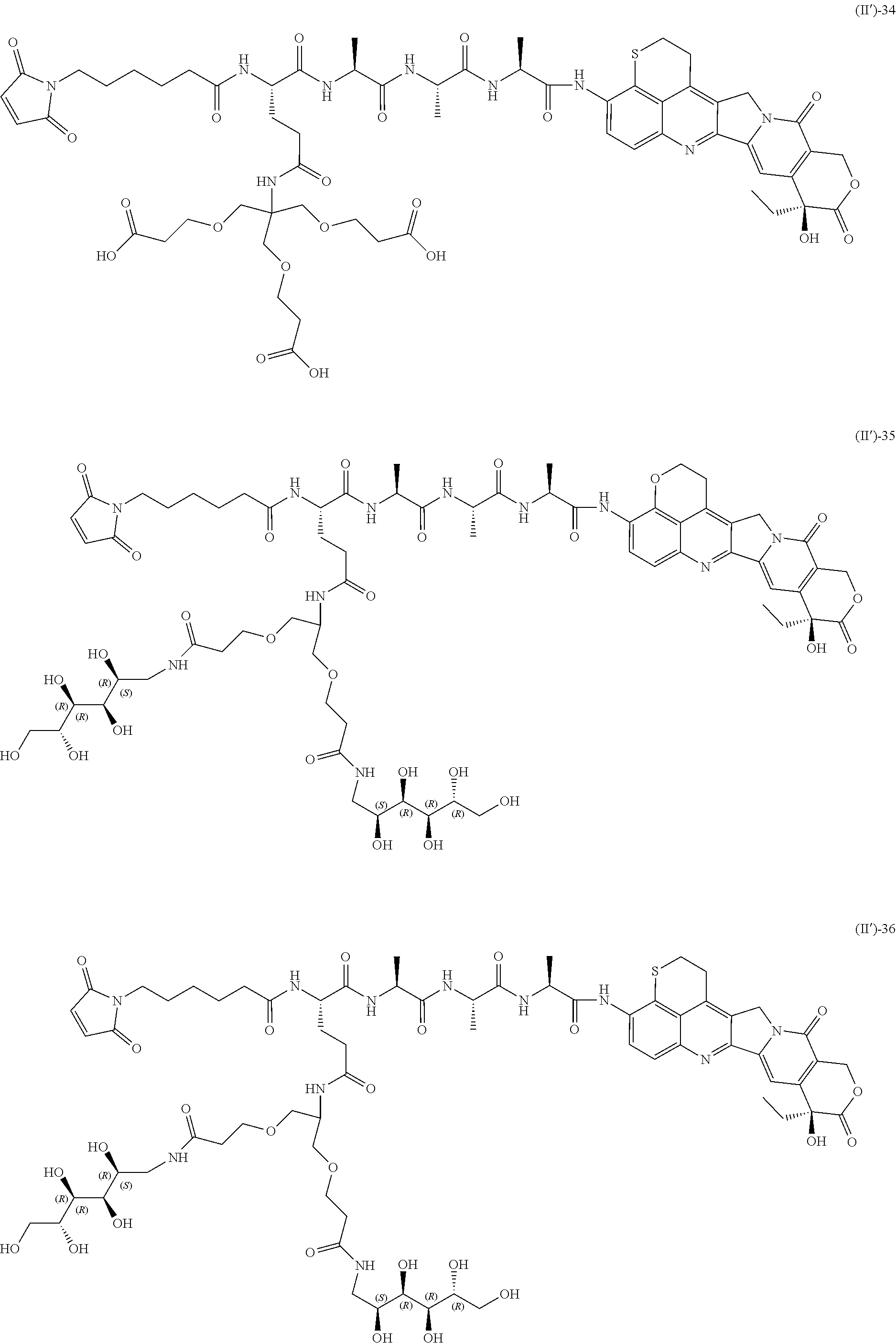

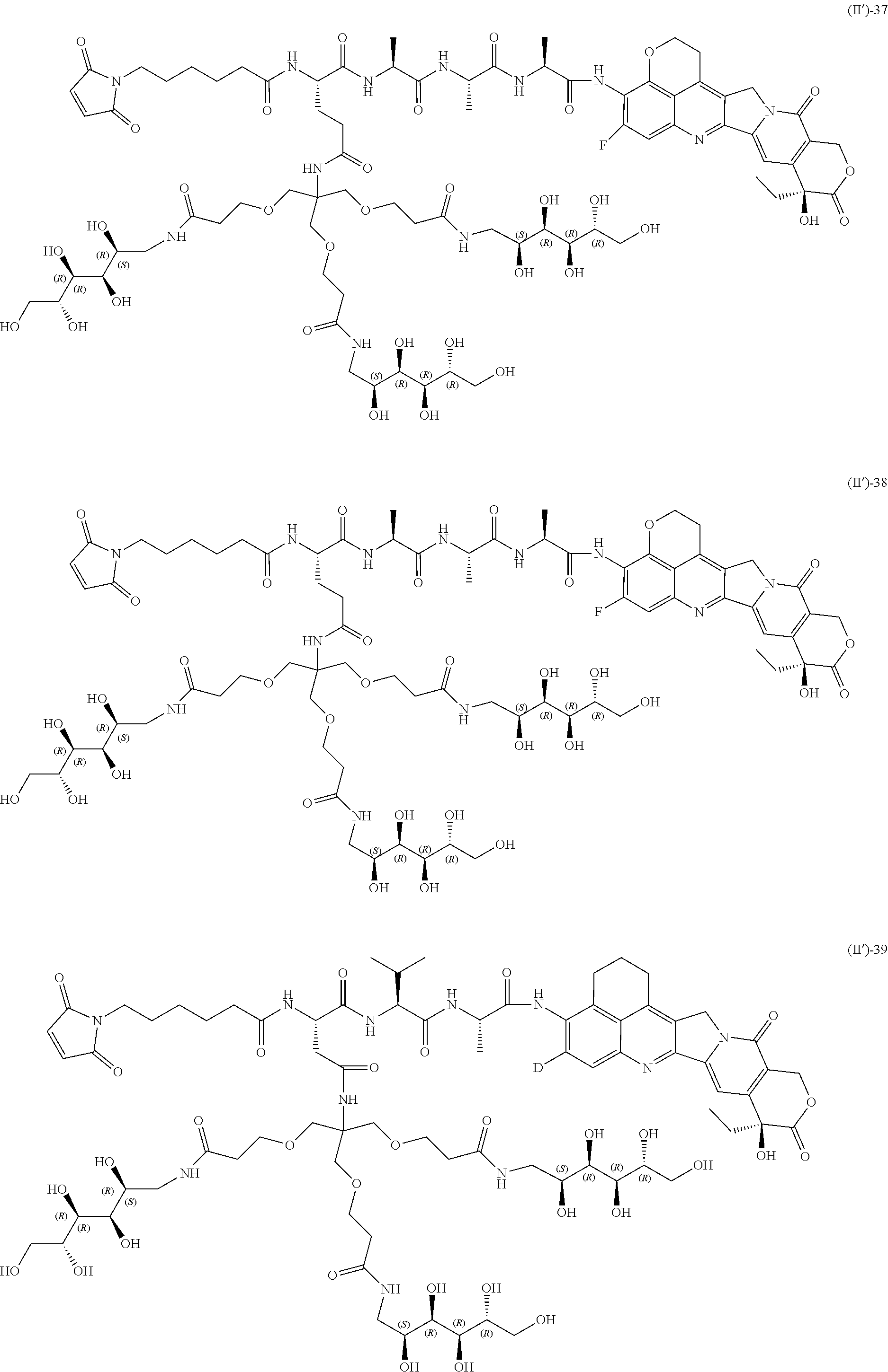

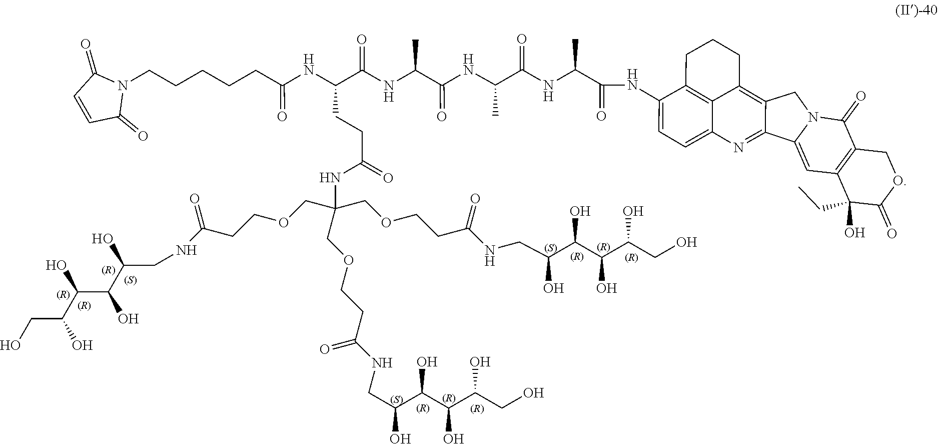

### Ligand-Drug Conjugates

As used herein, the term “ligand-drug conjugate” refers to a compound obtained by connecting a ligand and a therapeutic agent together using a linking agent compound, for example the compound of Formula (I). In the present disclosure, “ligand-drug conjugate” is preferably antibody-drug conjugate (ADC), which refers to a conjugate obtained by attaching an antibody (for example, a monoclonal antibody) or an antibody fragment to a therapeutic agent (for example, a cytotoxic drug) via a linking agent.

In a third aspect, the present invention is directed to a ligand-drug conjugate of Formula (III):

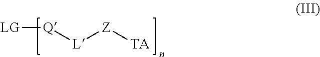

or a pharmaceutically acceptable salt, solvate, stereoisomer, or isotopic variant thereof, wherein
LG denotes to a ligand;
TA denotes to a therapeutic agent;
Z is absent or denotes to an auxiliary moiety connecting L′ to the therapeutic agent TA via a bond selected from the group consisting of disulfide, thioether, thioester, hydrazone, ester, ether, carbamate and amide bond;
Q′ denotes to a junction moiety coupled to the ligand LG via a bond selected from the group consisting of carbonyl, thioether, amide, disulfide and hydrazone bond;
L′ denotes to a linker moiety connecting Q to the therapeutic agent TA, and having a structure of:

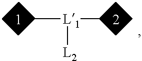

where L′₁ is a polypeptide residue consisting of three to eight amino acid residues which comprises at least one amino acid residue with a side chain carboxyl group, for example, glutamic acid residue or aspartic acid residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O)(CH₂CH₂O)ₘ(CH₂)ₚC(O)NHR^{L2d}, R^{L2d} is H or C₁₋₆ alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4 for example, 1, 2, 3, or 4;
[symbol] denotes to the N-terminal side of the polypeptide residue covalently attached to the junction moiety Q′;
[symbol] denotes to the C-terminal side of the polypeptide residue covalently attached to the therapeutic agent TA; and
n is a number in the range from 1 to 8.

As used herein, the term “ligand” refers to a macromolecular compound that recognizes and binds an antigen or receptor associated with a target cell. The role of the ligand is to present the therapeutic agent (the drug) to a population of target cells bound to the ligand, which includes, but is not limited to, protein-like hormones, lectins, growth factors, antibodies or other molecules capable of binding to cells. In some embodiments, the ligand may form a linking bond with the linking agent via a heteroatom on the ligand, preferably an antibody or antigen-binding fragment thereof, and the antibody is selected from a chimeric antibody, a humanized antibody, a fully human antibody or a mouse-derived antibody; preferably a monoclonal antibody.

In some embodiments, the ligand is coupled to the linking agent via a mercapto group (—SH) or amine group (—NH₂).

In some preferred embodiments, the antibody or antigen-binding fragment thereof is coupled to the linking agent via a mercapto group (—SH) or amine group (—NH₂) on the side chain of an amino acid residue.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

Preferably, the antibody described in the present disclosure refers to an immunoglobulin, which is a tetrapeptide chain structure consisting of two identical heavy chains and two identical light chains linked by interchain disulphide bonds. Immunoglobulins differ in the composition and order of amino acids in the constant region of the heavy chain, and therefore in their antigenicity. Accordingly, immunoglobulins can be divided into five classes, or isotypes of immunoglobulins, namely IgM, IgD, IgG, IgA and IgE, whose corresponding heavy chains are μ, δ, γ, α, and ε chains respectively. For example, IgG can be divided into IgG1, IgG2, IgG3 and IgG4. Light chains can be divided into κ or λ chains depending on their constant regions. Each of the five classes of Ig can have either a κ chain or a λ chain.

Preferably, the antibodies described in the present disclosure are specific antibodies against cell surface antigens on target cells, non-limiting embodiments being the following antibodies: anti-HER2 (ErbB2) antibody, anti-EGFR antibody, anti-TROP2 antibody, anti-B7-H3 antibody, anti-c-Met antibody, anti-HER3 (ErbB3) antibody, anti-HER4 (ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MUC1 antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody, anti-CTLA4 antibody, anti-OX40 antibody or one or more of anti-Mesothelin antibodies; preferably trastuzumab, pertuzumab, nimotuzumab, Enoblituzumab, Emibetuzumab, Inotuzumab, Pinatuzumab, Brentuximab, Gemtuzumab, Bivatuzumab, Lorvotuzumab, cBR96 and Glematumamab.

In some embodiments, the antibody is an antibody capable of binding to HER2, HER3, CD19, CD20, CD22, CD30, CD33, CD37, CD45, CD56, CD66e, CD70, CD74, CD79b, CD137, CD138, CD147, CD223, EpCAM, Mucin 1, STEAPI, GPNMB, FGF2, FOLR1, EGFR, EGFRvIII, Tissue factor, c-MET, FGFR, Nectin 4, AGS-16, Guanylyl cyclase C, Mesothelin, SLC44A4, PSMA, EphA2, AGS-5, GPC-3, c-KIT, ROR1, PD-L1, CD27L, 5T4, Mucin 16, NaPi2b, STEAP, SLITRK6, ETBR, BCMA, Trop-2, CEACAM5, SC-16, SLC39A6, Delta-like protein3, IL2RA, PTK7, or Claudin 18.2 tumor-associated antigen.

In some embodiments, the therapeutic agent TA is defined as described in the second aspect.

In some embodiments, the junction moiety Q′ has a structure of:

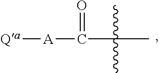

wherein Q′^{a} is a function group coupled to a ligand;
A is selected from optionally substituted C₃₋₈ alkylidene, optionally substituted C₃₋₈ alkenylidene, optionally substituted C₃₋₆ cycloalkenylidene, optionally substituted C₃₋₈ cycloalkylidene, optionally substituted diglycol to octaglycol acyl, where alkylidene, alkenylidene, cycloalkenylidene, cycloalkylidene, diglycol to octaglycol acyl is optionally substituted with 1 to 4 substituents independently selected from the group consisting of halogen, —CN, R^{Q′a1}, —OR^{Q′a1}, —SR^{Q′a1}, and —N(R^{Q′a1})₂, where each R^{Q′a1} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“ [symbol] ” denotes to the site covalently attached to the linker moiety L′.

In some preferred embodiments, the junction moiety Q′^{a} is selected from the group consisting of:

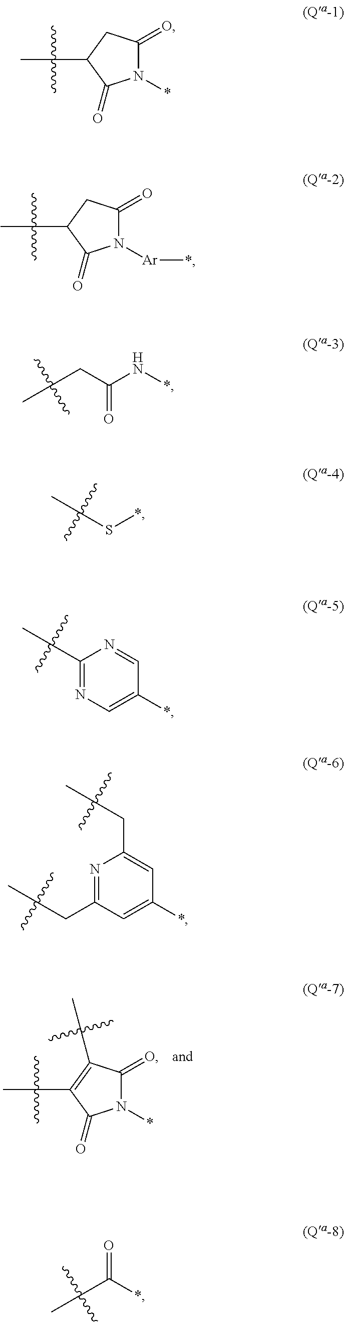

where, Ar is selected from the group consisting of optionally substituted C₅₋₆ cycloalkenyl, optionally substituted C₆ aryl and optionally substituted 5- to 6-membered heteroaryl, where cycloalkenyl, aryl and heteroaryl optionally substituted with 1 to 4 substituents independently selected from the group consisting of halogen, —CN, R^{Qa2}, —OR^{Qa2}, —SR^{Qa2}, and —N(R^{Qa2})₂, where each R^{Qa2} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl;
“*” denotes to the site covalently attached to A; and
“ [symbol] ” denotes to the site covalently attached to the ligand LG.

It should be understood that in the cases when the group “Ar” is inside of the molecular structure of a compound, it can also refer to a divalent substituent.

In some preferred embodiments, Ar is selected from the group consisting of optionally substituted cyclopentadienyl, optionally substituted phenyl, and optionally substituted 5- to 6-membered heteroaryl.

In some embodiments, the polypeptide residue L′ is defined as described in the second aspect.

In some embodiments, n denotes to the average amount of therapeutic agent (for example, a cytotoxic drug) loaded on each ligand (for example, an antibody) in a molecule of Formula (III). In the cases when the ligand is an antibody, it is also expressed as a ratio of the amount of drug to the amount of antibody, i.e., drug-to-antibody ratio (abbreviated as DAR). In some embodiments, n exemplarily may be a number of 1, 2, 3, 4, 5, 6, 7, 8. The average amount of drug per ADC molecule after the coupling reaction can be identified by conventional methods such as UV/visible spectroscopy, mass spectrometry, ELISA tests and HPLC characterization.

In some preferred embodiment, the present invention is directed to a ligand-drug conjugate of Formula (III′)

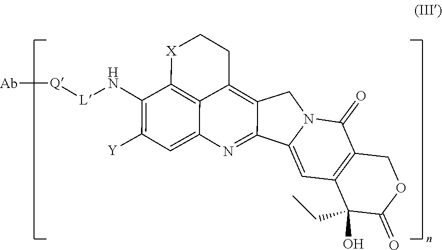

or a pharmaceutically acceptable salt, solvate, stereoisomer, or isotopic variant thereof, wherein
X is selected from the group consisting of —CH₂—, O, and S; Y is selected from the group consisting of H, D, and F;
Ab denotes to an antibody;
Q and L′ are as defined in the preceding claims; and
n is a number in the range from 1 to 8.

In some preferred embodiments, the compound of Formula (III′) of the present invention is selected from the group consisting of:

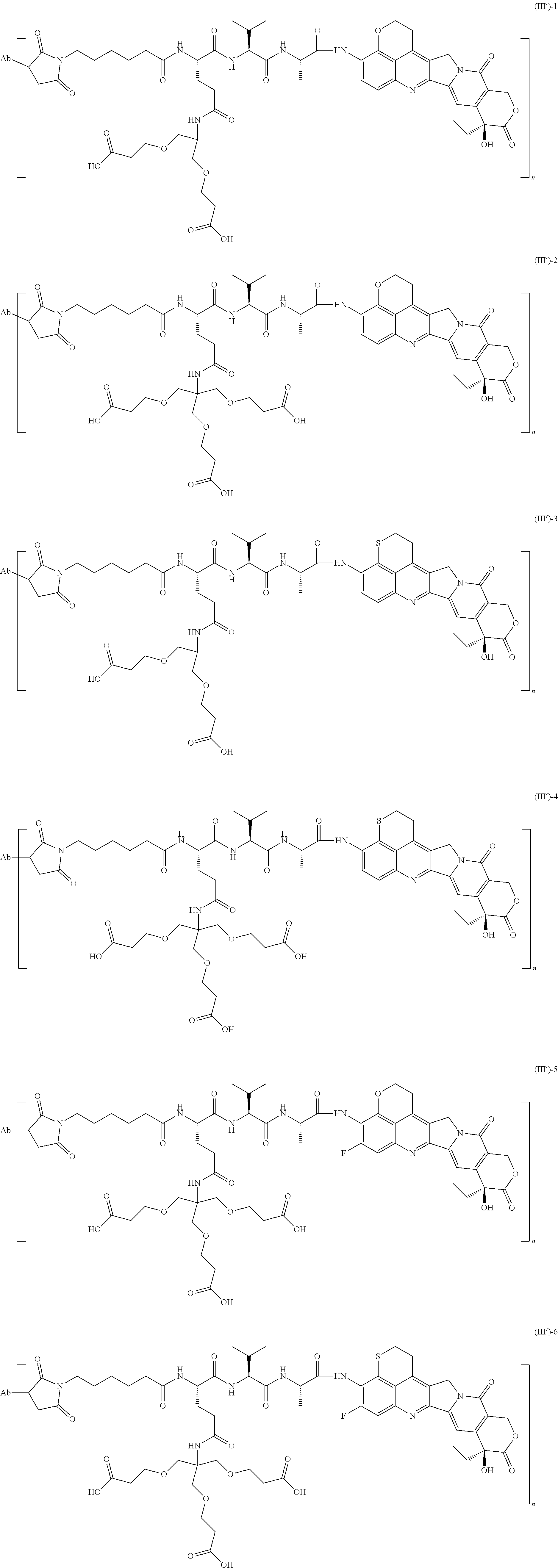

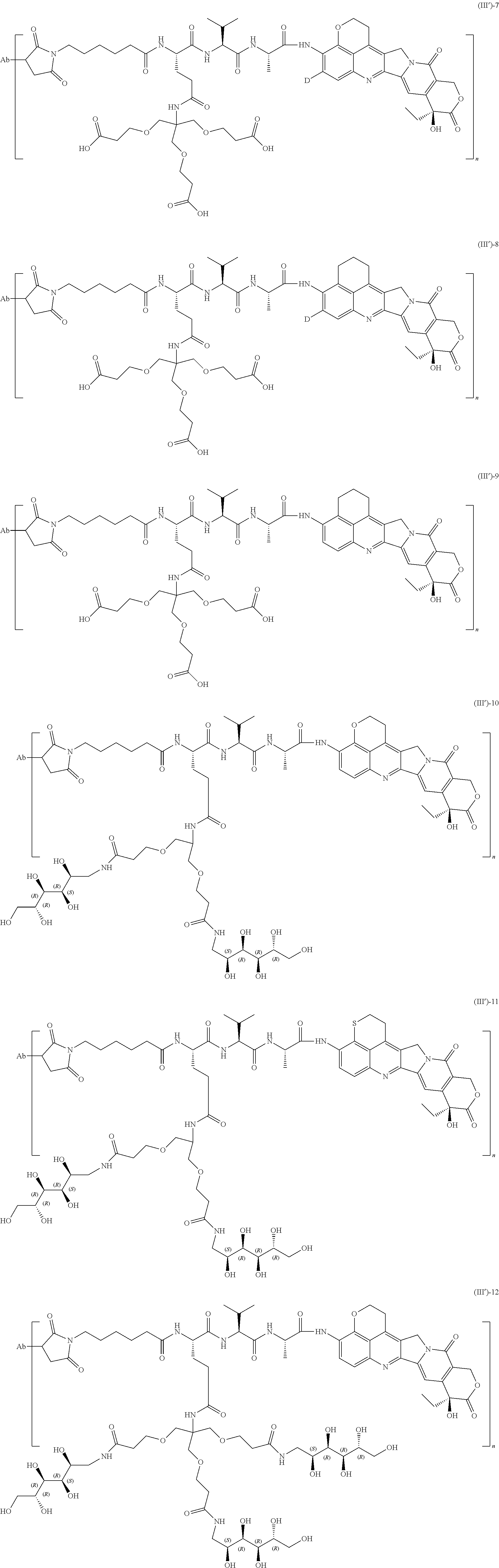

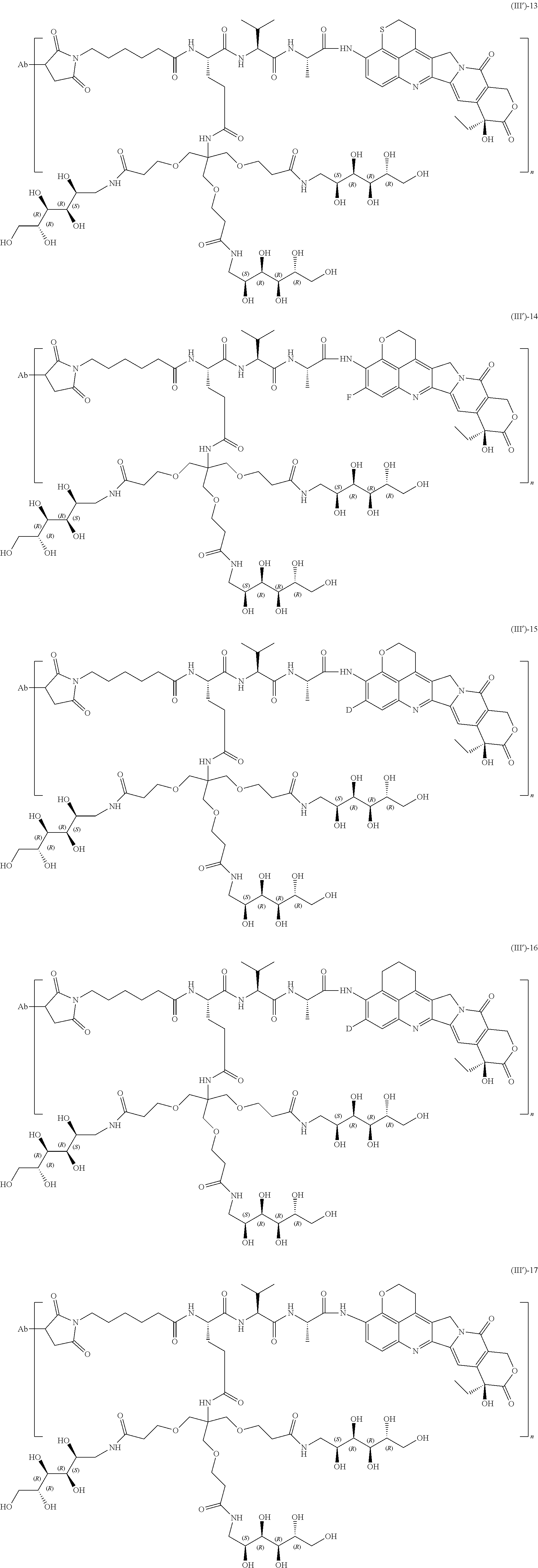

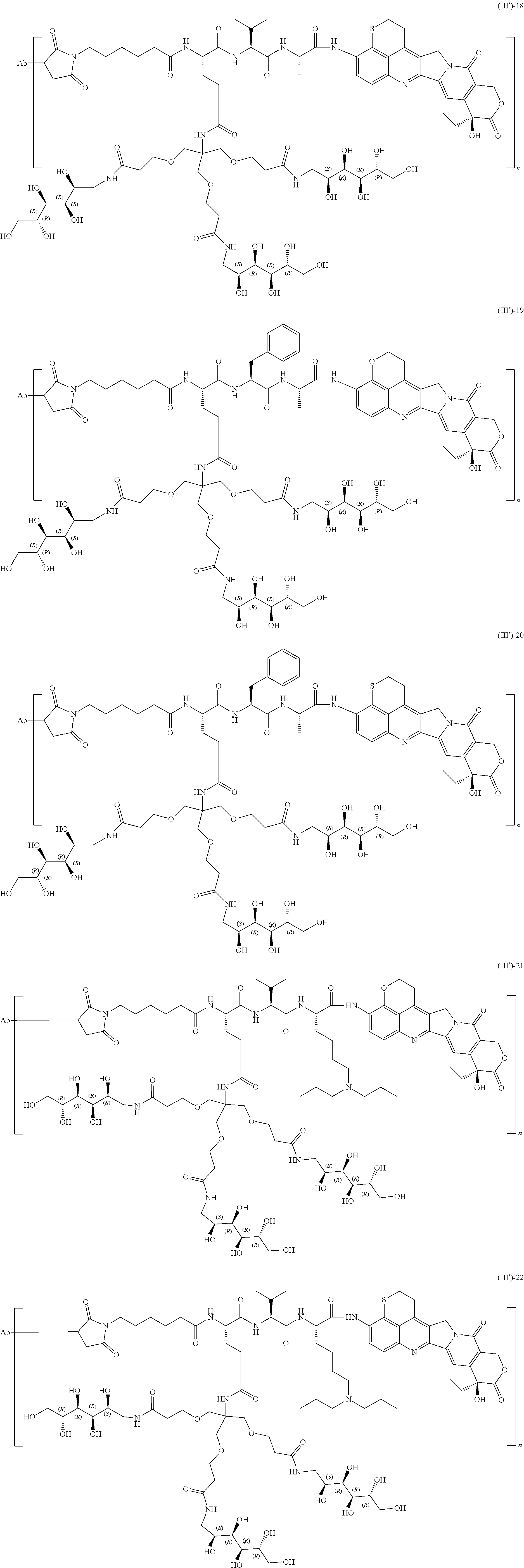

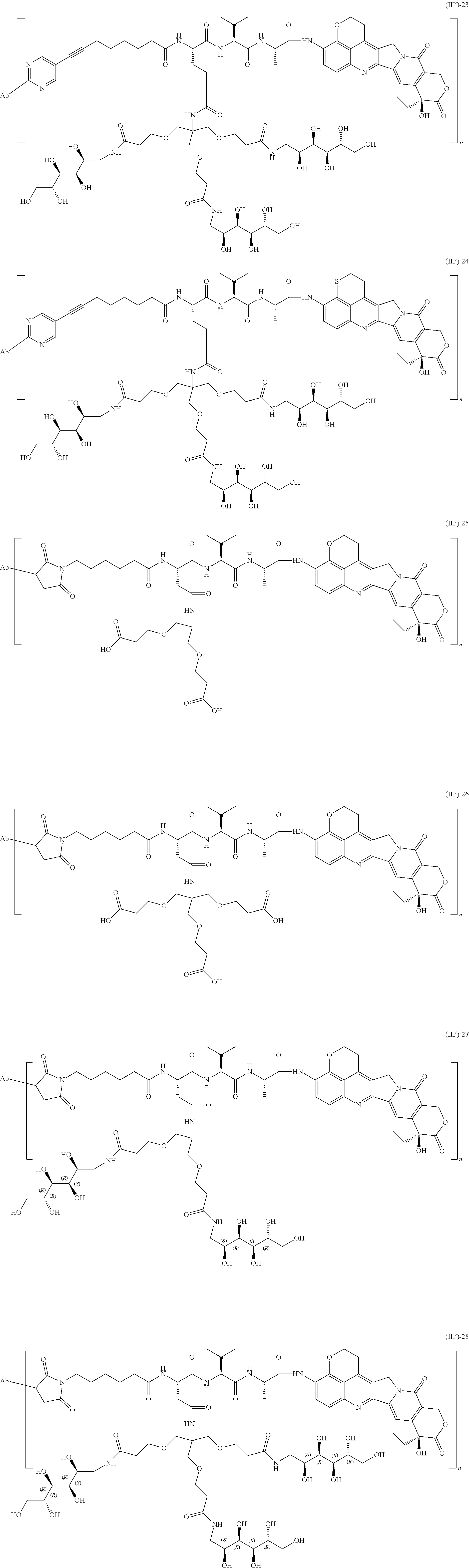

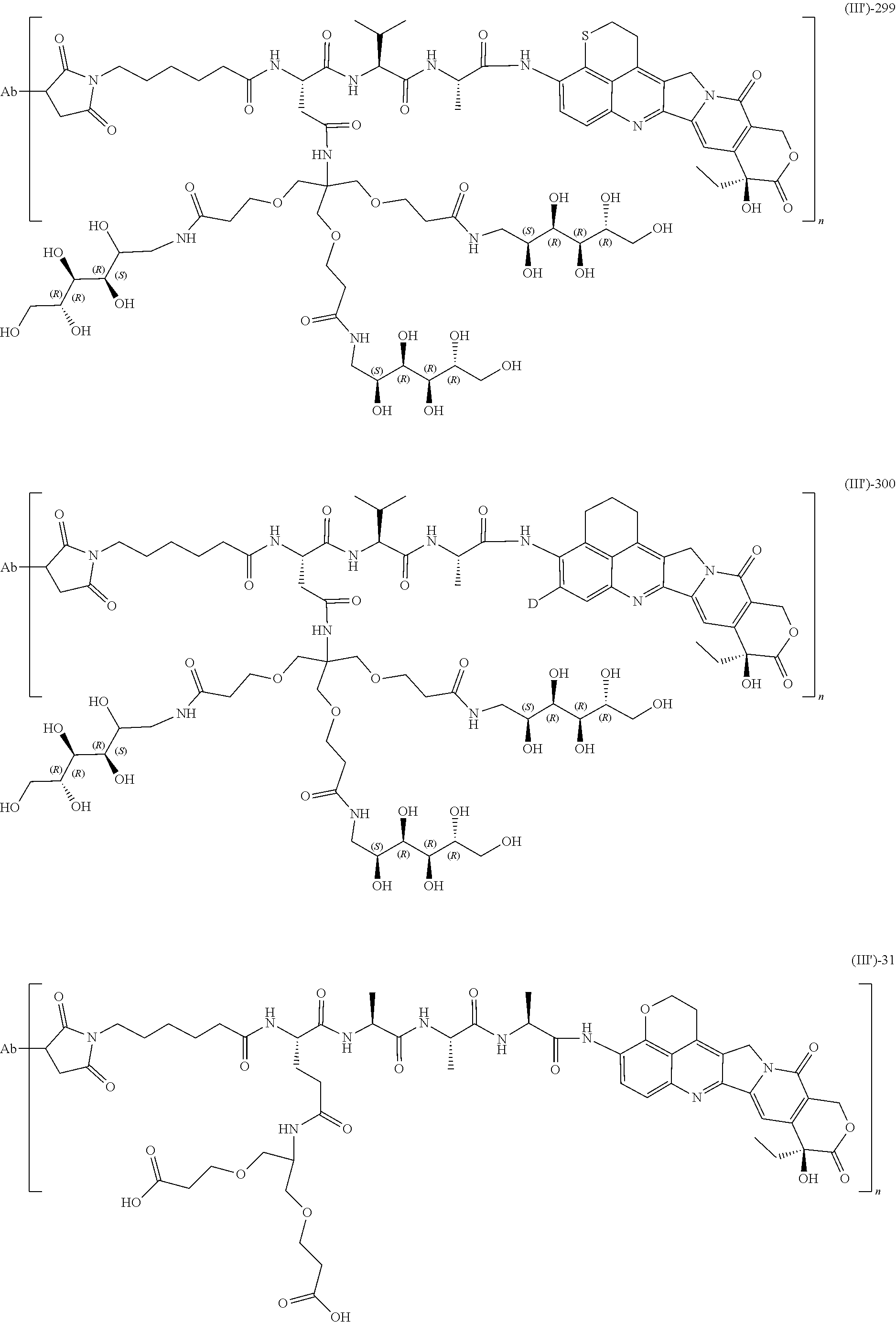

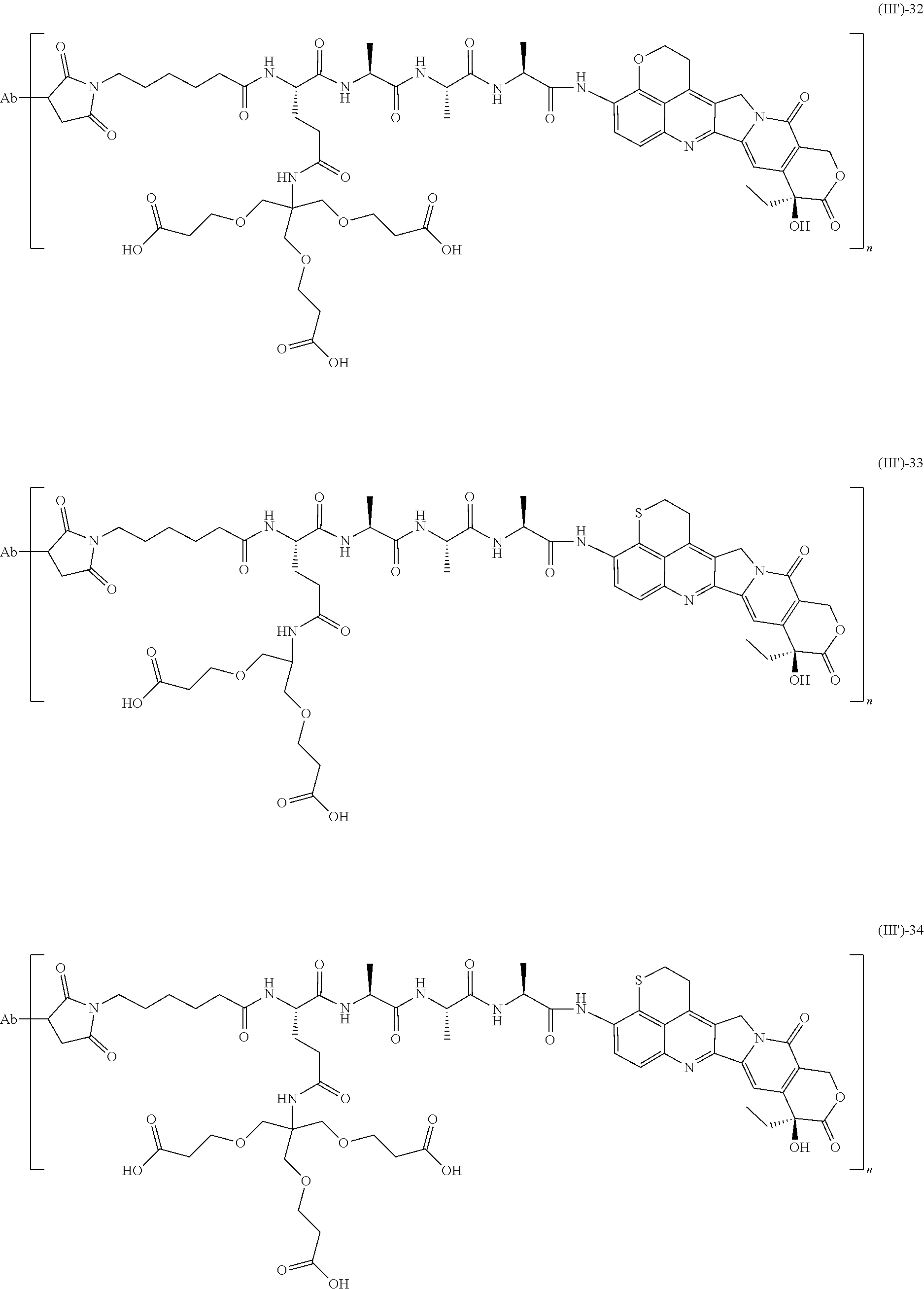

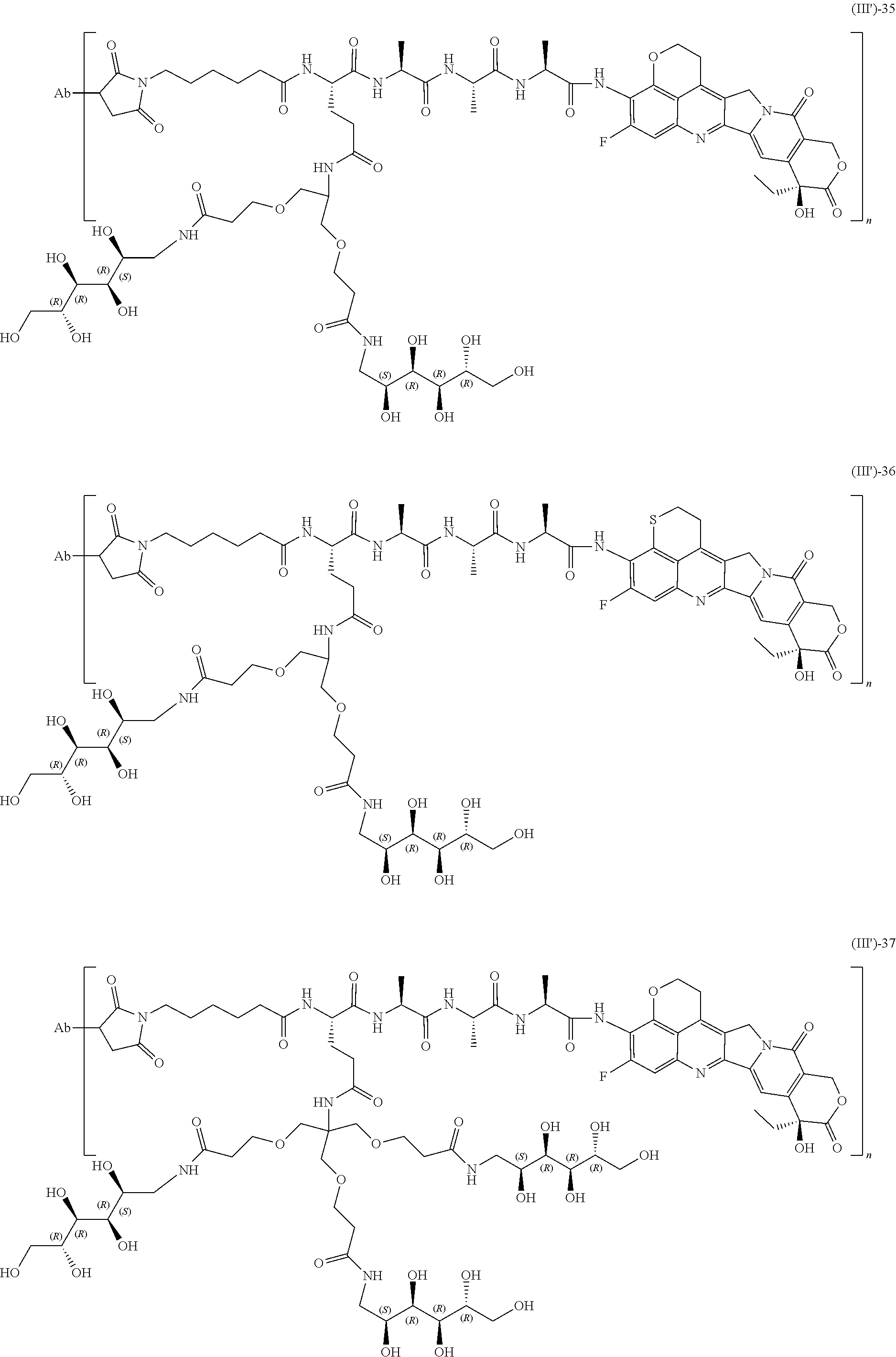

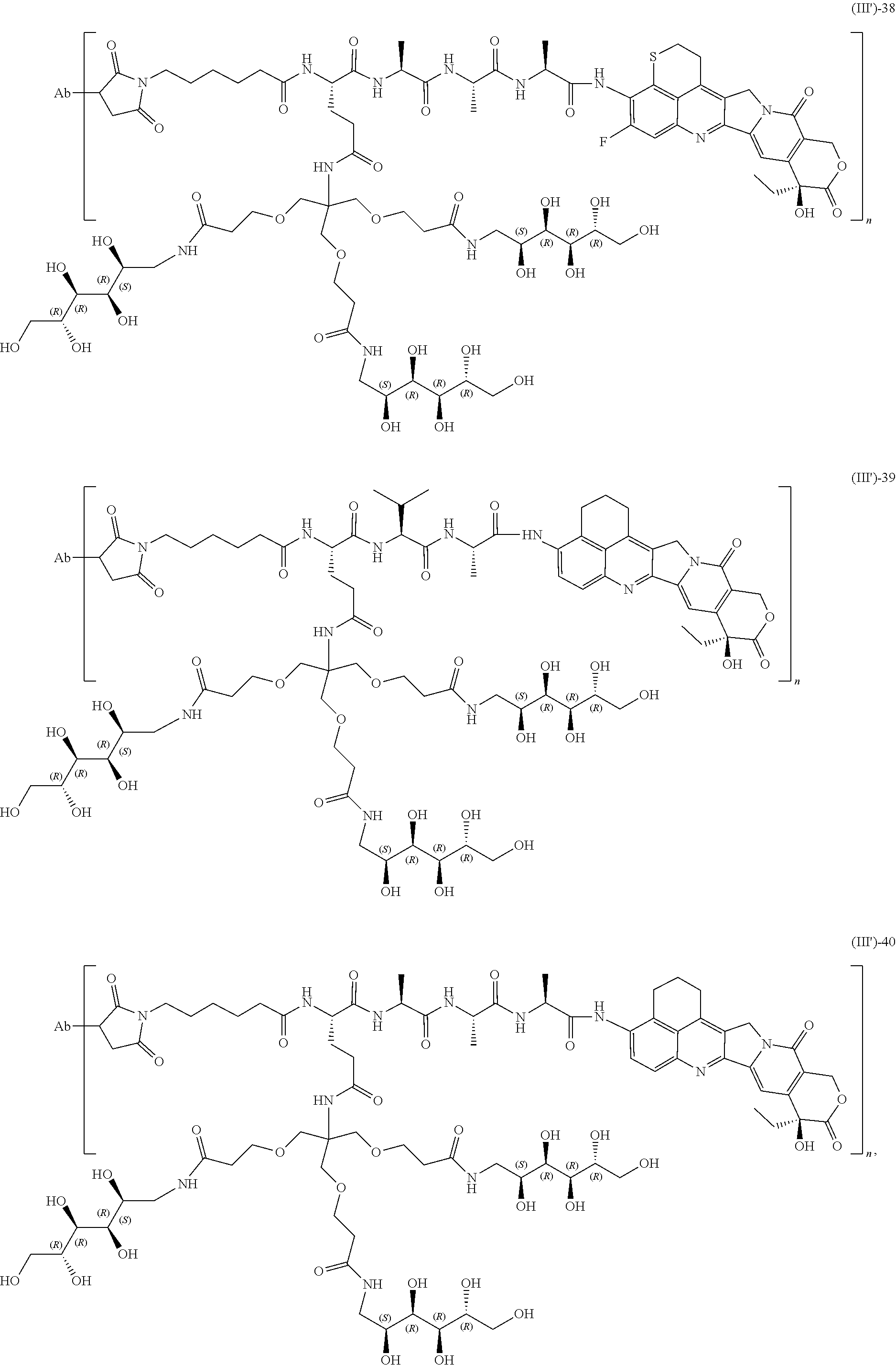

where n is a number in the range from 1 to 8.

### Camptothecin Compounds

In a fourth aspect, the present invention is directed to a camptothecin compound of Formula (IV):

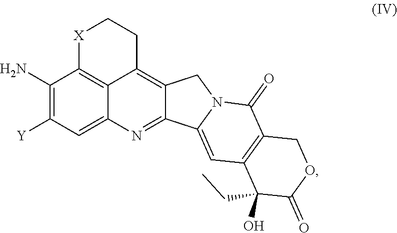

or a pharmaceutically acceptable salt, solvate, stereoisomer, or isotopic variant thereof, wherein
X is selected from the group consisting of —CH₂—, O, and S; and
Y is selected from the group consisting of H, D, and F.

### Usage and Administration

The compounds of the present invention—or a pharmaceutically acceptable salt, solvate, stereoisomer, or isotopic variant thereof, including mixtures thereof in all ratios—can be used as medicaments. They are found to exhibit pharmacological activity of inhibiting tumors.

Accordingly, the compounds of the present invention being anti-tumor agents are useful in particular in the treatment of cancer, in particular tumors including solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer pancreatic cancer, liver cancer, bladder cancer, stomach cancer or breast cancer. Without wishing to commit to any specific theory or explanation, it may be assumed that the compounds might be able to achieve this by direct effects on the cancer cells and/or indirectly by modulating the response of the immune system against the tumor.

The compounds of the present invention may be administered in an amount effective to treat the diseases or conditions as described herein. The compounds of the present invention can be administered as compound per se, or alternatively, as a pharmaceutically acceptable salt. For administration and dosing purposes, the compound of the present invention per se or pharmaceutically acceptable salt, solvate, stereoisomer, or isotopic variant thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, or topically.

As used herein, the terms “administration” and “administer” refer to absorbing, ingesting, injecting, inhaling, implanting, or otherwise introducing the compound of the invention, or a pharmaceutical composition thereof. The terms “treatment” and “treat” refer to reversing, alleviating, delaying the onset of, or inhibiting the progress of a “pathological condition” (e.g., a disease, disorder, or condition, or one or more signs or symptoms thereof) described herein. In certain embodiments, treatment may be administered after one or more signs or symptoms of a disease or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. As used herein, the terms “disease”, “disorder”, “condition”, and “pathological condition” are used interchangeably.

Dosage levels for administration can be determined by those skilled in the art by routine experimentation. The dosage regimen for the compounds of the invention and/or compositions comprising said compounds is based on a variety of factors, including the type, age, weight, sex, and medical condition of the patient; the severity of the condition; the route of administration; and the activity of the particular compound employed. Thus, the dosage regimen may vary widely. For example, dosage levels for the compounds of the invention may be from about 0.001 to about 100 mg/kg (i.e., mg per kilogram of body weight) per day. In certain embodiments, the total daily dose of a compound of the invention, administered in single or divided doses, may be from about 0.001 to about 10 mg/kg. It is not uncommon that the administration of the compounds of the invention may be repeated a plurality of times in a day.

In some embodiments, the compound of the invention may be administered in combination with one or more of additional therapeutical agents. In certain embodiments, non-limiting examples of the additional therapeutical agents may include an anti-tumor agent. The additional therapeutical agent(s) can be administered before, after, or at the same time that the compound of the present invention is administered.

As used herein, the term “anti-tumor agent” refers to any agent which is administered to a subject suffered from a cancer for the purposes of treating the cancer. Conventional surgery or radiotherapy or medicinal therapy may be used in combination with the compound of the present invention for cancer treatment.

### Pharmaceutical Compositions

In some aspect, the present invention is directed to a pharmaceutical composition comprising the compound of Formula (III) or a pharmaceutically acceptable salt, solvate, stereoisomer, or isotopic variant thereof as provided herein, and at least one pharmaceutically acceptable diluent, carrier or excipient.

As used herein, the term “pharmaceutically acceptable diluent, carrier or excipient” refers to a diluent, carrier or excipient which is useful for preparing a pharmaceutical composition that is generally safe, non-toxic, and neither biologically nor otherwise undesirable, and includes diluent, carrier or excipient that is acceptable for veterinary use as well as human pharmaceutical use. A pharmaceutically acceptable diluent, carrier or excipient as used herein includes both one and more than one such diluent, carrier or excipient. The particular diluent, carrier or excipient used will depend upon the means and purpose for which the compounds of the invention is being applied. Suitable diluents, carriers and excipients are well known to those skilled in the art and are described in detail in, e.g., Ansel, Howard C, et al., Ansel's Pharmaceutical Dosage Forms and Drug Delivery Systems. Philadelphia: Lippincott, Williams & Wilkins, 2004; Gennaro, Alfonso R., et al., Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. One or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, and other known additives may also be included to provide an elegant presentation of the drug (i.e., the compound or pharmaceutical composition as provided herein) or aid in the manufacturing of the pharmaceutical product (i.e., medicament).

The compositions of the present invention may be formulated in a variety of forms. These include, for example, liquid, semi-solid, and solid dosage forms, such as liquid solutions (e.g., injectable and infusible solutions), dispersions, or suspensions, tablets, pills, powders, liposomes, suppositories, etc. The form depends on the intended mode of administration and therapeutic application.

Pharmaceutical compositions of the present invention may be prepared by any of the well-known techniques of pharmacy, such as effective formulation and administration procedures. The above considerations in regard to effective formulations and administration procedures are well known in the art, and are described in standard textbooks. Formulation of pharmaceutical products is discussed in, e.g., Hoover, John E., Remington's Pharmaceutical Sciences, Mack Publishing Co., Easton, Pennsylvania, 1975; Liberman, et al., Eds., Pharmaceutical Dosage Forms, Marcel Decker, New York, N.Y., 1980; and Kibbe, et al., Eds., Handbook of Pharmaceutical Excipients, 3^{rd} Edition, American Pharmaceutical Association, Washington, 1999.

In certain embodiments, the pharmaceutical compositions comprise the compound of Formula (III) or a pharmaceutically acceptable salt, solvate, stereoisomer, or isotopic variant thereof as provided herein, in combination with one or more of additional therapeutical agents such as an anti-tumor agent, and at least one pharmaceutically acceptable diluent, carrier or excipient.

In a further aspect, the present invention relates to a kit for treating a tumor, which comprises a compound of Formula (III) or a pharmaceutically acceptable salt, solvate, stereoisomer, or isotopic variant thereof as provided herein, or a pharmaceutical composition comprising the compound of Formula (IIII) or a pharmaceutically acceptable salt, solvate, stereoisomer, or isotopic variant thereof as provided herein, a container, and optionally a package insert or label indicating treatment. In certain embodiments, the kit may further contain one or more of additional therapeutical agents such as an anti-tumor agent.

### Methods of Treatment

In a further aspect, the present invention is directed to a method of treating a tumor in a subject in need thereof, which comprises administering to the subject a therapeutically effective amount of the compound of Formula (III) or a pharmaceutically acceptable salt, solvate, stereoisomer, or isotopic variant thereof as provided herein.

As used herein, the term “subject in need thereof” is a subject having a tumor, or a subject having an increased risk of developing a tumor relative to the population at large. In certain embodiments, the subject is a warm-blooded animal. In certain embodiments, the warm-blooded animal is a mammal. In certain embodiments, the warm-blooded animal is a human.

The method of treating a tumor as described herein may be used as a monotherapy. As used herein, the term “monotherapy” refers to the administration of a single active or therapeutic compound to a subject in need thereof. In certain embodiments, monotherapy will involve administration of a therapeutically effective amount of one of the compounds of the present invention or a pharmaceutically acceptable salt, solvate, stereoisomer, or isotopic variant thereof, to a subject in need of such treatment.

Depending upon the particular disease or condition to be treated, the method of treating a tumor described herein may involve, in addition to administration of the compound of Formula (III), combination therapy of one or more additional therapeutic agent(s), for example, an anti-tumor agent. As used herein, the term “combination therapy” refers to the administration of a combination of multiple active therapeutic agents. In certain embodiments, the compound of the present invention or a pharmaceutically acceptable salt, solvate, stereoisomer, or isotopic variant thereof may be administered simultaneously, separately or sequentially to treatment with the one or more additional therapeutic agent(s). For example, the additional therapeutic agent(s) may be administered separately from the compound of the present invention, as part of a multiple dosage regimen. Alternatively, the additional therapeutic agent(s) may be part of a single dosage form, mixed with the compound of the present invention in a single composition.

In a further aspect, the present invention is directed to use of the compound of Formula (III) or a pharmaceutically acceptable salt, solvate, stereoisomer, or isotopic variant thereof as provided herein in the manufacture of a medicament for treating a tumor in a subject in need thereof.

### Synthesis

The compounds of the present invention may be prepared by general and specific methods described below, using the common general knowledge of those skilled in the art of synthetic organic chemistry. Such common general knowledge can be found in standard reference books, e.g., Barton and Ollis (Ed.), Comprehensive Organic Chemistry, Elsevier; Richard Larock, Comprehensive Organic Transformations: A Guide to Functional Group Preparations, John Wiley and Sons; and Compendium of Organic Synthetic Methods, Vol. I-XII, Wiley-Interscience.

The schemes described hereinafter are intended to provide a general description of the methodology employed in the preparation of the compounds of the present invention. Some of the compounds of the present invention may contain single or multiple chiral centers with the stereochemical designation (R) or (S). It will be apparent to those skilled in the art that all of the synthetic transformations can be conducted in a similar manner no whether the materials are enantioenriched or racemic. Moreover, the resolution to the desired optically active material may take place at any desired point in the procedure using well known methods such as those described herein and in the chemistry literature.

In some aspect, the present invention is directed to a method for preparation of a ligand-drug conjugate of Formula (III) or a pharmaceutically acceptable salt, solvate, stereoisomer, or isotopic variant thereof of the present invention, comprising the following steps of:
a) reacting a ligand with a reducing agent in a buffer to obtain a reduced ligand; and
b) coupling a compound of Formular (II) or a pharmaceutically acceptable salt, solvate, stereoisomer, or isotopic variant thereof of the present invention with the reduced ligand obtained in step a) in a mixture of a buffer and an organic solvent to obtain the ligand-drug conjugate;
or comprising the following step of:
coupling a compound of Formular (II) or a pharmaceutically acceptable salt, solvate, stereoisomer, or isotopic variant thereof of the present invention with a ligand in a mixture of a buffer and an organic solvent to obtain the ligand-drug conjugate.

### EXAMPLES

In order that the invention described herein may be more fully understood, the following examples are set forth. The examples described in this application are offered to illustrate the methods and compositions provided herein and are not to be construed in any way as limiting their scope.

All reagents and materials are purchased from commercial vendors or may be readily prepared by those skilled in the art. A list of abbreviations used may be found in the Table below, below.

Abbreviations of reagents or organic Moieties
Reagents or Organic Moieties

| Abbreviation | Full Name |
|---|---|
| DAR | Drug to antibody ratio; |
| PE | Petroleum ether; |
| EA | Ethyl acetate; |
| PPTS | Pyridinium p-Toluenesulfonate |
| DMF | N,N-Dimethylformamide |
| TCFH | N,N,N′,N′-Tetramethylchloroformamidinium- |
| | hexafluorophosphate |
| TFA | Trifluoroacetic acid; |
| DCM | Dichloromethane |
| EDTA | Ethylenediaminetetraacetic acid; |
| TCEP | Tris(2-carboxyethyl)phosphine; |
| HATU | 2- (7-Azabenzotriazol-1-yl)-N,N,N′,N′- |
| | tetramethyluronium hexafluorophosphate |
| HOBT | 1-Hydroxybenzotriazole |
| DIEA | N,N-Diisopropylethylamine |
| DCC | Dicyclohexylcarbodiimide |
| NMM | 4-Methylmorpholine |
| THF | Tetrahydrofuran |
| RT | Room Temperature; |
| DP | Desired Product |

### Example 1. Preparation of Compounds

¹HNMR spectra were recorded on a Bruker Ascend 400 spectrometer. Chemical shifts are expressed in parts per million (ppm, b units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Agilent 1290 with SQ Detectors using a Waters Xbridge C18, 4.6×50 mm, 3.5 μm using a gradient elution method.
Solvent A: 0.1% TFA in water;
Solvent B: 0.1% TFA in acetonitrile;
5-95% B over 1.3 min.

### Preparation Example 1.1. Synthesis of (S)-4-amino-9-ethyl-9-hydroxy-1,9,12,15-tetrahydro-13H-pyrano[3′,4′:6,7]indolizino[1,2-b]thiopyrano[4,3,2-de]quinoline-10,13(2H)-dione) (CPT-1)

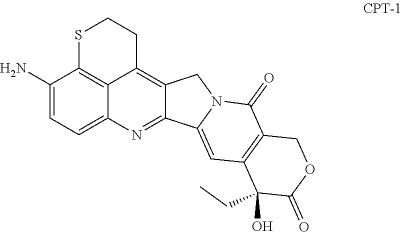 [SMILES: CC[C@@]1(O)C(=O)OCc2c1cc1n(c2=O)Cc2c-1nc1ccc(N)c3c1c2CCS3]

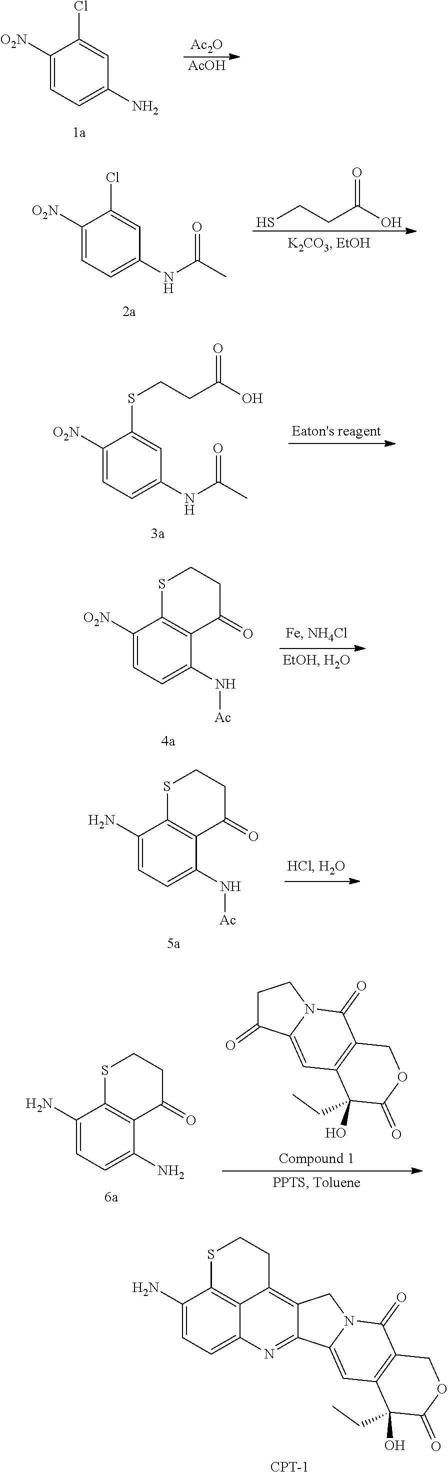

### Step 1: Synthesis of N-(3-chloro-4-nitrophenyl) acetamide (Compound 2a)

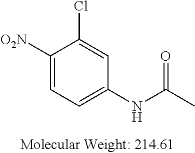

### Compound 2a

Ac₂O (39.185 mL, 417.222 mmol) was added to a solution of 3-chloro-4-nitroaniline (Compound 1a, 24 g, 139.074 mmol) in AcOH (50 mL) and the resulting mixture was stirred at 120° C. for 2 h. After the reaction was completed as confirmed by TLC (PE:EA=4:1), the AcOH was removed by vacuum distillation. Then, the residue was dissolved in water and the resulting solution was neutralized with Na₂CO₃ to weak alkaline before being extracted with EA to yield Compound 2a as a yellow solid (28 g, 130.475 mmol, yield: 93.82%), confirmed by LC-MS.

LC-MS: (ESI) m/z (M+H): 215.0.

### Step 2: Synthesis of 3-{[5-(acetylamino)-2-nitrophenyl]sulfanyl}propanoic acid (Compound 3a)

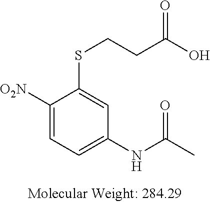 [SMILES: CC(=O)Nc1ccc([N+](=O)[O-])c(SCCC(=O)O)c1]

### Compound 3a

3-sulfanylpropanoic acid (16.62 g, 156.570 mmol) was added to a solution of Compound 2a (28 g, 130.475 mmol) and K₂CO₃ (54.09 g, 391.425 mmol) in EtOH (100 mL), and the resulting mixture was refluxing for overnight. Then, the mixture was cooled to RT and poured into ice water and the precipitate was filtered. Afterwards, the resulting solid product was washed with water and MeOH to yield Compound 3a as a yellow solid (28 g, 98.491 mmol, yield: 75.49%), confirmed by LC-MS.

LC-MS: (ESI) m/z (M+Na): 307.0.

### Step 3: Synthesis of N-(8-nitro-4-oxothiochroman-5-yl)acetamide (Compound 4a)

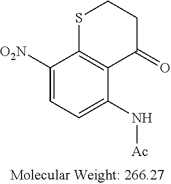 [SMILES: CC(=O)Nc1ccc([N+](=O)[O-])c2c1C(=O)CCS2]

### Compound 4a

A solution of Compound 3a (5 g, 17.588 mmol) in [(dioxo-λ5-phosphanyl)oxy]dioxo-λ5-phosphane methanesulfonic acid (20 mL) was stirred at RT for 1.5 h under a N₂ atmosphere. After LC-MS analysis showed 30% DP (desired product) was formed, the resulting mixture was then poured into ice water and the precipitate was filtered out and washed with Na₂CO₃ aqueous solution to yield Compound 4a as a faint yellow solid (680 mg, 2.554 mmol, yield: 14.52%), confirmed by LC-MS.

LC-MS: (ESI) m/z (M+Na): 289.0.

### Step 4: Synthesis of N-(8-amino-4-oxothiochroman-5-yl) acetamide (Compound 5a)

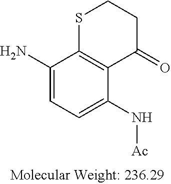 [SMILES: CC(=O)Nc1ccc(N)c2c1C(=O)CCS2]

### Compound 5a

A mixture of Compound 4a (640 mg, 2.404 mmol), Fe (536.86 mg, 9.614 mmol) and NH₄Cl (1285.67 mg, 24.036 mmol) in EtOH (8 mL) and water (4 mL) was stirred at 80° C. for 2 h. After the reaction was completed as confirmed by LC-MS, the resulting product was obtained by filtration and extraction with EA to yield Compound 5a as an orange solid (567.94 mg, 2.404 mmol, quantitative yield), confirmed by LC-MS.

LC-MS: (ESI) m/z (M+H): 237.0.

### Step 5: Synthesis of 5, 8-diaminothiochroman-4-one (Compound 6a)

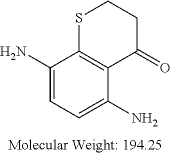 [SMILES: Nc1ccc(N)c2c1SCCC2=O]

### Compound 6a

A solution of Compound 5a (400 mg, 1.693 mmol) in hydrochloric acid solution (2.0N in water) was stirred at 90° C. for 2 h until the reaction was confirmed to be completed by LC-MS analysis. The mixture was then poured into water and the resulting solution was neutralized with Na₂CO₃ to weak alkaline before being extracted with EA to yield Compound 6a as an orange solid (328.83 mg, 1.693 mmol, quantitative yield), confirmed by LC-MS.

LC-MS: (ESI) m/z (M+H): 195.2.

### Step 6: Synthesis of CPT-1

PPTS (336.22 mg, 1.338 mmol) was added to a solution of Compound 6a (200 mg, 1.030 mmol) and (4S)-4-ethyl-4-hydroxy-3,4,6,7,8,10-hexahydro-1H-pyrano[3,4-f]indolizine-3,6,10-trione (Compound 1, 352.21 mg, 1.338 mmol) in toluene (10 mL). The resulting mixture was then stirred at 120° C. for overnight under a N₂ atmosphere. After the reaction was completed as detected by LC-MS, the toluene was removed by vacuum distillation and the residue was dissolved in DMF before being purified using preparative liquid chromatography with TFA as an eluent to yield CPT-1 as a dark brown solid (80 mg, 0.19 mmol, yield: 18.44%), confirmed by LC-MS and NMR.

LC-MS: (ESI) m/z (M+H):422.2.

¹HNMR (400 MHz, DMSO): δ 7.77 (d, J=9.2 Hz, 1H), 7.38 (d, J=9.2 Hz, 1H), 7.25 (s, 1H), 6.52 (s, 1H), 5.74 (s, 2H), 5.47 (s, 2H), 5.24 (s, 2H), 3.52-3.43 (m, 2H), 3.27-3.23 (m, 2H), 1.96-1.87 (m, 2H), 0.94 (t, J=7.2 Hz, 3H).

### Preparation Example 1.2. Synthesis of (S)-4-amino-9-ethyl-9-hydroxy-1,9,12,15-tetrahydro-13H-pyrano[4,3,2-de]pyrano[3′,4′:6,7]indolizino[1,2-b]quinoline-10,13(2H)-dione (CPT-2)

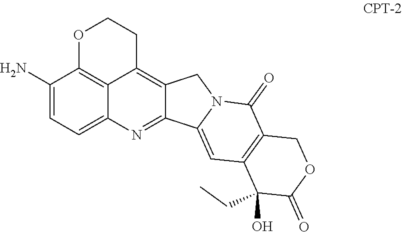 [SMILES: CC[C@@]1(O)C(=O)OCc2c1cc1n(c2=O)Cc2c-1nc1ccc(N)c3c1c2CCO3]

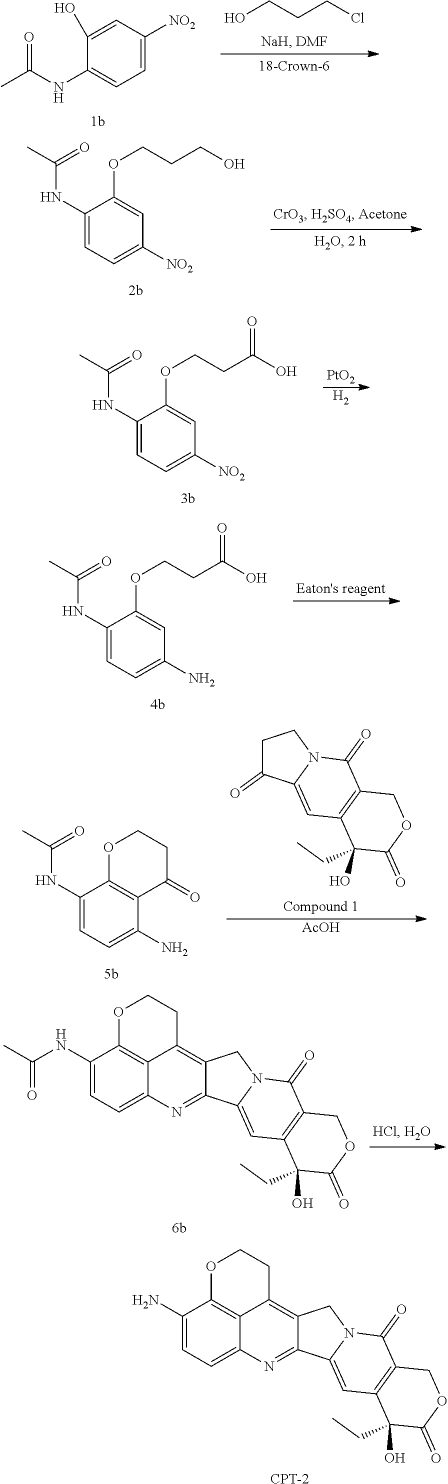

### Step 1: Synthesis of N-(2-(3-hydroxypropoxy)-4-nitrophenyl) acetamide (Compound 2b)

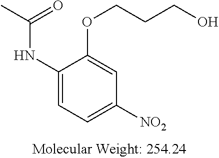 [SMILES: CC(=O)Nc1ccc([N+](=O)[O-])cc1OCCCO]

### Compound 2b

N-(2-hydroxy-4-nitrophenyl) acetamide (Compound 1b, 10 g, 50.979 mmol) was added to a suspension of NaH (1.25 g, 52.083 mmol) in DMF (200 mL) and the resulting mixture was stirred for 20 min. Then, 3-chloropropan-1-ol (6.372 mL, 76.158 mmol) and 18-crown-6 (0.1 g, 0.378 mmol) were added to the mixture which was heated to 80° C. for 2 days. After the reaction finished, the resulting mixture was poured to ice-water and then extracted with EA. Afterwards, the organic layers were washed with 10% NaOH aqueous solution and brine before being dried over Na₂SO₄, filtered and concentrated. The product Compound 2b was obtained by recrystallized (EtOH/PE) as a brown solid (6.34 g, 24.937 mmol, yield: 48.92%), confirmed by LC-MS.

LC-MS: (ESI) m/z (M+H): 255.2.

### Step 2: Synthesis of 3-(2-acetamido-5-nitrophenoxy) propanoic acid (Compound 3b)

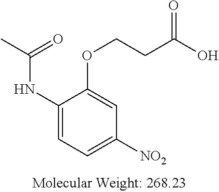 [SMILES: CC(=O)Nc1ccc([N+](=O)[O-])cc1OCCC(=O)O]

### Compound 3b

Jone's reagent (19.937 mL, 39.874 mmol) was added dropwise at 0° C. to a solution of Compound 2b (6.336 g, 24.921 mmol) in acetone (125 mL) and the resulting mixture was stirred at RT for overnight. After the reaction finished, the mixture was diluted with water and extracted with EAT. Then, the organic layers were washed with brine before being followed by dried over Na₂SO₄, filtered and concentrated. The product Compound 3b was obtained by recrystallized (MeOH/H₂O) as a yellow solid (5.819 g, 21.694 mmol, yield: 87.05%), confirmed by ¹HNMR.

¹HNMR (400 MHz, DMSO): δ 12.50 (s, 1H), 9.37 (s, 1H), 8.39 (d, J=8.8 Hz, 1H), 7.90 (dd, J=8.8, 2.4 Hz, 1H), 7.87 (d, J=2.4 Hz, 1H), 4.35 (t, J=6.0 Hz, 2H), 2.85 (t, J=6.0 Hz, 2H), 2.18 (s, 3H).

### Step 3: Synthesis of 3-(2-acetamido-5-aminophenoxy) propanoic acid (Compound 4b)

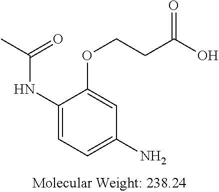 [SMILES: CC(=O)Nc1ccc(N)cc1OCCC(=O)O]

### Compound 4b

A suspension of Compound 3b (5 g, 18.641 mmol) and PtO₂ (0.59 g, 2.610 mmol) in EtOH (325 mL) and Water (75 mL) was stirred in an atmosphere of H₂ (1 atm) for overnight. After the reaction finished, the catalyst was removed by filtration, and the solution was concentrated. The product Compound 4b was obtained by recrystallized (MeOH—H₂O) as a brown solid (4.10 g, 17.210 mmol, yield: 92.32%), confirmed by NMR.

¹HNMR (400 MHz, DMSO): δ 8.60 (s, 1H), 7.37 (d, J=8.4 Hz, 1H), 6.28 (d, J=2.4 Hz, 1H), 6.11 (dd, J=8.8, 2.4 Hz, 1H), 4.06 (t, J=6.4 Hz, 2H), 2.71 (t, J=6.4 Hz, 2H), 1.97 (s, 3H).

### Step 4: Synthesis of N-(5-amino-4-oxochroman-8-yl) acetamide (Compound 5b)

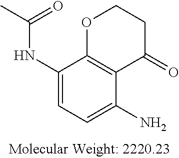 [SMILES: CC(=O)Nc1ccc(N)c2c1OCCC2=O]

### Compound 5b

Compound 4b (500 mg, 2.099 mmol) was added to a solution of Eaton's reagent (10 mL, 63.015 mmol) and then the mixture was heated to 60° C. under a N₂ atmosphere for 6 h. After the reaction finished, the resulting mixture was based with NaOH aq. and extracted with EA. Then, the combined organic phase was washed with brine before being dried over Na₂SO₄, filtered, and concentrated under reduced pressure. The product Compound 5b was obtained by Flash (PE:EA=1:9) as a yellow solid (73 mg, 0.331 mmol, yield: 15.79%), confirmed by LC-MS.

LC-MS: (ESI) m/z (M+H): 221.2.

### Step 5: Synthesis of (S)—N-(9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)acetamide (Compound 6b)

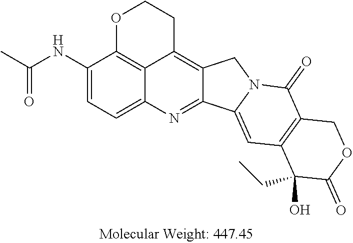 [SMILES: CC[C@@]1(O)C(=O)OCc2c1cc1n(c2=O)Cc2c-1nc1ccc(NC(C)=O)c3c1c2CCO3]

### Compound 6b

A solution of Compound 5b (73 mg, 0.331 mmol) and Compound 1 (95.99 mg, 0.365 mmol) in AcOH (3 mL) was heated to reflux under a N₂ atmosphere for 5 h. After the reaction finished, the mixture was concentrated under reduced pressure. The product Compound 6b was obtained by Flash (DCM:MeOH=95:5) as a yellow solid (42.3 mg, 0.104 mmol, yield: 31.48%), confirmed by LC-MS.

LC-MS: (ESI) m/z (M+H): 448.2.

### Step 6: Synthesis of CPT-2

Compound 6b (138 mg, 0.308 mmol) was added to a solution of 12M HCl (15 mL) and H₂O (15 mL). Then the mixture was heated to reflux under a N₂ atmosphere for 1 h. After the reaction finished, the mixture was concentrated under reduced pressure. The product CPT-2 was obtained by preparative liquid chromatography (TFA) as an orange solid (52 mg, 0.128 mmol, yield: 41.59%), confirmed by LC-MS and NMR.

LC-MS: (ESI) m/z (M+H):406.2.

¹HNMR (400 MHz, DMSO): δ 7.56 (d, J=9.2 Hz, 1H), 7.35 (d, J=9.2 Hz, 1H), 7.20 (s, 1H), 6.46 (s, 1H), 5.41 (s, 4H), 5.20 (s, 2H), 4.42 (t, J=5.6 Hz, 2H), 3.30-3.27 (m, 2H), 1.92-1.80 (m, 2H), 0.87 (t, J=7.2 Hz, 3H).

### Preparation Example 1.3. Synthesis of (S)-4-amino-9-ethyl-5-fluoro-9-hydroxy-1,9,12,15-tetrahydro-13H-pyrano[3′,4′:6,7]indolizino[1,2-b]thiopyrano[4,3,2-de]quinoline-10,13(2H)-dione) (CPT-3)

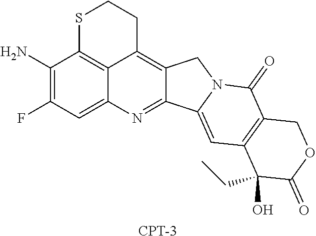 [SMILES: CC[C@@]1(O)C(=O)OCc2c1cc1n(c2=O)Cc2c-1nc1cc(F)c(N)c3c1c2CCS3 ; "CPT-3"]

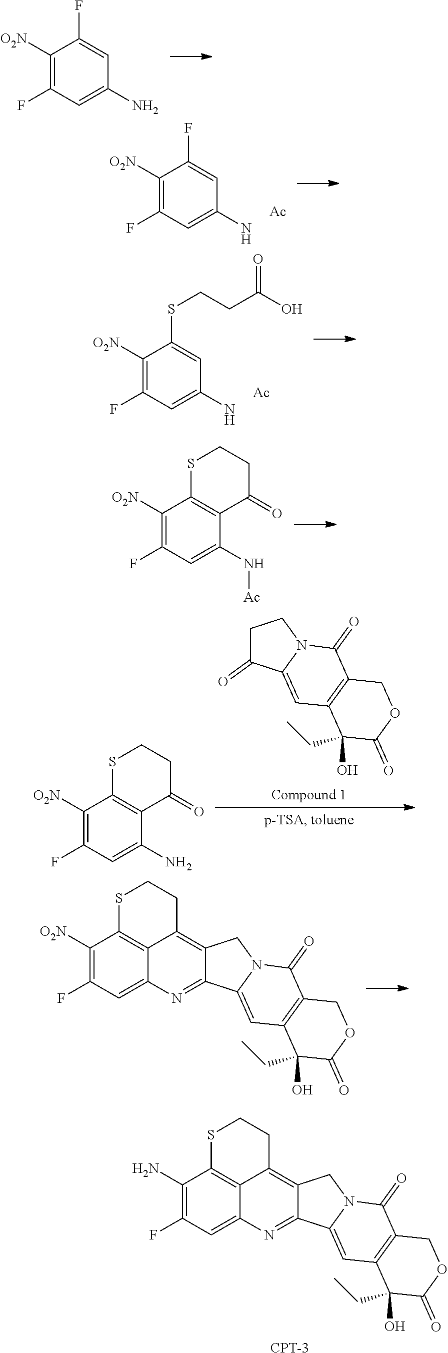

### Preparation Example 1.4. Synthesis of (S)-4-amino-9-ethyl-5-fluoro-9-hydroxy-1,9,12,15-tetrahydro-13H-pyrano[4,3,2-de]pyrano[3′,4′:6,7]indolizino[1,2-b]quinoline-10,13(2H)-dione (CPT-4)

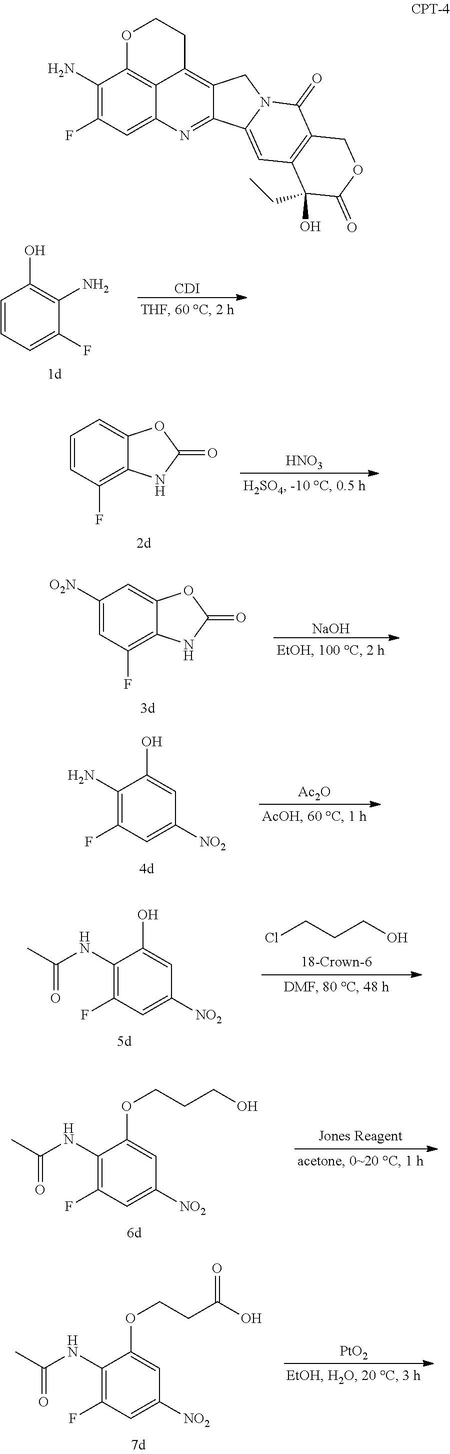

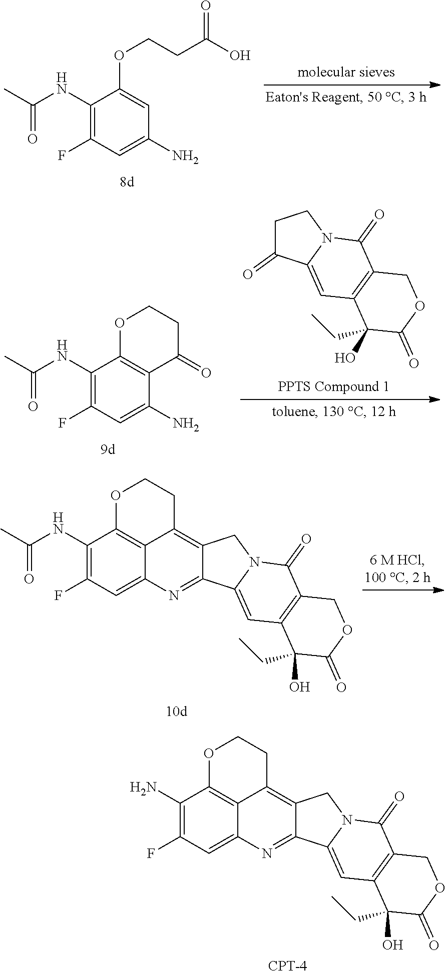 [SMILES: CC(=O)Nc1c(F)cc(N)cc1OCCC(=O)O ; CC(=O)Nc1c(F)cc(N)c2c1OCCC2=O ; CC[C@@]1(O)C(=O)OCc2c1cc1n(c2=O)CCC1=O ; CC[C@@]1(O)C(=O)OCc2c1cc1n(c2=O)Cc2c-1nc1cc(F)c(NC(C)=O)c3c1c2CCO3 ; CC[C@@]1(O)C(=O)OCc2c1cc1n(c2=O)Cc2c-1nc1cc(F)c(N)c3c1c2CCO3 ; "CPT-4"]

### Step 1: Synthesis of 4-fluorobenzo[d]oxazol-2(3H)-one (Compound 2d)

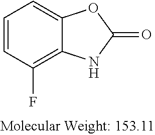 [SMILES: O=c1[nH]c2c(F)cccc2o1]

### Compound 2d

Di(imidazol-1-yl)methanone (21.05 g, 129.799 mmol) was added to the solution of 2-amino-3-fluorophenol (Compound 1d, 15 g, 117.999 mmol) in THF (300 mL) slowly at 0° C. and the reaction mixture was then stirred at 60° C. for 2 hr under a N₂ atmosphere. After the reaction finished, the reaction mixture was cooled to room temperature, diluted with H₂O (300 mL), and then the resulting solution was concentrated to remove most solvent before the residue was extracted with EA (300 mL*3). Afterwards, the combined organic phases were washed with 2 M HCl (300 mL*2), brine (900 mL*1) before being dried over anhydrous sodium sulfate, filtered and concentrated to give a residue. The residue (TLC-T2:DCM:MeOH=20:1, UV, product Rf=0.40) was purified by silica gel column (DCM:MeOH, from 2% to 4%) to yield the desired product Compound 2d which was brown solid (16.13 g, 105.349 mmol, yield: 89.28%) and confirmed by NMR (N230872-011-P1A, DMSO).

¹HNMR (400 MHz, DMSO): δ 12.25 (s, 1H), 7.21-7.13 (m, 1H), 7.13-7.05 (m, 2H)

### Step 2: Synthesis of 4-fluoro-6-nitrobenzo[d]oxazol-2(3H)-one (Compound 3d)

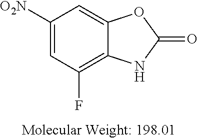 [SMILES: O=c1[nH]c2c(F)cc([N+](=O)[O-])cc2o1]

### Compound 3d

HNO₃ (7.659 mL, 110.617 mmol) was added to the solution of Compound 2d (16.13 g, 105.349 mmol) in H₂SO₄ (160 mL) slowly at −10° C., and the reaction mixture was stirred at −10° C. for 0.5 hr under a N₂ atmosphere. After the reaction finished, the reaction mixture was poured into ice water (300 mL) and then stirred at 0° C. for 30 min before being filtered. Afterwards, the filter cake was sequentially washed with H₂O (100 mL*3), collected, diluted with MeOH (160 mL) before the resulting solution was concentrated to yield the desired product 4-fluoro-6-nitro-2,3-dihydrobenzo[d][1,3]oxazol-2-one (Compound 3d, 20.87 g, 105.346 mmol, yield:100.00%) which was light brown solid and confirmed by ¹HNMR (N230872-016-P1A, DMSO).

¹HNMR (400 MHz, DMSO): δ 13.08 (s, 1H), 8.29-7.99 (m, 2H).

### Step 3: Synthesis of 2-amino-3-fluoro-5-nitrophenol (compound 4d)

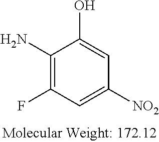 [SMILES: Nc1c(O)cc([N+](=O)[O-])cc1F]

### Compound 4d

NaOH (210.691 mL, 526.728 mmol) was added to the solution of Compound 3d (20.87 g, 105.346 mmol) in EtOH (800 mL) at 20° C., and the reaction mixture turned thicked, the color of which turned yellow. The reaction mixture was then stirred at 100° C. for 2 hr under a N₂ atmosphere. After the reaction finished, the reaction mixture was cooled to room temperature and then acidified by adding 2 M HCl to achieve a pH of 2-3. The reaction mixture turned yellow. Afterwards, the reaction mixture was neutralized with saturated NaHCO₃ to a pH of 8, filtered and the resulting filtrate was concentrated to remove solvent. The resulting residue was extracted with EA (400 mL*3), washed with brine (1 L*3), dried over anhydrous sodium sulfate, filtered and concentrated to yield Compound 4d (10.88 g, 63.212 mmol, yield: 60.00%) which was orange solid and confirmed by ¹HNMR (N230872-020-P1B, DMSO).

¹HNMR (400 MHz, DMSO): δ 10.52 (s, 1H), 7.55 (dd, J=11.1, 2.4 Hz, 1H), 7.42 (d, J=1.5 Hz, 1H), 6.12 (s, 2H).

### Step 4: Synthesis of N-(2-fluoro-6-hydroxy-4-nitrophenyl)acetamide (Compound 5d)

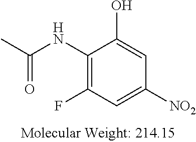

### Compound 5d

Acetic anhydride (7.718 mL, 82.175 mmol) was added to the solution of Compound 4d (10.88 g, 63.212 mmol) in AcOH (150 mL) slowly at 0° C. and the reaction mixture was stirred at 50° C. for 1 hr under a N₂ atmosphere. After the reaction finished, the reaction mixture was cooled to room temperature and H₂O (300 mL) was added. The resulting mixture was then acidified with 1 M HCl to a pH of 3-4 and stirred at 20° C. for 30 min before being filtered. Afterwards, the filter cake was successively washed with H₂O (50 mL*3), collected, diluted with MeOH (200 mL). The resulting mixture was concentrated to yield the product Compound 5d (9.91 g, 46.276 mmol, yield: 73.19%) which was light brown solid and confirmed by ¹HNMR (N230872-026-P1A, DMSO).

¹HNMR (400 MHz, DMSO): δ 11.02 (s, 1H), 9.62 (s, 1H), 7.60 (dd, J=9.6, 2.5 Hz, 1H), 7.55 (dd, J=2.4, 1.4 Hz, 1H), 2.06 (s, 3H).

### Step 5: Synthesis of N-(2-fluoro-6-(3-hydroxypropoxy)-4-nitrophenyl)acetamide (Compound 6d)

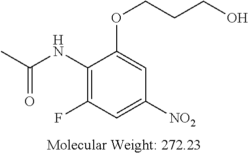 [SMILES: CC(=O)Nc1c(F)cc([N+](=O)[O-])cc1OCCCO]

### Compound 6d

NaH (2.06 g, 51.623 mmol) was added slowly to the solution of Compound 5d (10.05 g, 46.930 mmol) in DMF (200 mL) at 20° C. under a N₂ atmosphere, and the resulting mixture was stirred at 20° C. for 20 min under N₂. Then 3-chloropropan-1-ol (5.889 mL, 70.395 mmol) and 18-crown-6 (0.62 g, 2.346 mmol) was added slowly to the mixture at 20° C. under a N₂ atmosphere and the reaction mixture was stirred at 80° C. for 48 hr under a N₂ atmosphere. After the reaction finished, the reaction mixture was cooled to room temperature, and H₂O (200 mL) was added. Afterwards, the resulting mixture was extracted with EA (200 mL*3), and the organic layer was washed with 10% NaOH (500 mL*2) and then brine (500 mL*3) before being dried over anhydrous sodium sulfate, filtered and concentrated to obtain a residue. The residue was purified by silica gel column (DCM:MeOH, from 2% to 4%) to yield the desired product Compound 6d (3.71 g, 13.628 mmol, yield: 29.04%) which was light yellow solid and confirmed by LC-MS (N230872-029-P1) and ¹HNMR (N230872-029-P1A, DMSO).

LC-MS: m/z (ES+) (M+H)+=273.2, Rt=0.575 min.

¹HNMR (400 MHz, DMSO) δ 9.62 (s, 1H), 7.79 (dd, J=9.5, 2.3 Hz, 1H), 7.72 (s, 1H), 4.57 (s, 1H), 4.22 (t, J=6.3 Hz, 2H), 3.57 (s, 2H), 2.06 (s, 3H), 1.89 (p, J=6.2 Hz, 2H).

### Step 6: Synthesis of 3-(2-acetamido-3-fluoro-5-nitrophenoxy)propanoic acid (Compound 7d)

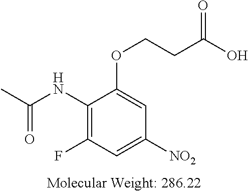 [SMILES: CC(=O)Nc1c(F)cc([N+](=O)[O-])cc1OCCC(=O)O]

### Compound 7d

Jones reagent (10.221 mL, 20.442 mmol) was added slowly to the solution of Compound 6d (3.71 g, 13.628 mmol) in acetone (60 mL) at 0° C. and the reaction mixture was stirred at 0-20° C. for 2 hr under a N₂ atmosphere. After the reaction finished, the reaction mixture was quenched with saturated Na₂SO₃ to a pH of 8, filtrated with celite and the filter cake was washed with H₂O (20 mL*3). The resulting filtrate was extracted with EA (100 mL*3) and the organic layer was then discarded. The aqueous layer was acidified with 1 M HCl to a pH of 3~4 before being extracted with EA (100 mL*3), washed with brine (300 mL*3), dried over anhydrous sodium sulfate, filtered and concentrated to yield the product Compound 7d (2.26 g, 7.896 mmol, yield: 57.94%) which was light yellow solid and confirmed by LC-MS (N230872-040-P1-1) and ¹HNMR (N230872-040-P1A, DMSO).

LC-MS: m/z (ES+) (M+H)+=287.2, Rt=0.573 min.

¹HNMR (400 MHz, DMSO) δ 12.44 (s, 1H), 9.61 (s, 1H), 7.82 (dd, J=9.5, 2.3 Hz, 1H), 7.76 (s, 1H), 4.36 (t, J=6.1 Hz, 2H), 2.74 (t, J=6.1 Hz, 2H), 2.05 (s, 3H).

### Step 7: Synthesis of 3-(2-acetamido-5-amino-3-fluorophenoxy)propanoic acid (Compound 8d)

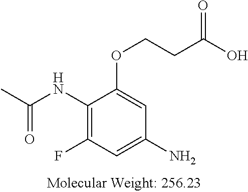 [SMILES: CC(=O)Nc1c(F)cc(N)cc1OCCC(=O)O]

### Compound 8d

Platinum dioxide (0.17 g, 0.737 mmol) was added to the solution of Compound 7d (2.11 g, 7.372 mmol) in EtOH (64 mL) and H₂O (16 mL) at 20° C. and the reaction mixture was stirred at 20° C. for 3 hr under H₂. After the reaction finished, the reaction mixture was filtrated with celite and the resulting filter was concentrated to obtain the residue. The residue was diluted with MeOH (2 mL), and H₂O (6 mL) was added before the resulting mixture was stirred at 20° C. for 1 hr under a N₂ atmosphere. Afterwards, the resulting mixture was filtrated and the filter cake was collected and concentrated to obtain the product Compound 8d (950 mg, 3.708 mmol, yield: 50.29%) which was light yellow solid and confirmed by ¹HNMR (N230872-050-PIA, DMSO).

¹HNMR (400 MHz, DMSO) δ 12.34 (s, 1H), 8.63 (s, 1H), 6.07 (s, 1H), 5.96 (dd, J=12.0, 2.1 Hz, 1H), 5.34 (s, 2H), 4.04 (t, J=6.4 Hz, 2H), 2.63 (t, J=6.4 Hz, 2H), 1.91 (s, 3H).

### Step 8: Synthesis of N-(5-amino-7-fluoro-4-oxochroman-8-yl)acetamide (Compound 9d)

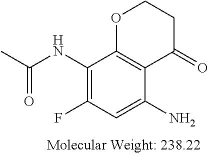 [SMILES: CC(=O)Nc1c(F)cc(N)c2c1OCCC2=O]

### Compound 9d

Molecular sieves (11.33 mg, 0.039 mmol) was added to the solution of Compound 8d (490 mg, 1.912 mmol) in Eaton's Reagent (30 mL) at 20° C. and the reaction mixture was stirred at 50° C. for 3 hr under a N₂ atmosphere. After the reaction finished, the reaction mixture was poured into ice water (100 mL), neutralized with 10% NaOH to a pH of 8~9, and extracted with EA (200 mL*3). The combined organic layers were washed with sat. NaHCO₃ (500 mL*2) and brine (500 mL*1) before being dried over anhydrous sodium sulfate, filtered and concentrated to yield the product Compound 9d (200 mg, 0.840 mmol, yield: 43.90%) which was yellow solid and confirmed by LC-MS (N230872-057-P1-1) and ¹HNMR (N230872-057-P1A, DMSO).

LC-MS: m/z (ES+) (M+H)+=239.4, Rt=0.674 min.

¹HNMR (400 MHz, DMSO) δ 8.89 (s, 1H), 7.55 (s, 2H), 6.10 (d, J=12.5 Hz, 1H), 4.41 (s, 2H), 2.68 (s, 2H), 1.95 (s, 3H).

### Step 9: Synthesis of (S)—N-(9-ethyl-5-fluoro-9-hydroxy-10,13-dioxo-1,2,9,10,13,15-hexahydro-12H-pyrano[4,3,2-de]pyrano[3′,4′:6,7]indolizino[1,2-b]quinolin-4-yl)acetamide (Compound 10d)

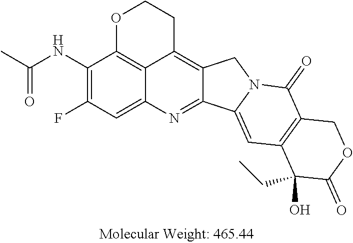 [SMILES: CC[C@@]1(O)C(=O)OCc2c1cc1n(c2=O)Cc2c-1nc1cc(F)c(NC(C)=O)c3c1c2CCO3]

### Compound 10d

Compound 1 (243.12 mg, 0.924 mmol) and PPTS (221.53 mg, 0.882 mmol) was added slowly to the solution of N-(5-amino-7-fluoro-4-oxo-3,4-dihydro-2H-chromen-8-yl)acetamide (200 mg, 0.840 mmol) in toluene (15 mL) at 20° C. and the reaction mixture was stirred at 130° C. for 2 hr under a N₂ atmosphere. After the reaction finished, the reaction mixture was concentrated to remove solvent and used for the next step directly.

### Step 10: Synthesis of CPT-4

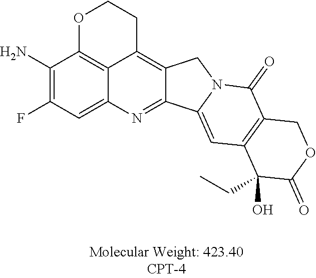 [SMILES: CC[C@@]1(O)C(=O)OCc2c1cc1n(c2=O)Cc2c-1nc1cc(F)c(N)c3c1c2CCO3]

The mixture of N-[(9S)-9-ethyl-5-fluoro-9-hydroxy-10,13-dioxo-1,2,9,10,12,15-hexahydropyrano[4,3,2-de]pyrano[3′,4′:6,7]indolizino[1,2-b]quinolin-4-yl]acetamide (390.76 mg, 0.840 mmol) in 6 M HCl (10 mL) was stirred at 100° C. for 1 hr under a N₂ atmosphere. After the reaction finished, the reaction mixture was concentrated to remove most solvent. The resulting residue was purified by reverse phase column (80 g C18 gel column, A/B=H2O/MeOH; 50%-80% B) to obtain the crude product. The crude product (TLC-T1: DCM:MeOH=10:1, UV, product Rf=0.35) was purified by silica gel column (DCM:MeOH, from 4% to 6%) to yield the desired product CPT-4 (0.6 mg, 0.001 mmol) which was brown solid and confirmed by ¹HNMR (N230872-061-P1-3, DMSO), LC-MS (N230872-061-P1-3) and HPLC (N230872-061-P1-3-254NM, N230872-061-P1-3-214NM).

LC-MS: m/z (ES+) (M+H)+=424.2, Rt=0.618 min.

¹HNMR (400 MHz, DMSO) δ 7.48 (d, J=12.3 Hz, 1H), 7.22 (s, 1H), 6.47 (s, 1H), 5.45 (s, 1H), 5.41 (s, 2H), 5.20 (s, 2H), 4.47 (t, J=5.7 Hz, 2H), 3.30 (s, 2H), 1.94-1.74 (m, 2H), 0.87 (t, J=7.4 Hz, 3H).

### Preparation Example 1.5. Synthesis of (S)—N5-((2R,3R,4R,5S,21S,22R,23R,24R)-1,2,3,4,5,21,22,23,24,25-decahydroxy-8,18-dioxo-13-((3-oxo-3-(((2S,3R,4R,5R)-2,3,4,5,6-pentahydroxyhexyl)amino)propoxy)methyl)-11,15-dioxa-7,19-diazapentacosan-13-yl)-2-(6-(2,5-dioxo-2,5-dihydro-1H-pyrrol-1-yl)hexanamido)-N1-((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)pentanediamide (Compound (II′)-1)

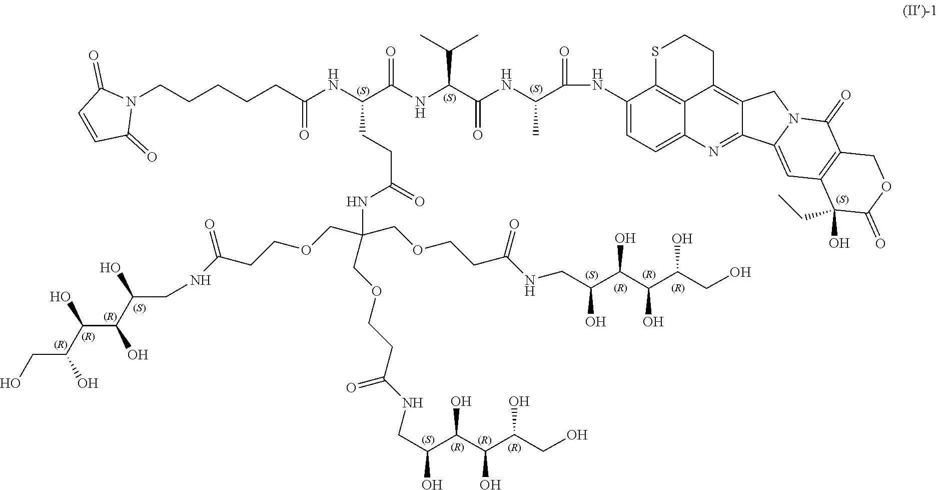

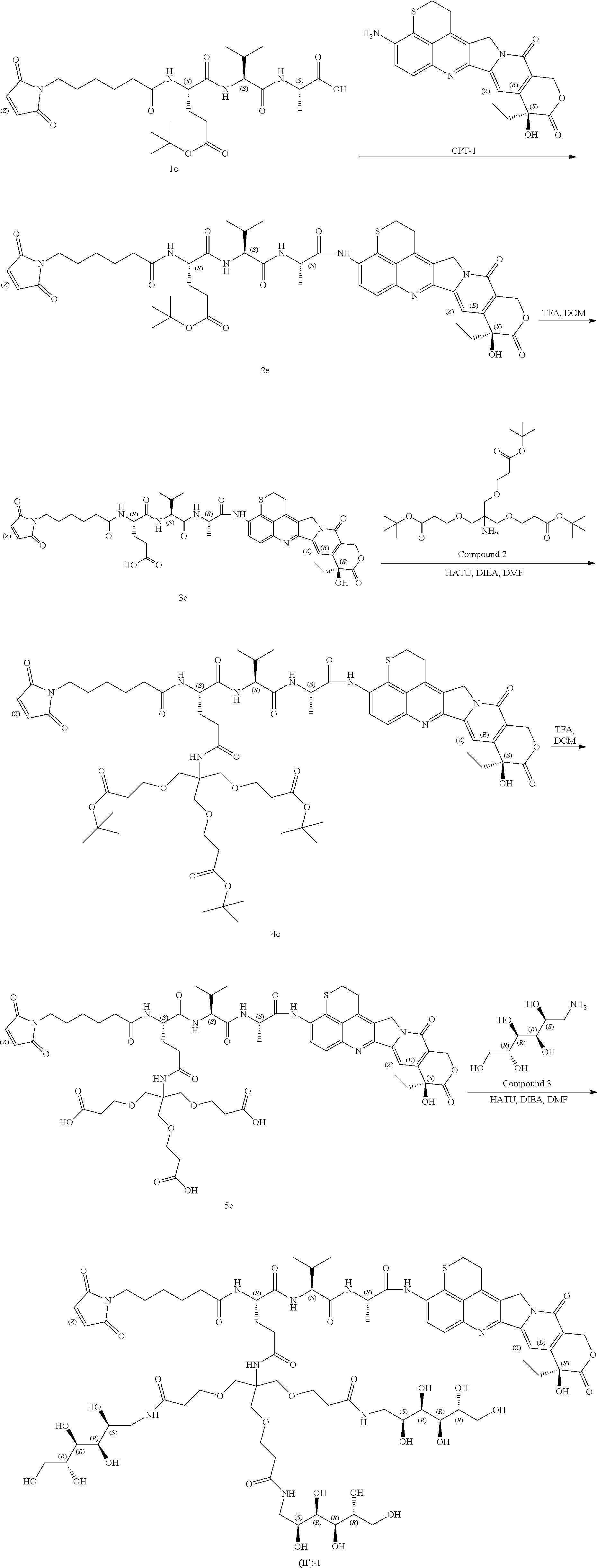

### Step 1: Synthesis of tert-butyl (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-1,2,9,10,13,15-hexahydro-12H-pyrano[3′,4′:6,7]indolizino[1,2-b]thiopyrano[4,3,2-de]quinolin-4-yl)amino)-1-oxopropan-2-yl)amino)-3-methyl-1-oxobutan-2-yl)amino)-5-oxopentanoate (Compound 2e)

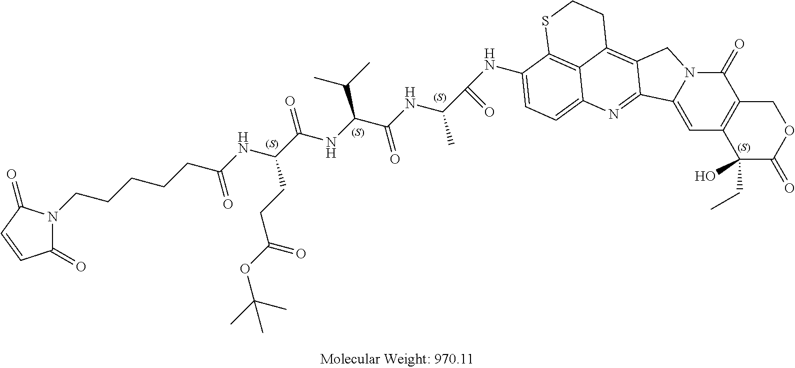

### Compound 2e

1-methylimidazole (0.020 mL, 0.249 mmol) was added to a mixture of N-[(2S,5S)-12-(2,5-dioxo-2,5-dihydro-1H-pyrrol-1-yl)-5-{3-[(2-methylprop-2-yl)oxy]-3-oxopropyl}-1,4,7-trioxo-2-(prop-2-yl)-3,6-diazadodec-1-yl]-L-alanine (Compound 1e, 67.22 mg, 0.119 mmol) and CPT-1 (50 mg, 0.119 mmol) in DMF (2.2 mL). Then, TCFH (39.93 mg, 0.142 mmol) was added to the mixture and the resulting mixture was stirred for 12 hours at 25° C. After the reaction was completed as confirmed by LC-MS, saturated salt water (10 mL) was added to the reaction solution and a large amount of solids were extracted. The resulting mixture was filtered through a cloth funnel and the solids were washed by water before the resulting residue was dissolved in DCM (30 mL). Afterwards, the organic layers were dried over sodium sulfate, filtered and then the filtrate was concentrated under reduced pressure to yield Compound 2e (70 mg, brown solid, yield: 60.82%) which was used to next step without purification.

LC-MS: (ESI) m/z (M+H), 971.2.

### Step 2: Synthesis of (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-1,2,9,10,13,15-hexahydro-12H-pyrano[3′,4′:6,7]indolizino[1,2-b]thiopyrano[4,3,2-de]quinolin-4-yl)amino)-1-oxopropan-2-yl)amino)-3-methyl-1-oxobutan-2-yl)amino)-5-oxopentanoic acid (Compound 3e)

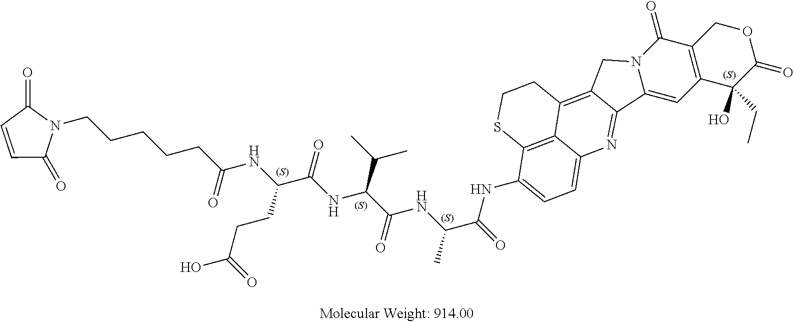

### Compound 3e

TFA (0.5 mL, 0.067 mmol) was added to a solution of 2-methylpropan-2-yl (4S)-4-{[6-(2,5-dioxo-2,5-dihydro-1H-pyrrol-1-yl)-1-oxohexyl]amino}-5-{[(2S)-1-{[(2S)-1-{[(9S)-9-ethyl-9-hydroxy-10,13-dioxo-1,2,9,10,12,15-hexahydrothiino[4,3,2-de]pyrano[3′,4′:6,7]indolizino[1,2-b]quinolin-4-yl]amino}-1-oxoprop-2-yl]amino}-3-methyl-1-oxobut-2-yl]amino}-5-oxopentanoate (65 mg, 0.067 mmol) in DCM (2.5 mL) at 0° C. for 5 minutes. The resulting mixture was then naturally warmed up to 15° C. and stirred for 1 hour. After the reaction was completed as confirmed by LC-MS, the resulting mixture was concentrated to yield Compound 3e which was used to next step without purification (61.24 mg, yellow solid, quantitative yield).

LC-MS: (ESI) m/z (M+H), 914.4.

### Step 3: Synthesis of tert-butyl (2S,5S,8S)-13,13-bis((3-(tert-butoxy)-3-oxopropoxy)methyl)-8-(6-(2,5-dioxo-2,5-dihydro-1H-pyrrol-1-yl)hexanamido)-1-(((S)-9-ethyl-9-hydroxy-10,13-dioxo-1,2,9,10,13,15-hexahydro-12H-pyrano[3′,4′:6,7]indolizino[1,2-b]thiopyrano[4,3,2-de]quinolin-4-yl)amino)-5-isopropyl-2-methyl-1,4,7,11-tetraoxo-15-oxa-3,6,12-triazaoctadecan-18-oate (Compound 4e)

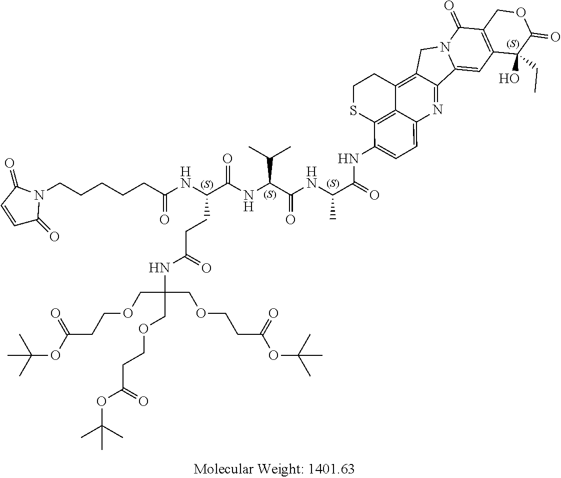

### Compound 4e

A mixture of Compound 3e (61 mg, 0.067 mmol), HATU (30.45 mg, 0.080 mmol), HOBT (10.82 mg, 0.080 mmol) and DIEA (0.033 mL, 0.200 mmol) in DMF (2 mL) was stirred at 0° C. for 5 minutes before adding 2-methylpropan-2-yl 3-{[9-amino-9-(7,7-dimethyl-5-oxo-2,6-dioxaoct-1-yl)-2,2-dimethyl-4-oxo-3,7-dioxadec-10-yl]oxy}propanoate (Compound 2, 33.75 mg, 0.067 mmol) to the reaction mixture. The resulting mixture was naturally warmed up to 15° C. and stirred for 1 hour until the reaction completed as confirmed by LC-MS. Afterwards, saturated salt water (3 mL) was added to the reaction solution and a large amount of solids were extracted. The resulting mixture was then filtered through a cloth funnel and the solids were washed by water before the residue being dissolved in DCM (10 mL). The organic layers were dried over sodium sulfate, filtered, and the filtrate was concentrated under reduced pressure to yield Compound 4e (90 mg, brown solid, yield: 96.2%) which was used to next step without purification.

LC-MS: (ESI) m/z (M+H), 617.3.

### Step 4: Synthesis of (2S,5S,8S)-13,13-bis((2-carboxyethoxy)methyl)-8-(6-(2,5-dioxo-2,5-dihydro-1H-pyrrol-1-yl)hexanamido)-1-(((S)-9-ethyl-9-hydroxy-10,13-dioxo-1,2,9,10,13,15-hexahydro-12H-pyrano[3′,4′:6,7]indolizino[1,2-b]thiopyrano[4,3,2-de]quinolin-4-yl)amino)-5-isopropyl-2-methyl-1,4,7,11-tetraoxo-15-oxa-3,6,12-triazaoctadecan-18-oic acid (Compound 5e)

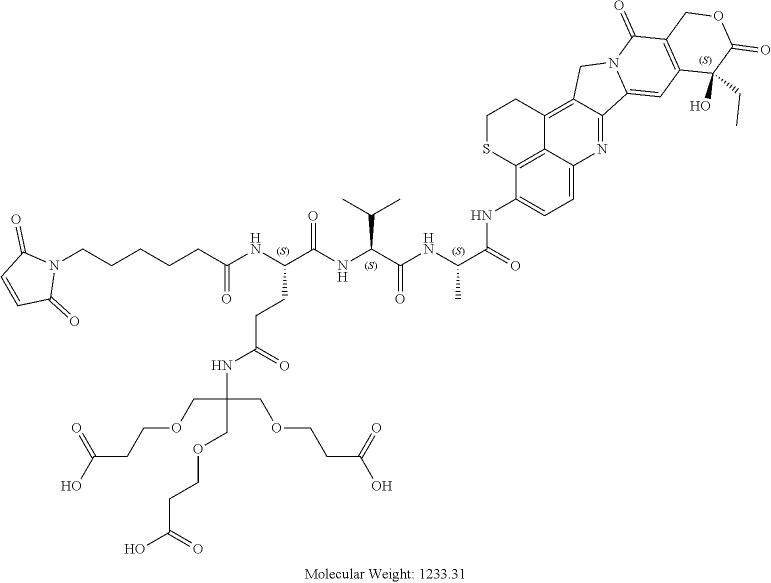

### Compound 5e

TFA (0.5 mL, 0.064 mmol) in DCM (2.5 mL) was added to a solution of 2-methylpropan-2-yl 3-{[(2S,5S,8S)-13,13-bis(7,7-dimethyl-5-oxo-2,6-dioxaoct-1-yl)-8-{[6-(2,5-dioxo-2,5-dihydro-1H-pyrrol-1-yl)-1-oxohexyl]amino}-1-{[(9S)-9-ethyl-9-hydroxy-10,13-dioxo-1,2,9,10,12,15-hexahydrothiino[4,3,2-de]pyrano[3′,4′:6,7]indolizino[1,2-b]quinolin-4-yl]amino}-2-methyl-1,4,7,11-tetraoxo-5-(prop-2-yl)-3,6,12-triazatetradec-14-yl]oxy}propanoate (90 mg, 0.064 mmol) in DCM (2.5 mL) at 0° C. for 5 minutes. The resulting mixture was then naturally warmed to 15° C. and stirred for 1 hour before removing the solvent under vacuum. The resulting residue was used to next step without purification (80 mg, yellow solid, quantitative yield).

LC-MS: (ESI) m/z (½M+H), 617.2.

### Step 5: Synthesis of Compound (II′)-1

A mixture of 3-{[(2S,5S,8S)-13,13-bis{[(2-carboxyethyl)oxy]methyl}-8-{[6-(2,5-dioxo-2,5-dihydro-1H-pyrrol-1-yl)-1-oxohexyl]amino}-1-{[(9S)-9-ethyl-9-hydroxy-10,13-dioxo-1,2,9,10,12,15-hexahydrothiino[4,3,2-de]pyrano[3′,4′:6,7]indolizino[1,2-b]quinolin-4-yl]amino}-2-methyl-1,4,7,11-tetraoxo-5-(prop-2-yl)-3,6,12-triazatetradec-14-yl]oxy}propanoic acid (80 mg, 0.065 mmol), HATU (76.46 mg, 0.201 mmol), HOBT (27.17 mg, 0.201 mmol) and DIEA (0.064 mL, 0.389 mmol) in DMF (2 mL) was stirred at 0° C. for 20 minutes and (2R,3R,4R,5S)-6-aminohexane-1,2,3,4,5-pentol (Compound 3, 36.43 mg, 0.201 mmol) was then added to the mixture. The resulting mixture was naturally warmed up to 15° C. and stirred for 1 hour. The solvent was removed under vacuum and the residue was purified by Prep-HPLC (TFA) to afford the title Compound (II′)-1 (11 mg, yellow solid, yield: 9.84%).

LC-MS: (ESI) m/z (½M+H), 862.2.

¹HNMR (400 MHz, DMSO): δ 9.69 (s, 1H), 8.35 (d, J=6.4 Hz, 1H), 8.02 (d, J=8.0 Hz, 1H), 7.91 (d, J=9.2 Hz, 1H), 7.78-7.76 (m, 4H), 7.58 (d, J=9.2 Hz, 1H), 7.32 (s, 1H), 7.13 (s, 1H), 6.99 (s, 2H), 6.52 (s, 1H), 5.44 (s, 2H), 5.29 (s, 2H), 4.62-4.54 (m, 2H), 4.29-4.21 (m, 4H), 3.58-3.55 (m, 22H), 3.50-3.43 (m, 11H), 3.28-3.23 (m, 11H), 3.13-2.96 (m, 5H), 2.33 (t, J=6.4 Hz, 6H), 2.25-1.92 (m, 8H), 1.91-1.83 (m, 3H), 1.69-1.65 (m, 1H), 1.55-1.46 (m, 4H), 1.42-1.40 (m, 3H), 1.32-1.20 (m, 4H), 0.90-0.83 (m, 9H).

### Preparation Example 1.6. Synthesis of (S)—N5-((2R,3R,4R,5S,21S,22R,23R,24R)-1,2,3,4,5,21,22,23,24,25-decahydroxy-8,18-dioxo-13-((3-oxo-3-(((2S,3R,4R,5R)-2,3,4,5,6-pentahydroxyhexyl)amino)propoxy)methyl)-11,15-dioxa-7,19-diazapentacosan-13-yl)-2-(6-(2,5-dioxo-2,5-dihydro-1H-pyrrol-1-yl)hexanamido)-N—((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)pentanediamide (Compound(II′)-2)

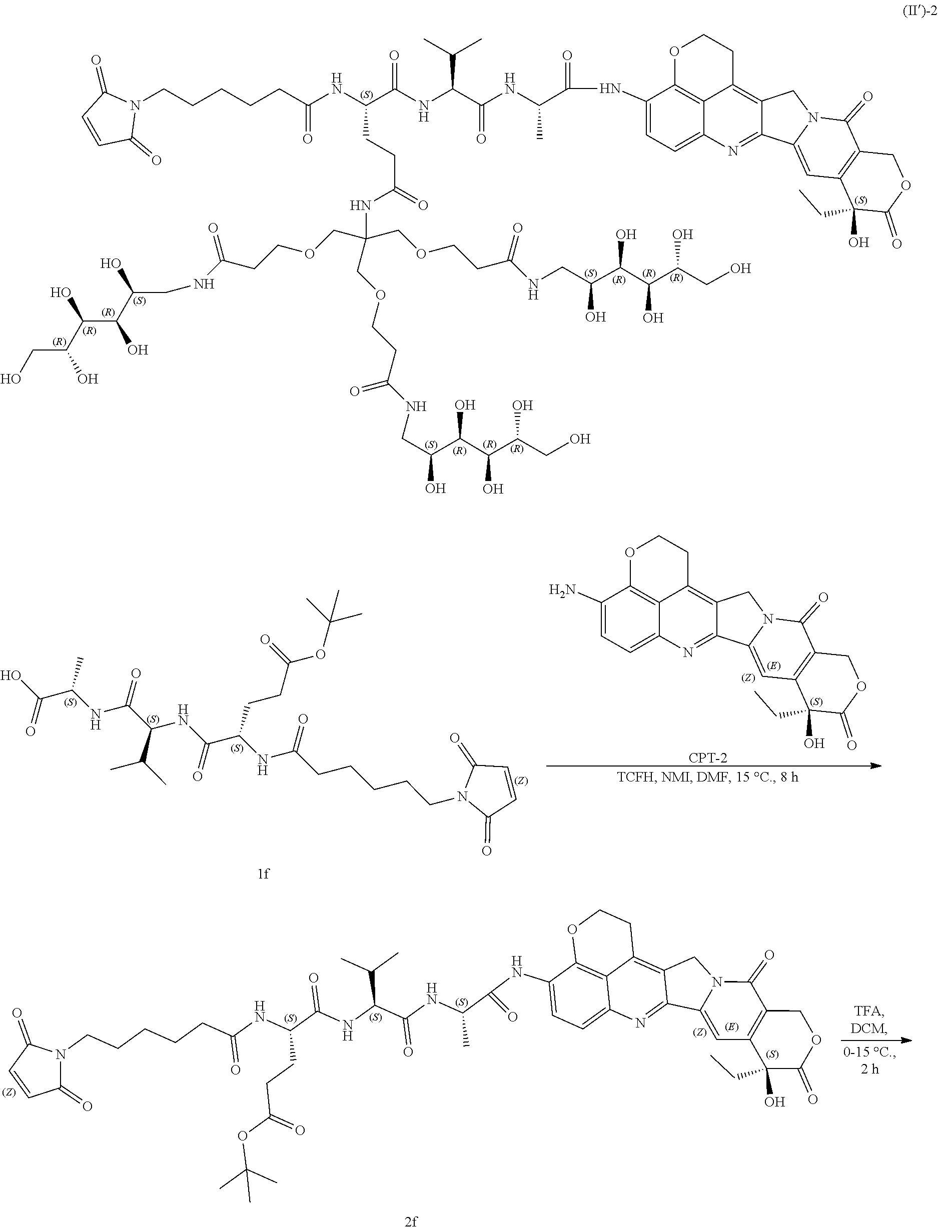

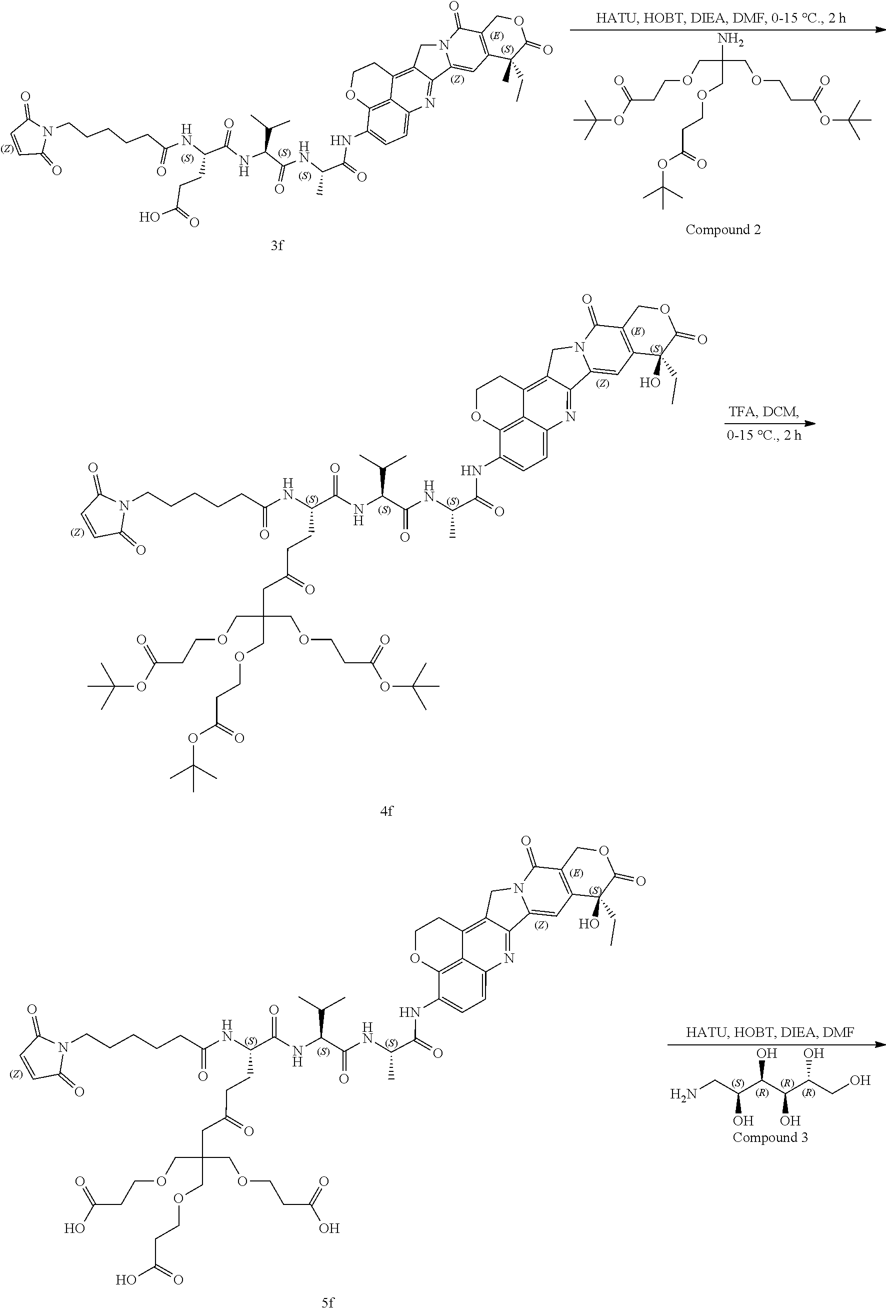

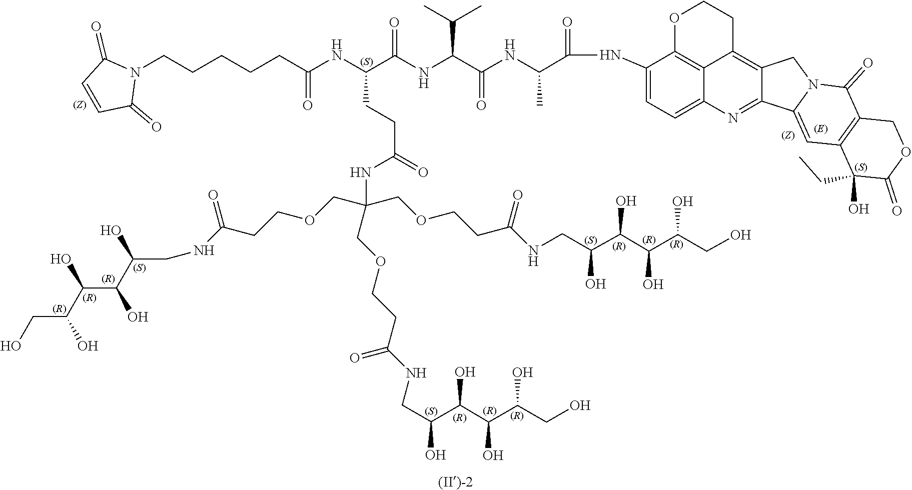

### Step 1: Synthesis of tert-butyl (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoate (Compound 2f)

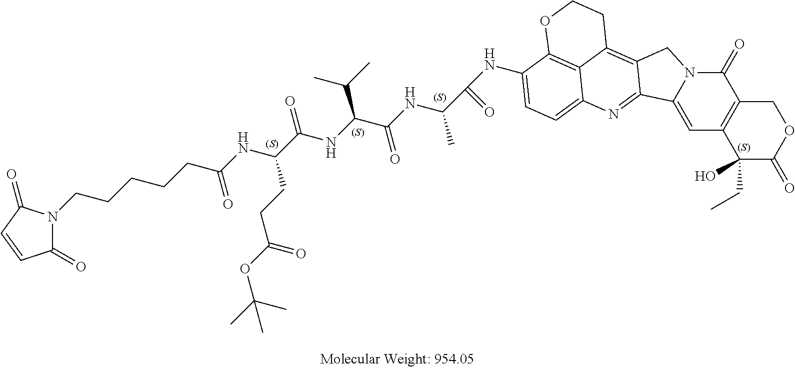

1-methylimidazole (0.043 mL, 0.537 mmol) was added to a mixture of N-[(2S,5S)-12-(2,5-dioxo-2,5-dihydro-1H-pyrrol-1-yl)-5-{3-[(2-methylprop-2-yl)oxy]-3-oxopropyl}-1,4,7-trioxo-2-(prop-2-yl)-3,6-diazadodec-1-yl]-L-alanine (Compound if, 166.82 mg, 0.294 mmol) and CPT-2 (115 mg, 0.285 mmol) in DMF (1.9 mL) and then TCFH (86.04 mg, 0.307 mmol) was added to the mixture. Then, the resulting mixture was stirred for 12 hours at 25° C. After the reaction was completed as confirmed by LC-MS, saturated salt water (6 mL) was added to the reaction solution and a large amount of solids were extracted. The resulting mixture was then filtered through a cloth funnel and the solids were washed by water. Afterwards, the residue was dissolved in DCM (30 mL). The organic layers were dried over sodium sulfate, filtered, and the filtrate was concentrated under reduced pressure to obtain the title Compound 2f which was used to next step without purification (155 mg, brown solid, yield: 63.6%).

LC-MS: (ESI) m/z (M+H), 954.5.

### Step 2: Synthesis of (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoic acid (Compound 3f)

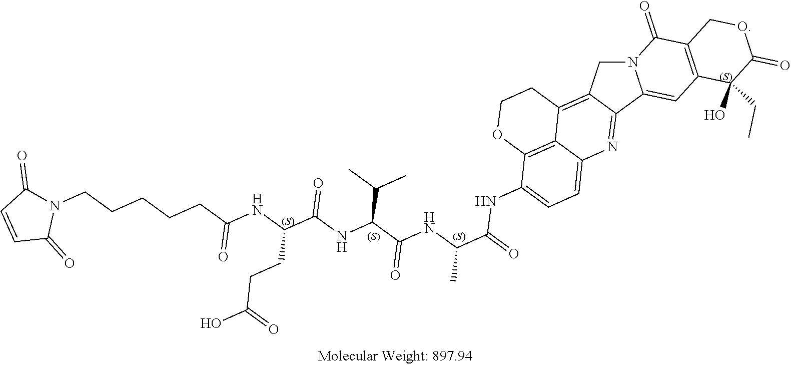

2,2,2-trifluoroacetic acid (0.85 mL, 8.1 mmol) was added to a solution of Compound 2f (155 mg, 0.162 mmol) in DCM (4.25 mL) at 0° C. for 5 minutes and the resulting mixture was naturally warmed up to 15° C. and stirred for 2 hours. After the reaction was completed as confirmed by LC-MS, the mixture was concentrated to yield the title Compound 3f which was used to next step without purification (145.88 mg, yellow solid, quantitative yield).

LC-MS: (ESI) m/z (M+1), 898.6.

### Step 3: Synthesis of tert-butyl (2S,5S,8S)-13,13-bis((3-(tert-butoxy)-3-oxopropoxy)methyl)-8-(6-(2,5-dioxo-2,5-dihydro-1H-pyrrol-1-yl)hexanamido)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oate (Compound 4f)

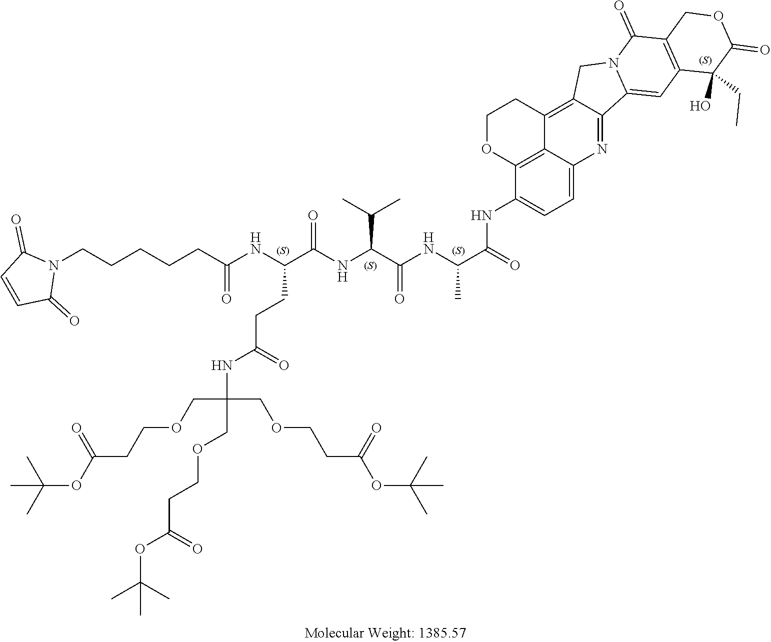

A mixture of Compound 3f (145.88 mg, 0.162 mmol), HATU (74.13 mg, 0.195 mmol), HOBT (26.34 mg, 0.195 mmol) and DIEA (0.081 mL, 0.487 mmol) in DMF (4 mL) was stirred at 0° C. for 5 minutes. 2-methylpropan-2-yl 3-{[9-amino-9-(7, 7-dimethyl-5-oxo-2, 6-dioxaoct-1-yl)-2, 2-dimethyl-4-oxo-3, 7-dioxadec-10-yl]oxy}propanoate (Compound 2, 82.15 mg, 0.162 mmol) was then added to the mixture and the resulting mixture was naturally warmed up to 15° C. and stirred for 1 hour. After the reaction was completed as confirmed by LC-MS, saturated salt water (12 mL) was added to the reaction solution and a large amount of solids were extracted. Then, the mixture was filtered through a cloth funnel and the solids were washed by water followed by the resulting residue being dissolved in DCM (10 mL). Afterwards, the organic layers were dried over sodium sulfate, filtered, and the filtrate was concentrated under reduced pressure to yield the title Compound 4f which was used to next step without purification (188 mg, brown solid, yield: 83.5%).

LC-MS: (ESI) m/z (M+1), 1386.6.

### Step 4: Synthesis of (2S,5S,8S)-13,13-bis((2-carboxyethoxy)methyl)-8-(6-(2,5-dioxo-2,5-dihydro-1H-pyrrol-1-yl)hexanamido)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oic acid (Compound 51)

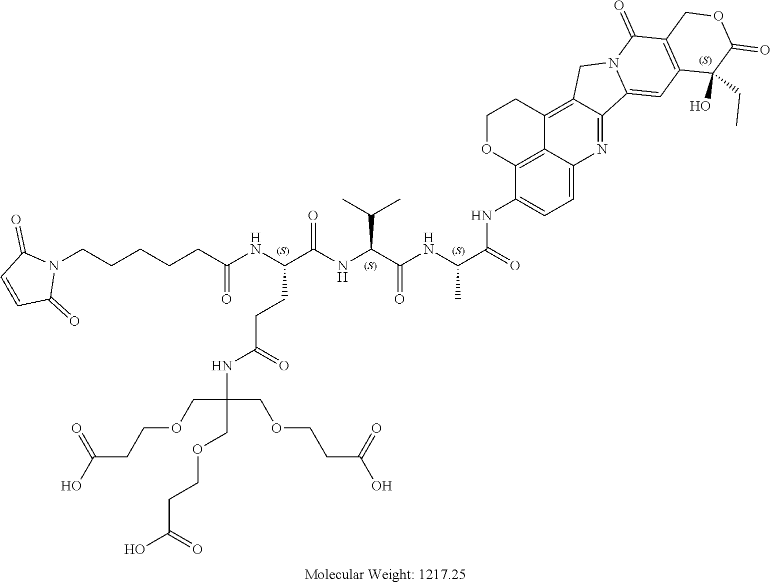

2,2,2-trifluoroacetic acid (0.769 mL, 7.327 mmol) was added to a solution of Compound 4f (188 mg, 0.122 mmol) in DCM (3.18 mL) at 0° C. for 5 minutes. The resulting mixture was then naturally warmed up to 15° C. and stirred for 2 hours. After the reaction was completed as confirmed by LC-MS, the mixture was concentrated to yield Compound 5f which was used to next step without purification (148.65 mg, yellow solid, quantitative yield).

LC-MS: (ESI) m/z (M+1), 1217.6.

### Step 5: Synthesis of Compound (II′)-2

A mixture of Compound 5f (133.785 mg, 0.110 mmol), HATU (158.81 mg, 0.418 mmol), HOBT (56.44 mg, 0.418 mmol) and DIEA (0.145 mL, 0.879 mmol) in DMF (2.8 mL) was stirred at 0° C. for 20 minutes. (2R, 3R, 4R, 5S)-6-aminohexane-1, 2, 3, 4, 5-pentol (Compound 3, 75.67 mg, 0.418 mmol) was then added to the mixture. The resulting mixture was naturally warmed up to 15° C. and stirred for 1 hour before the solvent was removed under vacuum and the residue was purified by Prep-HPLC (TFA) to yield the title compound (II′)-2 (20.0 mg, yellow solid, yield: 12.0%).

LC-MS: (ESI) m/z 1706.8. 1707.8 (M+H).

¹HNMR (400 MHz, DMSO): δ 9.51 (s, 1H), 8.48-8.44 (m, 2H), 8.02 (d, J=7.6 Hz, 1H), 7.78 (t, J=5.2 Hz, 3H), 7.73 (d, J=9.6 Hz, 1H), 7.59 (d, J=8.8 Hz, 1H), 7.32 (s, 1H), 7.13 (s, 1H), 6.99 (s, 2H), 6.49 (s, 1H), 5.44 (s, 2H), 5.28 (s, 2H), 4.64-4.60 (m, 1H), 4.54 (t, J=5.6 Hz, 2H), 4.30-4.20 (m, 4H), 3.61-3.46 (m, 12H), 3.49-3.45 (m, 10H), 3.42-3.36 (m, 17H), 3.33-3.24 (m, 8H), 3.07-2.97 (m, 5H), 2.35-2.32 (m, 6H), 2.13-2.10 (m, 4H), 2.04-2.00 (m, 1H), 1.91-1.84 (m, 3H), 1.71-1.65 (m, 1H), 1.51-1.46 (m, 5H), 1.37 (d, J=7.2 Hz, 3H), 1.24-1.18 (m, 2H), 0.91-0.84 (m, 9H).

### Preparation Example 1.7. Synthesis of (2S,5S,8S)-13,13-bis((2-carboxyethoxy)methyl)-8-(6-(2,5-dioxo-2,5-dihydro-1H-pyrrol-1-yl) hexanamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oic acid (Compound(II′)-3)

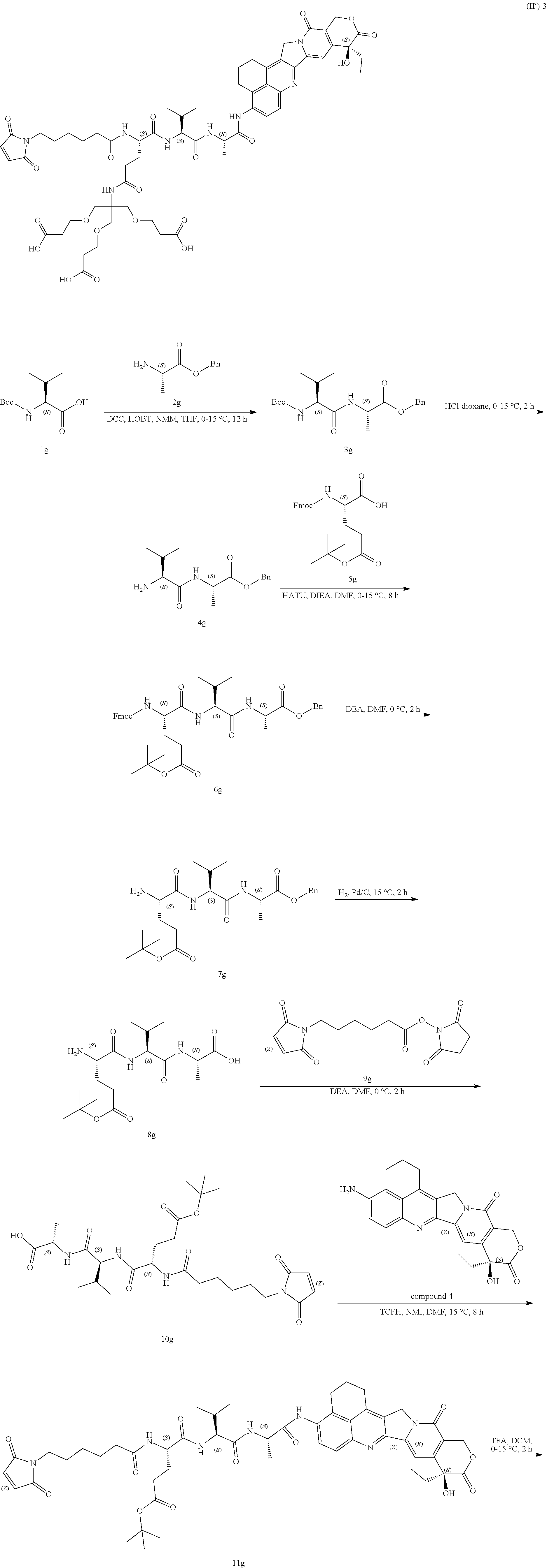

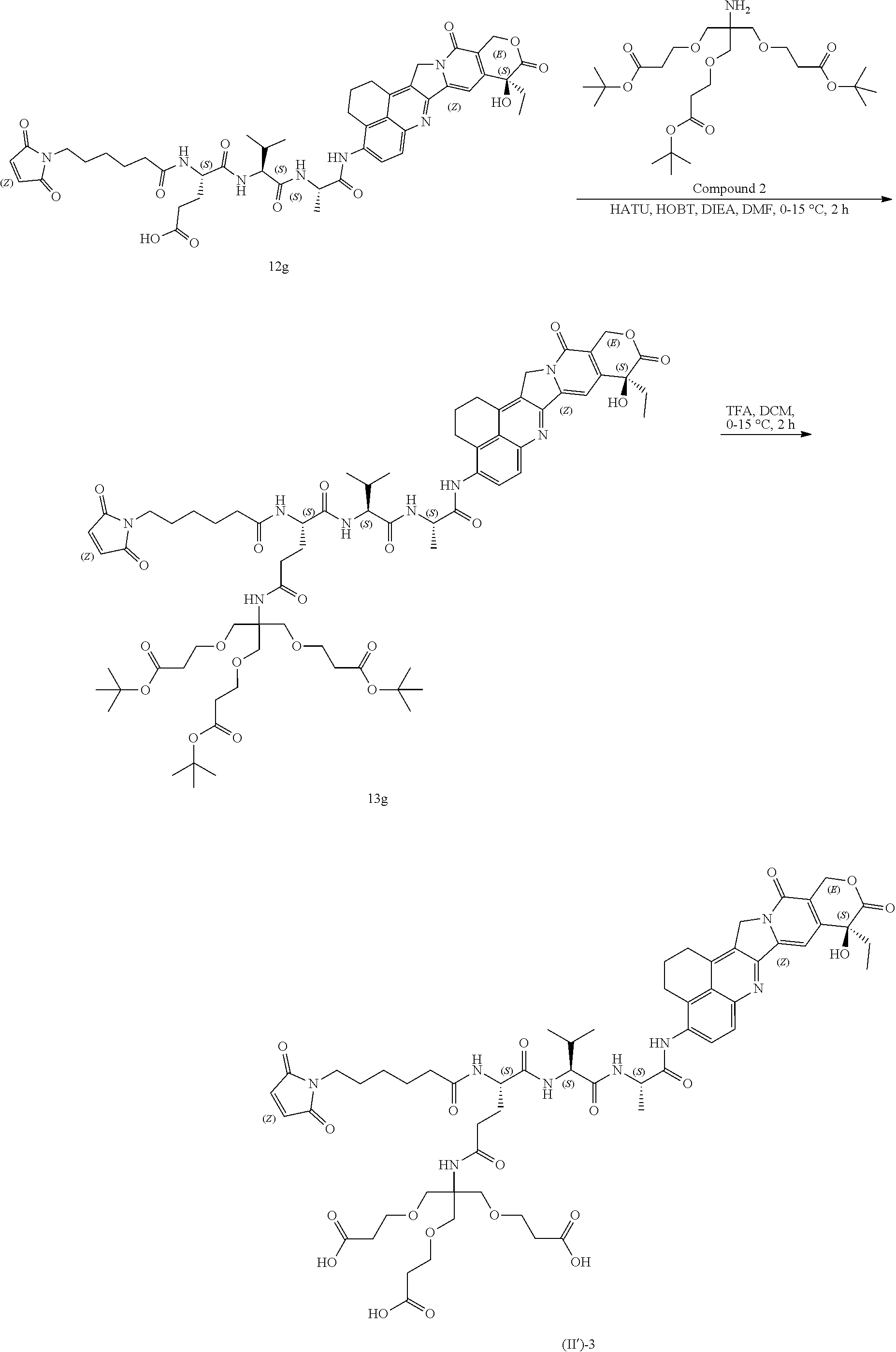

### Step 1: Synthesis of benzyl (tert-butoxycarbonyl)-L-valyl-L-alaninate (Compound 3g)

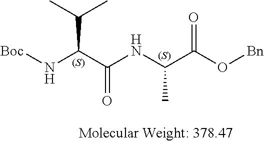

### Compound 3g

A mixture of N-{[(2-methylprop-2-yl) oxy]carbonyl}-L-valine (Compound 1g, 5 g, 23.014 mmol), DCC (5.70 g, 27.617 mmol), HOBT (3.27 g, 24.165 mmol) in THF (40 mL) was stirred at 0° C. for 30 minutes. Benzyl DL-alaninate hydrochloride (Compound 2g, 4.47 g, 20.713 mmol) and NMM (3.793 mL, 34.521 mmol) in THF (40 mL) was then added to the mixture. The resulting mixture was then naturally warmed up to 15° C. and stirred for 12 hours. After the reaction was completed as confirmed by TLC and LC-MS, the mixture was concentrated at reduced pressure and the residue was dissolved in DCM (200 mL) and the resulting solution was then filtered. Then, the filtrate was partitioned between DCM (100*2 mL) and water (50 mL) followed by the aqueous layer being extracted with DCM (50 mL). Afterwards, the combined organic layers were dried over sodium sulfate, filtered, and the resulting filtrate was concentrated under reduced pressure. Finally, the residue was purified by flash chromatography (160 g silica gel column, PE/DCM with DCM from 0-100% DCM/MeOH with MeOH from 0-5%) to yield Compound 3g (6.0 g, white solid, yield: 68.9%).

LC-MS: (ESI) m/z (M+Na), 401.2.

### Step 2: Synthesis of benzyl L-valyl-L-alaninate (Compound 4g)

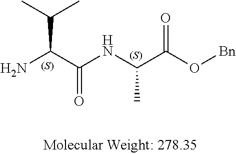

### Compound 4g

Hydrogen chloride (61.564 mL, 246.255 mmol) was added to a solution of Compound 3g (4.66 g, 12.313 mmol) in 1,4-dioxane (62 mL) at 0° C. and the mixture was then stirred at 0° C. for 2 hours. After the reaction was completed as confirmed by LC-MS, the resulting mixture was concentrated to yield Compound 4g which was used to next step without purification (3.42 g, white solid, quantitative yield).

### Step 3: Synthesis of benzyl (5S,8S,11S)-5-(3-(tert-butoxy)-3-oxopropyl)-1-(9H-fluoren-9-yl)-8-isopropyl-11-methyl-3,6,9-trioxo-2-oxa-4,7,10-triazadodecan-12-oate(Compound 6g)

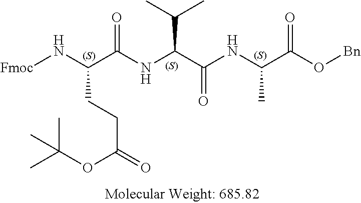

### Compound 6g

A mixture of (2S)-2-({[(9H-fluoren-9-ylmethyl)oxy]carbonyl}amino)-5-[(2-methylprop-2-yl)oxy]-5-oxopentanoic acid (Compound 4g, 5.20 g, 12.215 mmol), HATU (4.88 g, 12.826 mmol), DIEA (6.057 mL, 36.645 mmol) in DMF (40 mL) was stirred at 0° C. for 30 minutes and Benzyl N-[(2S)-2-amino-3-methyl-1-oxobutyl]-L-alaninate (Compound 5g, 3.4 g, 12.215 mmol) was then added to the mixture. The resulting mixture was then naturally warmed up to 15° C. and stirred for 12 hours. Upon the reaction completed as confirmed by LC-MS, the mixture was concentrated at reduced pressure and the resulting residue was partitioned between DCM (120 mL) and water (40 mL) followed by the aqueous layer being extracted with DCM (40 mL). Afterwards, the combined organic layers were dried over sodium sulfate, filtered, and the resulting filtrate was concentrated under reduced pressure. Finally, the residue was purified by flash chromatography (30 g silica gel column, PE/DCM with DCM from 0-100% DCM/MeOH with MeOH from 0~2%) to yield the title product Compound 6g (6.5 g, white solid, yield: 77.6%).

LC-MS: (ESI) m/z (M+Na), 708.4.

### Step 4: Synthesis of tert-butyl (S)-4-amino-5-(((S)-1-(((S)-1-(benzyloxy)-1-oxopropan-2-yl) amino)-3-methyl-1-oxobutan-2-yl)amino)-5-oxopentanoate (Compound 7g)

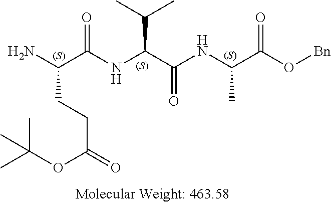

### Compound 7g

Diethylamine (12.641 mL, 122.190 mmol) was added to a solution of benzyl N-[(5S,8S)-1-(9H-fluoren-9-yl)-5-{3-[(2-methylprop-2-yl)oxy]-3-oxopropyl}-3,6,9-trioxo-8-(prop-2-yl)-4,7-diaza-2-oxanon-9-yl]-L-alaninate (8.38 g, 12.219 mmol) in DMF (12 mL) at 0° C. and the resulting mixture was stirred for 2 hours at 0° C. After the reaction was completed as confirmed by LC-MS, the mixture was concentrated at reduced pressure and the resulting residue was dissolved in DMF (6 mL). Then, the resulting solution was purified by reverse phase column (120 g C18 gel column, H₂O/CH₃CN with CH₃CN from 0~54%) to yield Compound 7g (3.6 g, white solid, yield: 37.2%).

LC-MS: (ESI) m/z (M+Na), 464.2.

### Step 5: Synthesis of ((2S, 5S)-5-amino-8-(tert-butoxy)-2-isopropyl-4,8-dioxooctanoyl)-L-alanine (Compound 8g)

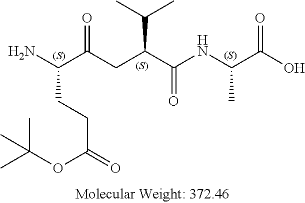

### Compound 8g

Palladium (0) (1.03 g, 0.971 mmol) was added to a solution of benzyl N-[(7S, 10 S)-7-amino-2,2-dimethyl-4,8,11-trioxo-10-(prop-2-yl)-9-aza-3-oxaundec-11-yl]-L-alaninate (1.5 g, 3.236 mmol) in MeOH (60 mL) and the mixture was stirred for 2 hours at 15° C. After the reaction was completed as confirmed by LC-MS, the mixture was filtered and the resulting filtrate was concentrated to yield the title Compound 8g which was used to next step without purification (1.2 g, white solid, quantitative yield).

LC-MS: (ESI) m/z (M+H), 374.2.

### Step 6: Synthesis of ((S)-5-(tert-butoxy)-2-(6-(2,5-dioxo-2,5-dihydro-1H-pyrrol-1-yl) hexanamido)-5-oxopentanoyl)-L-valyl-L-alanine (Compound 10g)

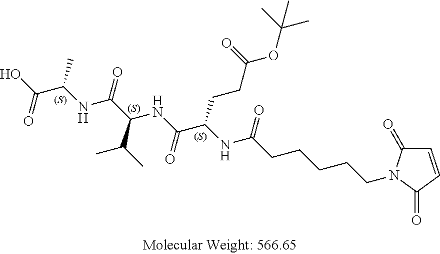

### Compound 10g

A mixture of N-[(7S,10S)-7-amino-2,2-dimethyl-4,8,11-trioxo-10-(prop-2-yl)-9-aza-3-oxaundec-11-yl]-L-alanine (400 mg, 1.071 mmol) and DIEA (0.531 mL, 3.213 mmol) in DMF (8 mL) was stirred at 0° C. for 5 minutes. 1-{6-[(2, 5-dioxotetrahydro-1H-pyrrol-1-yl) oxy]-6-oxohexyl}pyrrole-2, 5-dione (Compound 9g, 495.31 mg, 1.607 mmol) was added to the mixture and the resulting mixture was stirred for 1 hour at 0° C. After the reaction was completed as confirmed by LC-MS, the reaction solvent was removed under vacuum and the residue was purified by Prep-HPLC (C18, 20-60% acetonitrile in H₂O with 0.1% formic acid) to yield Compound 10g (265 mg, white solid, 44.5% yield).

LC-MS: (ESI) m/z (M+1), 567.4.

### Step 7: Synthesis of tert-butyl (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoate (Compound 11g)

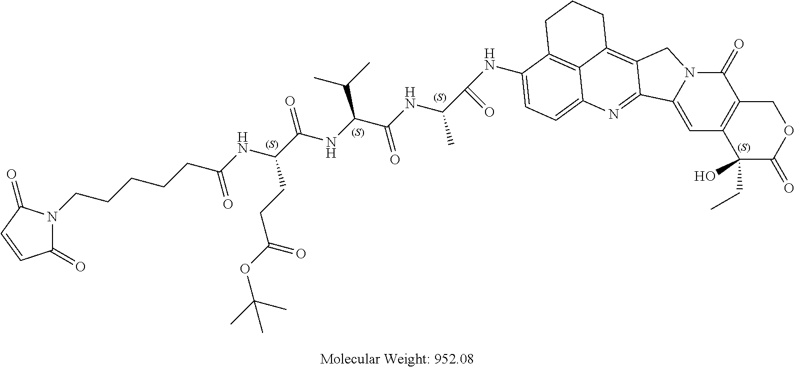

### Compound 11g

1-methylimidazole (0.048 mL, 0.599 mmol) was added to a mixture of N-[(2S,5S)-12-(2,5-dioxo-2,5-dihydro-1H-pyrrol-1-yl)-5-{3-[(2-methylprop-2-yl)oxy]-3-oxopropyl}-1,4,7-trioxo-2-(prop-2-yl)-3,6-diazadodec-1-yl]-L-alanine (228.03 mg, 0.342 mmol) and (9S)-4-amino-9-ethyl-9-hydroxy-1,2,3,9,10,12,13,15-octahydrocyclohexa[1,2,3-de]pyrano[3′,4′:6,7]indolizino[1,2-b]quinoline-10,13-dione (Compound 4, 115 mg, 0.285 mmol) in DMF (2.2 mL). TCFH (95.97 mg, 0.342 mmol) was then added to the mixture and the resulting mixture was stirred for 12 hours at 25° C. After the reaction was completed as confirm by LC-MS, saturated salt water (10 mL) was added to the reaction solution and a large amount of solids were extracted. Afterwards, the resulting mixture was filtered through a cloth funnel and the solids were washed by water before the residue was dissolved in DCM (30 mL). Finally, the organic layers were dried over sodium sulfate, filtered, and the filtrate was concentrated under reduced pressure to yield the title Compound 11g which was used to next step without purification (240 mg, brown solid, yield: 88.4%).

LC-MS: (ESI) m/z (M+H), 952.6.

### Step 8: Synthesis of (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoic acid (Compound 12g)

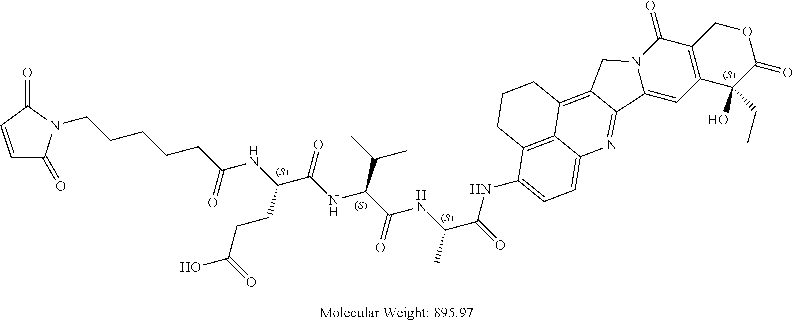

### Compound 12g

2,2,2-trifluoroacetic acid (0.132 mL, 1.260 mmol) was added to a solution of 2-methylpropan-2-yl (4S)-4-{[6-(2,5-dioxo-2,5-dihydro-1H-pyrrol-1-yl)-1-oxohexyl]amino}-5-{[(2S)-1-{[(2S)-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-1-oxoprop-2-yl]amino}-3-methyl-1-oxobut-2-yl]amino}-5-oxopentanoate (20 mg, 0.021 mmol) in DCM (0.65 mL) at 0° C. for 5 minutes. Then, the resulting mixture was naturally heated to 15° C. and stirred for 1 hour. After the reaction was completed as confirmed by LC-MS, the mixture was concentrated to yield Compound 12g which was used to next step without purification (18.82 mg, yellow solid, quantitative yield).

LC-MS: (ESI) m/z (M+1), 896.6.

### Step 9: Synthesis of tert-butyl (2S,5S,8S)-13,13-bis((3-(tert-butoxy)-3-oxopropoxy)methyl)-8-(6-(2,5-dioxo-2,5-dihydro-1H-pyrrol-1-yl)hexanamido)-1-(((S)-9-ethyl-9-hydroxy-10,13-dioxo-2,3,9,10,13,15-hexahydro-1H,12H-benzo [de] pyrano [3′,4′:6,7]indolizino[1,2-b]quinolin-4-yl)amino)-5-isopropyl-2-methyl-1,4,7,11-tetraoxo-15-oxa-3,6,12-triazaoctadecan-18-oate (Compound 13g)

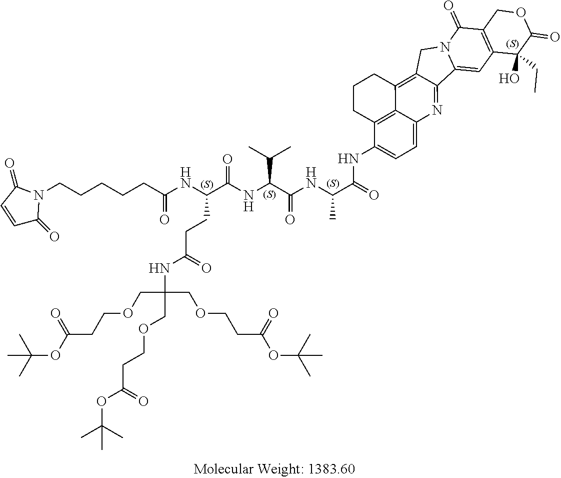

### Compound 13g

A mixture of (4S)-4-{[6-(2,5-dioxo-2,5-dihydro-1H-pyrrol-1-yl)-1-oxohexyl]amino}-5-{[(2S)-1-{[(2S)-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-1-oxoprop-2-yl]amino}-3-methyl-1-oxobut-2-yl]amino}-5-oxopentanoic acid (18.82 mg, 0.021 mmol), HATU (9.58 mg, 0.025 mmol), HOBT (3.41 mg, 0.025 mmol) and DIEA (0.010 mL, 0.063 mmol) in DMF (0.5 mL) was stirred at 0° C. for 5 minutes. Then, Compound 2 (10.62 mg, 0.021 mmol) was added to the mixture before the mixture was naturally warmed up to 15° C. and stirred for 1 hour. After the reaction was completed as confirmed by LC-MS, saturated salt water (3 mL) was added to the reaction solution and a large amount of solids were extracted. The resulting mixture was filtered through a cloth funnel and the solids were washed by water. Afterwards, the residue was dissolved in DCM (10 mL) before the organic layers were then dried over sodium sulfate, filtered, and the filtrate was concentrated under reduced pressure to yield the title Compound 13g which was used to next step without purification (28 mg, brown solid, yield: 96.3%).

### Step 10: Synthesis of (II′)-3

2,2,2-trifluoroacetic acid (0.057 mL, 0.540 mmol) was added to a solution of 2-methylpropan-2-yl 3-{[(2S,5S,8S)-13,13-bis(7,7-dimethyl-5-oxo-2,6-dioxaoct-1-yl)-8-{[6-(2,5-dioxo-2,5-dihydro-1H-pyrrol-1-yl)-1-oxohexyl]amino}-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-2-methyl-1,4,7,11-tetraoxo-5-(prop-2-yl)-3,6,12-triazatetradec-14-yl]oxy}propanoate (12 mg, 0.009 mmol) in DCM (0.345 mL) at 0° C. for 5 minutes and the mixture was then naturally heated to 15° C. and stirred for 1 hour. Afterwards, the solvent was removed under vacuum and the residue was purified by Prep-HPLC (TFA) to yield Compound (II′)-3 (6.51 mg, yellow solid, yield: 26.5%).

LC-MS: (ESI) m/z (½M+H), 608.4.

¹HNMR (400 MHz, DMSO): δ 12.15 (s, 3H), 9.77 (s, 1H), 8.34 (d, J=6.8 Hz, 1H), 8.00-7.95 (m, 2H), 7.82 (d, J=9.2 Hz, 1H), 7.59 (d, J=8.8 Hz, 1H), 7.31 (s, 1H), 7.09 (s, 1H), 6.99 (s, 2H), 6.50 (s, 1H), 5.43 (s, 2H), 5.26 (s, 2H), 4.55-4.52 (m, 1H), 4.28-4.19 (m, 2H), 3.57-3.54 (m, 9H), 3.18-3.15 (m, 3H), 2.99-2.95 (m, 3H), 2.40 (d, J=6.4 Hz, 6H), 2.13-2.07 (m, 5H), 2.04-2.01 (m, 3H), 1.89-1.84 (m, 4H), 1.69-1.63 (m, 1H), 1.51-1.45 (m, 4H), 1.42-1.39 (d, J=6.8 Hz, 3H), 1.24-1.14 (m, 3H), 0.90-0.84 (m, 9H).

### Preparation Example 1.8. Synthesis of (S)—N5-((2R,3R,4R,5S,21S,22R,23R,24R)-1,2,3,4,5,21,22,23,24,25-decahydroxy-8,18-dioxo-13-((3-oxo-3-(((2S,3R,4R,5R)-2,3,4,5,6-pentahydroxyhexyl)amino)propoxy)methyl)-11,15-dioxa-7,19-diazapentacosan-13-yl)-2-(6-(2,5-dioxo-2,5-dihydro-1H-pyrrol-1-yl)hexanamido)-N1-((S)-1-(((S)-1-(((S)-9-ethyl-9-hydroxy-10,13-dioxo-2,3, 9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)pentanediamide (Compound (II′)-4)

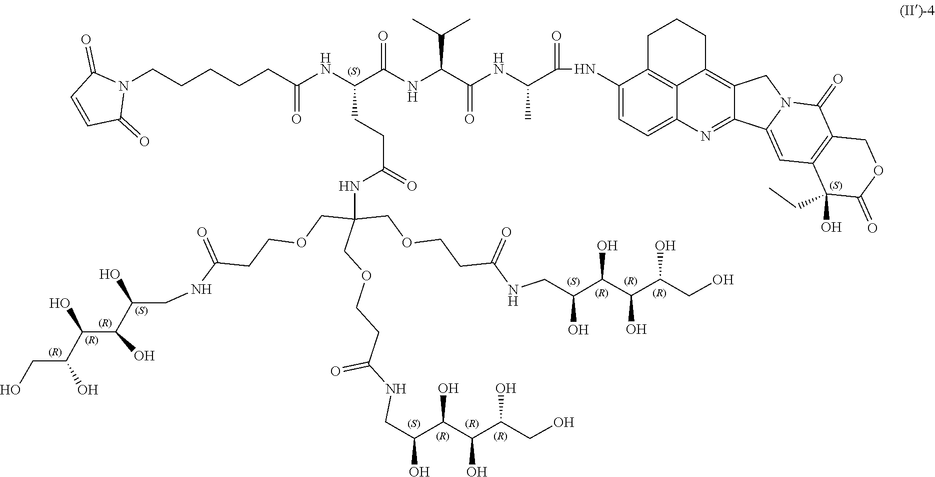

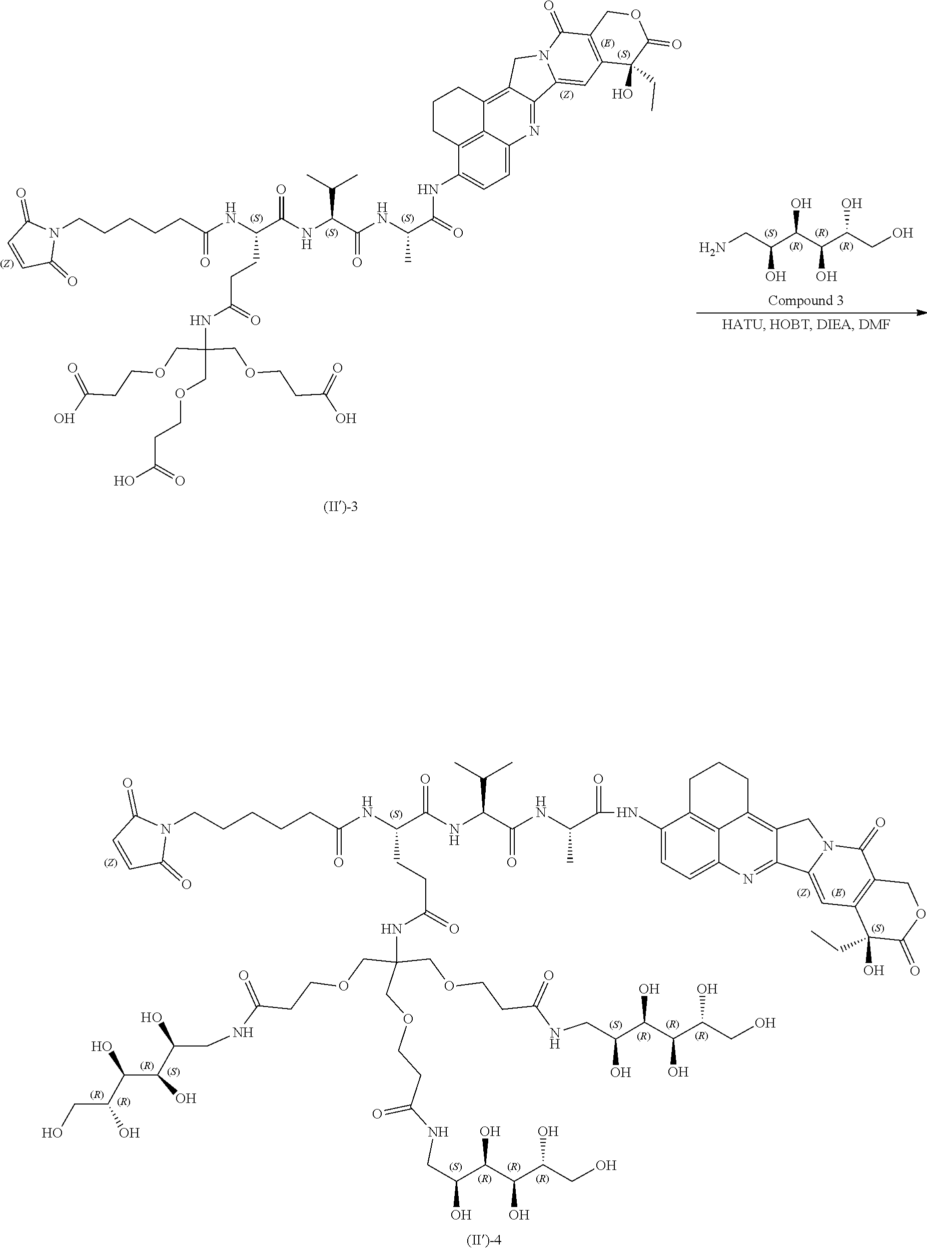

### Step 1: Synthesis of (II′)-4

A mixture of 3-{[(2S,5S,8S)-13,13-bis{[(2-carboxyethyl)oxy]methyl}-8-{[6-(2,5-dioxo-2,5-dihydro-1H-pyrrol-1-yl)-1-oxohexyl]amino}-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-2-methyl-1,4,7,11-tetraoxo-5-(prop-2-yl)-3,6,12-triazatetradec-14-yl]oxy}propanoic acid (122.97 mg, 0.101 mmol), HATU (146.21 mg, 0.385 mmol), HOBT (51.96 mg, 0.385 mmol) and DIEA (0.134 mL, 0.809 mmol) in DMF (2 mL) was stirred at 0° C. for 20 minutes and Compound 3 (69.67 mg, 0.385 mmol) was then added to the mixture before the mixture was naturally heated to 15° C. and stirred for 1 hour. The solvent was removed under vacuum and the residue was purified by Prep-HPLC (TFA) to yield Compound (II′)-4 (35.2 mg, yellow solid, yield: 20.4%).

LC-MS: (ESI) m/z (½M+H), 853.2.

¹HNMR (400 MHz, DMSO): δ 9.78 (s, 1H), 8.35 (d, J=6.8 Hz, 1H), 8.02 (d, J=7.2 Hz, 1H), 7.96 (d, J=9.2 Hz, 1H), 7.82 (d, J=9.2 Hz, 1H), 7.77 (t, J=5.6 Hz, 3H), 7.60 (d, J=8.8 Hz, 1H), 7.31 (s, 1H), 7.13 (s, 1H), 6.99 (s, 2H), 6.50 (s, 1H), 5.43 (s, 2H), 5.27 (s, 2H), 4.58-4.49 (m, 2H), 4.28-4.18 (m, 4H), 3.60-3.48 (m, 14H), 3.49-3.45 (m, 8H), 3.42-3.34 (m, 13H), 3.33-3.24 (m, 6H), 3.18-3.15 (m, 3H), 3.07-2.93 (m, 7H), 2.33 (t, J=6.8 Hz, 6H), 2.15-1.97 (m, 8H), 1.91-1.82 (m, 3H), 1.70-1.65 (m, 1H), 1.52-1.45 (m, 4H), 1.40 (d, J=6.8 Hz, 3H), 1.24-1.18 (m, 4H), 0.90-0.83 (m, 9H).

### Preparation Example 1.9. Synthesis of Compound LD-12 ((2S,5S,8S)-13-((2-carboxyethoxy)methyl)-8-(6-(2,5-dioxo-2, 5-dihydro-1H-pyrrol-1-yl)hexanamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oic acid)(Compound (II′)-5)

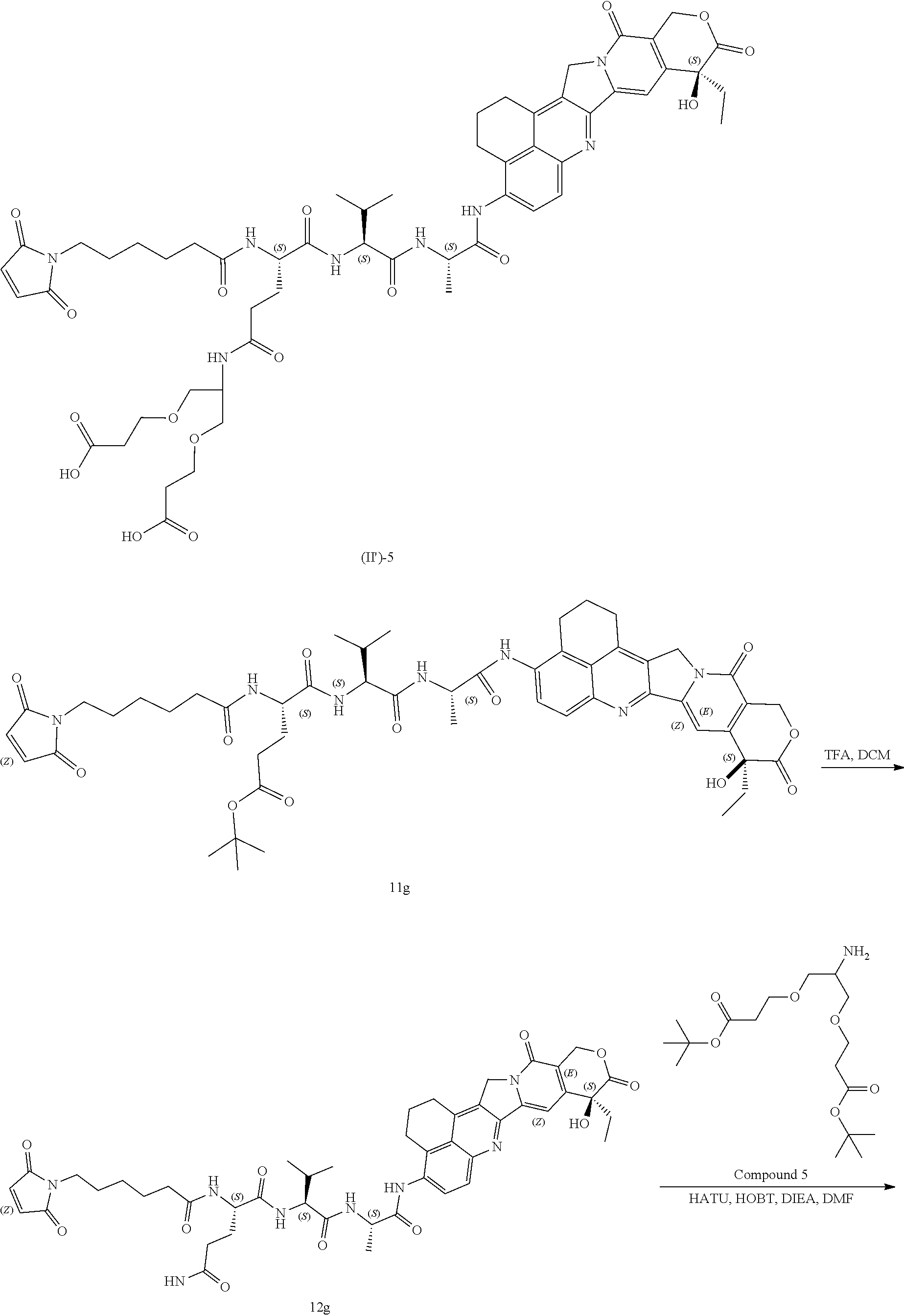

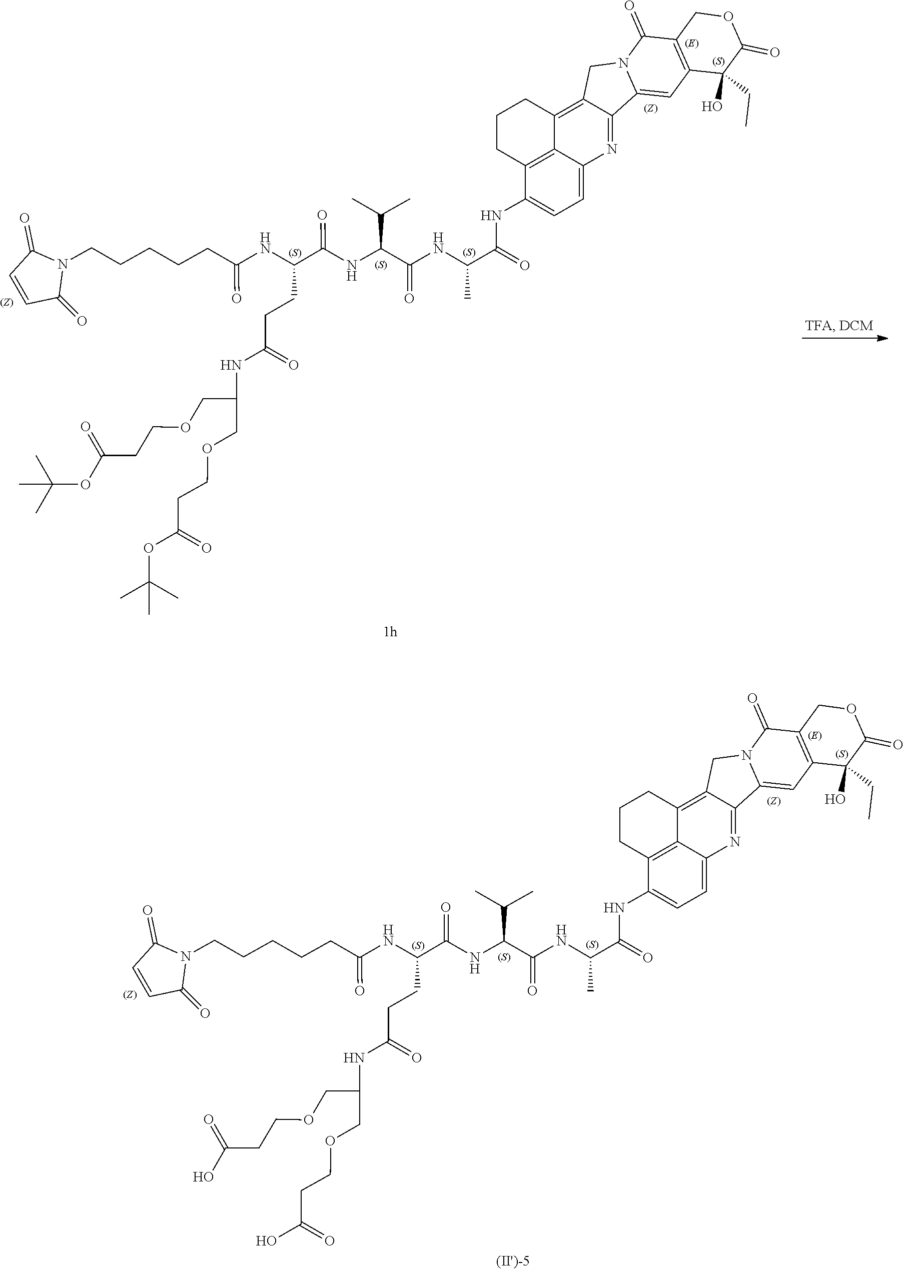

### Step 1: Synthesis of (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoic acid (Compound 12g)

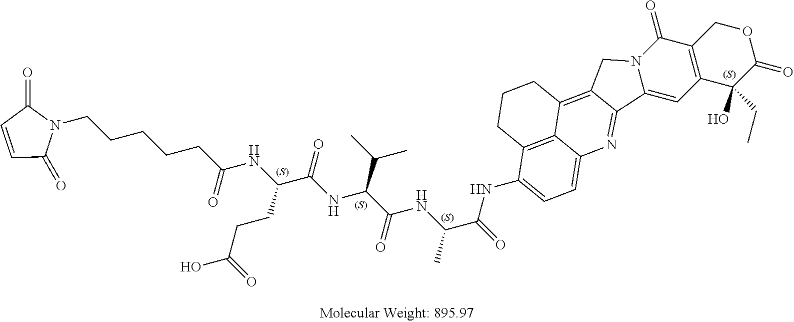

### Compound 12g

2,2,2-trifluoroacetic acid (0.132 mL, 1.260 mmol) was added to a solution of 2-methylpropan-2-yl (4S)-4-{[6-(2,5-dioxo-2,5-dihydro-1H-pyrrol-1-yl)-1-oxohexyl]amino}-5-{[(2S)-1-{[(2S)-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-1-oxoprop-2-yl]amino}-3-methyl-1-oxobut-2-yl]amino}-5-oxopentanoate (20 mg, 0.021 mmol) in DCM (0.65 mL) at 0° C. for 5 minutes. The resulting mixture was then naturally warmed up to 15° C. and stirred for 1 hour. After the reaction was completed as confirmed by LC-MS, the mixture was concentrated to yield Compound 12g which was used to next step without purification (18.82 mg, yellow solid, quantitative yield).

LC-MS: (ESI) m/z (M+1), 896.6.

### Step 2: Synthesis of tert-butyl (2S,5S,8S)-13-((3-(tert-butoxy)-3-oxopropoxy)methyl)-8-(6-(2,5-dioxo-2,5-dihydro-1H-pyrrol-1-yl)hexanamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oate (Compound 1h)

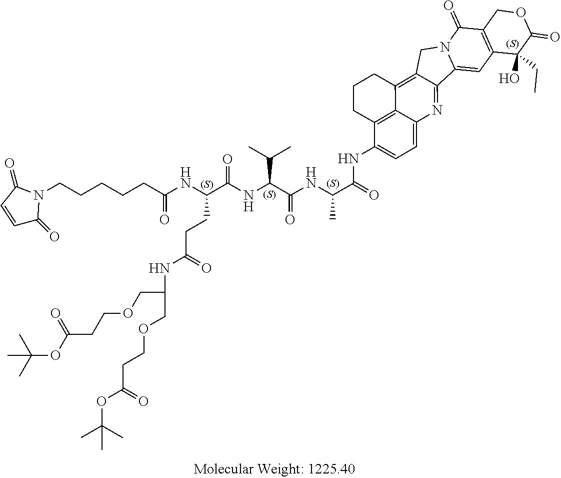

### Compound 1h

A mixture of (4S)-4-{[6-(2,5-dioxo-2,5-dihydro-1H-pyrrol-1-yl)-1-oxohexyl]amino}-5-{[(2S)-1-{[(2S)-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-1-oxoprop-2-yl]amino}-3-methyl-1-oxobut-2-yl]amino}-5-oxopentanoic acid (18.82 mg, 0.021 mmol), HATU (9.58 mg, 0.025 mmol), HOBT (3.41 mg, 0.025 mmol) and DIEA (0.010 mL, 0.063 mmol) in DMF (1 mL) was stirred at 0° C. for 5 minutes. Compound 5 (7.30 mg, 0.021 mmol) was then added to the mixture and the resulting mixture was naturally warmed up to 15° C. and stirred for 1 hour. After the reaction was completed as confirmed by LC-MS, saturated salt water (3 mL) was added to the reaction solution and a large amount of solids were extracted. The mixture was then filtered through a cloth funnel and the solids were washed by water. Afterwards, the resulting residue was dissolved in DCM (10 mL) before the organic layers were dried over sodium sulfate, filtered, and the filtrate was concentrated under reduced pressure to yield Compound 1h which was used to next step without purification (24 mg, brown solid, yield: 93.2%).

LC-MS: (ESI) m/z (M+1), 1226.6.

### Step 3: Synthesis of (2S,5S,8S)-13-((2-carboxyethoxy)methyl)-8-(6-(2,5-dioxo-2,5-dihydro-1H-pyrrol-1-yl)hexanamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oic acid ((II′)-5)

2,2,2-trifluoroacetic acid (0.126 mL, 1.200 mmol) was added to a solution of 2-methylpropan-2-yl3-{[(2S,5S,8S)-13-(7,7-dimethyl-5-oxo-2,6-dioxaoct-1-yl)-8-{[6-(2,5-dioxo-2,5-dihydro-1H-pyrrol-1-yl)-1-oxohexyl]amino}-1-{[(9S)-9-ethyl-9-hydroxy-10,13-dioxo-2,3,9,10,12,15-hexahydro-1H-cyclohexa[1,2,3-de]pyrano[3′,4′:6,7]indolizino[1,2-b]quinolin-4-yl]amino}-2-methyl-1,4,7,11-tetraoxo-5-(prop-2-yl)-3,6,12-triazatetradec-14-yl]oxy}propanoate (24 mg, 0.020 mmol) in DCM (0.63 mL) at 0° C. for 5 minutes. Then, the mixture was naturally warmed up to 15° C. and stirred for 1 hour before the solvent removed under vacuum. Afterwards, the resulting residue was purified by Prep-HPLC (TFA) to yield the Compound (II′)-5 (5.13 mg, yellow solid, yield: 23.5%).

LC-MS: (ESI) m/z (M+H), 1113.6.

¹HNMR (400 MHz, DMSO) δ 12.17 (s, 2H), 9.78 (s, 1H), 8.30 (d, J=6.4 Hz, 1H), 8.00-7.95 (m, 2H), 7.82 (d, J=9.2 Hz, 1H), 7.67 (d, J=8.4 Hz, 2H), 7.31 (s, 1H), 6.99 (s, 2H), 6.50 (s, 1H), 5.43 (s, 2H), 5.26 (s, 2H), 4.58-4.50 (m, 1H), 4.27-4.21 (m, 2H), 3.95-3.93 (m, 1H), 3.59-3.51 (m, 5H), 3.18-3.15 (m, 2H), 2.99-2.96 (m, 2H), 2.44-2.41 (m, 4H), 2.17-2.07 (m, 5H), 2.03-2.01 (m, 4H), 1.91-1.84 (m, 3H), 1.73-1.70 (m, 1H), 1.51-1.45 (m, 4H), 1.39 (d, J=7.2 Hz, 3H), 1.23-1.20 (m, 5H), 0.89-0.83 (m, 9H).

### Example 2. Preparation of Antibodies

### Preparation of Anti-HER2 Antibody

The antibodies herein can be prepared according to the conventional methods for antibodies. For example, the variable region sequences were cloned into a vector containing a sequence encoding the human IgG1 constant region for antibody expression. The binding affinity of the expressed antibody was verified using FACS.

The sequences of light chain and heavy chain of Trastuzumab are shown as follows:

Light chain
(SEQ ID NO: 1)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Heavy chain
(SEQ ID NO: 2)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

### Preparation of Anti-TROP2/EGFR Bispecific Antibody

Anti-TROP2/EGFR bispecific antibody has an anti-TROP2 antigen binding domain (T-6F7, VH SEQ ID NO: 3, VL SEQ ID NO: 4) and an anti-EGFR antigen binding domain (E-6C4 VH SEQ ID NO: 5, VL SEQ ID NO: 4). These antigen binding domains can be paired to form bispecific antibody (e.g. T-6F7-E-6C4).

Vectors encoding the light chain and heavy chain of the anti-TROP2/EGFR antibodies were constructed. CHO-S cells were co-transduced with three vectors, including a first vector encoding the heavy chain of anti-TROP2 binding arm, a second vector encoding the heavy chain of anti-EGFR binding arm, and a third vector encoding the common light chain. After 14 days of culture, the cell supernatant was collected and purified by Protein A affinity chromatography. Knobs-into-holes mutations were introduced in the Fe regions of the anti-TROP2 arm heavy chain and the anti-EGFR arm heavy chain. In T-6F7-E-6C4, the heavy chain constant region of T-6F7 includes knob mutations, and the heavy chain constant region of E-6C4 includes hole mutations.

The sequences of the light chain constant region, the heavy chain constant region with knob mutations, and the heavy chain constant region with hole mutations are shown in SEQ ID NO: 6, SEQ ID NO: 7 and SEQ ID NO: 8, respectively.

### Example 3. Preparation of ADCs

The prepared tris(2-carboxyethyl)phosphine aqueous solution was added to the antibody Trastuzumab analog in 1×PBS buffer solution (pH 7.4) (TCEP:antibody=8:1(molar ratio)), and the mixture was placed in a water bath shaker and shaken at 22° C. for 2.5 h, and then the reaction was terminated.

Compounds (II′)-1, (II′)-2, (II′)-3, (II′)-4, (II′)-5, VA-AZ0132 (Mal-PEG8-amide-Val-Ala-(4-NH2)-Exatecan, MedChemExpress, Cat #: HY-145399), GGFG-Dxd (Deruxtecan, MedChemExpress, Cat #: HY-13631E) or VC-MMAE (MedChemExpress, HY-15575) were dissolved in DMSO (the molar ratios of compound to antibody was shown in the table below) and the resulting solution was added to the above solution, respectively. The resulting mixture was then placed in a water bath shaker and shaken at 25° C. for 1.5 hours, followed by termination of the reaction. Afterwards, the reaction solution was desalted and purified by replacing the buffer solution using a Sephadex G25 gel column or UF/DF to remove excessive unconjugated compounds. Finally, T-(II′)-1, T-(II′)-2, T-(II′)-2(DAR4), T-(II′)-3, T-(II′)-4, T-(II′)-5, T-VA-AZ0132 and T-GGFG-Dxd was obtained, respectively, and stored at −20° C./−80° C. The mean n was calculated by LC-MS. In the control group, the antibody Trastuzumab analog was replaced by human IgG1 protein (hIgG1) and the hIgG1-(II′)-1, hIgG1-(II′)-2, hIgG1-(II′)-3, hIgG1-(II′)-4, hIgG1-(II′)-5, hIgG1-VA-AZ0132, or hIgG1-GGFG-Dxd was obtained, respectively. Details are shown in the table below.

TABLE 1

| | Compound: | | |
|---|---|---|---|
| | Antibody | | |
| | (molar | | Mean |
| ADCs | ratio) | Structure | n |
| T-(II')-1 | 14:1 | 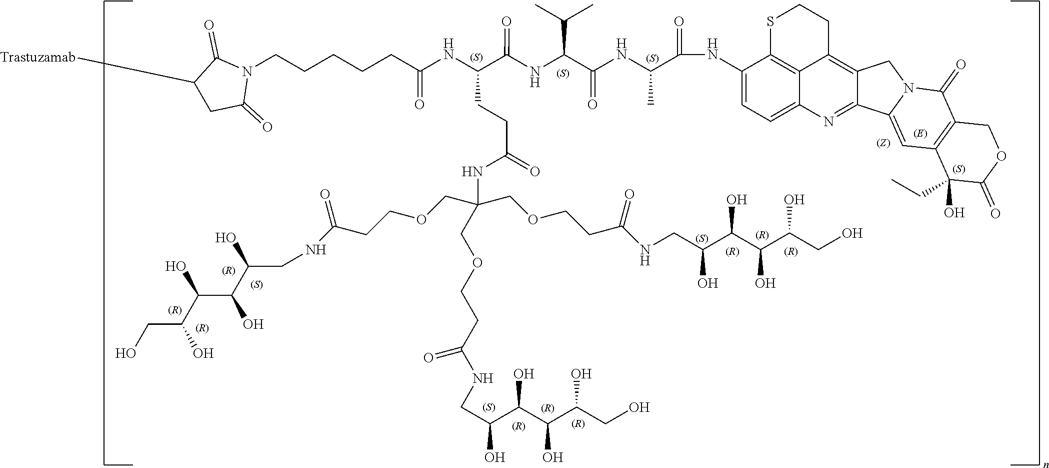 | 7.74 |
| hIgG1- (II')-1 | 14:1 | 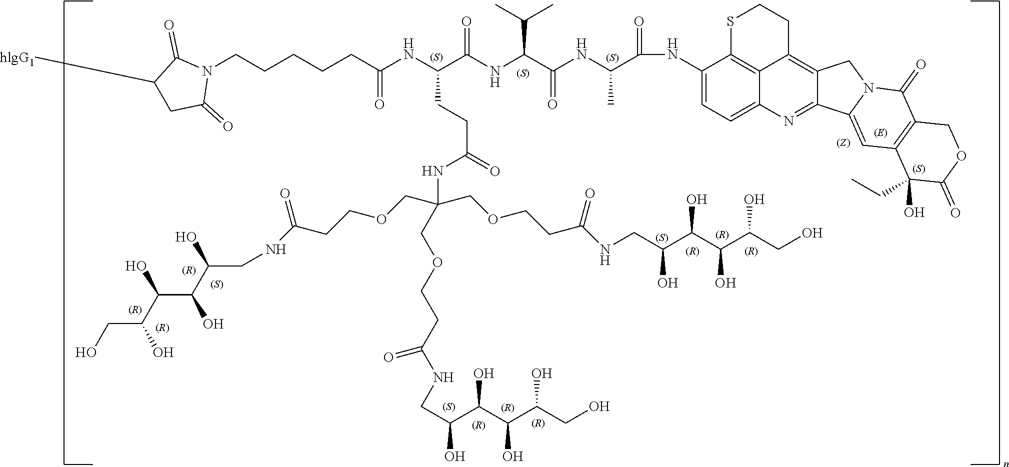 | 7.24 |
| T-(II')-2 | 16:1 | 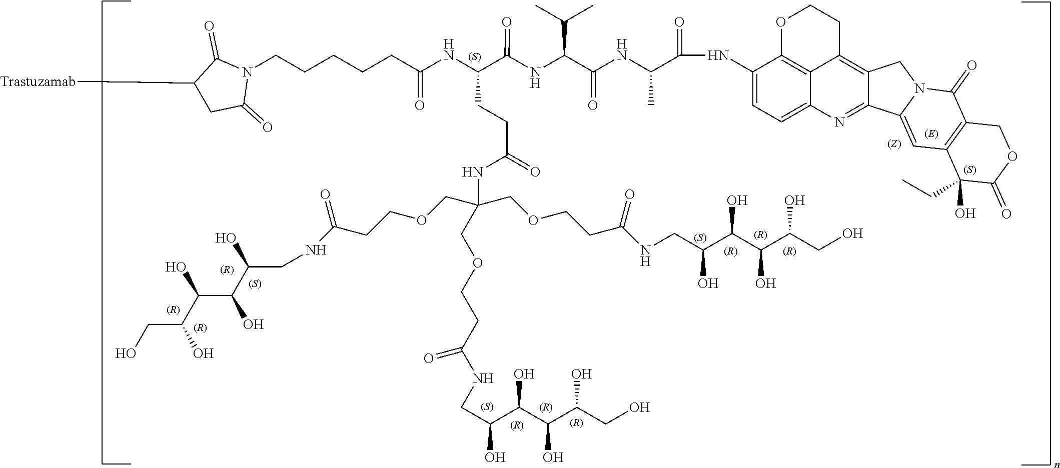 | 7.59 |
| hIgG1- (II')-2 | 14:1 | 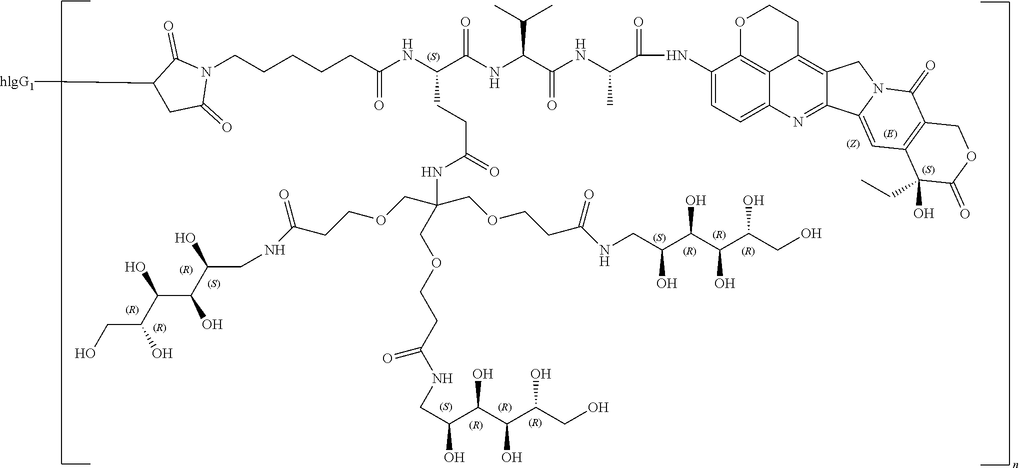 | 7.28 |
| T-(II')-3 | 14:1 | 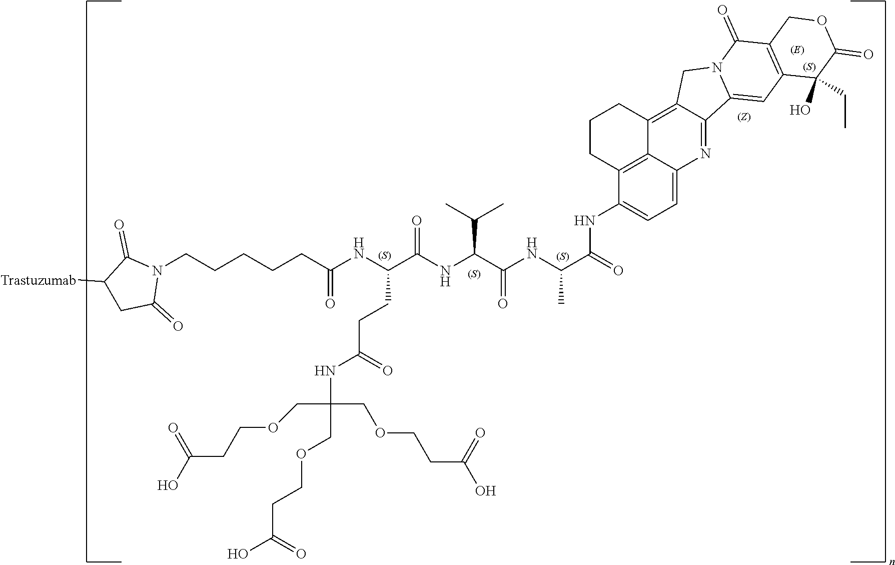 | 8.00 |
| T-(II')-4 | 14:1 | 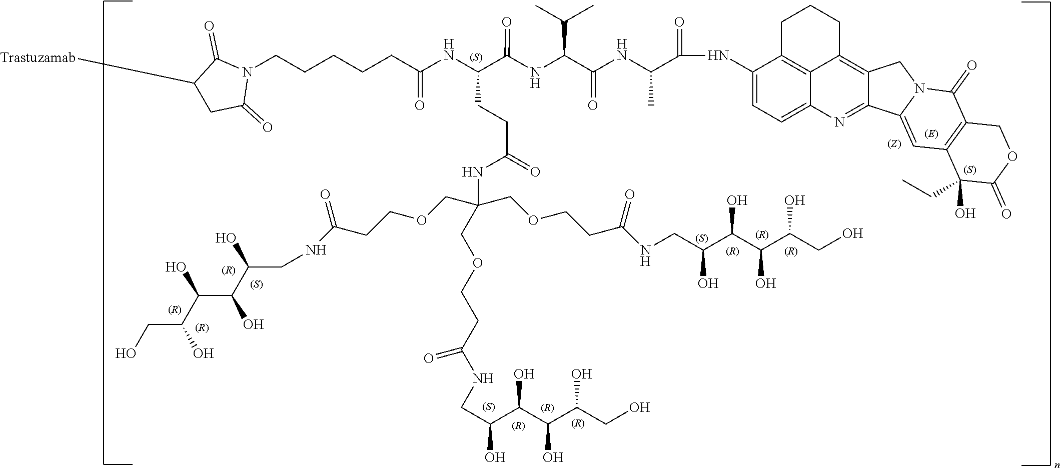 | 7.57 |
| hIgG1- (II')-4 | 14:1 | 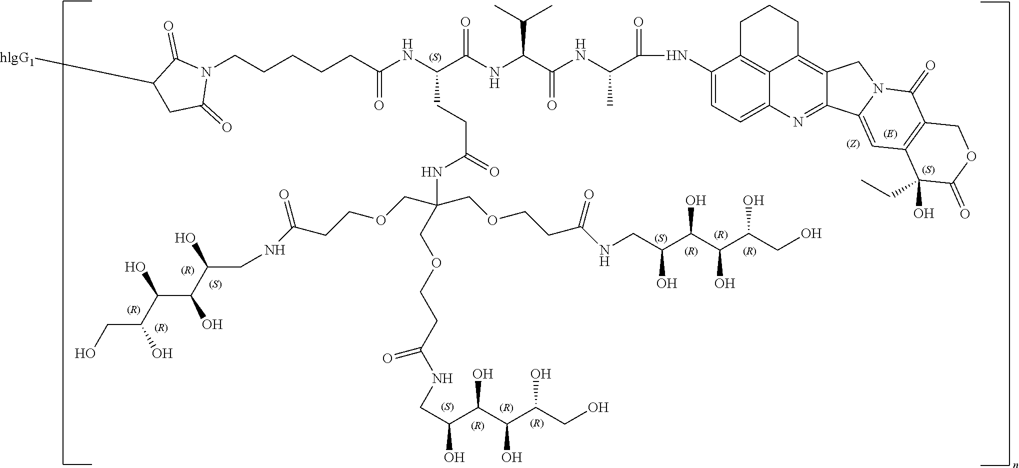 | 7.19 |
| T-(II')-5 | 14:1 | 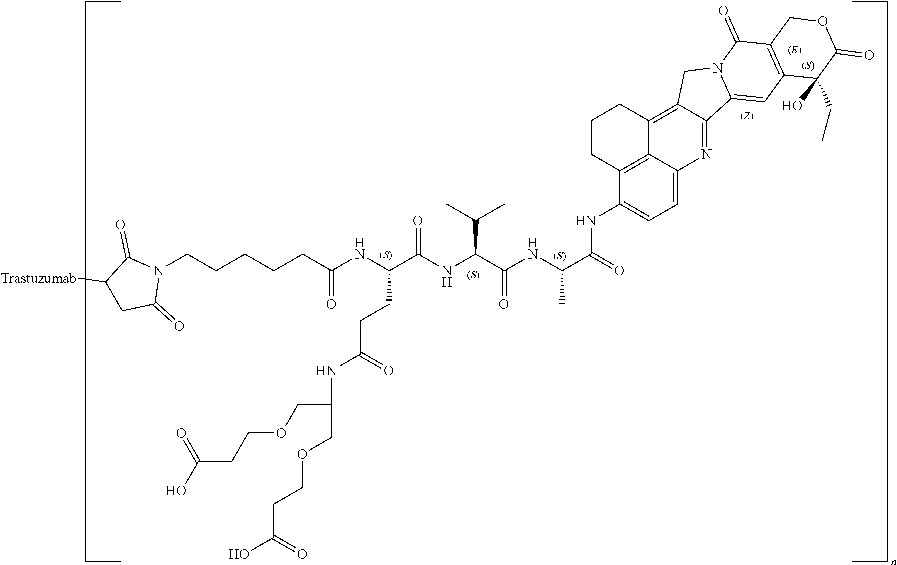 | 8.07 |
| T-VA- AZ0132 | 14:1 | 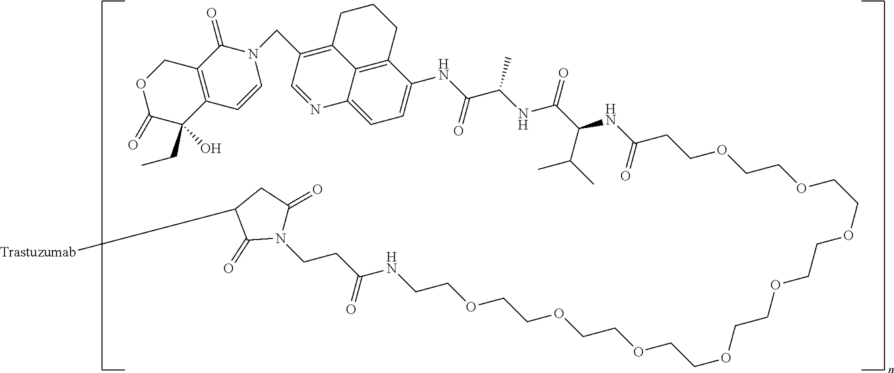 | 7.56 |
| hIgG1- VA- AZ0132 | 14:1 | 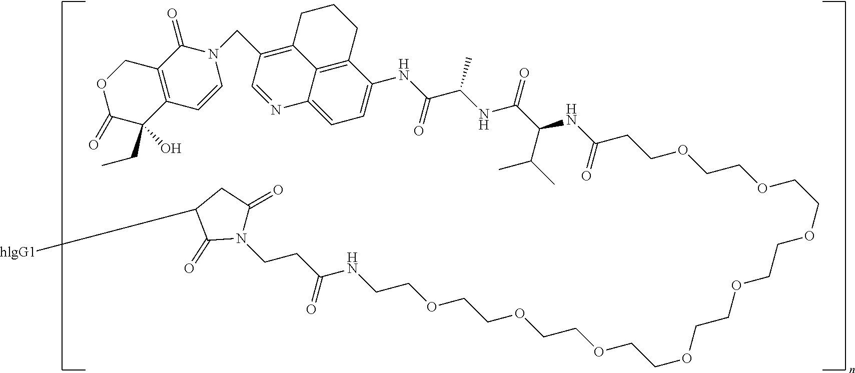 | 7.59 |
| T-GGFG- Dxd | 14:1 | 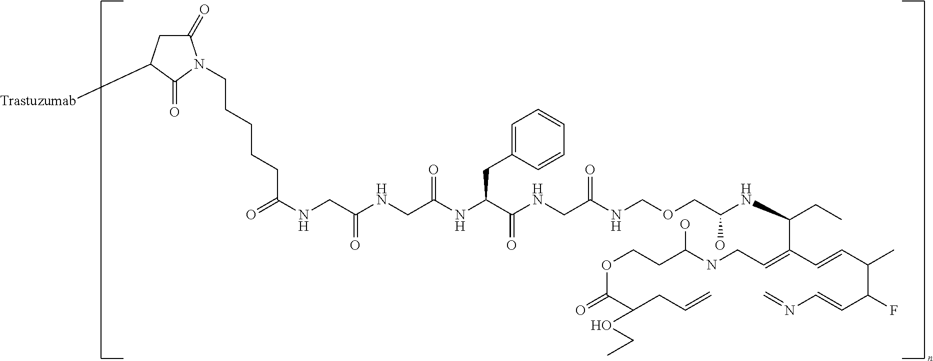 | 8.14 |
| hIgG1- GGFG- Dxd | 14:1 | 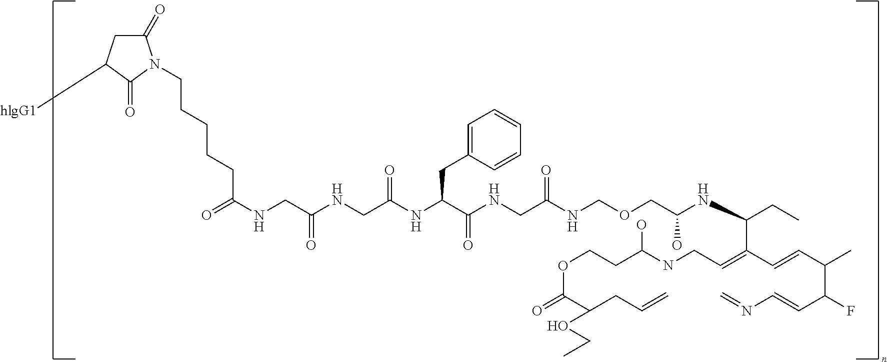 | 8.30 |
| T-(II')-2 (DAR4) | 14:1 | 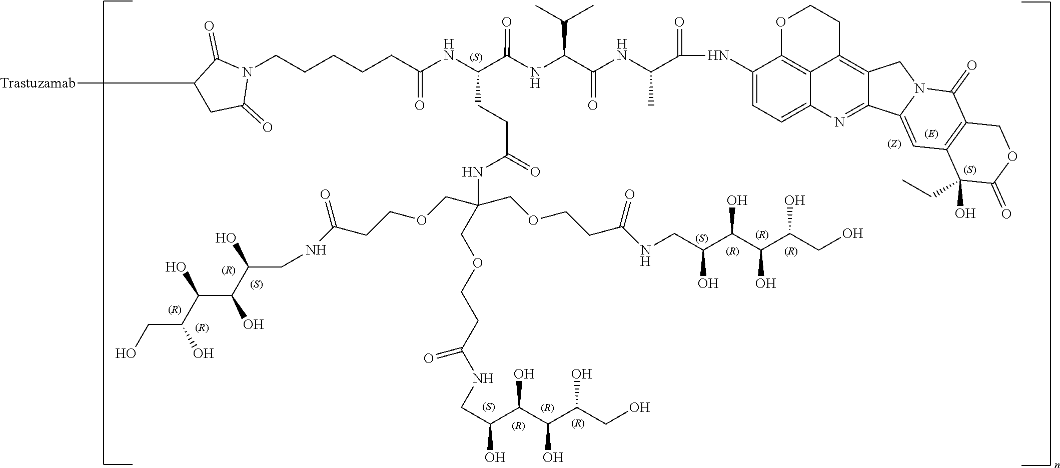 | 4.27 |
| T-VC- MMAE | 6:1 | 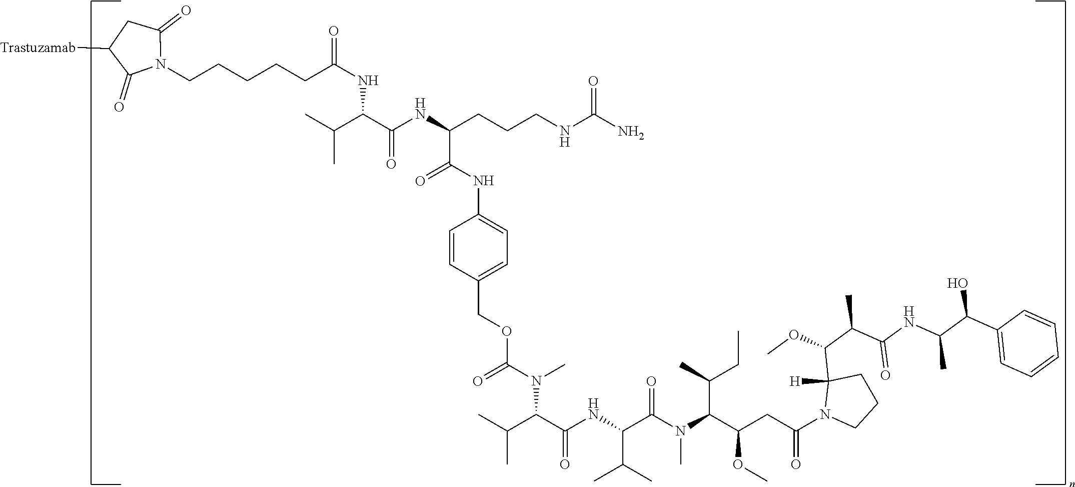 | 4.0 |

In another similar experiment, the purified antibody T-6F7-E-6C4 was coupled with compounds (II′)-1, (II′)-2, (II′)-3, (II′)-4, or (II′)-5. For the names of antibody-drug conjugates, (II′)-1, (II′)-2, (II′)-3, (II′)-4, or (II′)-5 is added directly after the antibody name. For example, if T-61F7-E-6C4 is coupled to compounds (II′)-1, it is named as T-6F7-E-6C4-(II′)-1. As another example, if T-6F7-E-6C4 is coupled to (II′)-2, it is named as T-6F7-E-6C4-(II′)-2. Exemplary ADCs obtained by this method included: T-6F7-E-6C4-(II′)-1, T-6F7-E-6C4-(II′)-2, T-6F7-E-6C4-(II′)-3, T-6F7-E-6C4-(II′)-4, and T-6F7-E-6C4-(II′)-5.

MS (Mass Spectrometry) was used to detect the coupling of antibodies with drug molecules. A human IgG1 molecule was coupled to (II′)-1, (II′)-2, (II′)-3, (II′)-4, or (II′)-5 to form isotype-(II′)-1 (ISO-(II′)-1), isotype-(II′)-2(ISO-(II′)-2), isotype-(II′)-3(ISO-(II′)-3), isotype-(II′)-4(ISO-(II′)-4) or isotype-(II′)-5(ISO-(I′)-5) as the isotype controls. The results showed that the drug-to-antibody ratio (DAR) of the ADCs was about 4 or 8. With regard to the ADC names, if the DAR of T-6F7-E-6C4-(II′)-2 is about 4, the ADC is named T-6F7-E-6C4-(II′)-2 (DAR4). If the DAR of T-6F7-E-6C4-(II′)-2 is about 8, the ADC is named T-61F7-E-6C4-(II′)-2 (DAR8).

In another similar experiment, the purified antibody T-61F7-E-6C4 was coupled with MMAE (monomethyl auristatin E) through a maleimidocaproyl-valine-citrulline-p-aminobenzyloxycarbonyl (VC) linker. For the names of antibody-drug conjugates, “ADC” is added directly after the antibody name when the antibody is coupled to MMAE. For example, if T-61F7-E-6C4 with IgG1 constant region is coupled to MMAE, it is named as T-6F7-E-6C4-ADC.

### Example 4. In Vitro Killing Activity

Different concentrations of compounds CPT-1, CPT-2, CPT-3, CPT-4, Dxd (MedChem Express, Cat #: HY-13631D) and SN38 (MedChem Express, Cat #: HY-13704) were used to treat different human tumor cell lines, mice tumor cell lines, rat tumor cell lines or dog tumor cell lines cultured in a cell culture plate, respectively. And the killing activity was detected after 72 h-120 h incubation by IncuCyte (Sartorius AG, IncuCyte® S3) or MicroplateReader. The results are summarized in the table below.

TABLE 2

| | Species | Cells | Time of Incubation | CPT-1 | CPT-2 | Dxd | SN38 |
|---|---|---|---|---|---|---|---|
| IC50 (nM) | Human | NCI-H1975 | 120 | 1.707 | 1.968 | 9.915 | 13.21 |
| | | NCI-H226 | 120 | 2.369 | 2.774 | 22.92 | 20.37 |
| | | NCI-H1781 | 120 | 0.297 | 0.399 | 0.724 | N/A |
| | | NCI-H2030 | 120 | 1.102 | 1.315 | 6.432 | N/A |
| | | HuH-7 | 120 | 0.742 | 0.662 | 1.390 | 2.407 |
| | | Hela | 120 | 1.028 | 1.405 | 6.693 | 6.608 |
| | | SW620 | 120 | 0.750 | 0.824 | 3.894 | 4.596 |
| | | LOVO | 120 | 1.518 | 1.152 | 8.646 | 10.19 |
| | | OVKATE | 120 | 2.238 | 2.045 | 12.33 | N/A |
| | | SKOV-3 | 120 | 1.802 | 1.963 | 16.46 | N/A |
| | | PC-3 | 120 | 0.859 | 1.966 | 6.399 | N/A |
| | | U-2-OS | 120 | 2.655 | 1.405 | 41.88 | — |
| | | HCC1954 | 120 | 1.307 | 1.14 | 3.313 | N/A |
| | | BT474 | 120 | 6.219 | 11.93 | 10.92 | N/A |
| | | SW156 | 120 | 0.373 | 0.615 | 1.836 | N/A |
| | | NCI-N87 | 120 | 1.202 | 1.478 | 7.025 | 7.388 |
| | | HS-746T | 120 | 0.523 | 0.550 | 1.048 | N/A |
| | | NCI-H69 | 120 | 0.2986 | 0.3175 | 0.2986 | N/A |
| | | Panc 02.03 | 120 | 0.266 | 0.301 | 3.178 | N/A |
| | | BxPC-3 | 120 | 0.213 | 0.289 | 1.873 | N/A |
| | | A-431 | 120 | 2.972 | 2.839 | 28.12 | N/A |
| | Dog | D17 | 120 | 0.841 | 1.288 | 6.084 | N/A |
| | | MP-0094-2 | 120 | 2.687 | 4.687 | 9.672 | N/A |
| | | MP-0094-3 | 120 | 3.920 | 7.151 | 22.02 | N/A |
| | | MP-1044-1 | 72 | 8.627 | 8.526 | 41.72 | N/A |
| | | MP-1092-1 | 120 | 0.988 | 1.752 | 7.133 | N/A |
| | | CMT-U27 | 120 | 0.937 | 1.276 | 3.371 | N/A |
| | | DCOL1 (S+L−) | 120 | 1.329 | 2.294 | 9.052 | N/A |
| | Mice | MC38 | 120 | 6.719 | 9.566 | 38.47 | N/A |
| | | CT26 | 96 | 3.844 | 5.634 | 94.20 | N/A |
| | | B16F10 | 75 | 3.291 | 5.469 | 26.16 | N/A |
| | Rat | L6 | 120 | 3.689 | 3.885 | 13.77 | 20.83 |
| | | PC12 | 120 | 1.665 | 1.770 | 9.192 | 5.655 |

(“N/A” means not detected, “—” means no killing)

The results showed that CPT-1 and CPT-2 had good in vitro killing activity on multiple cell lines, and exhibited a higher tumor cell inhibition effect than Dxd and SN38.

### Example 5. Anti-Tumor Activity in Human Pancreatic PDX Model

The ADCs were tested for their effects on tumor growth in vivo in human pancreatic tumor PDX models. Immunofluorescence staining of patient-derived tumor fragments was performed and the images were analyzed via HALO 3.2 version. The results showed that Her2 positive cells constituted 86.34% of total cells in the human pancreatic tumor tissues.

Specifically, B-NDG mice (Biocytogen Pharmaceuticals (Beijing) Co., Ltd., Cat #: B-CM-002) were engrafted in the right flank with patient-derived pancreatic tumor fragments (2 mm×2 mm×2 mm). When the tumors in the mice reached a volume of about 200-300 mm3, the mice were randomly placed into different groups based on the volume of the tumor. The mice were then injected with PBS or ADCs by intravenous (i.v.) administration at a dosage of 3 mg/kg or 6 mg/kg (1 administration in total). Details are shown in the table below.

TABLE 3

| Group | No. of mice | ADCs | Dosage | Route |
|---|---|---|---|---|
| G1 | 5 | PBS | — | i.v. |
| G2 | 5 | T-GGFG-Dxd | 3 mg/kg | i.v. |
| G3 | 5 | T-GGFG-Dxd | 6 mg/kg | i.v. |
| G4 | 5 | T-(II′)-4 | 3 mg/kg | i.v. |
| G5 | 5 | T-(II′)-4 | 6 mg/kg | i.v. |

The lengths of the long axis and the short axis of the tumor were measured and the volume of the tumor was calculated as 0.5×(long axis)×(short axis)₂. The tumor growth inhibition (TGI) was calculated using the following formula: TGI (%)=[1−(Ti−T0)/(Vi−V0)]×100%. Ti is the average tumor volume in the treatment group on day i. T0 is the average tumor volume in the treatment group on day zero. Vi is the average tumor volume in the control group on day i. V0 is the average tumor volume in the control group on day zero. T-test was performed for statistical analysis. A TGI higher than 60% indicates clear suppression of tumor growth. P<0.05 is a threshold to indicate significant difference. The body weights of the mice were also measured throughout the entire administration period.

The table below summarizes the results for this experiment, including the tumor volumes on the day of grouping (Day 0), 14 days after grouping (Day 14), and 21 days after grouping (Day 21); TGI (%); and the statistical differences (P value) of tumor volumes between the treatment and control groups.

TABLE 4

| | Tumor volume (mm³) | | | TGI (%) | P value |
|---|---|---|---|---|---|
| Group | Day 0 | Day 14 | Day 21 | (Day 21) | (Day 21) |
| G1 | 243 ± 10 | 994 ± 94 | 1772 ± 204 | NA | NA |
| G2 | 242 ± 17 | 737 ± 81 | 1305 ± 130 | 30.5 | 0.090 |
| G3 | 242 ± 13 | 731 ± 89 | 1426 ± 134 | 22.6 | 0.194 |
| G4 | 243 ± 17 | 393 ± 53 | 850 ± 109 | 60.3 | 0.004 |
| G5 | 242 ± 14 | 252 ± 14 | 457 ± 52 | 86.0 | 2.488E−04 |

The results showed that T-(II′)-4 had good tumor inhibitory effects in human pancreatic tumor and inhibited tumor growth with a higher TGI % than that of the positive control T-GGFG-Dxd.

In addition, T-VC-MMAE was also used as a control in the experiment with a TGI % of 44.9% and 60.7% on Day21 at a dose level of 3 mg/kg and 6 mg/kg respectively, indicating that T-(II′)-4 exhibited better tumor inhibitory effect than T-VC-MMAE.

### Example 6. Anti-Tumor Activity in Human Breast PDX Model

The ADCs were tested for their effects on tumor growth in vivo in human breast tumor PDX models. Immunofluorescence staining of patient-derived tumor fragments was performed and the images were analyzed via HALO 3.2 version. The results showed that Her2 positive cells constituted 0.65% of total cells in the human breast tumor tissues.

Specifically, B-NDG mice were engrafted in the right flank with patient-derived pancreatic tumor fragments (2 mm×2 mm×2 mm). When the tumors in the mice reached a volume of about 200-300 mm3, the mice were randomly placed into different groups based on the volume of the tumor. The mice were then injected with PBS or ADCs by intravenous (i.v.) administration at a dosage of 6 mg/kg. The frequency of administration was once every two weeks (2 administrations in total). Details are shown in the table below.

TABLE 5

| Group | No. of mice | ADCs | Dosage | Route | Frequency |
|---|---|---|---|---|---|
| G1 | 5 | PBS | — | i.v. | 2W |
| G2 | 5 | hIgG1-GGFG-Dxd | 6 mg/kg | i.v. | 2W |
| G3 | 5 | hIgG1-(II′)-4 | 6 mg/kg | i.v. | 2W |
| G4 | 5 | T-GGFG-Dxd | 6 mg/kg | i.v. | 2W |
| G5 | 5 | T-(II′)-4 | 6 mg/kg | i.v. | 2W |

The table below summarizes the results for this experiment, including the tumor volumes on the day of grouping (Day 0), 18 days after grouping (Day 18) and 28 days after grouping (Day 28); TGI (%); and the statistical differences (P value) of tumor volumes between the treatment and control groups.

TABLE 6

| | Tumor volume (mm³) | | | TGI (%) | P value |
|---|---|---|---|---|---|
| Group | Day 0 | Day 18 | Day 28 | (Day 28) | (Day 28) |
| G1 | 214 ± 18 | 1242 ± 85 | 2155 ± 156 | NA | NA |
| G2 | 215 ± 11 | 804 ± 114 | 1917 ± 277 | 12.3 | 0.560 |
| G3 | 214 ± 10 | 908 ± 141 | 1475 ± 94 | 35.1 | 0.011 |
| G4 | 214 ± 11 | 638 ± 64 | 1251 ± 149 | 46.6 | 0.008 |
| G5 | 214 ± 15 | 383 ± 37 | 579 ± 63 | 81.2 | 3.081E−05 |

The results showed that T-(II′)-4 had good tumor inhibitory effects in human breast tumor and inhibited tumor growth with a higher TGI % than that of the positive control T-GGFG-Dxd.

### Example 7. Anti-Tumor Activity in Human PDX Models

The ADCs were tested for their effects on tumor growth in vivo in human lung, colorectal or gastric tumor PDX models. Immunofluorescence staining of patient-derived tumor fragments was performed and the images were analyzed via HALO 3.2 version.

Specifically, B-NDG mice were engrafted in the right flank with patient-derived lung, colorectal or gastric tumor fragments (2 mm×2 mm×2 mm). When the tumors in the mice reached a volume of about 200-300 mm3, the mice were randomly placed into different groups based on the volume of the tumor. The mice were then injected with T-(II′)-1, T-(II′)-2, T-(II′)-3, T-(II′)-4, T-(II′)-5, T-VA-AZ0132, T-GGFG-Dxd, hIgG1-(II′)-1, hIgG1-(II′)-2, hIgG1-(II′)-3, hIgG1-(II′)-4, hIgG1-(II′)-5, hIgG1-VA-AZ0132, or hIgG1-GGFG-Dxd by intravenous (i.v.) administration at a dosage of 3 mg/kg or 6 mg/kg.

The results showed that T-(II′)-1, T-(II′)-2, T-(II′)-3, T-(II′)-4, and T-(II′)-5 had good tumor inhibitory effects.

### Anti-Tumor Activity in Human Lung Cancer PDX Model

The immunofluorescence staining of patient-derived tumor fragments results showed that HER2 positive cells constituted 89.49% of total cells in the human lung tumor tissues. The mice were injected with PBS or ADCs by intravenous (i.v.) administration at a dosage of 6 mg/kg (1 administration in total). Details are shown in the table below.

TABLE 7

| Group | No. of mice | ADCs | Dosage | Route |
|---|---|---|---|---|
| G1 | 5 | PBS | — | i.v. |
| G2 | 5 | T-GGFG-Dxd | 3 mg/kg | i.v. |
| G3 | 5 | T-(II′)-4 | 3 mg/kg | i.v. |
| G4 | 5 | T-(II′)-1 | 3 mg/kg | i.v. |
| G5 | 5 | T-(II′)-2(DAR4) | 3 mg/kg | i.v. |
| G6 | 5 | T-(II′)-2 | 3 mg/kg | i.v. |
| G7 | 5 | T-GGFG-Dxd | 6 mg/kg | i.v. |
| G8 | 5 | T-(II′)-4 | 6 mg/kg | i.v. |
| G9 | 5 | T-(II′)-1 | 6 mg/kg | i.v. |
| G10 | 5 | T-(Il′)-2(DAR4) | 6 mg/kg | i.v. |
| G11 | 5 | T-(II′)-2 | 6 mg/kg | i.v. |

The tumor volumes on the day of grouping (Day 0), 13 days after grouping (Day 13) and 23 days after grouping (Day 23); TGI (%); and the statistical differences (P value) of tumor volumes between the treatment and control groups are shown in the table below, which showed that T-(II′)-1, T-(II′)-2, T-(II′)-2(DAR4) and T-(II′)-4 all exhibited tumor inhibitory effects in human lung tumor in a dose-dependent manner. In addition, T-(II′)-1, T-(II′)-2 and T-(II′)-4 exhibited better tumor inhibitory effects than T-GGFG-Dxd both at 3 mg/kg and 6 mg/kg dosage.

TABLE 8

| | Tumor volume (mm³) | | | TGI (%) | P value |
|---|---|---|---|---|---|
| Group | Day 0 | Day 13 | Day 23 | (Day 23) | (Day 23) |
| G1 | 184 ± 11 | 1501 ± 212 | 1829 ± 663 | NA | NA |
| G2 | 184 ± 23 | 779 ± 172 | 1558 ± 323 | 16.5 | 0.692 |
| G3 | 184 ± 17 | 246 ± 86 | 571 ± 160 | 76.5 | 0.036 |
| G4 | 184 ± 13 | 261 ± 56 | 1075 ± 222 | 45.8 | 0.200 |
| G5 | 184 ± 14 | 608 ± 120 | 1555 ± 336 | 16.7 | 0.694 |
| G6 | 184 ± 26 | 467 ± 173 | 1223 ± 331 | 36.8 | 0.399 |
| G7 | 184 ± 16 | 503 ± 47 | 1364 ± 109 | 28.2 | 0.293 |
| G8 | 184 ± 15 | 129 ± 57 | 485 ± 198 | 81.7 | 0.039 |
| G9 | 184 ± 19 | 201 ± 59 | 722 ± 145 | 67.3 | 0.049 |
| G10 | 184 ± 22 | 241 ± 73 | 903 ± 248 | 56.3 | 0.150 |
| G11 | 184 ± 14 | 261 ± 54 | 882 ± 170 | 57.6 | 0.091 |

### Anti-Tumor Activity in Human Colorectal Cancer PDX Model

The immunofluorescence staining of patient-derived tumor fragments results showed that HER2 positive cells constituted 13.28% of total cells in the human colorectal tumor tissues. The mice were injected with PBS or ADCs by intravenous (i.v.) administration at a dosage of 6 mg/kg (1 administration in total). Details are shown in the table below.

TABLE 9

| | No. of | | | |
|---|---|---|---|---|
| Group | mice | ADCs | Dosage | Route |
| G1 | 5 | PBS | — | i.v. |
| G2 | 5 | T-GGFG-Dxd | 6 mg/kg | i.v. |
| G3 | 5 | T-(II′)-4 | 6 mg/kg | i.v. |
| G4 | 5 | T-(II′)-1 | 6 mg/kg | i.v. |
| G5 | 5 | T-(II′)-2(DAR4) | 6 mg/kg | i.v. |
| G6 | 5 | T-(II′)-2 | 6 mg/kg | i.v. |

The table below summarizes the results for this experiment, including the tumor volumes on the day of grouping (Day 0), 14 days after grouping (Day 14) and 28 days after grouping (Day 28); TGI (%); and the statistical differences (P value) of tumor volumes between the treatment and control groups.

TABLE 10

| | Tumor volume (mm³) | | | TGI (%) | P value |
|---|---|---|---|---|---|
| Group | Day 0 | Day 14 | Day 28 | (Day 28) | (Day 28) |
| G1 | 145 ± 6 | 490 ± 70 | 960 ± 147 | NA | NA |
| G2 | 146 ± 5 | 214 ± 45 | 506 ± 115 | 55.8 | 0.041 |
| G3 | 146 ± 7 | 177 ± 13 | 345 ± 44 | 75.5 | 0.004 |
| G4 | 146 ± 6 | 197 ± 29 | 383 ± 66 | 70.9 | 0.007 |
| G5 | 146 ± 7 | 227 ± 23 | 566 ± 62 | 48.5 | 0.038 |
| G6 | 146 ± 6 | 196 ± 29 | 413 ± 80 | 67.2 | 0.011 |

The results showed that T-(II′)-1, T-(II′)-2, T-(II′)-2(DAR4) and T-(II′)-4 all had good tumor inhibitory effects in human colorectal tumor. In addition, T-(II′)-1, T-(II′)-2 and T-(II′)-4 exhibited better tumor inhibitory effect than T-GGFG-Dxd at a dose level of 6 mg/kg.

### Anti-Tumor Activity in Human Gastric Cancer PDX Model

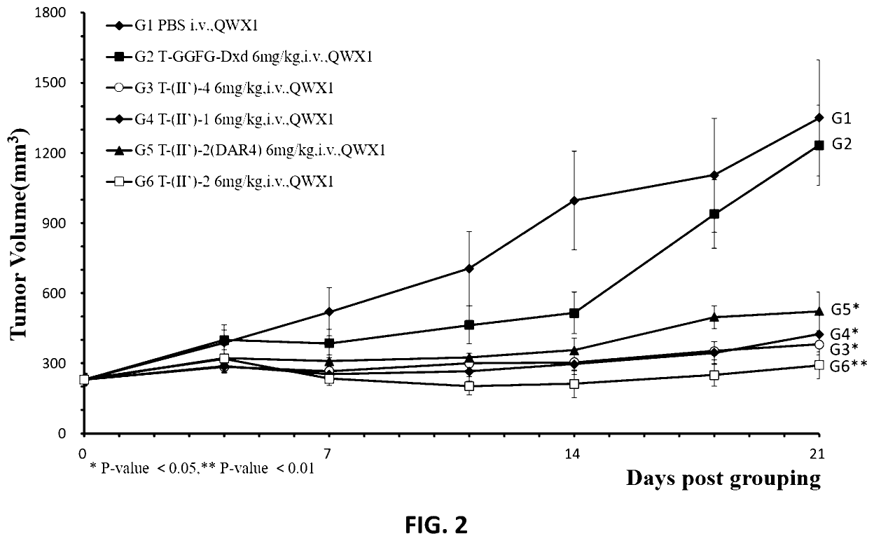

The immunofluorescence staining of patient-derived tumor fragments results showed that HER2 positive cells constituted 0.08%% of total cells in the human gastric tumor tissues. The mice were injected with PBS or ADCs by intravenous (i.v.) administration at a dosage of 6 mg/kg (1 administration in total). The tumor size in groups treated with PBS or ADCs are shown in FIG. 2, which showed that T-(II′)-1, T-(II′)-2, T-(II′)-2(DAR4) and T-(II′)-4 all exhibited good tumor inhibitory effects in human gastric cancer.

### Example 8. Anti-Tumor Activity in Hep-G2 Xenograft Model

The ADCs were tested for their effects on tumor growth in vivo in a xenograft model of human liver cancer. Specifically, about 1×10⁷ Hep-G2 cells (ATCC, Cat #: HB-8065) were injected subcutaneously in B-NDG mice. When the tumors in the mice reached a volume of about 300 mm³, the mice were randomly placed into different groups based on tumor volume. The mice were then injected with PBS or ADCs by intravenous (i.v.) administration. Details are shown in the table below.

TABLE 11

| | No. of | | | |
|---|---|---|---|---|
| Group | mice | ADCs | Dosage | Route |
| G1 | 5 | PBS | — | i.v. |
| G2 | 5 | hIgG1-(II′)-4 | 3 mg/kg | i.v. |
| G3 | 5 | hIgG1-(II′)-1 | 3 mg/kg | i.v. |
| G4 | 5 | hIgG1-(II′)-2 | 3 mg/kg | i.v. |
| G5 | 5 | T-(II′)-4 | 3 mg/kg | i.v. |
| G6 | 5 | T-(II′)-1 | 3 mg/kg | i.v. |
| G7 | 5 | T-(II′)-2(DAR4) | 3 mg/kg | i.v. |
| G8 | 5 | T-(II′)-2(DAR4) | 6 mg/kg | i.v. |
| G9 | 5 | T-(II′)-2 | 3 mg/kg | i.v. |

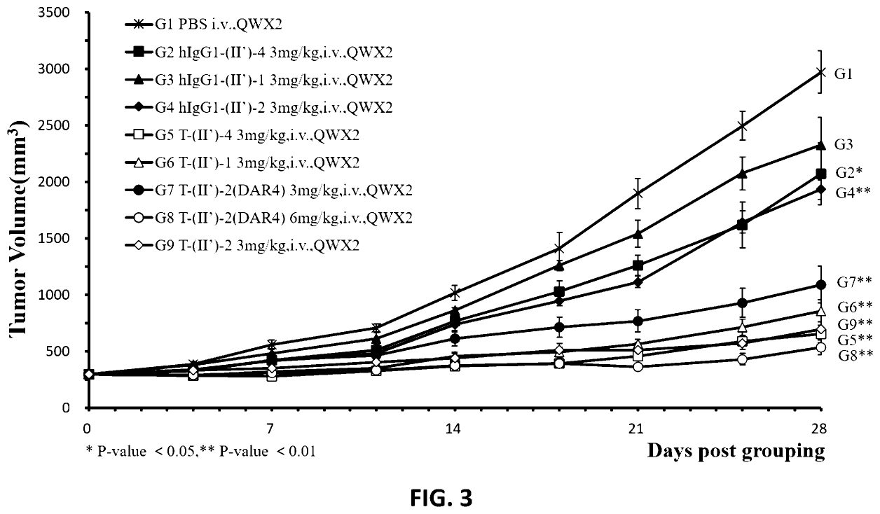

The tumor size in groups treated with PBS or ADCs are shown in FIG. 3, which showed that T-(II′)-1, T-(II′)-2, T-(II′)-2(DAR4) and T-(II′)-4 all exhibited significant tumor inhibitory effects in human liver cancer, however, hIgG1-(II′)-1, hIgG1-(II′)-2, and hIgG1-(II′)-4 did not exhibit significant tumor inhibitory effects. In addition, T-(II′)-2(DAR4) exhibited good tumor inhibitory effects in a dose-dependent manner.

### Example 9. Pharmacokinetic Profiles and Plasma stability

The pharmacokinetic clearance rates of the anti-HER2 ADCs were determined in C57BL/6 mice. Specifically, the mice were placed into nine groups (8 mice per group), and administered with hIgG1-GGFG-Dxd (G1, 3 mg/kg), T-GGFG-Dxd (G2, 3 mg/kg; G3, 10 mg/kg), hIgG1-(II′)-4(G4, 3 mg/kg; G5, 10 mg/kg), T-(II′)-4(G6, 3 mg/kg; G7, 10 mg/kg) or Trastuzumab analog (G8, 3 mg/kg; G9, 10 mg/kg) by intravenous injection. Blood samples were collected before administration and 15 minutes, 4 h, 24 h, 72 h, 10 days, 14 days, 21 days after administration to detect the serum levels of total antibody by sandwich ELISA and free payload by MS (Mass Spectrometry).

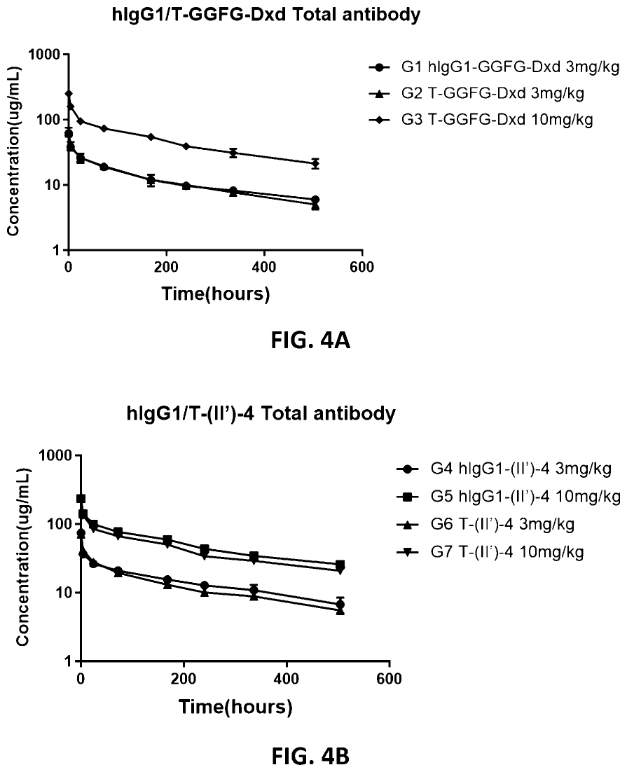

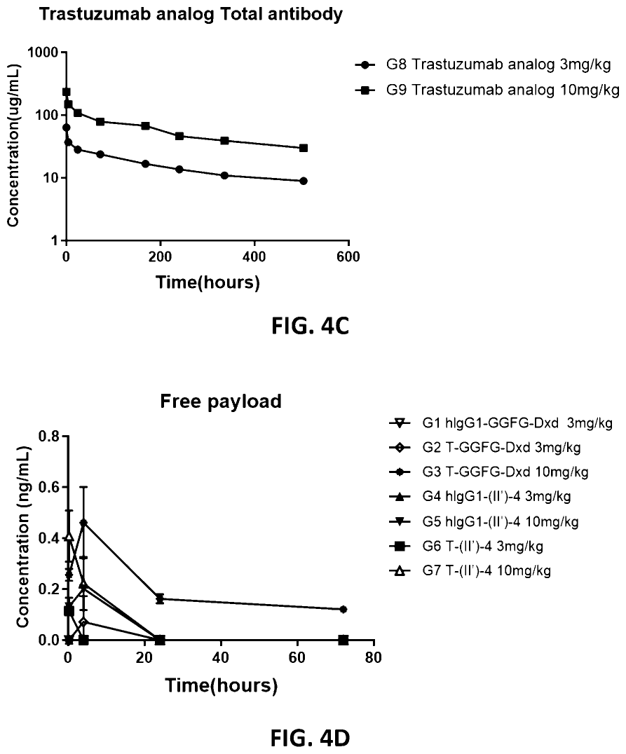

The results are shown in the table below and FIGS. 4A-4D, which showed that T-(II′)-4 has a longer half-life than T-GGFG-Dxd. In addition, at a dose level of 3 mg/kg, the free payload in G6 group could only be detected at 15 minutes after administration, while in G2 group the free payload could still be detected at 4 hours after administration, and at a dose level of 10 mg/kg, the free payload in G7 group could be detected at 4 hours after administration, while in G3 group could still be detected at 72 hours after administration, indicating that T-(II′)-4 exhibited better stability than T-GGFG-Dxd.

TABLE 12

| | | Dose | | T_{1/2} |
|---|---|---|---|---|
| Group | ADCs | (mg/kg) | LBA | (hour) |
| G1 | hIgG1-GGFG-Dxd | 3 | Antibody | 384.109 |
| G2 | T-GGFG-Dxd | 3 | Antibody | 265.249 |
| G3 | T-GGFG-Dxd | 10 | Antibody | 285.293 |
| G4 | hIgG1-(II′)-4 | 3 | Antibody | 264.135 |
| G5 | hIgG1-(II′)-4 | 10 | Antibody | 333.236 |
| G6 | T-(II′)-4 | 3 | Antibody | 276.952 |
| G7 | T-(II′)-4 | 10 | Antibody | 376.527 |
| G8 | Trastuzumab analog | 3 | Antibody | 411.691 |
| G9 | Trastuzumab analog | 10 | Antibody | 421.503 |

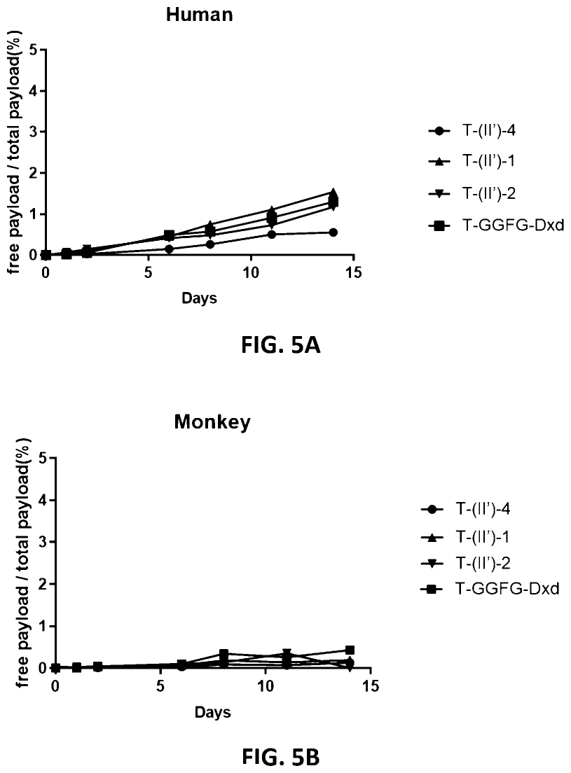

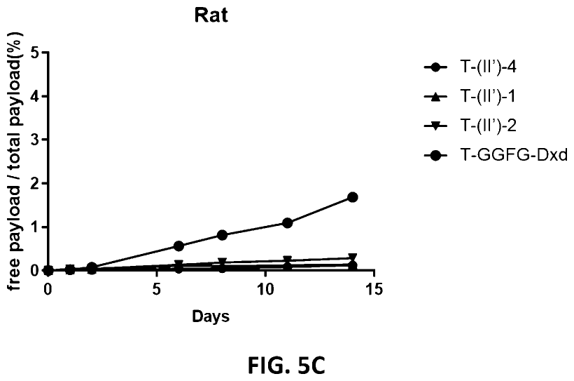

In another experiment, the plasma stability of T-(II′)-1, T-(II′)-2, and T-(II′)-4 were determined in human plasma, monkey (Macaca fascicularis) plasma, and rat (SD rat) plasma. Specifically, T-(II′)-1, T-(II′)-2, T-(II′)-4 or T-GGFG-Dxd were added to human, monkey, or rat plasma, respectively, to a terminal concentration of 100 μg/mL. In the control group, the plasma was replaced by PBS with 0.5% BSA. The contents of free payload and ADC were determined in 0 day, 1 day, 2 days, 6 days, 8 days, 11 days and 14 days after adding the ADCs, and the ratios of free payload to the total payload were calculated. The results are shown in FIGS. 5A-5C, which indicated that T-(II′)-1, T-(II′)-2 and T-(II′)-4 were relatively stable in human, monkey, and rat plasma, with a release rate of free CPT2 no more than 2.0% at the highest.

### Example 10. Antibody Drug Conjugates

### In Vitro Killing Activities

Different concentrations of purified antibodies or ADCs were used to treat HCC827 cells, NCI-H292 cells, A431 cells, or Panc 02.03 cells cultured in a cell culture plate, and the killing activities were detected after 7 days of incubation using CellCounting-Lite 2.0 Kit Luminescent cell Viability Assay (Vazyme Biotech Co., Ltd., Cat #: DD1101-02). The results are shown in the table below.

Sacituzumab govitecan, from Immunomedics, Inc, is a humanized anti-TROP2 monoclonal antibody-drug conjugate.

Cetuximab is an EGFR-targeting chimeric monoclonal IgG1 antibody from Merck.

TABLE 13

| | IC50 (nM) | | | |
|---|---|---|---|---|
| ADCs | HCC827 | NCI-H292 | A431 | Panc 02.03 |
| T-6F7-E−6C4-(II′)-2 (DAR8) | 0.0598 | 0.6129 | 0.5048 | 14.12 |
| CPT2 | 1.6890 | 0.7275 | 1.3750 | 0.9806 |
| Sacituzumab govitecan | 0.3963 | — | — | 0.6639 |
| Cetuximab | 5.1250 | — | — | NA |

(NA means no in vitro killing activity; — means not tested)
The above results showed that T-6F7-E−6C4-(II′)-2 (DAR8) had good in vitro killing activity in HCC827 cells, NCI-H292 cells, A431 cells, and Panc 02.03 cells.

### Internalization of Anti-TROP2/EGFR Bispecific Antibody and ADCs

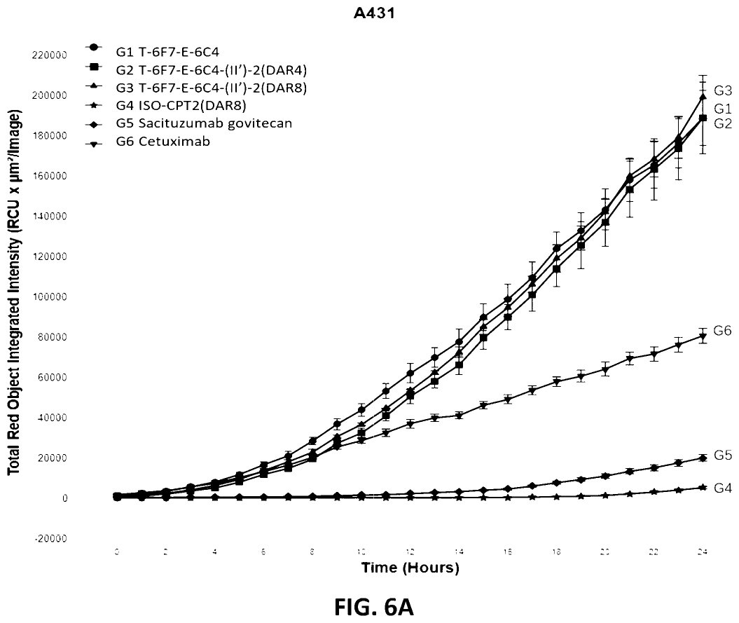

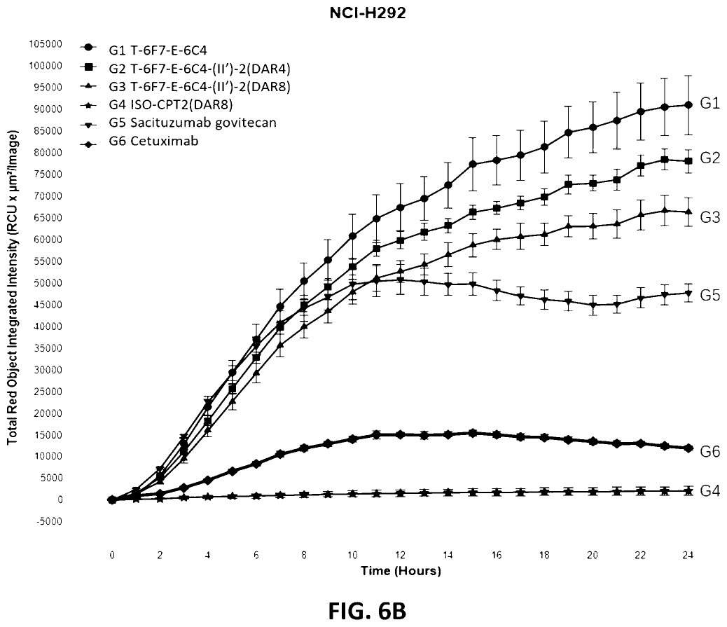

The anti-TROP2/EGFR bispecific antibody and ADCs (as shown in the table below) were used to treat A431 cells or NCI-H292 cells cultured in a cell culture plate, and the internalization activity was monitored over a 24-hour period after incubation using IncuCyte (Sartorius AG, IncuCyte® S3), with images captured every hour. The results are shown in FIGS. 6A-6B, which showed that the endocytosis activities of T-6F7-E-6C4-(II′)-2 (DAR4), T-6F7-E-6C4-(II′)-2 (DAR8) and T-6F7-E-6C4 were better than Sacituzumab govitecan and Cetuximab.

TABLE 14

| Group | Antibodies/ADCs |
|---|---|
| G1 | T-6F7-E−6C4 |
| G2 | T-6F7-E−6C4-(II′)-2(DAR4) |
| G3 | T-6F7-E−6C4-(II′)-2(DAR8) |
| G4 | ISO-(II′)-2(DAR8) |
| G5 | Sacituzumab govitecan |
| G6 | Cetuximab |

### Binding Activities of Anti-TROP2/EGFR Bispecific Antibody and ADCs

This experiment was performed to test the binding activities of the anti-TROP2/EGFR bispecific antibody and ADCs to tumor cell lines.

Specifically, A431 cells or human lung cancer HCC827 cells (ATCC, Cat #: CRL-2868) were transferred to a 96-well plate at a density of 2×10⁵ cells/well, respectively. Serially diluted anti-TROP2/EGFR bispecific antibody or ADCs (the highest concentration:130 nM, diluted in a 2-fold series for 9 gradients) was added to the 96-well plate, and incubated at 4° C. for 25-30 minutes. Then, the cells were incubated with the secondary antibody Alexa Fluor® 647-conjugated AffiniPure F(ab′)₂ Fragment Goat Anti-Human IgG, Fcγ Fragment Specific (Jackson Immuno Research Laboratories, Inc., Cat #: 109-606-170) at 4° C. in the dark for 25-30 minutes before flow cytometry analysis. The results shown in the table below demonstrated that T-6F7-E-6C4-(II′)-2(DAR4), T-6F7-E-6C4-(II′)-2(DAR8), and T-6F7-E-6C4 can bind to A431 cells and HCC827 cells with high affinity.

TABLE 15

| | EC50 (nM) | |
|---|---|---|
| Antibodies/ADCs | A431 | HCC827 |
| T-6F7-E−6C4-(II′)-2(DAR4) | 1.914 | 1.582 |
| T-6F7-E−6C4-(II′)-2(DAR8) | 2.074 | 1.493 |
| T-6F7-E−6C4 | 2.504 | 1.987 |

### Example 11. Anti-Tumor Activity in Patient-Derived Breast Cancer Xenograft Model

B-NDG mice were engrafted in the right flank with breast cancer patient-derived tumor tissue fragments (2 mm×2 mm×2 mm). Immunofluorescence staining of patient-derived breast tumor fragments was performed and the images were analyzed via HALO 3.2 version. The results showed that EGFR-positive cells and TROP2-positive cells in the tumor fragments were 96.92% and 49.87%, respectively. When the tumors in the mice reached a volume of about 200-300 mm³, the mice were randomly placed into different groups based on the tumor volume. The mice were then injected with PBS or ADCs by i.v. administration. Details of the dosing schedule, route, and frequency are shown in the table below.

TABLE 16

| | No. of | | | | | Total No. of |
|---|---|---|---|---|---|---|
| Group | mice | ADCs | Dosage | Route | Frequency | administration |
| G1 | 5 | PBS | — | i.v. | QW | 1 |
| G2 | 5 | T-6F7-E-6C4-ADC | 3 mg/kg | i.v. | QW | 1 |
| G3 | 5 | T-6F7-E-6C4-(II′)-2(DAR4) | 3 mg/kg | i.v. | QW | 1 |
| G4 | 5 | T-6F7-E-6C4-(II′)-2(DAR4) | 6 mg/kg | i.v. | QW | 1 |
| G5 | 5 | T-6F7-E-6C4-(II′)-2(DAR4) | 10 mg/kg | i.v. | QW | 1 |
| G6 | 5 | T-6F7-E-6C4-(II′)-2(DAR8) | 3 mg/kg | i.v. | QW | 1 |
| G7 | 5 | T-6F7-E-6C4-(II′)-2(DAR8) | 6 mg/kg | i.v. | QW | 1 |
| G8 | 5 | T-6F7-E-6C4-(II′)-2(DAR8) | 10 mg/kg | i.v. | QW | 1 |

The table below summarizes the results for this experiment, including the tumor volumes on the day of grouping (Day 0), 17 days after grouping (Day 17) and 35 days after grouping (Day 35); TGI (0%); the ratio of tumor-free mice on Day 35; and the statistical differences (P value) of tumor volume between the treatment and control groups.

TABLE 17

| | Tumor volume (mm³) | | | Tumor-free | TGI (%) | P value |
|---|---|---|---|---|---|---|
| Group | Day 0 | Day 17 | Day 35 | (Day 35) | (Day 35) | (Day 35) |
| G1 | 205 ± 20 | 899 ± 132 | 1684 ± 345 | 0/5 | NA | NA |
| G2 | 204 ± 31 | 144 ± 41 | 629 ± 226 | 0/5 | 71.2 | 0.036 |
| G3 | 205 ± 29 | 152 ± 54 | 450 ± 193 | 0/5 | 83.4 | 0.014 |
| G4 | 205 ± 26 | 122 ± 24 | 370 ± 88 | 0/5 | 88.8 | 0.003 |
| G5 | 205 ± 33 | 43 ± 9 | 126 ± 45 | 0/5 | 105.3 | 0.001 |
| G6 | 205 ± 28 | 111 ± 36 | 332 ± 152 | 0/5 | 91.4 | 0.006 |
| G7 | 204 ± 30 | 29 ± 6 | 37 ± 15 | 2/5 | 111.3 | 0.001 |
| G8 | 203 ± 33 | 56 ± 25 | 48 ± 17 | 1/5 | 110.4 | 0.001 |

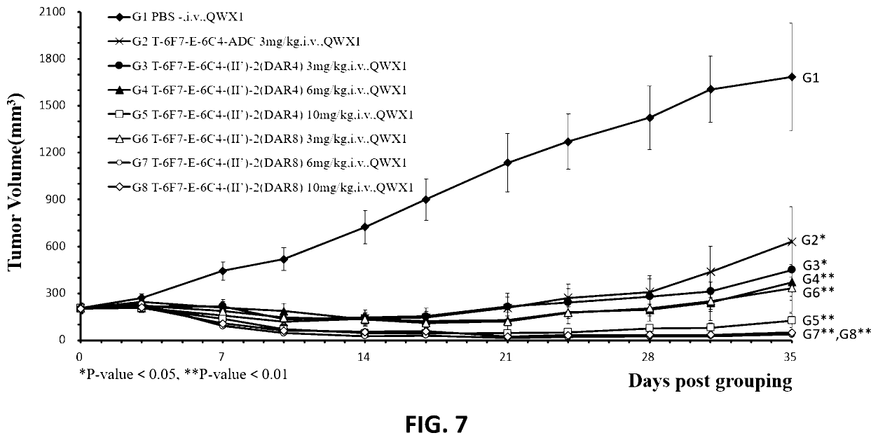

The tumor size in groups treated with PBS or ADCs are shown in FIG. 7. The results showed that T-6F7-E-6C4-(II′)-2 with DAR4 and DAR8 both exhibited good tumor inhibitory effects in a dose-dependent manner. In addition, T-6F7-E-6C4-(II′)-2 with DAR4 exhibited better tumor inhibitory effect than T-6F7-E-6C4-ADC at the dosage of 3 mg/kg.

### Example 12. Anti-Tumor Activity in Patient-Derived Pancreatic Cancer Xenograft Model

B-NDG mice were engrafted in the right flank with pancreatic cancer patient-derived tumor tissue fragments (2 mm×2 mm×2 mm). The immunofluorescence staining results showed that EGFR-positive cells and TROP2-positive cells in the pancreatic tumor fragments were 71.08% and 89.09%, respectively. When the tumors in the mice reached a volume of about 200-300 mm³, the mice were randomly placed into different groups based on the tumor volume. The mice were then injected with PBS or ADCs by i.v. administration. Details are shown in the table below.

TABLE 18

| | No. of | | | | | Total No. of |
|---|---|---|---|---|---|---|
| Group | mice | ADCs | Dosage | Route | Frequency | administration |
| G1 | 5 | PBS | — | i.v. | QW | 1 |
| G2 | 5 | ISO-(II′)-2(DAR4) | 6 mg/kg | i.v. | QW | 1 |
| G3 | 5 | T-6F7-E-6C4-(II′)-2(DAR4) | 1 mg/kg | i.v. | QW | 1 |
| G4 | 5 | T-6F7-E-6C4-(II′)-2(DAR4) | 3 mg/kg | i.v. | QW | 1 |
| G5 | 5 | T-6F7-E-6C4-(II′)-2(DAR4) | 6 mg/kg | i.v. | QW | 1 |
| G6 | 5 | T-6F7-E-6C4-(II′)-2(DAR8) | 1 mg/kg | i.v. | QW | 1 |
| G7 | 5 | T-6F7-E-6C4-(II′)-2(DAR8) | 3 mg/kg | i.v. | QW | 1 |
| G8 | 5 | T-6F7-E-6C4-(II′)-2(DAR8) | 6 mg/kg | i.v. | QW | 1 |

The table below summarizes the results for this experiment, including the tumor volumes on the day of grouping (Day 0), 14 days after grouping (Day 14) and 32 days after grouping (Day 32); TGI (0%); and the statistical differences (P value) of tumor volume between the treatment and control groups.

TABLE 19

| | Tumor volume (mm³) | | | TGI (%) | P value |
|---|---|---|---|---|---|
| Group | Day 0 | Day 14 | Day 32 | (Day 32) | (Day 32) |
| G1 | 233 ± 14 | 730 ± 84 | 1676 ± 310 | NA | NA |
| G2 | 233 ± 19 | 454 ± 41 | 1315 ± 163 | 25.0 | 0.294 |
| G3 | 233 ± 14 | 606 ± 109 | 1545 ± 146 | 9.1 | 0.721 |
| G4 | 233 ± 22 | 281 ± 67 | 1066 ± 292 | 42.3 | 0.225 |
| G5 | 233 ± 23 | 181 ± 22 | 807 ± 137 | 60.3 | 0.024 |
| G6 | 233 ± 30 | 531 ± 71 | 1592 ± 235 | 5.9 | 0.833 |
| G7 | 233 ± 19 | 226 ± 42 | 1111 ± 128 | 39.2 | 0.093 |
| G8 | 233 ± 27 | 67 ± 26 | 378 ± 142 | 89.9 | 0.005 |

The results showed that T-6F7-E-6C4-(II′)-2 with DAR4 and DAR8 both exhibited tumor inhibitory effects in a dose-dependent manner.

### Example 13. Anti-Tumor Activity in SKOV-3 Xenograft Model

The ADCs were tested for their effects on tumor growth in vivo in a xenograft model of ovarian adenocarcinoma. Specifically, about 5×10⁶ SKOV-3 cells (ATCC, Cat #: HTB-77) were injected subcutaneously in B-NDG mice. When the tumors in the mice reached a volume of about 300 mm³, the mice were randomly placed into different groups based on tumor volume. The mice were then injected with PBS or ADCs by intravenous (i.v.) administration. Details are shown in the table below.

TABLE 20

| | No. of | | | | | Total No. of |
|---|---|---|---|---|---|---|
| Group | mice | ADCs | Dosage | Route | Frequency | administration |
| G1 | 5 | PBS | — | i.v. | QW | 2 |
| G2 | 5 | T-6F7-E-6C4-(II′)-2(DAR4) | 3 mg/kg | i.v. | QW | 2 |
| G3 | 5 | T-6F7-E-6C4-(II′)-2(DAR4) | 6 mg/kg | i.v. | QW | 2 |
| G4 | 5 | T-6F7-E-6C4-(II′)-2(DAR4) | 10 mg/kg | i.v. | QW | 2 |
| G5 | 5 | T-6F7-E-6C4-(II′)-2(DAR8) | 3 mg/kg | i.v. | QW | 2 |
| G6 | 5 | T-6F7-E-6C4-(II′)-2(DAR8) | 6 mg/kg | i.v. | QW | 2 |
| G7 | 5 | T-6F7-E-6C4-(II′)-2(DAR8) | 10 mg/kg | i.v. | QW | 2 |

The table below summarizes the results for this experiment, including the tumor volumes on the day of grouping (Day 0), 17 days after grouping (Day 17) and 35 days after grouping (Day 35); TGI (%); and the statistical differences (P value) of tumor volume between the treatment and control groups.

TABLE 21

| | Tumor volume (mm³) | | | TGI (%) | P value |
|---|---|---|---|---|---|
| Group | Day 0 | Day 17 | Day 35 | (Day 35) | (Day 35) |
| G1 | 263 ± 17 | 1112 ± 79 | 2813 ± 243 | NA | NA |
| G2 | 263 ± 11 | 652 ± 59 | 1410 ± 208 | 55.0 | 0.002 |
| G3 | 263 ± 13 | 523 ± 38 | 919 ± 92 | 74.3 | 8.494E−05 |
| G4 | 263 ± 12 | 372 ± 21 | 686 ± 93 | 83.4 | 3.747E−05 |
| G5 | 263 ± 9 | 528 ± 18 | 858 ± 95 | 76.7 | 6.959E−05 |
| G6 | 264 ± 13 | 421 ± 49 | 749 ± 34 | 81.0 | 3.033E−05 |
| G7 | 263 ± 10 | 219 ± 24 | 247 ± 25 | 100.6 | 5.877E−06 |

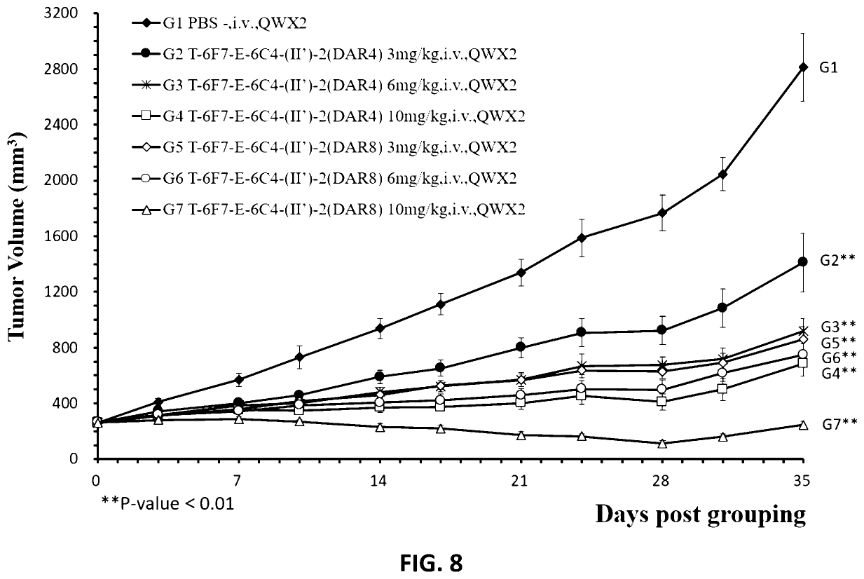

The tumor size in groups treated with PBS or ADCs are shown in FIG. 8. The results showed that T-6F7-E-6C4-(II′)-2 with DAR4 and DAR8 both exhibited tumor inhibitory effects in ovarian adenocarcinoma model in a dose-dependent manner, and T-6F7-E-6C4-(II′)-2(DAR8) exhibited better tumor inhibitory effects than T-6F7-E-6C4-(II′)-2 (DAR4).

### Example 14. Anti-Tumor Activity in A431 Xenograft Model

The ADCs were tested for their effects on tumor growth in vivo in a xenograft model of epidermoid carcinoma. Specifically, about 1×10⁶ A431 cells were injected subcutaneously in B-NDG mice. When the tumors in the mice reached a volume of about 200 mm³, the mice were randomly placed into different groups based on tumor volume. The mice were then injected with PBS, antibody, or ADCs by intravenous (i.v.) administration. Details are shown in the table below.

TABLE 22

| | No. of | | | | | Total No. of |
|---|---|---|---|---|---|---|
| Group | mice | Antibodies/ADCs | Dosage | Route | Frequency | administration |
| G1 | 5 | PBS | — | i.v. | QW | 2 |
| G2 | 5 | ISO-(II′)-2(DAR4) | 10 mg/kg | i.v. | QW | 2 |
| G3 | 5 | ISO-(II′)-2(DAR8) | 10 mg/kg | i.v. | QW | 2 |
| G4 | 5 | Sacituzumab govitecan | 10 mg/kg | i.v. | BIW | 4 |
| G5 | 5 | T-6F7-E-6C4-(II′)-2(DAR4) | 3 mg/kg | i.v. | QW | 2 |
| G6 | 5 | T-6F7-E-6C4-(II′)-2(DAR4) | 6 mg/kg | i.v. | QW | 2 |
| G7 | 5 | T-6F7-E-6C4-(II′)-2(DAR4) | 10 mg/kg | i.v. | QW | 2 |
| G8 | 5 | Cetuximab | 10 mg/kg | i.v. | BIW | 4 |
| G9 | 5 | T-6F7-E-6C4-(II′)-2(DAR8) | 3 mg/kg | i.v. | QW | 2 |
| G10 | 5 | T-6F7-E-6C4-(II′)-2(DAR8) | 6 mg/kg | i.v. | QW | 2 |
| G11 | 5 | T-6F7-E-6C4-(II′)-2(DAR8) | 10 mg/kg | i.v. | QW | 2 |

The body weights were measured twice a week. During the experiment, body weights of mice in all groups increased and there was no significant difference in body weights among groups, indicating that the tested ADCs were well tolerated and not obviously toxic to the mice.

The table below summarizes the results for this experiment, including the tumor volumes on the day of grouping (Day 0), 17 days after grouping (Day 17) and 31 days after grouping (Day 31); TGI (%); and the statistical differences (P value) of tumor volume between the treatment and control groups.

TABLE 23

| | Tumor volume (mm³) | | | TGI (%) | P value |
|---|---|---|---|---|---|
| Group | Day 0 | Day 17 | Day 31 | (Day 31) | (Day 31) |
| G1 | 197 ± 10 | 1204 ± 66 | 2079 ± 191 | NA | NA |
| G2 | 197 ± 11 | 946 ± 123 | 1684 ± 258 | 21.0 | 0.253 |
| G3 | 196 ± 3 | 634 ± 69 | 1439 ± 168 | 34.0 | 0.036 |
| G4 | 197 ± 6 | 682 ± 71 | 1554 ± 144 | 27.9 | 0.059 |
| G5 | 197 ± 4 | 601 ± 54 | 1446 ± 175 | 33.6 | 0.040 |
| G6 | 197 ± 7 | 209 ± 23 | 657 ± 79 | 75.5 | 1.247E−04 |
| G7 | 196 ± 5 | 135 ± 13 | 189 ± 29 | 100.4 | 9.814E−06 |
| G8 | 197 ± 10 | 373 ± 23 | 806 ± 59 | 67.6 | 2.145E−04 |
| G9 | 196 ± 4 | 282 ± 31 | 1166 ± 139 | 48.5 | 0.005 |
| G10 | 197 ± 4 | 155 ± 17 | 277 ± 35 | 95.7 | 1.451E−05 |
| G11 | 197 ± 5 | 103 ± 6 | 67 ± 7 | 106.9 | 5.681E−06 |

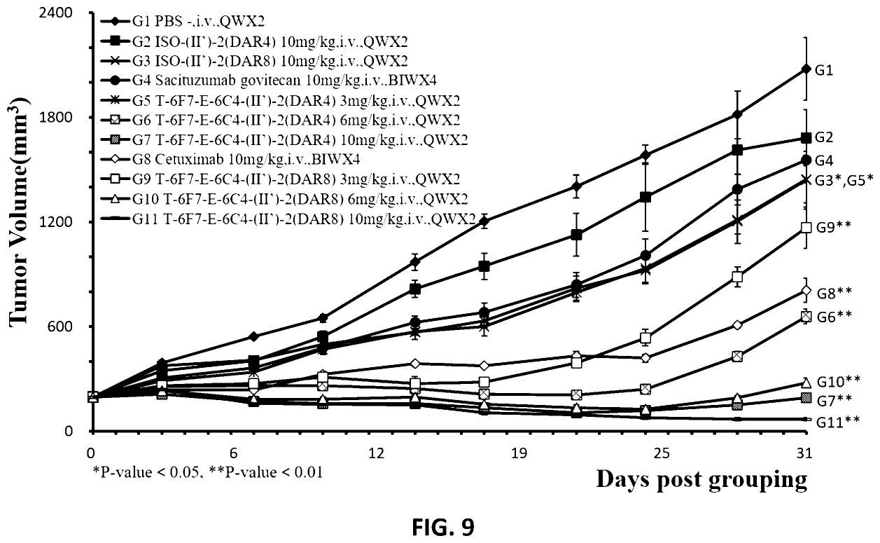

The tumor size in groups treated with PBS, antibody, or ADCs are shown in FIG. 9. The results showed that T-6F7-E-6C4-(II′)-2 with DAR4 and DAR8 both exhibited better tumor inhibitory effects than Sacituzumab govitecan or Cetuximab, in a dose-dependent manner. Further, the experiment was continued until 49 days after grouping (Day 49), and T-6F7-E-6C4-(II′)-2 with DAR4 and DAR8 of 6 mg/kg or 10 mg/kg still showed tumor inhibitory effects.

### Example 15. Anti-Tumor Activity in NCI-1H292 Xenograft Model

The antibody or ADCs were tested for their effects on tumor growth in vivo in a xenograft model of lung cancer. Specifically, about 5×10⁶ NCI-H292 cells were injected subcutaneously in B-NDG mice. When the tumors in the mice reached a volume of about 300 mm³, the mice were randomly placed into different groups based on tumor volume. The mice were then injected with PBS, antibody, or ADCs by intravenous (i.v.) administration. Details are shown in the table below.

TABLE 24

| | No. of | | | | | Total No. of |
|---|---|---|---|---|---|---|
| Group | mice | Antibodies/ADCs | Dosage | Route | Frequency | administration |
| G1 | 5 | PBS | — | i.v. | QW | 1 |
| G2 | 5 | ISO-(II′)-2(DAR4) | 10 mg/kg | i.v. | QW | 1 |
| G3 | 5 | ISO-(II′)-2(DAR8) | 10 mg/kg | i.v. | QW | 1 |
| G4 | 5 | Sacituzumab govitecan | 10 mg/kg | i.v. | BIW | 2 |
| G5 | 5 | T-6F7-E-6C4-(II′)-2(DAR4) | 3 mg/kg | i.v. | QW | 1 |
| G6 | 5 | T-6F7-E-6C4-(II′)-2(DAR4) | 6 mg/kg | i.v. | QW | 1 |
| G7 | 5 | T-6F7-E-6C4-(II′)-2(DAR4) | 10 mg/kg | i.v. | QW | 1 |
| G8 | 5 | Cetuximab | 10 mg/kg | i.v. | BIW | 2 |
| G9 | 5 | T-6F7-E-6C4-(II′)-2(DAR8) | 3 mg/kg | i.v. | QW | 1 |
| G10 | 5 | T-6F7-E-6C4-(II′)-2(DAR8) | 6 mg/kg | i.v. | QW | 1 |
| G11 | 5 | T-6F7-E-6C4-(II′)-2(DAR8) | 10 mg/kg | i.v. | QW | 1 |

The table below summarizes the results for this experiment, including the tumor volumes on the day of grouping (Day 0), 21 days after grouping (Day 21) and at the end of the experiment (Day 39); the survival rate of the mice; TGI (%); and the statistical differences (P value) of tumor volume between the treatment and control groups.

TABLE 25

| | Tumor volume (mm³) | | | TGI (%) | P value |
|---|---|---|---|---|---|
| Group | Day 0 | Day 21 | Day 39 | (Day 39) | (Day 39) |
| G1 | 297 ± 7 | 1227 ± 99 | 2172 ± 243 | NA | NA |
| G2 | 297 ± 6 | 880 ± 68 | 1723 ± 237 | 23.9 | 0.222 |
| G3 | 297 ± 7 | 361 ± 38 | 1051 ± 93 | 59.8 | 0.003 |
| G4 | 297 ± 11 | 428 ± 51 | 893 ± 62 | 68.2 | 0.001 |
| G5 | 297 ± 9 | 692 ± 109 | 1231 ± 83 | 50.2 | 0.006 |
| G6 | 297 ± 9 | 177 ± 34 | 812 ± 54 | 72.5 | 0.001 |
| G7 | 297 ± 7 | 24 ± 4 | 351 ± 52 | 97.1 | 8.164E−05 |
| G8 | 297 ± 11 | 592 ± 24 | 1390 ± 99 | 41.7 | 0.018 |
| G9 | 297 ± 10 | 499 ± 40 | 1051 ± 34 | 59.8 | 0.002 |
| G10 | 297 ± 8 | 52 ± 3 | 417 ± 61 | 93.6 | 1.126E−04 |
| G11 | 297 ± 8 | 22 ± 6 | 254 ± 25 | 102.3 | 2.242E−04 |

The results showed that T-6F7-E-6C4-(II′)-2 with DAR4 and DAR8 both exhibited good tumor inhibitory effects in lung cancer model in a dose-dependent manner. In addition, T-6F7-E-6C4-(II′)-2 with DAR4 and DAR8 both exhibited good tumor inhibitory effects with higher TGI than that of Sacituzumab govitecan or Cetuximab at the dose level of 10 mg/kg.

### Example 16. Anti-Tumor Activity in Patient-Derived Xenograft Models

T-6F7-E-6C4-(II′)-2 (DAR8) was tested for its effect on tumor growth in a head and neck squamous cell carcinoma model, an esophageal cancer model, a colorectal cancer model, or a gastric cancer model. Specifically, BALB/c nude mice were engrafted with patient-derived tumor tissue fragments (2 mm×2 mm×2 mm). When the tumors in the mice reached a volume of about 100-200 mm³, the mice were randomly placed into different groups based on tumor volumes (3 mice in each group). The mice were then injected with saline (G1, control) or 6 mg/kg T-6F7-E-6C4-(II′)-2(DAR8) (G2) (1 injection in total).

Immunohistochemistry (IHC) staining of different patient-derived tumor tissues was performed, and the table below show the histochemistry score (H-score) of EGFR or TROP2 expression level in the patient-derived tumor tissues. Table 26 below also summarizes the TGI (%) of different patient-derived xenograft models.

TABLE 26

| | H-score | | Days after | TGI |
|---|---|---|---|---|
| PDX model | EGFR | TROP2 | grouping | (%) |
| Esophageal cancer PDX | 217.11 | 205.27 | Day21 | 113.59 |
| Head and neck squamous | / | / | Day27 | 117.34 |
| cell carcinoma PDX | | | | |
| Colorectal cancer PDX1 | 173.83 | 212.69 | Day25 | 94.41 |
| Colorectal cancer PDX2 | 231.38 | 39.98 | Day21 | 45.74 |
| Gastric cancer PDX1 | 109.65 | 34.06 | Day27 | 111.9 |
| Gastric cancer PDX2 | 248.67 | 278.37 | Day27 | 131.44 |

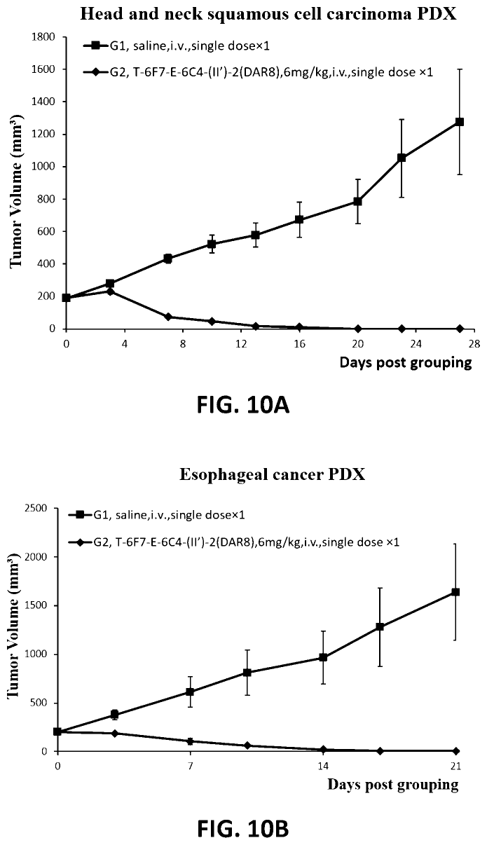

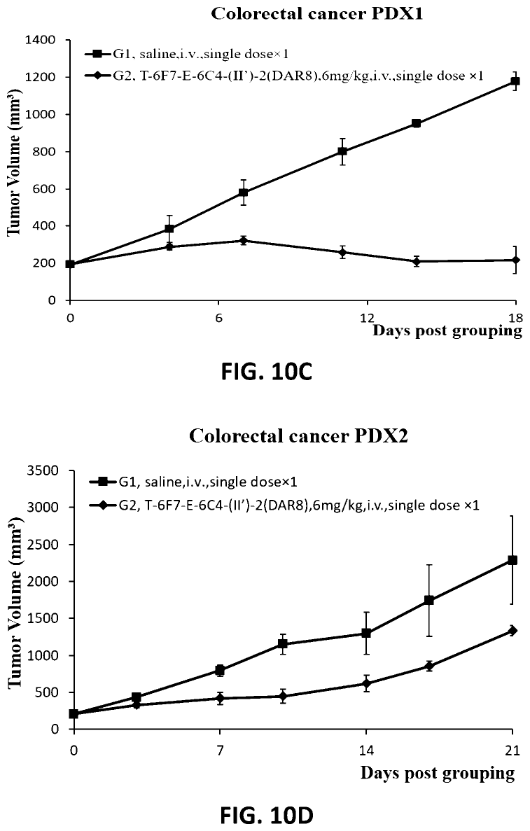

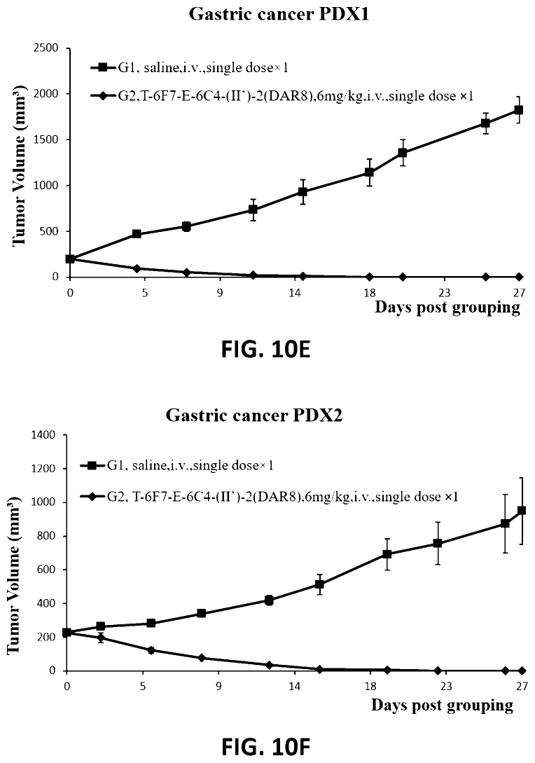

The tumor size in groups treated with saline or T-6F7-E-6C4-(II′)-2(DAR8) are shown in FIGS. 10A-10F, which showed that T-6F7-E-6C4-(II′)-2(DAR8) exhibited good tumor growth inhibition effects in head and neck squamous cell carcinoma, esophageal cancer, colorectal cancer, and gastric cancer.

### Example 17. Pharmacokinetic Profiles and Plasma Stability

The pharmacokinetic clearance rates of the anti-EGFR/TROP2 bispecific ADCs were determined in B-NDG mice. Specifically, about 1×10⁶ A431 cells were injected subcutaneously in B-NDG mice. When the tumors in the mice reached a volume of about 300 mm³, the mice were randomly placed into different groups based on tumor volume (3 mice per group) and then administered with PBS (G2), T-6F7-E-6C4-(II′)-2(DAR4) (G3-G10, 10 mg/kg), or T-6F7-E-6C4-(II′)-2(DAR8) (G11-G18, 10 mg/kg) by intravenous injection (one administration in total). G1 group was used as a blank control. Blood samples and tumor tissue samples of mice in group G3-G10 and G11-G18 were collected at 15 minutes, 2 hours, 6 hours, 1 day, 3 days, 5 days, 7 days, and 14 days after administration. Blood samples and tumor tissue samples of mice in G1 group were collected 1 hour before administration, while those of mice in G2 group were collected 14 days after administration. These collected samples were used to detect the total antibody levels in serum and tumor tissue by sandwich ELISA as well as free payload by MS (Mass Spectrometry).

The levels of total antibody were determined by sandwich ELISA. Briefly, Goat Anti-Human IgG (H+L) (Jackson ImmunoResearch Inc., Cat #: 109-005-088) was diluted to a final concentration of 2000 ng/mL, added to a 96-well plate (ELISA plate) at 100 μL/well, and then incubated overnight at 2-8° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL of blocking buffer (2% BSA) was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 μL/well to each well of the plate, and incubated at 37° C. for 1 hour for determining the concentration of total antibody and payload CPT2. After washing the plate, tetramethylbenzidine (TMB) solution was added at 100 μL/well to the 96-well plate as the substrate. After incubating at room temperature in the dark, 100 μL stop solution (Beyotime, Cat #: P0215) was added to each well. Luminescent signals of the plate were measured at 450 nm and 630 nm to calculate the concentrations. The absorbance value and corresponding concentration of the calibration sample prepared by each test product was used to create a standard curve with four parameters (i.e., T_{1/2}, Cₘₐₓ, AUC_{0-21day}, and CL). The standard curve was used to calculate the antibody or ADC concentration of each serum sample. A drug concentration-time curve was created using the calculated sample concentration at each time point. Phoenix™ WinNolin 8.3 was used to calculate the pharmacokinetic parameters.

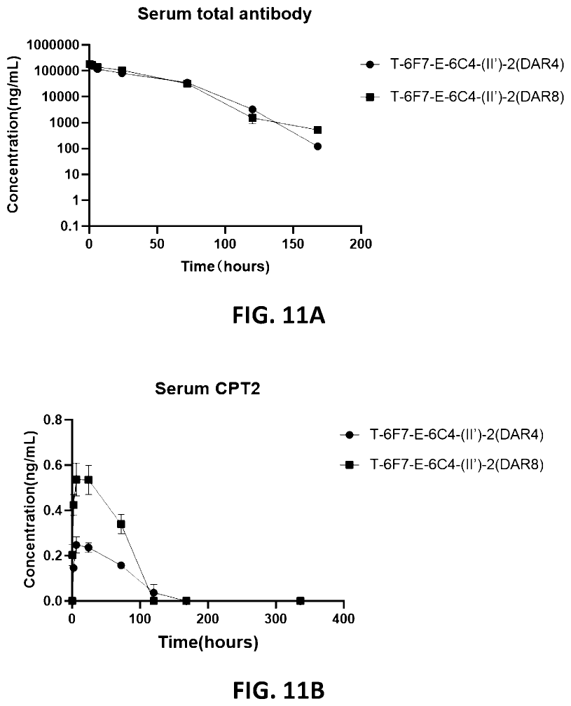

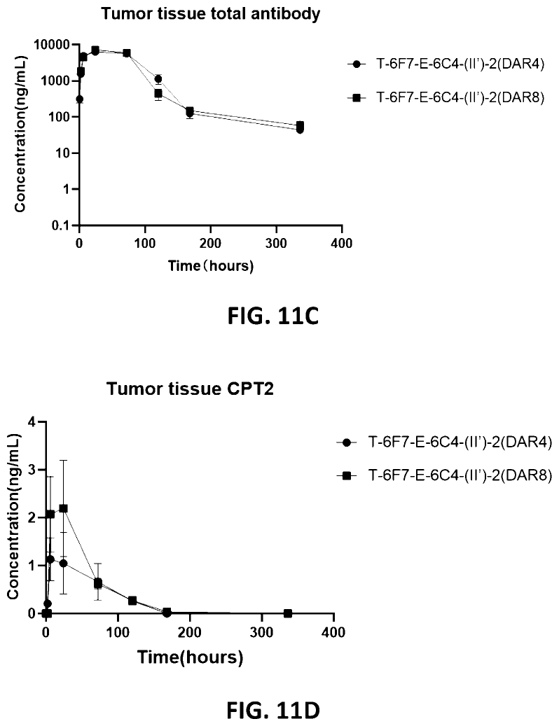

The results are shown in the table below and FIGS. 11A-11D, which showed that T-6F7-E-6C4-(II′)-2(DAR4) and T-6F7-E-6C4-(II′)-2(DAR8) exhibited expected PK behavior.

TABLE 27

| | Dose | | Cₘₐₓ | AUC₀₋ₗₐₛₜ | CL |
|---|---|---|---|---|---|
| ADCs | (mg/kg) | LBA | (ng/mL) | (hour*ng/mL) | (mL/hour/kg) |
| T-6F7- | 10 | Serum CPT2 | 0.25 | 21.33 | 2839.57 |
| E−6C4-(II′)-2 | | Serum antibody | 1206.14 | 42589.25 | 0 |
| (DAR4) | | Tumor tissue CPT2 | 3.96 | 357.1 | 237.47 |
| | | Tumor tissue | 22267.93 | 2136100.75 | 4.66 |
| | | antibody | | | |
| T-6F7- | 10 | Serum CPT2 | 0.54 | 33.18 | NA |
| E−6C4-(II′)-2 | | Serum antibody | 181302.67 | 7350021.62 | 1.36 |
| (DAR8) | | Tumor tissue CPT2 | 7.68 | 522.32 | 366.82 |
| | | Tumor tissue | 25055.33 | 2168750.96 | 4.56 |
| | | antibody | | | |

Cₘₐₓ: Max Concentration;
AUC₀₋ₗₐₛₜ: Area Under the Curve from Time Zero to Last Quantifiable Concentration
CL: Clearance

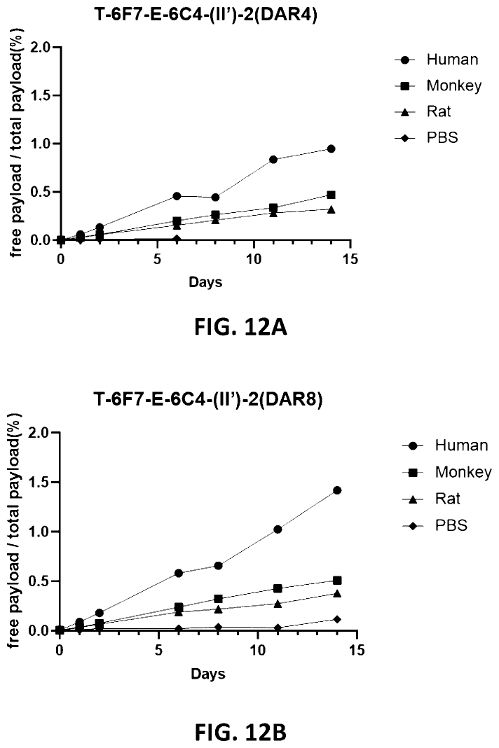

In another experiment, the plasma stability of T-6F7-E-6C4-(II′)-2(DAR4) and T-6F7-E-6C4-(II′)-2 (DAR8) were determined in human plasma, monkey (Macaca fascicularis) plasma, and rat (SD rat) plasma. Specifically, T-6F7-E-6C4-(II′)-2(DAR4) or T-6F7-E-6C4-(II′)-2(DAR8) were added to human, monkey, or rat plasma, respectively, to a terminal concentration of 100 μg/mL. In the control group, the plasma was replaced by PBS with 0.5% BSA. The contents of free payload CPT2 and ADC were determined in 0 day, 1 day, 2 days, 6 days, 8 days, 11 days and 14 days after adding the ADCs, and the ratios of free CPT2 to the total ADC were calculated. The results are shown in FIGS. 12A-12B, which indicated that T-6F7-E-6C4-(II′)-2(DAR4) and T-6F7-E-6C4-(II′)-2(DAR8) were relatively stable in human, monkey and rat plasma, with a release rate of free CPT2 no more than 2.0% at the highest.

### Example 18. Toxicology Evaluation

In a preliminary experiment, to investigate the safety and toxicokinetics (TK) profile, T-6F7-E-6C4-(II′)-2(DAR8) was administered by i.v. injection to cynomolgus monkeys, three times with a 3-week interval (on Day 1, Day 22, and Day 43). The dose fomulation is shown in the table below. Then, animals were sacrificed on Day 50 for gross and histopathological examination. Mortality/moribundity, general observations, body weights, food consumption, clinical pathology (hematology, coagulation, serum chemistry, and urinalysis), and gross lesions were evaluated. Blood samples were also collected for TK analysis and the primary TK parameters, e.g., Tmax, Cmax, and AUC (0-t) for payload, total antibody, and ADC were calculated. As a result, it was found that T-6F7-E-6C4-CPT2(DAR8) has a favorable safety profile.

TABLE 28

| | | Dose (mg/kg) | | |
|---|---|---|---|---|
| ADC | Group | Day 1 | Day 22 | Day 43 |
| T-6F7-E−6C4-(II′)-2(DAR8) | G1 | 5 | 20 | 20 |
| | G2 | 10 | 10 | 10 |
| | G3 | 30 | 30 | 30 |

### Example 19. Biophysical Analysis of Antibody Drug Conjugates

The biophysical properties of ADCs were evaluated. Specifically, the following tests were performed: (1) determining and quantifying the level of aggregates and fragments of purified antibodies/ADCs by Size-Exclusion-Ultra Performance Liquid Chromatography (SEC-UPLC) (indicated as the retention time of the main peak (SEC, min)); (2) detecting the apparent hydrophobicity of the antibodies/ADCs using the Hydrophobic Interaction Chromatography-High Performance Liquid Chromatography (HIC-HPLC) method (indicated as the retention time of the main peak (HIC, min)); (3) comparing the hydrophilicity of different antibodies/ADCs using the Reverse Phase-High Performance Liquid Chromatography (RP-HPLC) method, as demonstrated by the retention time of the heavy chain and the light chain of antibodies/ADC.

In the SEC-UPLC experiments, the samples were diluted to 1 mg/mL with purified water and an Agilent 1290 chromatograph system (connected with XBridge Protein BEH SEC column (200 Å, Waters Corporation)) was used. The following parameters were used: mobile phase: 25 mM phosphate buffer (PB)+300 mM NaCl, pH6.8; flow rate: 1.8 mL/min; column temperature: 25° C.; detection wavelength: 280 nm, 370 nm; injection volume: 10 μL; sample tray temperature: about 4° C.; and running time: 7 minutes.

In the HIC-HPLC experiments, the samples were diluted to 1 mg/mL with purified water and an Agilent 1260 chromatograph system (connected with Proteomix HIC phenyl column (4.6×250 mm, 5 μm, Sepax Technologies)) was used. The following parameters were used: mobile phase A: 1.5 M ammonium sulfate, 20 mM phosphate buffer (PB) pH 7.0; mobile phase B: 25% isopropanol, 20 mM phosphate buffer (PB) pH 7.0; flow rate: 0.6 mL/min; gradient: 0 min 100% A, 2 min 100% A, 20 min 100% B, 25 min 100% B, 25.1 min 100% A, and 30 min 100% A; column temperature: 25° C.; detection wavelength: 280 nm, 370 nm; injection mass: 20 μg; and running time: 40 minutes.

In the RP-HPLC experiments, the samples were treated with Tris-HCl and Dithiothreitol (DTT) and an Agilent 1260 chromatograph system (connected with UPLC: PLRP-S column (8 μm, 2.1×150 mm, 1000 Å, Agilent)) was used. The following parameters were used: mobile phase A: 0.05% trifluoroacetic acid aqueous solution; mobile phase B: 0.05% trifluoroacetic acid in acetonitrile solution; flow rate: 0.8 mL/min; gradient: 0 min 70.5% A-29.5% B, 3 min 70.5% A-29.5% B, 17 min 58% A-42% B, 19 min 5% A-95% B, 20 min 70.5% A-29.5% B, and 22 min 70.5% A-29.5% B; column temperature: 80° C.; detection wavelength: 280 nm, 370 nm; injection mass: 8 μg.

The results at the detection wavelength of 280 nm are summarized in the table below which indicated that T-(II′)-2 exhibited superhydrophilic properties similar to Trastuzumab analog, and significantly better than T-GGFG-Dxd. In addition, after the conjugation of compound (II′)-2, both with DAR4 and DAR8, T-6F7-E-6C4-(II′)-2 showed no aggregation and exhibited good hydrophilicity which is similar to T-6F7-E-6C4.

TABLE 29

| | SEC | HIC-HPLC | RP-HPLC(min) | | |
|---|---|---|---|---|---|
| Antibody | (min) | (min) | Light chain | Heavy chain | |
| T-(II′)-2 | — | 9.56 | 6.16 | 9.29 | / |
| T-GGFG-Dxd | — | 16.01 | 7.68 | 11.76 | / |
| Trastuzumab analog | — | 9.11 | 5.73 | 9.26 | / |
| T-6F7-E−6C4 | 3.966 | 10.56 | 6.45 | 9.89 | 11.02 |
| T-6F7-E−6C4-(II′)-2 (DAR4) | 3.920 | 10.73 | 6.66 | 9.92 | 11.02 |
| T-6F7-E−6C4-(II′)-2 (DAR8) | 3.857 | 10.69 | 6.63 | 9.89 | 10.99 |

(— means not detected)

1. A compound of Formula (I) 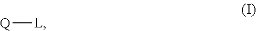
or a pharmaceutically acceptable salt, solvate, stereoisomer, or isotopic variant thereof, wherein
Q denotes to a junction moiety capable of being coupled to a ligand via a bond selected from the group consisting of carbonyl, thioether, amide, disulfide and hydrazone bond;
L denotes to a linker moiety capable of connecting Q to a therapeutic agent, and having a structure of: 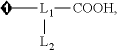
where L₁ is a polypeptide residue consisting of three to eight amino acid residues which comprises at least one amino acid residue with a side chain carboxyl group, where “—COOH” denotes carboxyl group of an amino acid residue at C-terminal of the polypeptide residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O)(CH₂CH₂O)ₘ(CH₂)ₚC(O)NHR^{L2d}, R^{L2d} is H or C₆ alkyl optionally substituted with 1 to 6 hydroxy groups, each m is independently an integer from 0 to 10, and each p is independent an integer from 1 to 4; and [symbol]
denotes to the N-terminal side of the polypeptide residue covalently attached to the junction moiety Q.

2. The compound or a pharmaceutically acceptable salt, solvate, stereoisomer, or isotopic variant thereof according to claim 1, wherein the junction moiety Q has a structure of: 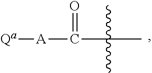
wherein Q^{a} is a function group capable of being coupled to a ligand;
A is selected from optionally substituted C₃₋₈ alkylidene, optionally substituted C₃₋₈ alkenylidene, optionally substituted C₃₋₆ cycloalkenylidene, optionally substituted C₃₋₈ cycloalkylidene, optionally substituted diglycol to octaglycol acyl, where alkylidene, alkenylidene, cycloalkenylidene, cycloalkylidene, diglycol to octaglycol acyl is optionally substituted with 1 to 4 substituents independently selected from the group consisting of halogen, —CN, R^{Qa1}, —OR^{Qa1}, —SR^{Qa1}, and —N(R^{Qa1})₂, where each R^{Qa1} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“ [symbol] ” denotes to the site covalently attached to the linker moiety L.

3. The compound or a pharmaceutically acceptable salt, solvate, stereoisomer, or isotopic variant thereof according to claim 2, wherein the function group Q^{a} is selected from the group consisting of 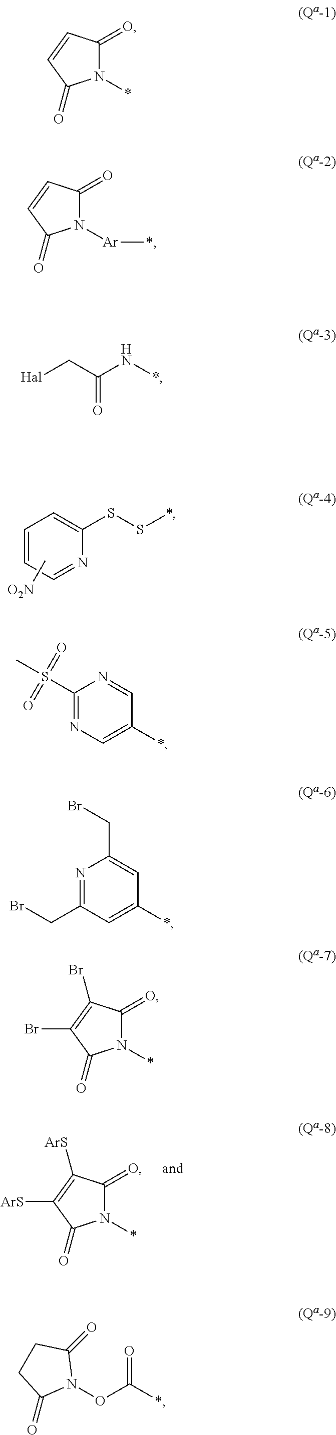
where Hal is a halogen selected from the group consisting of Cl, Br and I;
Ar is selected from the group consisting of optionally substituted C₅₋₆ cycloalkenyl, optionally substituted C₆ aryl and optionally substituted 5- to 6-membered heteroaryl, where cycloalkenyl, aryl and heteroaryl optionally substituted with 1 to 4 substituents independently selected from the group consisting of halogen, —CN, R^{Qa2}, —OR^{Qa2}, —SR^{Qa2}, and —N(R^{Qa2})₂, where each R^{Qa2} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“*” denotes to the site covalently attached to A.

4. The compound or a pharmaceutically acceptable salt, solvate, stereoisomer, or isotopic variant thereof according to any of claims 1 to 3, wherein the polypeptide residue L₁ has a sequence as shown below: 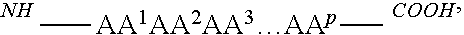
wherein each AA¹, AA², AA³, . . . AA^{p} is independently optionally substituted amino acid residue, and at least one of AA¹, AA², AA³, . . . AA^{p} is an amino acid residue with a side chain carboxyl group, preferably Glu or Asp;
p is an integer from 3 to 8, preferably from 3 to 5;
“NH—” denotes to N-terminal side of the polypeptide residue;
“—COOH” denotes to C-terminal side of the polypeptide residue.

5. The compound or a pharmaceutically accepted salt, solvate, stereoisomer, or isotopic variant thereof according to claim 4, wherein each AA1, AA2, AA3, . . . AAp independently optionally substituted amino acid residue selected from the group consisting of Glu, Asp, Pro, Nva, Leu, Ile, Met, Tyr, Trp, Ser, Thr, Cys, Asn, Gln, Arg, Phe, Lys, Val, Ala, Cit, Gly, and N-alkyl amino acids, and at least one of AA1, AA2, AA3, . . . AAp is Glu or Asp.

6. The compound or a pharmaceutically acceptable salt, solvate, stereoisomer, or isotopic variant thereof according to claim 4 or 5, wherein AA¹ is an amino acid residue with a side chain carboxyl group, preferably Glu or Asp; each AA², AA³, . . . AA^{p} is independently optionally substituted amino acid residue selected from the group consisting of Pro, Nva, Leu, Ile, Met, Tyr, Trp, Ser, Thr, Cys, Asn, Gln, Arg, Phe, Lys, Val, Ala, Cit, and Gly.

7. The compound or a pharmaceutically acceptable salt, solvate, stereoisomer, or isotopic variant thereof according to any of claims 1 to 6, wherein the polypeptide residue L₁ is selected from the group consisting of ^{NH}-Glu-Phe-Lys(NR^{Lys1}R^{Lys2})—^{COOH}, ^{NH}-Glu-Val-Lys(NR^{Lys1}R^{Lys2})—^{COOH}, ^{NH}-Glu-Ala-Ala-Ala-^{COOH}, ^{NH}-Glu-Ala-Ala-^{COOH}, ^{NH}-Glu-Val-Ala-^{COOH}, ^{NH}-Glu-Val-Cit-^{COOH}, ^{NH}-Glu-Gly-Gly-Phe-Gly-^{COOH}, ^{NH}-Asp-Phe-Lys-^{COOH}, ^{NH}-Asp-Ala-Ala-Ala-^{COOH}, ^{NH}-Asp-Val-Ala-^{COOH}, ^{NH}-Asp-Val-Cit-^{COOH}, ^{NH}-Asp-Gly-Gly-Phe-Gly-^{COOH} and ^{NH}-Asp-Val-Lys(NR^{Lys1}R^{Lys2})—^{COOH}, where R^{Lys1} and R^{Lys2} are each independently H or C₁₋₆ alkyl.

8. The compound or a pharmaceutically acceptable salt, solvate, stereoisomer, or isotopic variant thereof according to any of claims 1 to 7, wherein the hydrophilic group L₂ has a structure of —NHC(R^{L2a})(R^{L2b})(R^{L2c}), where R^{L2a}, R^{L2b}, and R^{L2c} are each independently selected from the group consisting of H, —CH₂O(CH₂)₂C(O)OH, and —CH₂O(CH₂)₂C(O)NHR^{L2d}, and at most two of R^{L2a}, R^{L2b}, and R^{L2c} are simultaneously H, R^{L2d} is C₄₋₆ alkyl substituted with 3 to 5 hydroxy groups, preferably R^{L2d} is C₄₋₆ alkyl substituted with 3 to 5 hydroxy groups, where no more than one hydroxyl group is substituted on each carbon atom, for example, R^{L2d} is selected from the group consisting of:

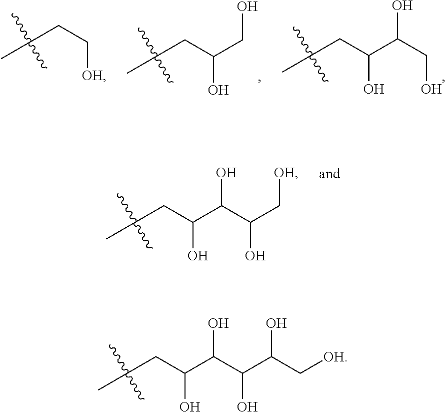

9. The compound or a pharmaceutically acceptable salt, solvate, stereoisomer, or isotopic variant thereof according to any of claims 1 to 8, wherein the hydrophilic group L₂ is selected from the group consisting of: 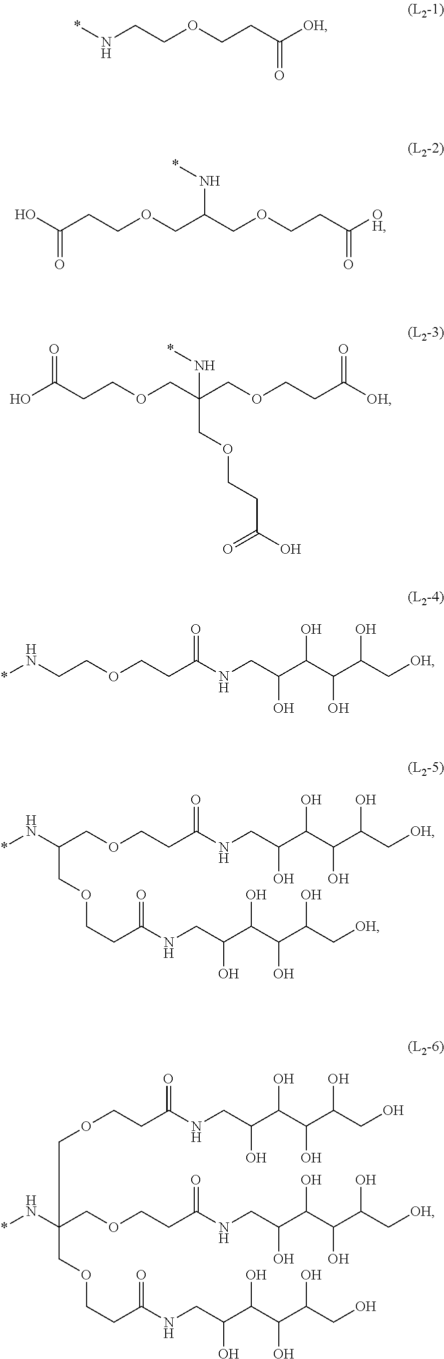 [SMILES: *NCCOCCC(=O)O ; *NC(COCCC(=O)O)COCCC(=O)O ; *NC(COCCC(=O)O)(COCCC(=O)O)COCCC(=O)O ; *NCCOCCC(=O)NCC(O)C(O)C(O)C(O)CO ; *NC(COCCC(=O)NCC(O)C(O)C(O)C(O)CO)COCCC(=O)NCC(O)C(O)C(O)C(O)CO ; *NC(COCCC(=O)NCC(O)C(O)C(O)C(O)CO)(COCCC(=O)NCC(O)C(O)C(O)C(O)CO)COCCC(=O)NCC(O)C(O)C(O)C(O)CO]
“*” denotes the site covalently attached to polypeptide residue L₁.

10. The compound or a pharmaceutically acceptable salt, solvate, stereoisomer, or isotopic variant thereof according to any of claims 1 to 9, wherein the compound is selected from the group consisting of

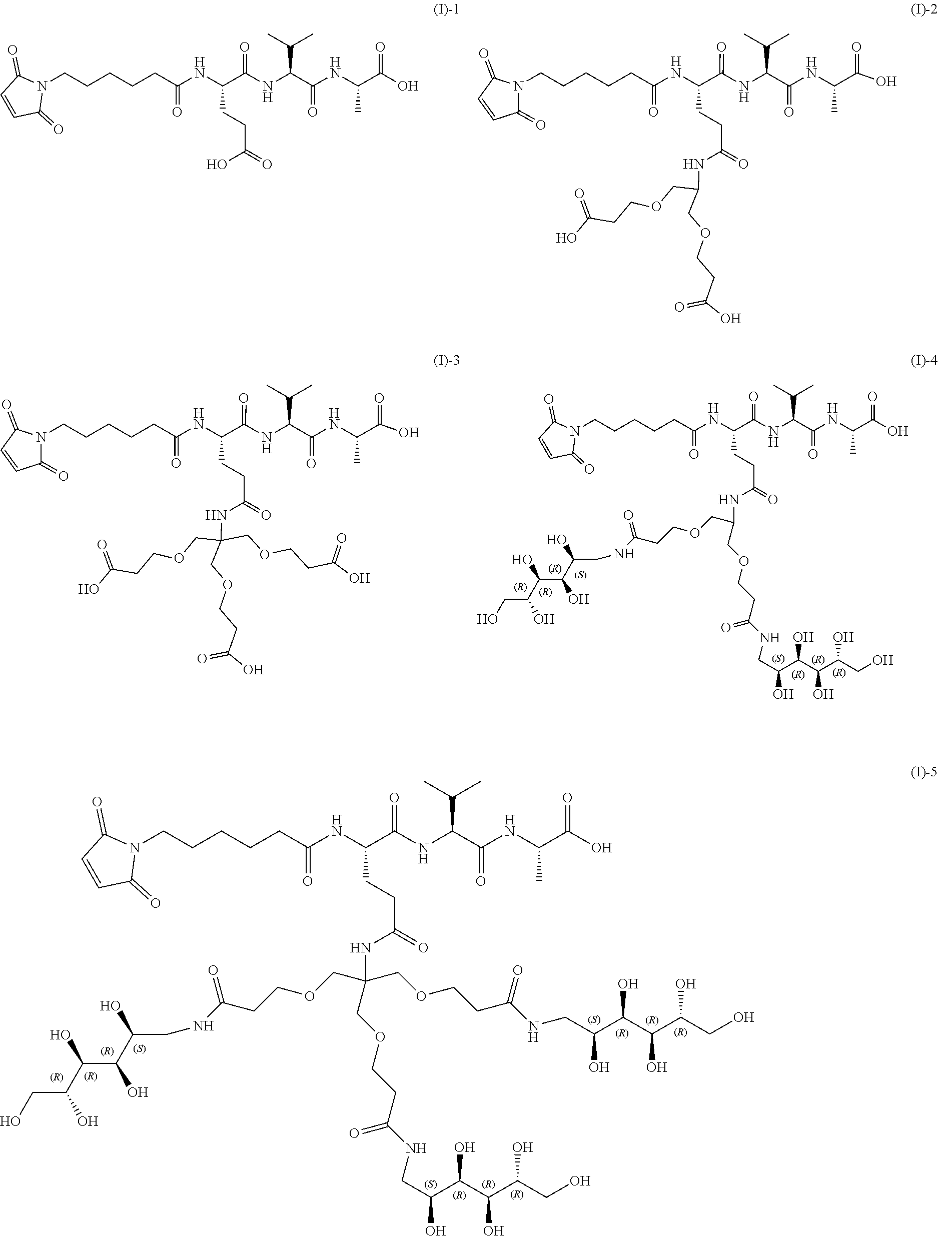

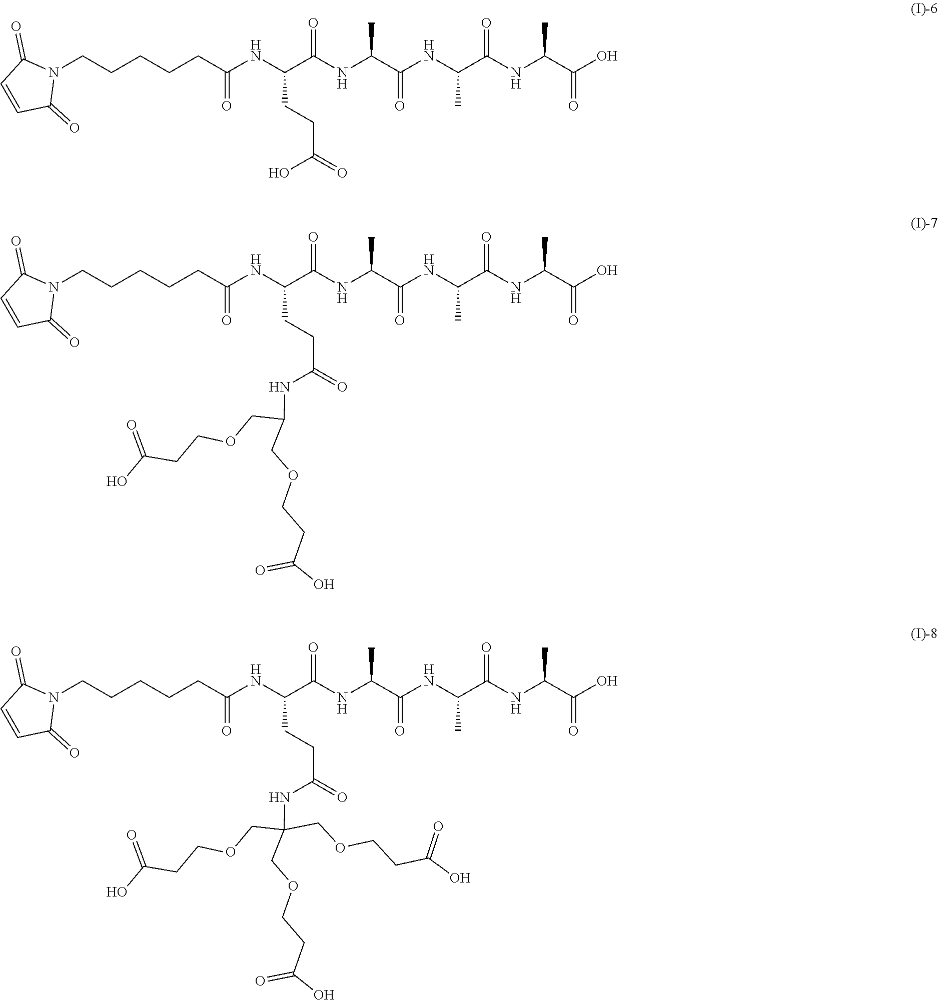 [SMILES: C[C@H](NC(=O)[C@H](C)NC(=O)[C@H](C)NC(=O)[C@H](CCC(=O)O)NC(=O)CCCCCN1C(=O)C=CC1=O)C(=O)O ; C[C@H](NC(=O)[C@H](C)NC(=O)[C@H](C)NC(=O)[C@H](CCC(=O)NC(COCCC(=O)O)COCCC(=O)O)NC(=O)CCCCCN1C(=O)C=CC1=O)C(=O)O ; C[C@H](NC(=O)[C@H](C)NC(=O)[C@H](C)NC(=O)[C@H](CCC(=O)NC(COCCC(=O)O)(COCCC(=O)O)COCCC(=O)O)NC(=O)CCCCCN1C(=O)C=CC1=O)C(=O)O]

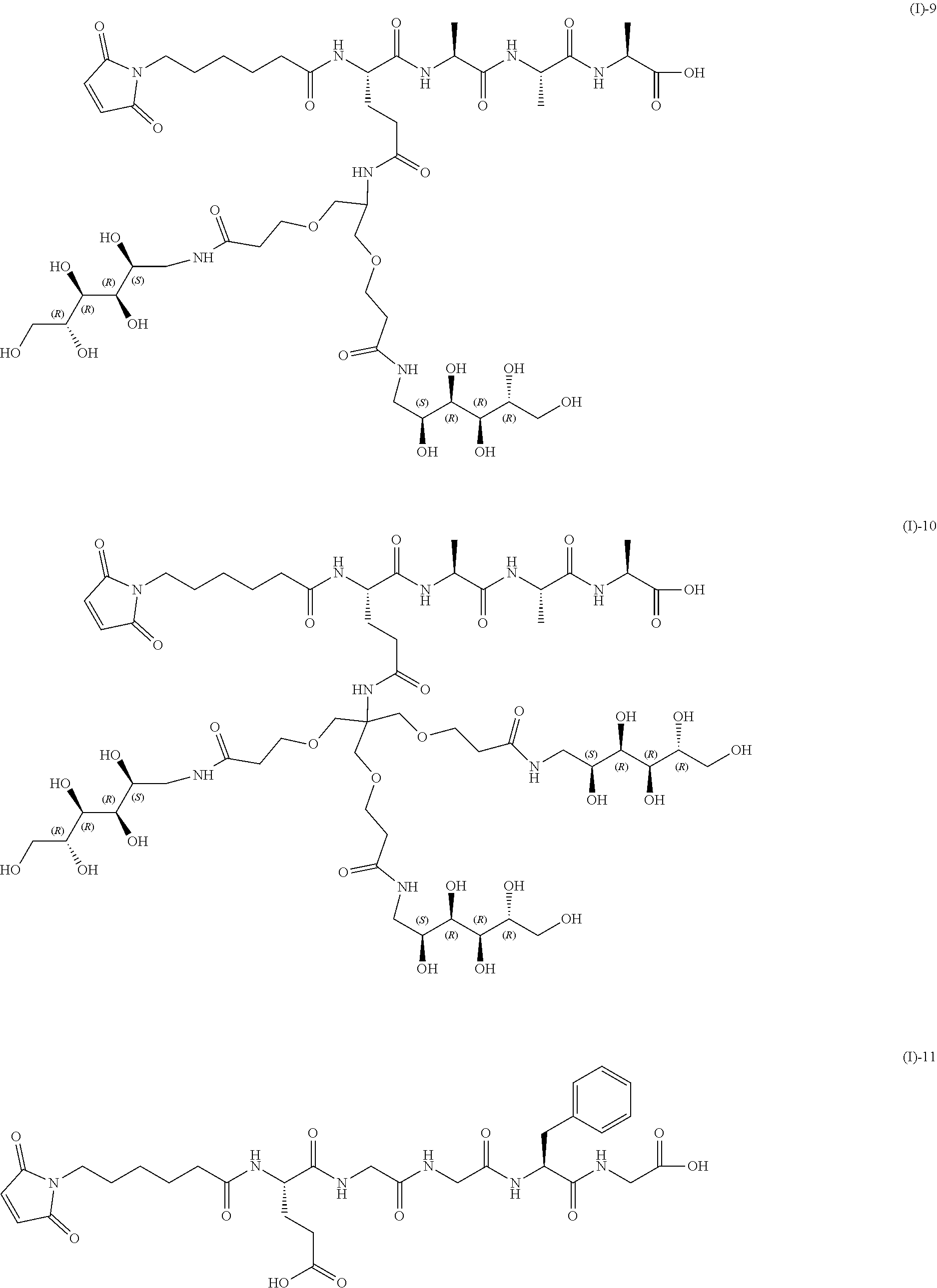

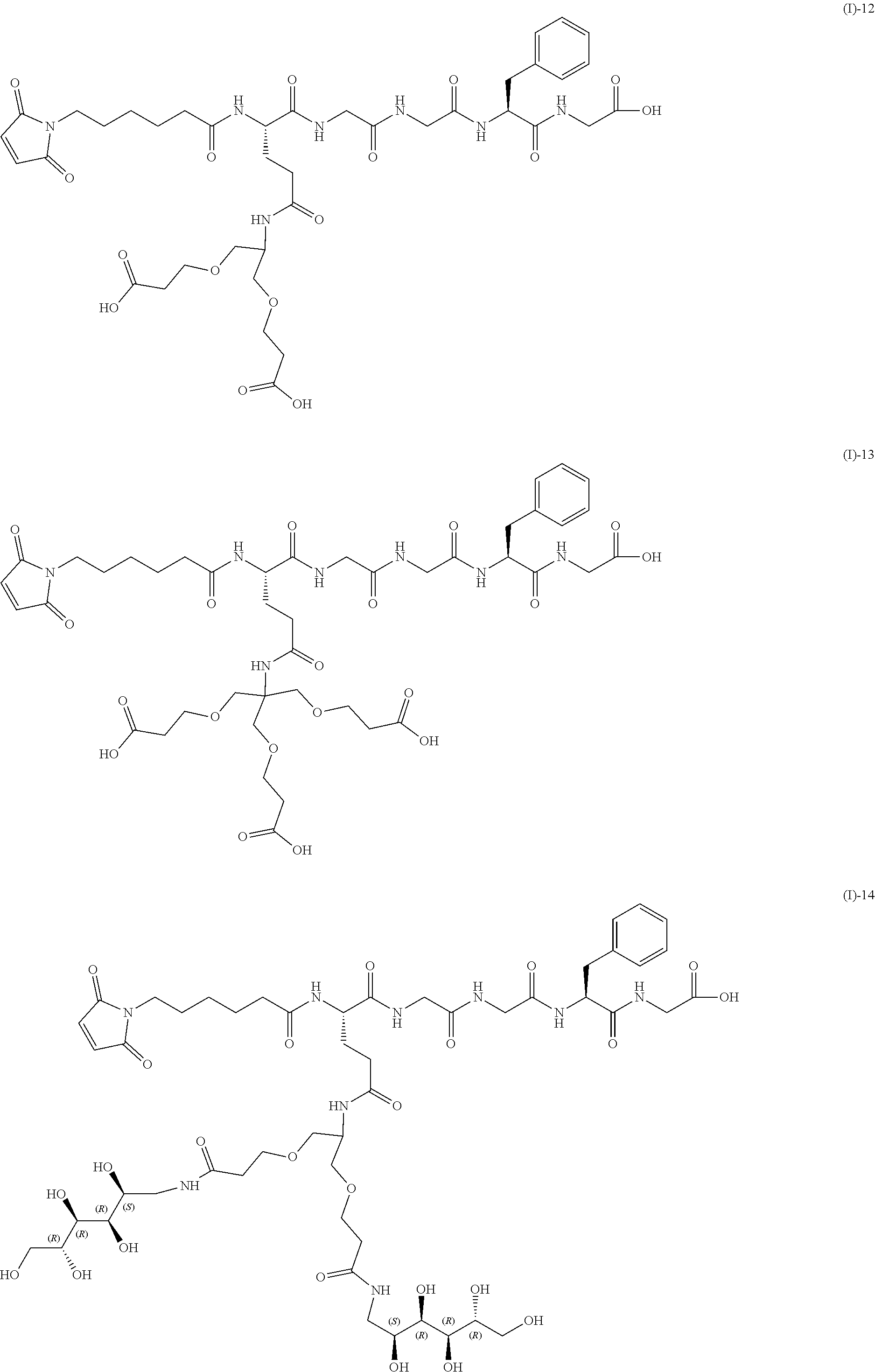

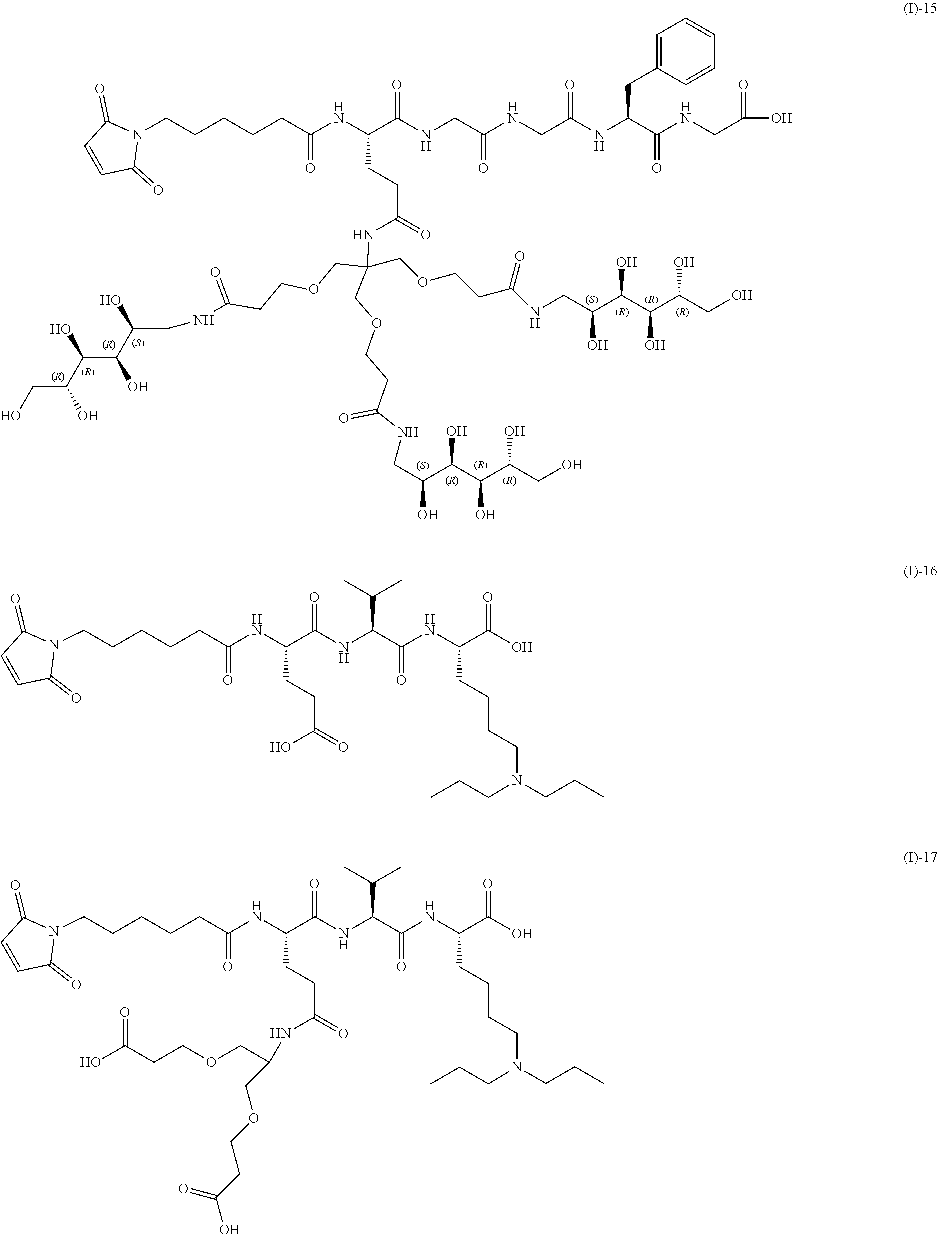

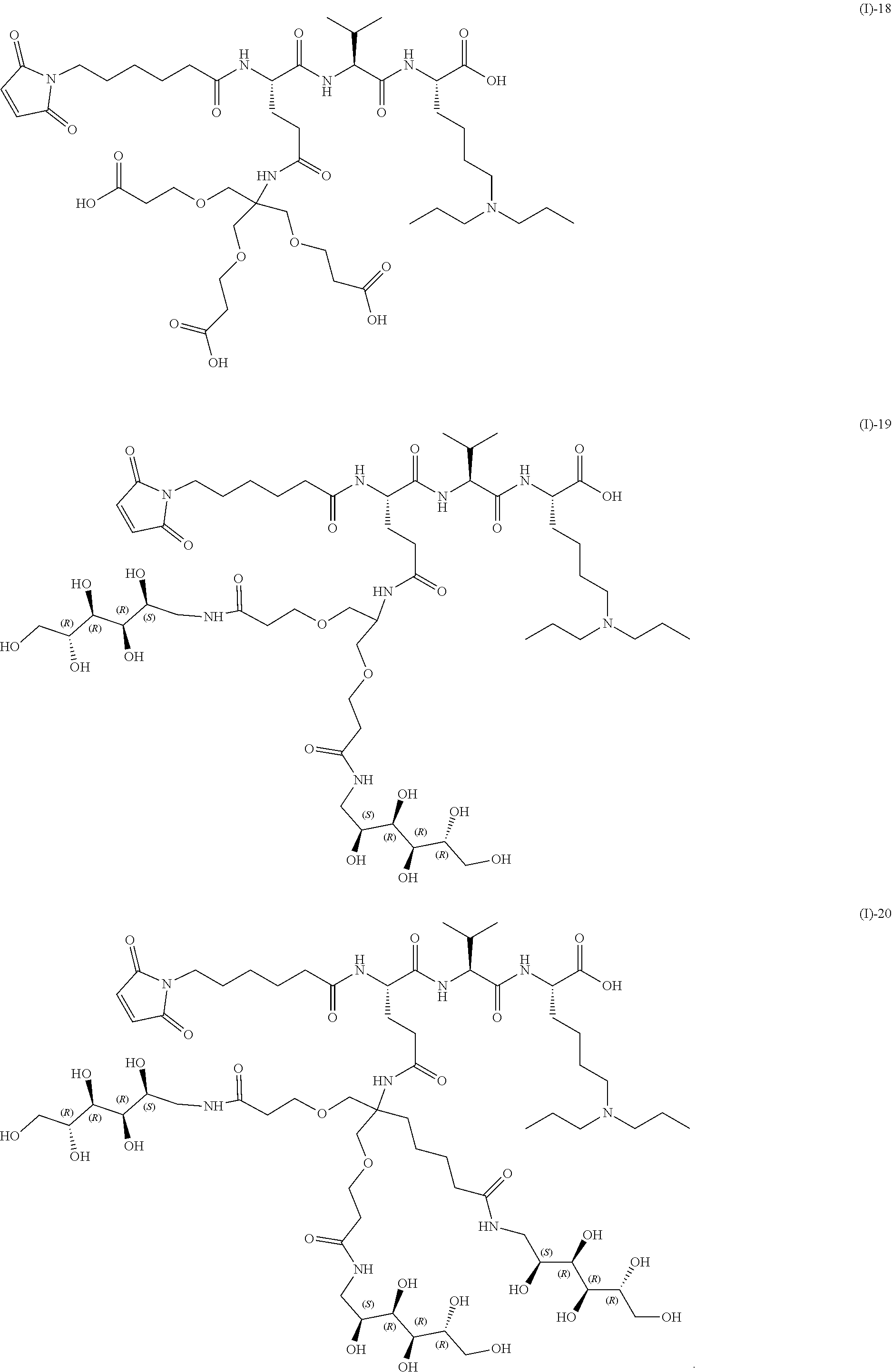

11. A compound of Formula (II) 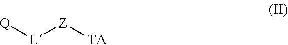
or a pharmaceutically acceptable salt, solvate, stereoisomer, or isotopic variant thereof, wherein
TA denotes to a therapeutic agent;
Z is absent or denotes to an auxiliary moiety connecting L′ to the therapeutic agent TA via a bond selected from the group consisting of disulfide, thioether, thioester, hydrazone, ester, ether, carbamate and amide bond;
Q denotes to a junction moiety capable of being coupled to a ligand via a bond selected from the group consisting of carbonyl, thioether, amide, disulfide and hydrazone bond;
L′ denotes to a linker moiety connecting Q to the therapeutic agent TA, and having a structure of: 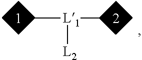
where L′₁ is a polypeptide residue consisting of three to eight amino acid residues which comprises at least one amino acid residue with a side chain carboxyl group;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O)(CH₂CH₂O)ₘ(CH₂)ₚC(O)NHR^{L2d}, R^{L2d} is H or C₁₋₆ alkyl optionally substituted with 1 to 6 hydroxy groups, each m is independently an integer from 0 to 10, and each p is independent an integer from 1 to 4; [symbol]
denotes to the N-terminal side of the polypeptide residue covalently attached to the junction moiety Q; and [symbol]
denotes to the C-terminal side of the polypeptide residue covalently attached to the auxiliary moiety Z or the therapeutic agent TA.

12. The compound a pharmaceutically acceptable salt, solvate, stereoisomer, or isotopic variant thereof according to claim 11, wherein the junction moiety Q has a structure of: 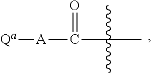
wherein Q^{a} is a function group capable of being coupled to a ligand;
A is selected from optionally substituted C₃₋₈ alkylidene, optionally substituted C₃₋₈ alkenylidene, optionally substituted C₃₋₆ cycloalkenylidene, optionally substituted C₃₋₈ cycloalkylidene, optionally substituted diglycol to octaglycol acyl, where alkylidene, alkenylidene, cycloalkenylidene, cycloalkylidene, diglycol to octaglycol acyl is optionally substituted with 1 to 4 substituents independently selected from the group consisting of halogen, —CN, R^{Qa1}, —OR^{Qa1}, —SR^{Qa1}, and —N(R^{Qa1})₂, where each R^{Qa1} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“ [symbol] ” denotes to the site covalently attached to the linker moiety L′.

13. The compound or a pharmaceutically acceptable salt, solvate, stereoisomer, or isotopic variant thereof according to claim 12, wherein the function group Q^{a} is selected from the group consisting of 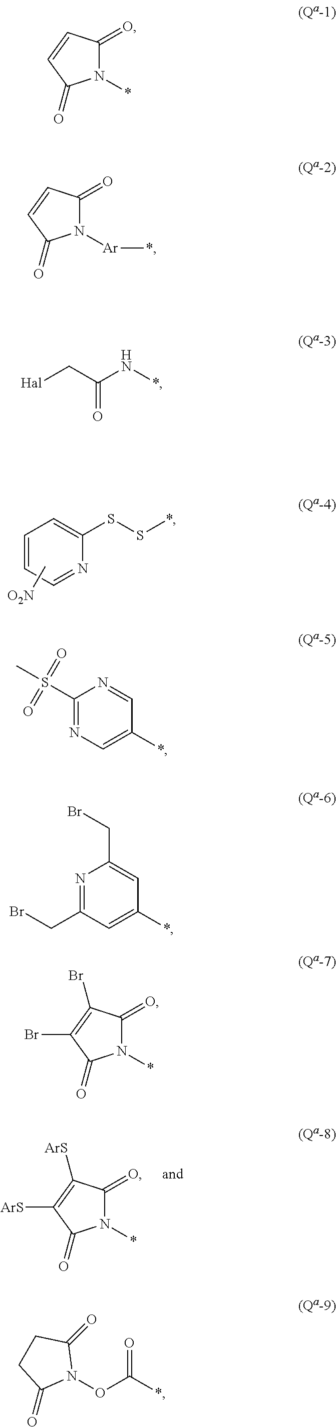
where Hal is a halogen selected from the group consisting of Cl, Br and I;
Ar is selected from the group consisting of optionally substituted C₅₋₆ cycloalkenyl, optionally substituted C₅₋₆ aryl and optionally substituted 5- to 6-membered heteroaryl, where cycloalkenyl, aryl and heteroaryl optionally substituted with 1 to 4 substituents independently selected from the group consisting of halogen, —CN, R^{Qa2}, —OR^{Qa2}, —SR^{Qa2}, and —N(R^{Qa2})₂, where each R^{Qa2} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“*” denotes to the site covalently attached to A.

14. The compound a pharmaceutically acceptable salt, solvate, stereoisomer, or isotopic variant thereof according to any of claims 11 to 13, wherein the polypeptide residue L₁ has a sequence as shown below: 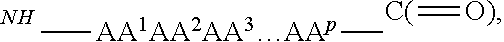
wherein each AA¹, AA², AA³, . . . AA^{p} is independently optionally substituted amino acid residue, and at least one of AA¹, AA², AA³, . . . AA^{p} is an amino acid residue with a side chain carboxyl group, preferably Glu or Asp;
p is an integer from 3 to 8, preferably from 3 to 5;
“NH—” denotes to N-terminal side of the polypeptide residue;
“—C(═O)” denotes to C-terminal side of the polypeptide residue.

15. The compound or a pharmaceutically accepted salt, solvate, stereoisomer, or isotopic variant thereof according to claim 14, wherein each AA¹, AA², AA³, . . . AA^{p} independently optionally substituted amino acid residue selected from the group consisting of Glu, Asp, Pro, Nva, Leu, Ile, Met, Tyr, Trp, Ser, Thr, Cys, Asn, Gln, Arg, Phe, Lys, Val, Ala, Cit, Gly, and N-alkyl amino acids, and at least one of AA¹, AA², AA³, . . . AA^{p} is Glu or Asp.

16. The compound a pharmaceutically acceptable salt, solvate, stereoisomer, or isotopic variant thereof according to claim 14 or 15, wherein AA¹ is an amino acid residue with a side chain carboxyl group, preferably Glu or Asp; each AA², AA³, . . . AA^{p} is independently optionally substituted amino acid residue selected from the group consisting of Pro, Nva, Leu, Ile, Met, Tyr, Trp, Ser, Thr, Cys, Asn, Gln, Arg, Phe, Lys, Val, Ala, Cit, and Gly.

17. The compound or a pharmaceutically acceptable salt, solvate, stereoisomer, or isotopic variant thereof according to any of claims 11 to 16, wherein the polypeptide residue L′₁ is selected from the group consisting of ^{NH}-Glu-Phe-Lys(NR^{Lys1}R^{Lys2})—^{C(═O)}, ^{NH}-Gu-Val-Lys(NR^{Lys1}R^{Lys2})—^{C(═O)}, ^{NH}-Glu-Ala-Ala-Ala-^{C(═O)}, ^{NH}-Glu-Ala-Ala-^{C(═O)}, ^{NH}-Glu-Val-Ala-^{C(═O)}, ^{NH}-Glu-Val-Cit-^{C(═O)}, ^{NH}-Glu-Gly-Gly-Phe-Gly-^{C(═O)}, ^{NH}-Asp-Phe-Lys-^{C(═O)}, ^{NH}-Asp-Ala-Ala-Ala-^{C(═O)}, ^{NH}-Asp-Val-Ala-^{C(═O)}, ^{NH}-Asp-Val-Cit-^{C(═O)}, ^{NH}-Asp-Gly-Gly-Phe-Gly-^{C(═O)}, and ^{NH}-Asp-Val-Lys(NR^{Lys1}R^{Lys2})—^{C(═O)}, where R^{Lys1} and R^{Lys2} are each independently H or C₁₋₆ alkyl.

18. The compound or a pharmaceutically acceptable salt, solvate, stereoisomer, or isotopic variant thereof according to any of claims 11 to 17, wherein the hydrophilic group L₂ has a structure of —NHC(R^{L2a})(R^{L2b})(R^{L2c}), where R^{L2a}, R^{L2b}, and R^{L2c} are each independently selected from the group consisting of H, —CH₂O(CH₂)₂C(O)OH, and —CH₂O(CH₂)₂C(O)NHR^{L2d}, and at most two of R^{L2a}, R^{L}₂b, and R^{L2c} are simultaneously H, R^{L2d} is C₄₋₆ alkyl substituted with 3 to 5 hydroxy groups, preferably R^{L2d} is C₄₋₆ alkyl substituted with 3 to 5 hydroxy groups, where no more than one hydroxyl group is substituted on each carbon atom, for example, R^{L2d} is selected from the group consisting of:

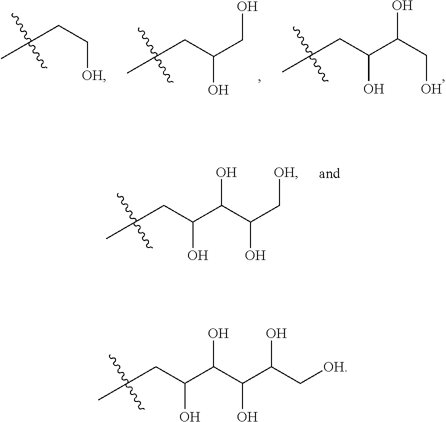

19. The compound or a pharmaceutically acceptable salt, solvate, stereoisomer, or isotopic variant thereof according to any of claims 11 to 18, wherein the hydrophilic group L₂ is selected from the group consisting of: 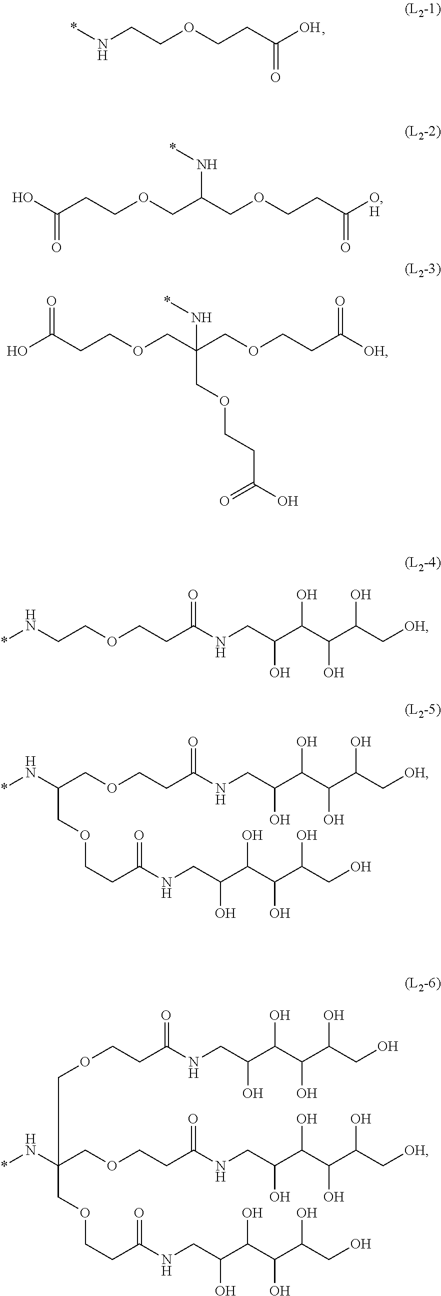
“*” denotes to the site covalently attached to polypeptide residue L′₁.

20. The compound or a pharmaceutically acceptable salt, solvate, stereoisomer, or isotopic variant thereof according to any of claims 11 to 19, wherein the auxiliary moiety Z is selected from the group consisting of mercapto, disulfide, amino, carboxyl, aldehyde, maleimide, haloacetyl, hydrazide and hydroxyl groups.

21. The compound or a pharmaceutically acceptable salt, solvate, stereoisomer, or isotopic variant thereof according to any of claims 11 to 20, wherein the therapeutic agent is a cytotoxic or cytostatic agent, e.g. a camptothecin compound, an analogue or a derivative thereof.

22. The compound or a pharmaceutically acceptable salt, solvate, stereoisomer, or isotopic variant thereof according to any of claims 10 to 19, wherein the compound is a compound of Formula (II′): 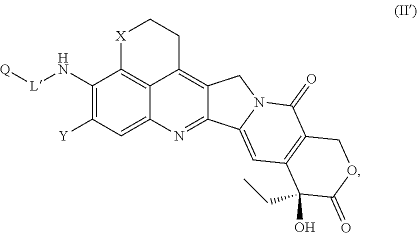
X is selected from the group consisting of —CH₂—, O and S; Y is selected from the group consisting of H, D, and F;
Q and L′ are as defined in the preceding claims.

23. The compound or a pharmaceutically acceptable salt, solvate, stereoisomer, or isotopic variant thereof according to any of claims 10 to 20, wherein the compound is selected from the group consisting of:

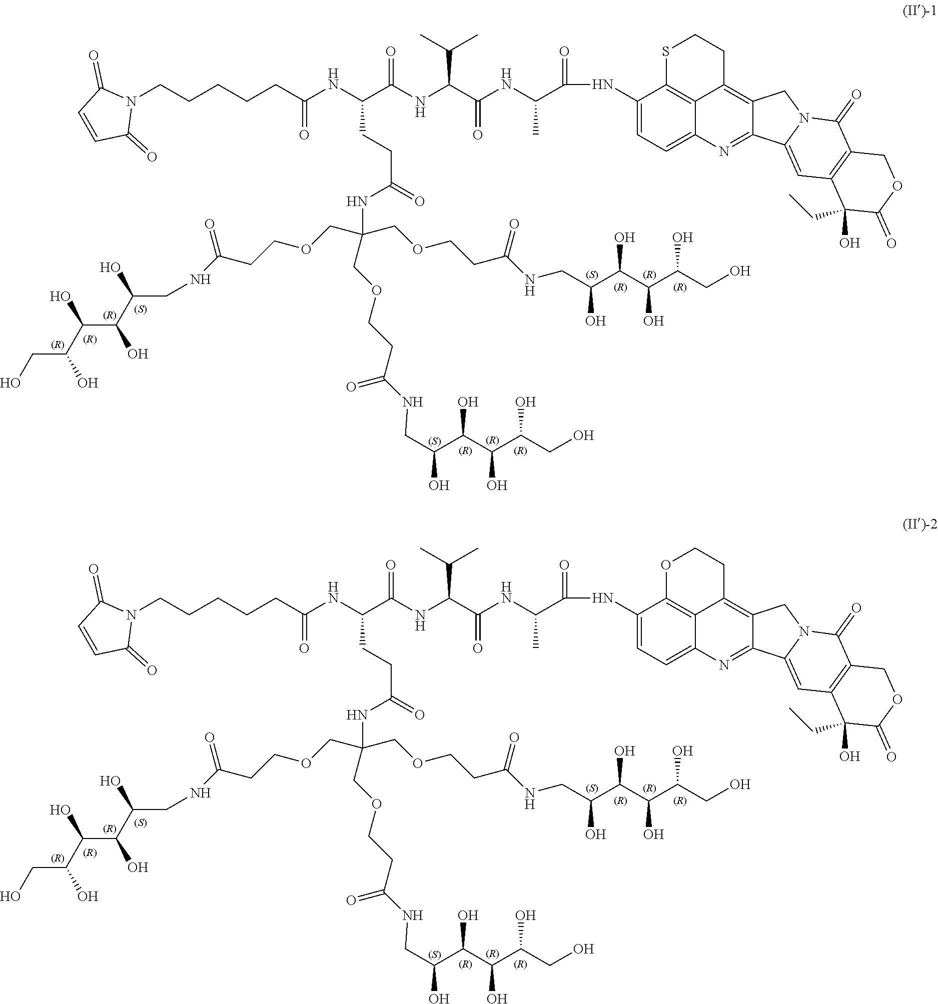

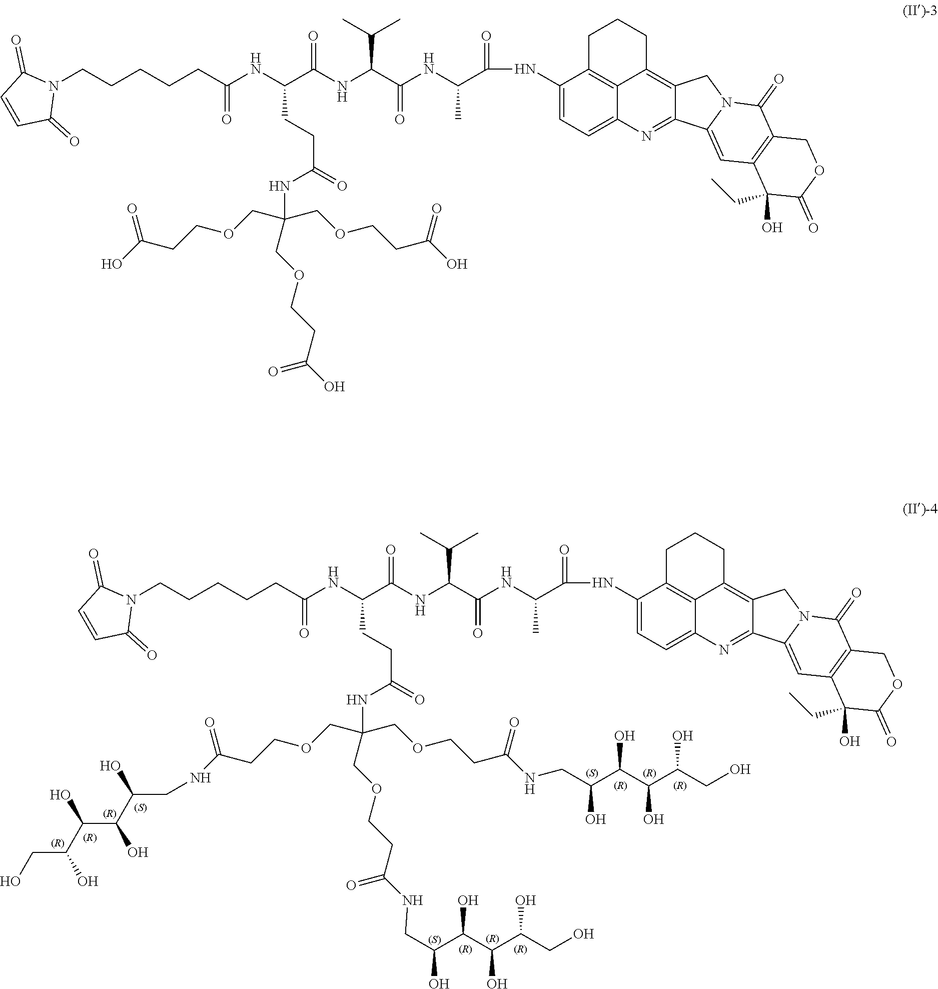

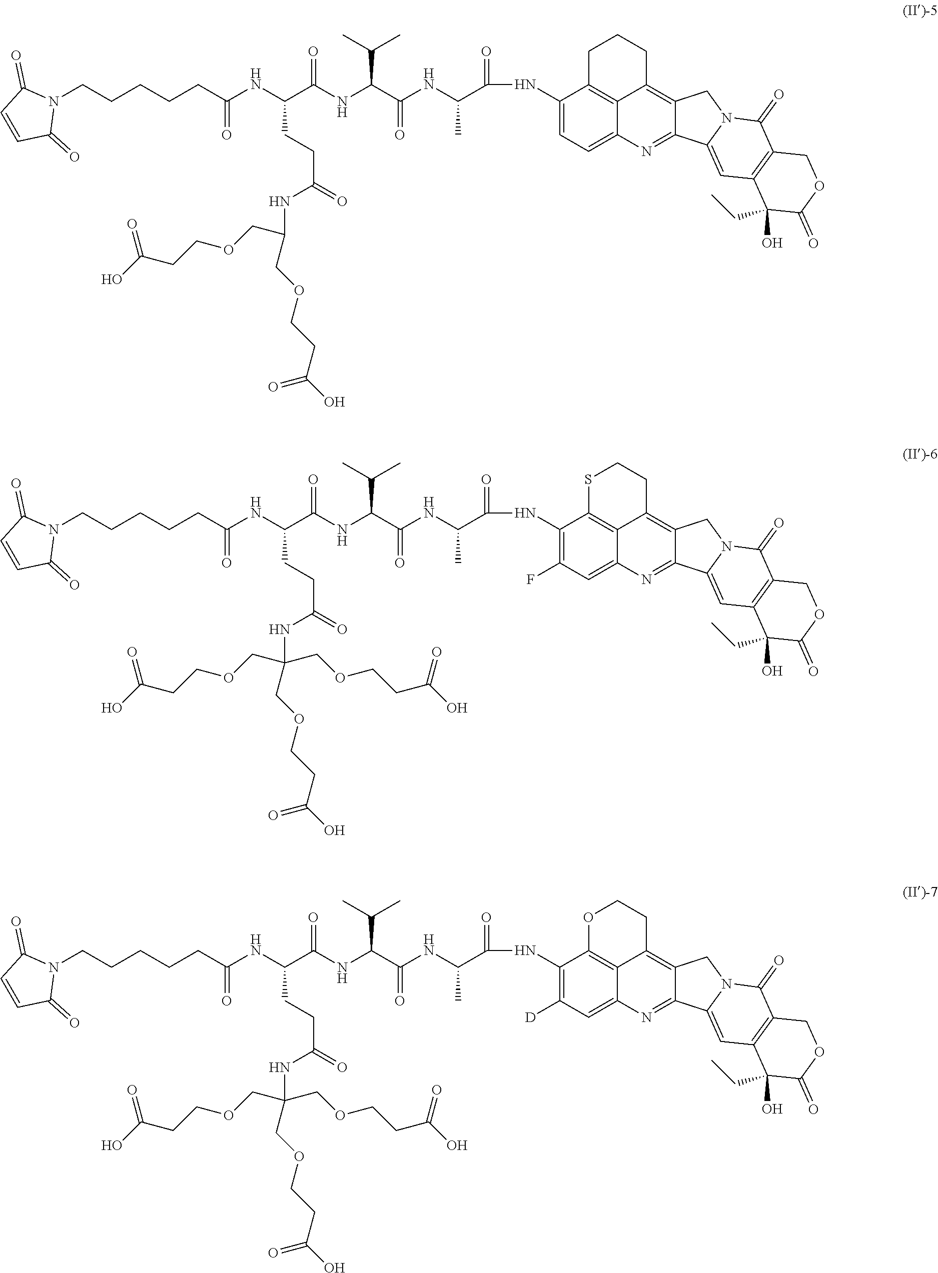

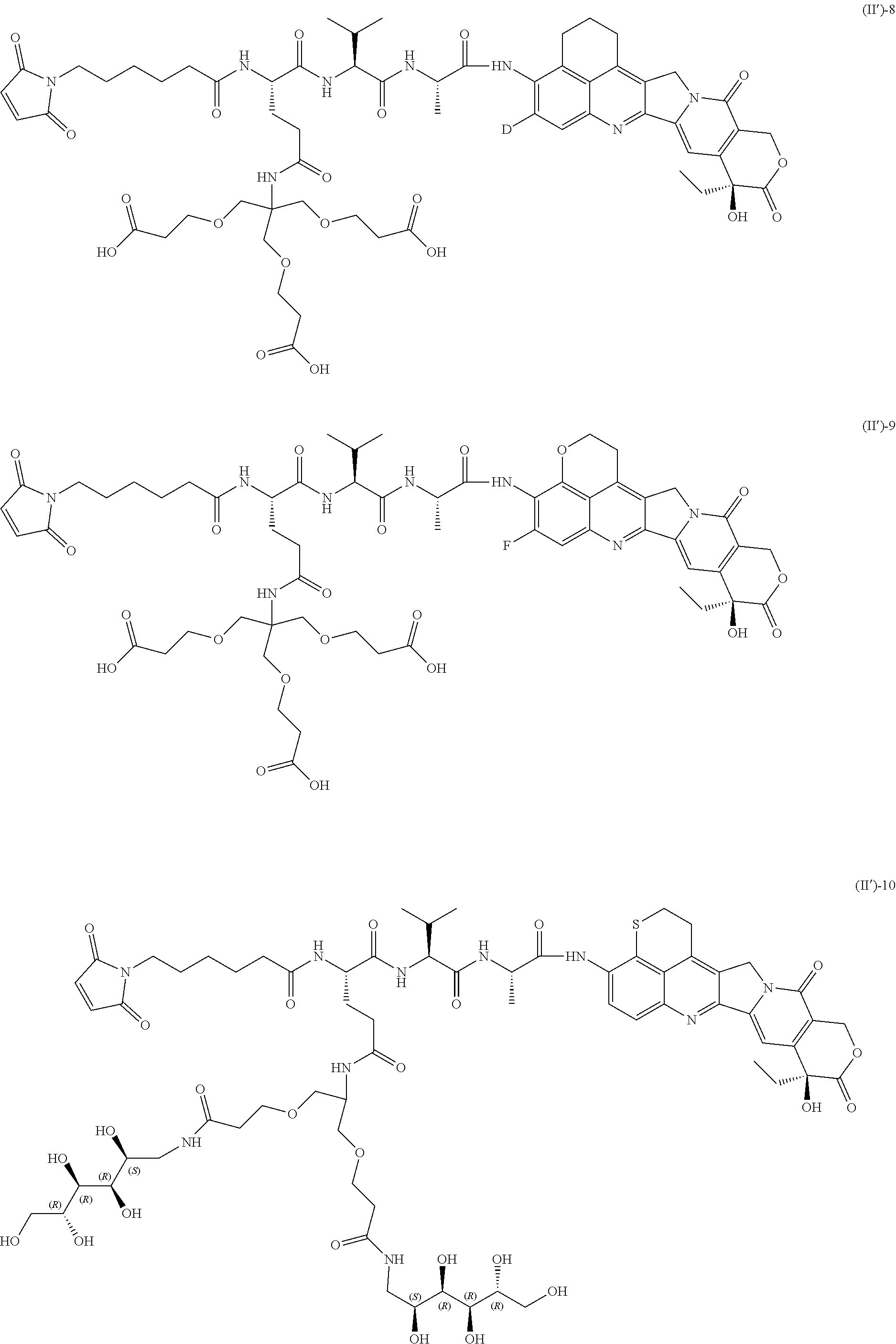

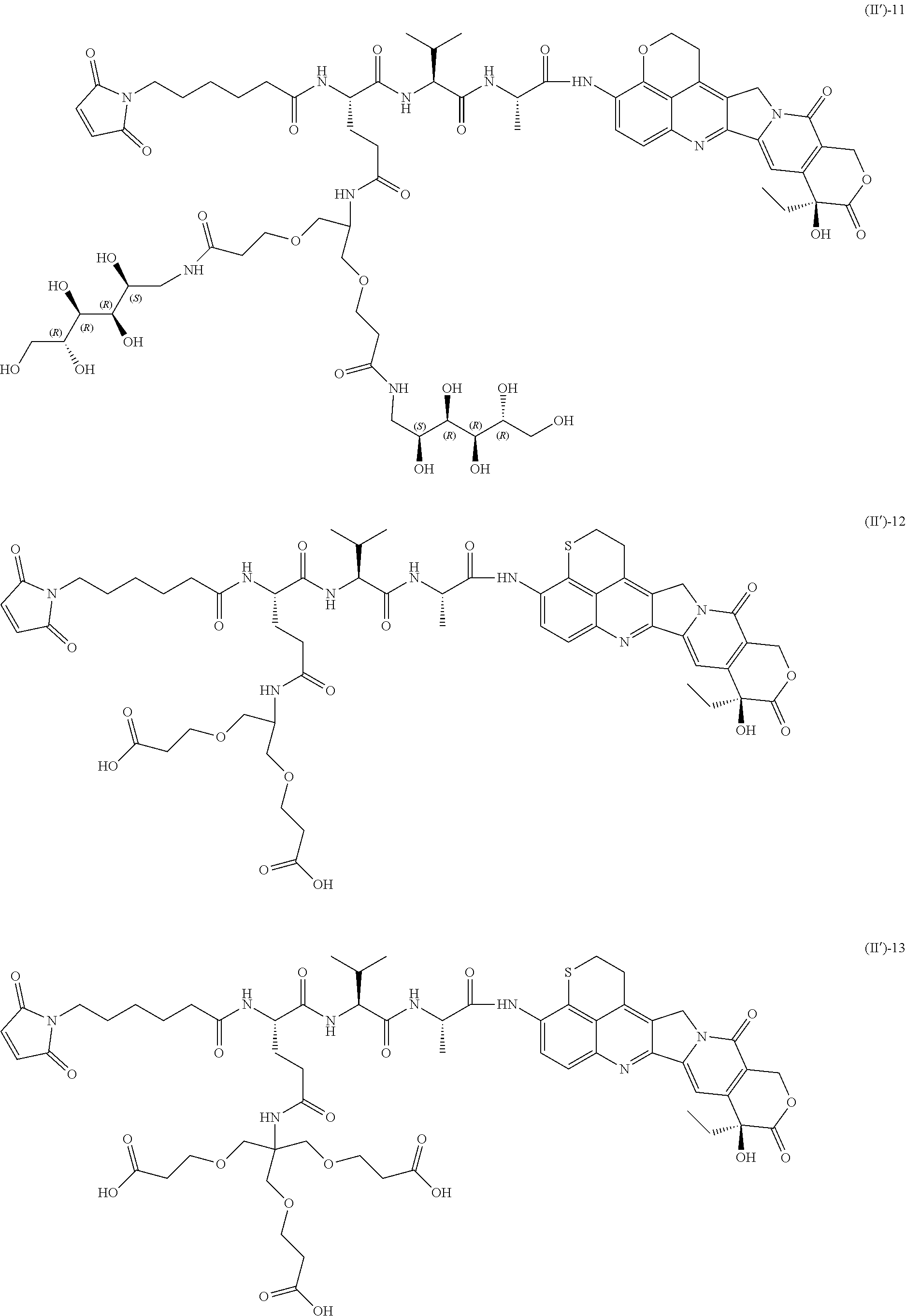

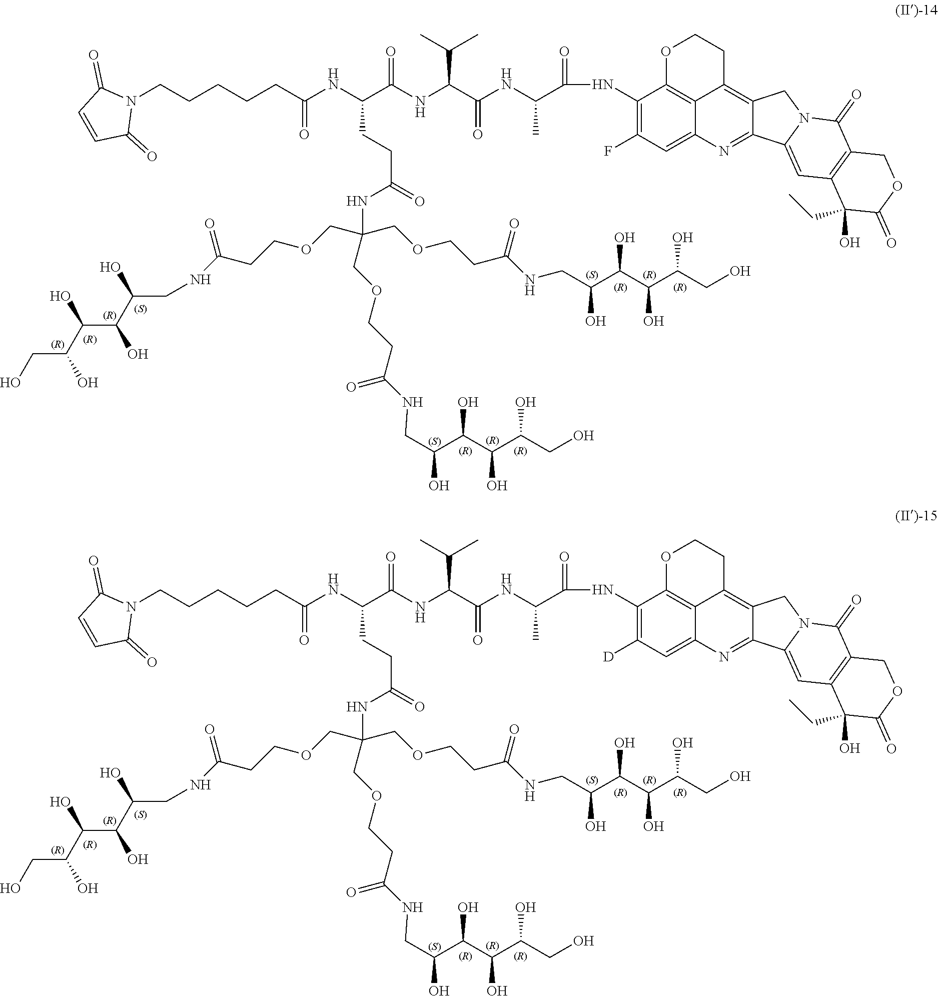

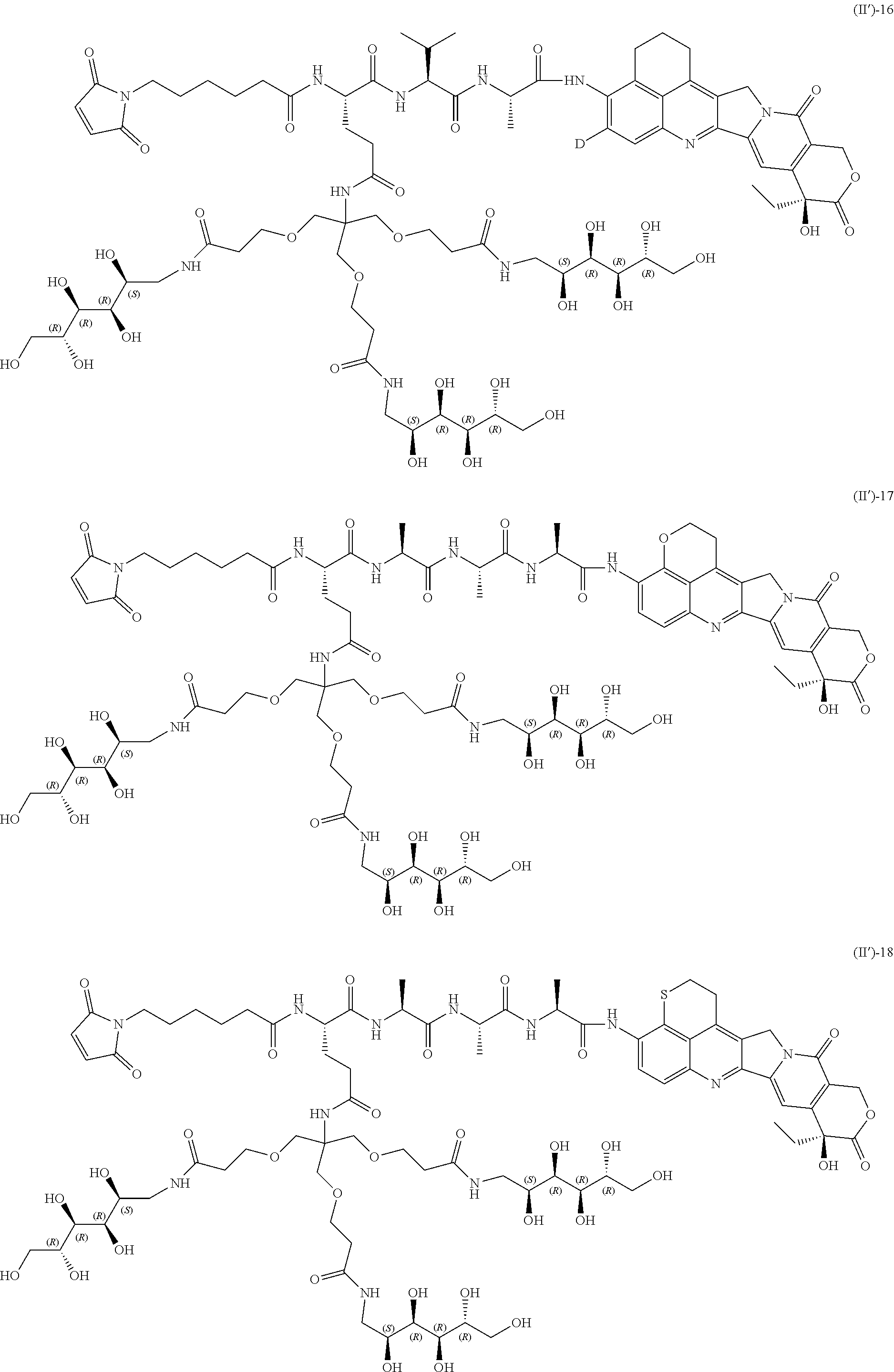

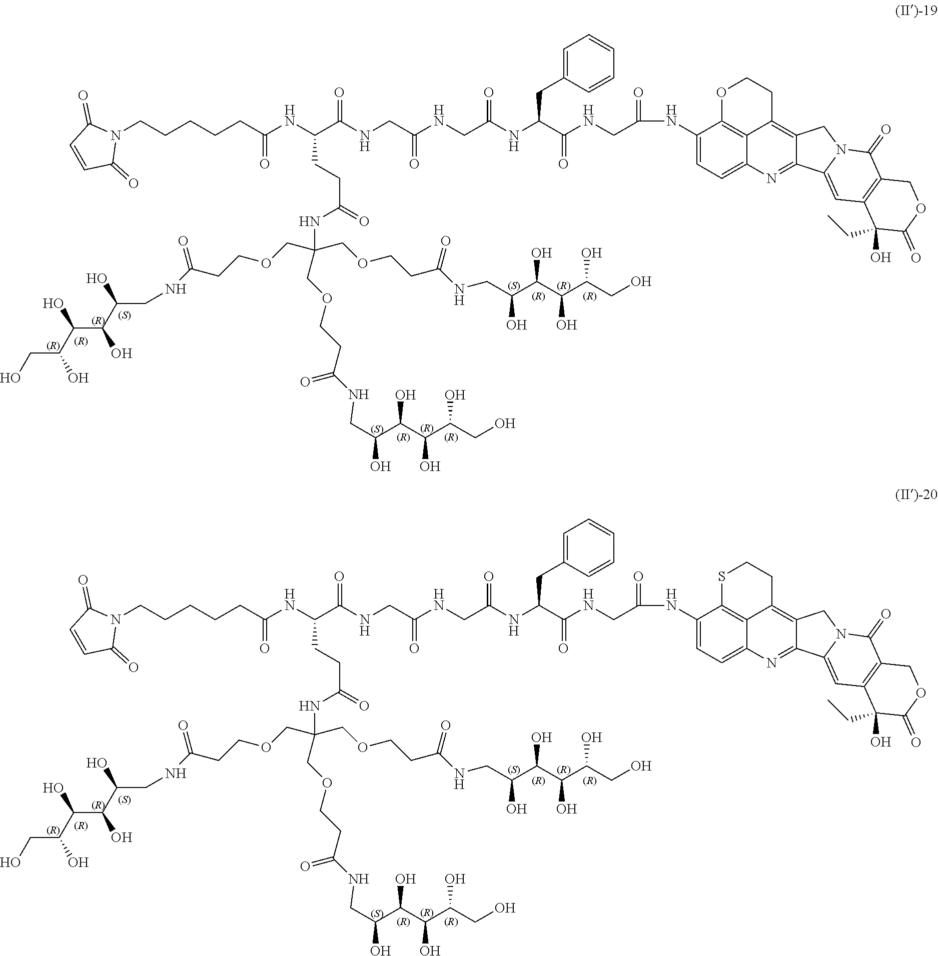

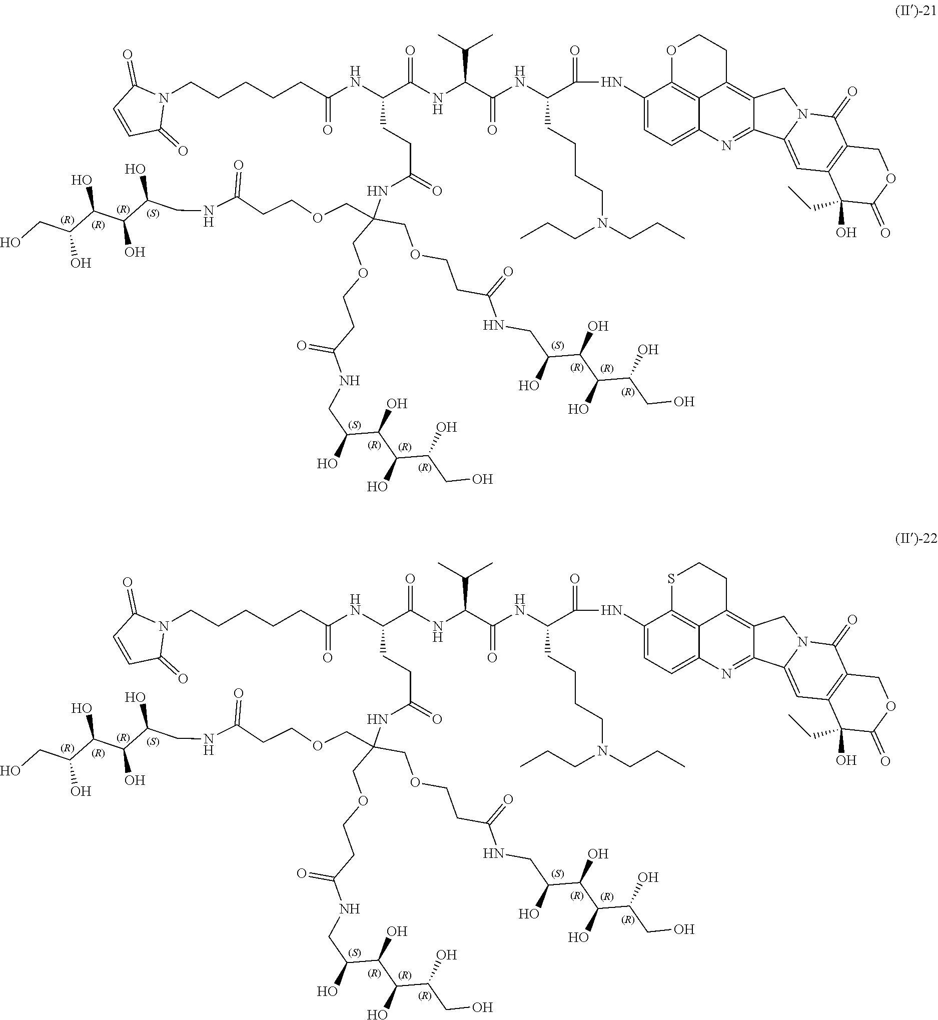

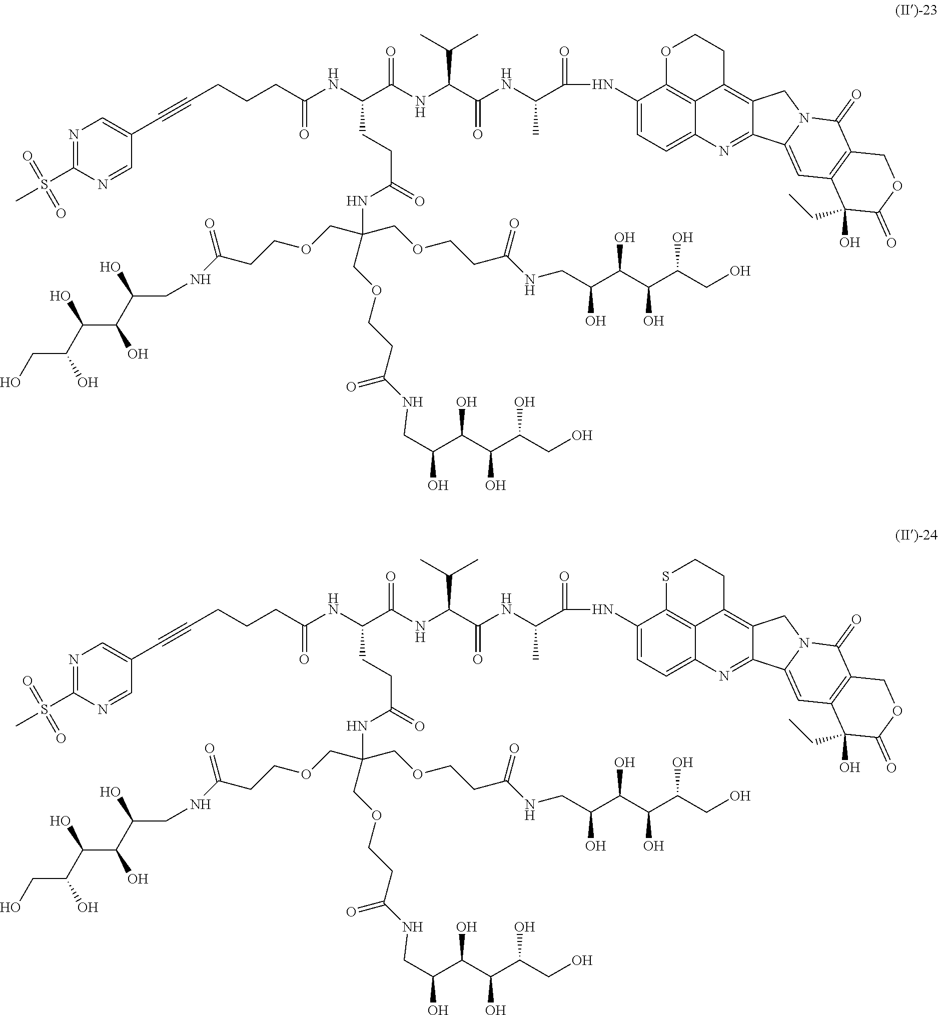

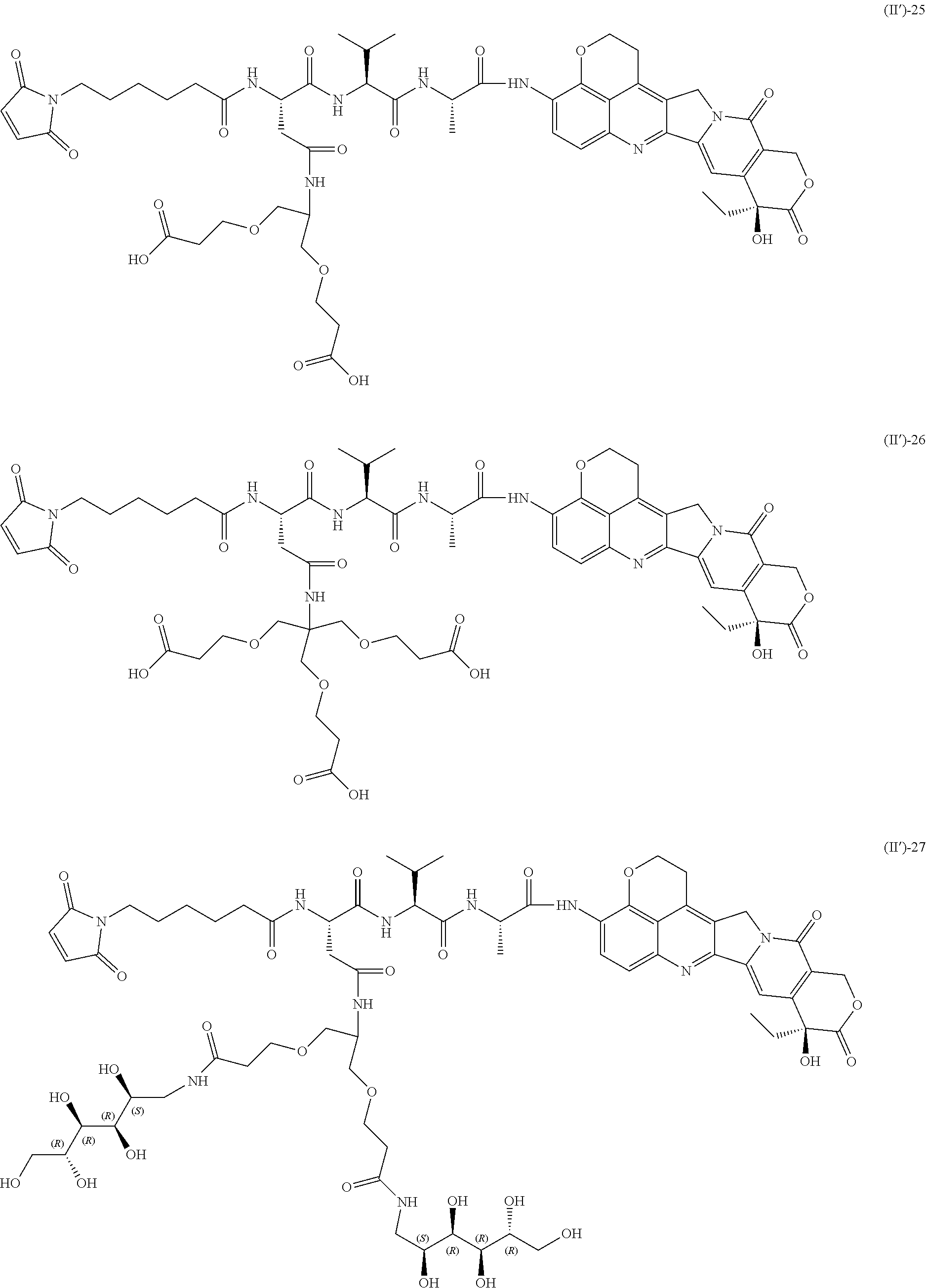

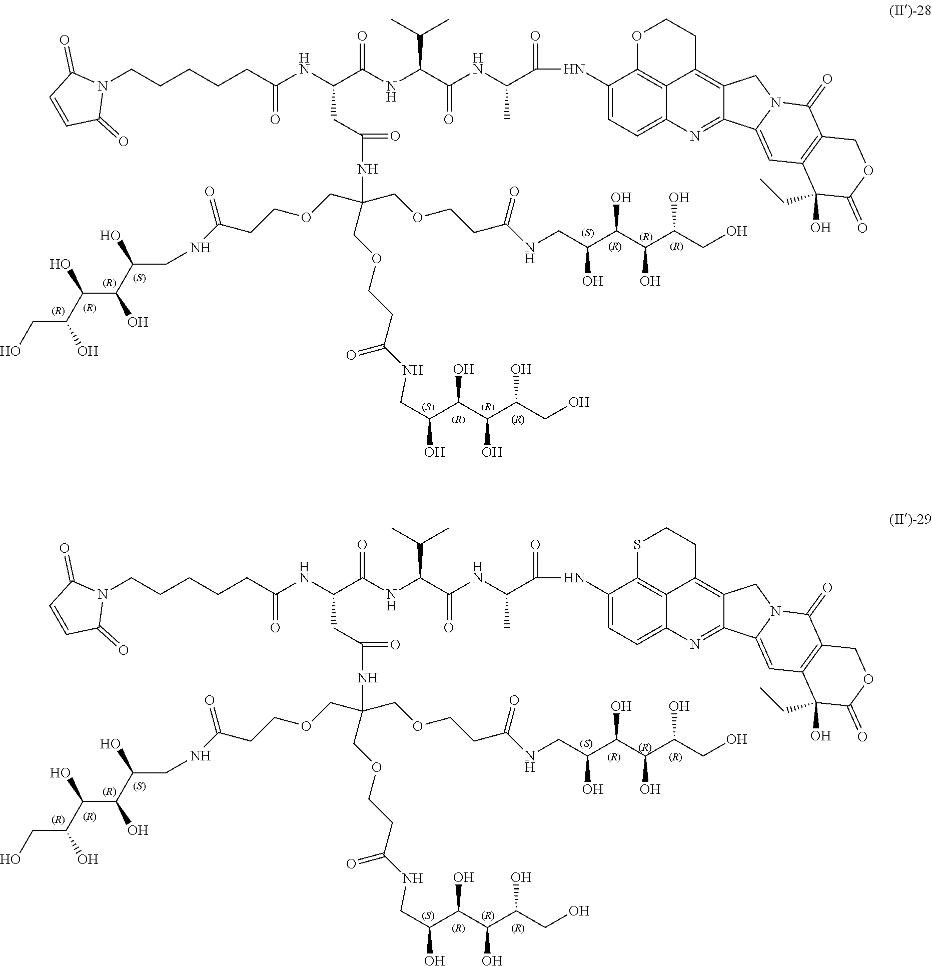

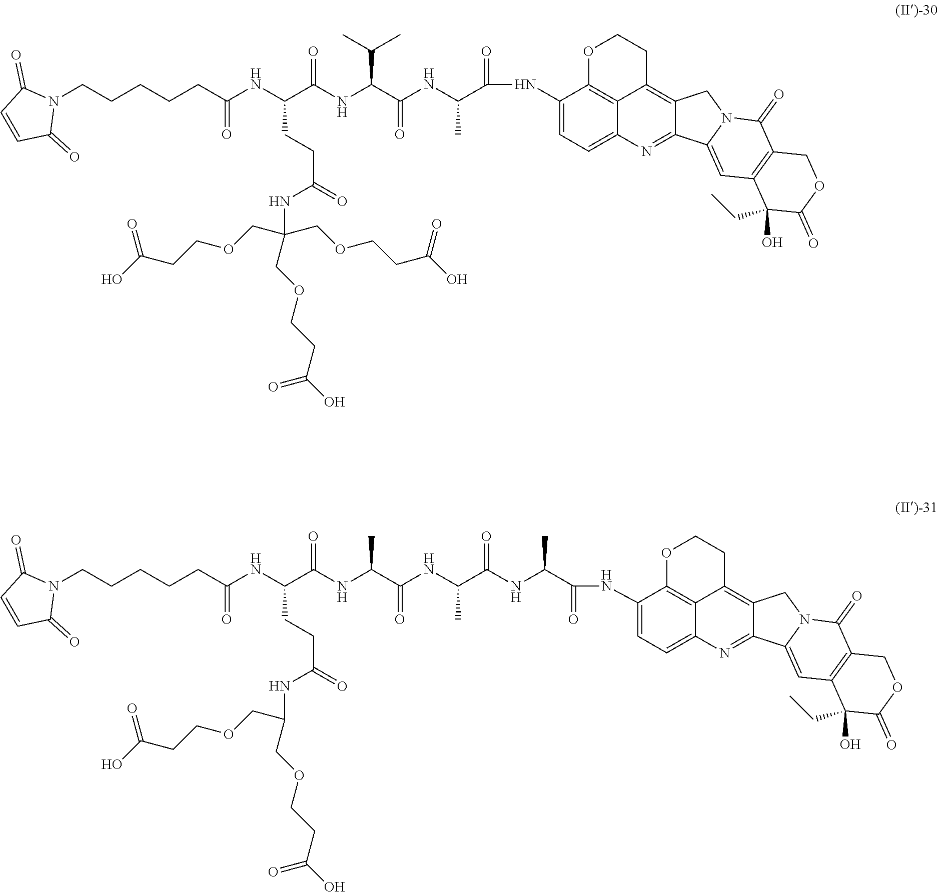 [SMILES: CC[C@@]1(O)C(=O)OCc2c1cc1n(c2=O)Cc2c-1nc1ccc(NC(=O)[C@H](C)NC(=O)[C@@H](NC(=O)[C@H](CCC(=O)NC(COCCC(=O)O)(COCCC(=O)O)COCCC(=O)O)NC(=O)CCCCCN3C(=O)C=CC3=O)C(C)C)c3c1c2CCO3 ; CC[C@@]1(O)C(=O)OCc2c1cc1n(c2=O)Cc2c-1nc1ccc(NC(=O)[C@H](C)NC(=O)[C@H](C)NC(=O)[C@H](C)NC(=O)[C@H](CCC(=O)NC(COCCC(=O)O)COCCC(=O)O)NC(=O)CCCCCN3C(=O)C=CC3=O)c3c1c2CCO3]

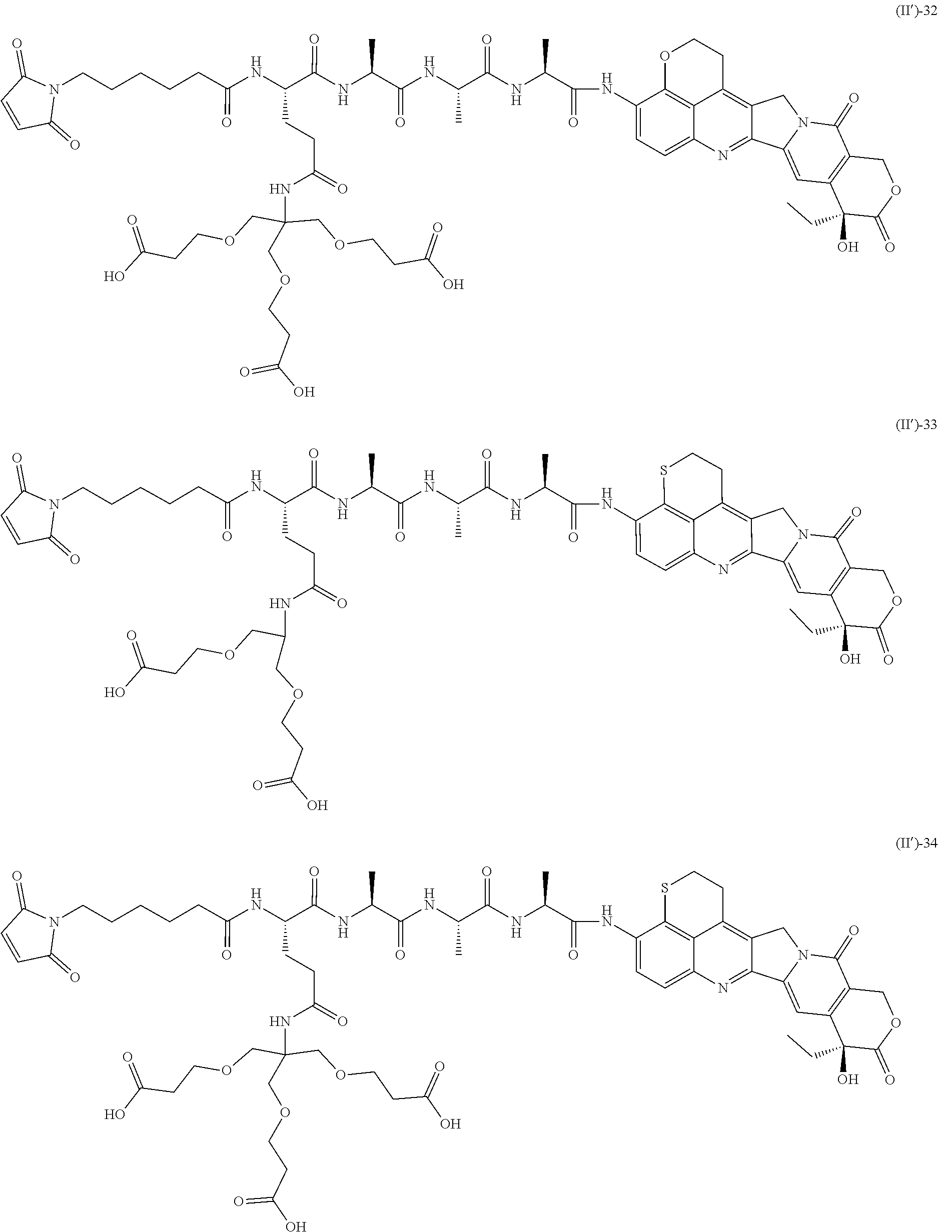

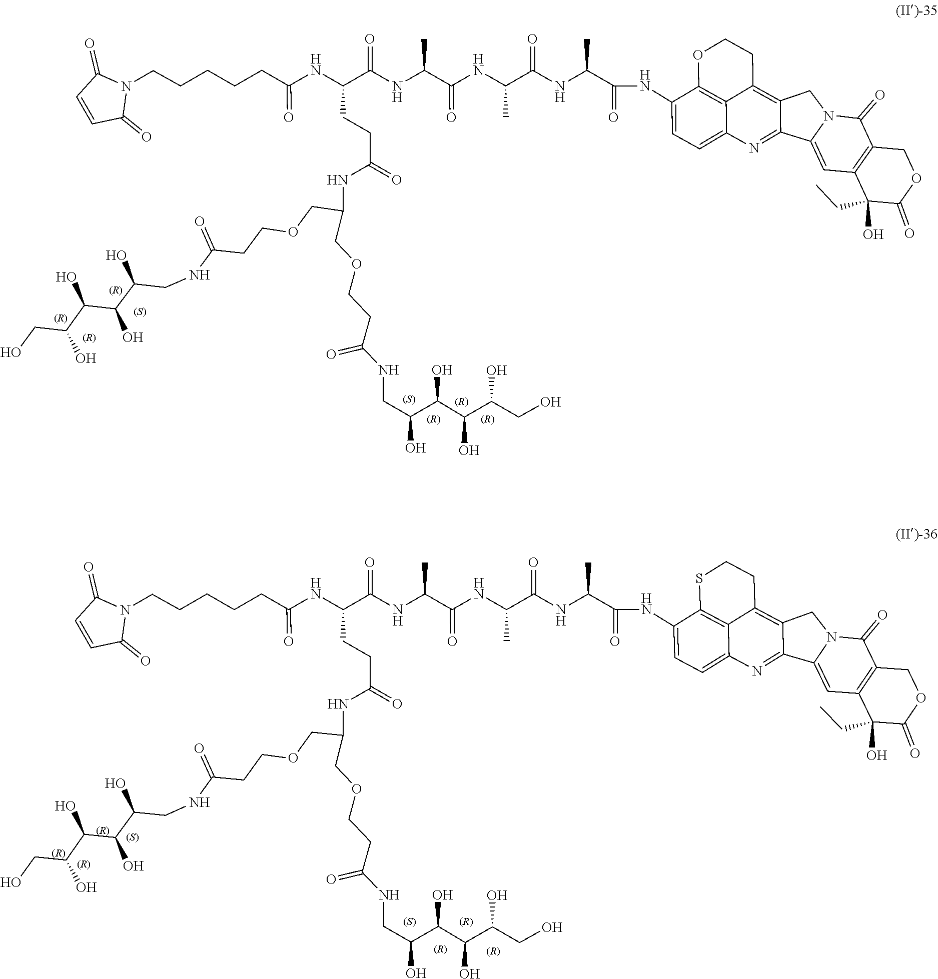

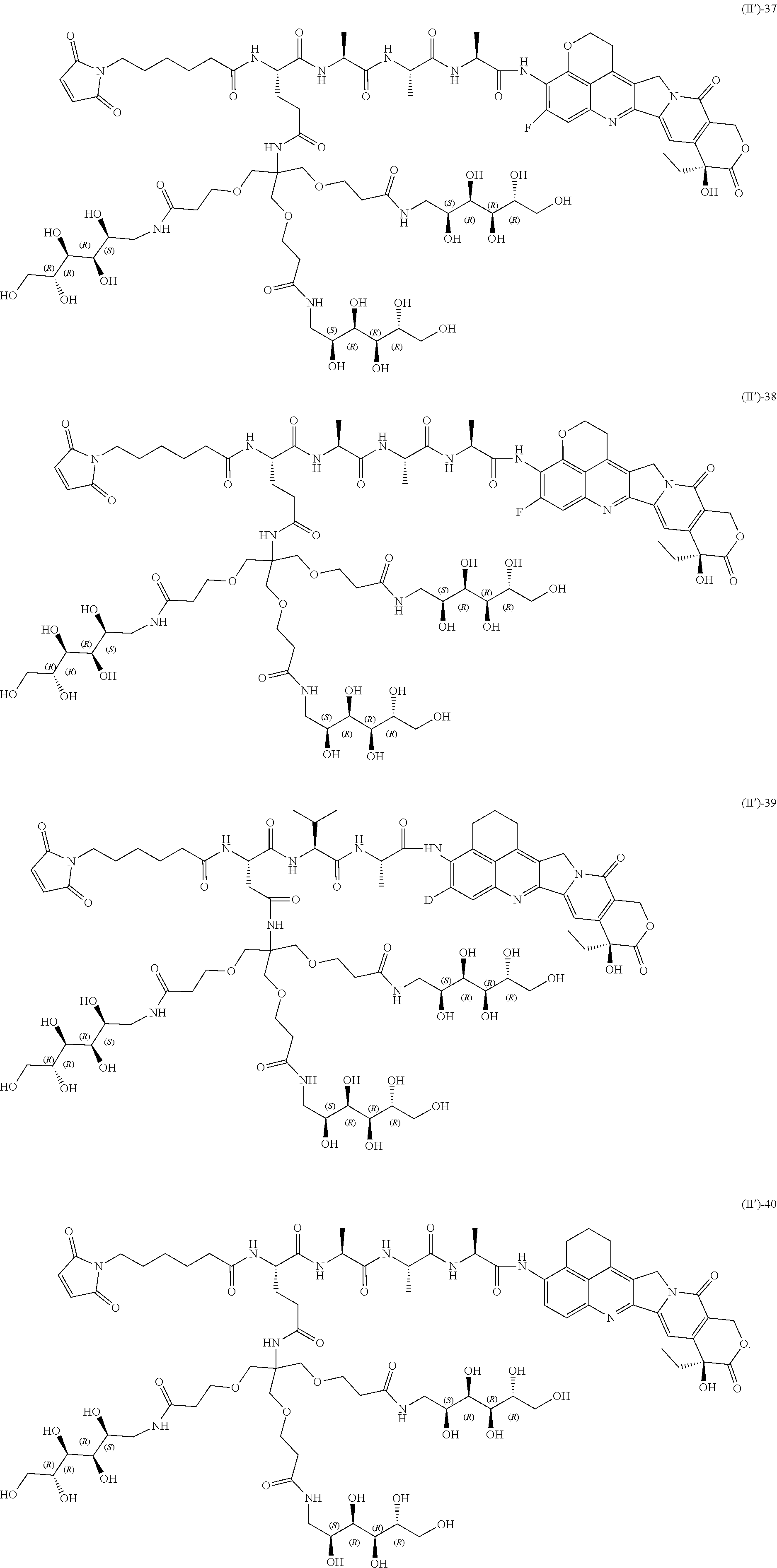

24. A ligand-drug conjugate of Formula (III): 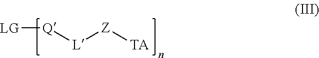
or a pharmaceutically acceptable salt, solvate, stereoisomer, or isotopic variant thereof, wherein
LG denotes to a ligand;
TA denotes to a therapeutic agent;
Z is absent or denotes to an auxiliary moiety connecting L′ to the therapeutic agent TA via a bond selected from the group consisting of disulfide, thioether, thioester, hydrazone, ester, ether, carbamate and amide bond;
Q′ denotes to a junction moiety coupled to the ligand LG via a bond selected from the group consisting of carbonyl, thioether, amide, disulfide and hydrazone bond;
L′ denotes to a linker moiety connecting Q to the therapeutic agent TA, and having a structure of: 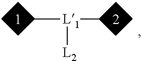
where L′₁ is a polypeptide residue consisting of three to eight amino acid residues which comprises at least one amino acid residue with a side chain carboxyl group;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O)(CH₂CH₂O)ₘ(CH₂)ₚC(O)NHR^{L2d}, R^{L2d} is H or C₁₋₆ alkyl optionally substituted with 1 to 6 hydroxy groups, each m is independently an integer from 0 to 10, and each p is independent an integer from 1 to 4; [symbol]
denotes to the N-terminal side of the polypeptide residue covalently attached to the junction moiety Q′; [symbol]
denotes to the C-terminal side of the polypeptide residue covalently attached to the therapeutic agent TA; and
n is a number in the range from 1 to 8.

25. The ligand-drug conjugate or a pharmaceutically acceptable salt, solvate, stereoisomer, or isotopic variant thereof according to claim 24, wherein the junction moiety Q′ has a structure of: 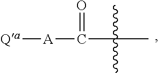
wherein Q′^{a} is a function group capable of being coupled to a ligand;
A is selected from optionally substituted C₃-s alkylidene, optionally substituted C₃₋₈ alkenylidene, optionally substituted C₃₋₆ cycloalkenylidene, optionally substituted C₃₋₈ cycloalkylidene, optionally substituted diglycol to octaglycol acyl, where alkylidene, alkenylidene, cycloalkenylidene, cycloalkylidene, diglycol to octaglycol acyl is optionally substituted with 1 to 4 substituents independently selected from the group consisting of halogen, —CN, R^{Q′a1}, —OR^{Q′a1}, —SR^{Q′a1}, and —N(R^{Q′a1})₂, where each R^{Q′a1} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl; and
“ [symbol] ” denotes to the site covalently attached to the linker moiety L′.

26. The ligand-drug conjugate or a pharmaceutically acceptable salt, solvate, stereoisomer, or isotopic variant thereof according to claim 25, wherein the junction moiety Q′^{a} is selected from the group consisting of: 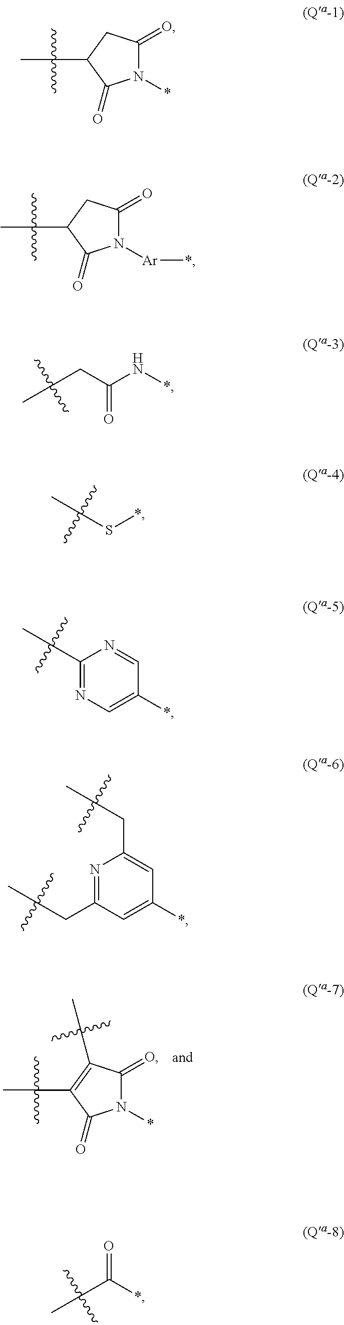
where, Ar is selected from the group consisting of optionally substituted C₅₋₆ cycloalkenyl, optionally substituted C₅₋₆ aryl and optionally substituted 5- to 6-membered heteroaryl, where cycloalkenyl, aryl and heteroaryl optionally substituted with 1 to 4 substituents independently selected from the group consisting of halogen, —CN, R^{Qa2}, —OR^{Qa2}, —SR^{Qa2}, and —N(R^{Qa2})₂, where each R^{Qa2} is independently selected from the group consisting of C₁₋₆ alkyl, C₁₋₆ haloalkyl, C₃₋₆ cycloalkyl, 3- to 10-membered heterocyclyl, C₆₋₁₀ aryl, and 5- to 10-membered heteroaryl;
“*” denotes to the site covalently attached to A;
“ [symbol] ” denotes to the site covalently attached to the ligand LG.

27. The ligand-drug conjugate or a pharmaceutically acceptable salt, solvate, stereoisomer, or isotopic variant thereof according to any of claims 24 to 26, wherein the polypeptide residue L′₁ has a sequence as shown below: 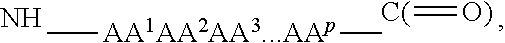
wherein each AA¹, AA², AA³, . . . AA^{p} is independently optionally substituted amino acid residue, and at least one of AA¹, AA², AA³, . . . AA^{p} is an amino acid residue with a side chain carboxyl group, preferably Glu or Asp;
p is an integer from 3 to 8, preferably from 3 to 5;
“NH—” denotes to N-terminal side of the polypeptide residue;
“—C(═O)” denotes to C-terminal side of the polypeptide residue.

28. The ligand-drug conjugate or a pharmaceutically accepted salt, solvate, stereoisomer, or isotopic variant thereof according to claim 27, wherein each AA¹, AA², AA³, . . . AA^{p} independently optionally substituted amino acid residue selected from the group consisting of Glu, Asp, Pro, Nva, Leu, Ile, Met, Tyr, Trp, Ser, Thr, Cys, Asn, Gln, Arg, Phe, Lys, Val, Ala, Cit, Gly, and N-alkyl amino acids, and at least one of AA¹, AA², AA³, . . . AA^{p} is Glu or Asp.

29. The ligand-drug conjugate or a pharmaceutically acceptable salt, solvate, stereoisomer, or isotopic variant thereof according to claim 27 or 28, wherein AA¹ is an amino acid residue with a side chain carboxyl group, preferably Glu or Asp; each AA², AA³, . . . AA^{p} is independently optionally substituted amino acid residue selected from the group consisting of Pro, Nva, Leu, Ile, Met, Tyr, Trp, Ser, Thr, Cys, Asn, Gln, Arg, Phe, Lys, Val, Ala, Cit, and Gly.

30. The ligand-drug conjugate or a pharmaceutically acceptable salt, solvate, stereoisomer, or isotopic variant thereof according to any of claims 24 to 29, wherein the polypeptide residue L′₁ is selected from the group consisting of ^{NH}-Glu-Phe-Lys(NR^{Lys1}R^{Lys2})—^{C(═O)}, ^{NH}-Glu-Val-Lys(NR^{Lys1}R^{Lys2})—^{C(═O)}, ^{NH}-Glu-Ala-Ala-Ala-^{C(═O)}, ^{NH}-Glu-Ala-Ala-^{C(═O)}, ^{NH}-Glu-Val-Ala-^{C(═O)}, ^{NH}-Glu-Val-Cit-^{C(═O)}, ^{NH}-Gu-Gly-Gly-Phe-Gly-^{C(═O)}, ^{NH}-Asp-Phe-Lys-^{C(═O)}, ^{NH}-Asp-Ala-Ala-Ala-^{C(═O)}, ^{NH}-Asp-Val-Ala-^{C(═O)}, ^{NH}-Asp-Val-Cit-^{C(═O)}, ^{NH}-Asp-Gly-Gly-Phe-Gly-^{C(═O)}, and ^{NH}-Asp-Val-Lys(NR^{Ly1}R^{Lys2})—^{C(═O)}, where R^{Lys1} and R^{Lys2} are each independently H or C₁₋₆ alkyl.

31. The ligand-drug conjugate or a pharmaceutically acceptable salt, solvate, stereoisomer, or isotopic variant thereof according to any of claims 24 to 30, wherein the hydrophilic group L₂ has a structure of —NHC(R^{L2a})(R^{L2b})(R^{L2c}), where R^{L2a}, R^{L2b}, and R^{L2c} are each independently selected from the group consisting of H, —CH₂O(CH₂)₂C(O)OH, and —CH₂O(CH₂)₂C(O)NHR^{L2d}, and at most two of R^{L2a}, R^{L2b}, and R^{L2c} are simultaneously H, R^{L2d} is C₄₋₆ alkyl substituted with 3 to 5 hydroxy groups, preferably R^{L2d} is C₄₋₆ alkyl substituted with 3 to 5 hydroxy groups, where no more than one hydroxyl group is substituted on each carbon atom, for example, R^{L2d} is selected from the group consisting of:

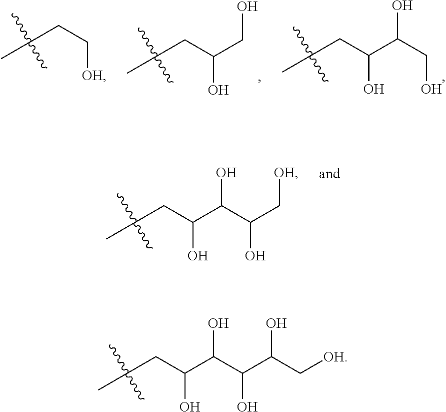

32. The ligand-drug conjugate or a pharmaceutically acceptable salt, solvate, stereoisomer, or isotopic variant thereof according to any of claims 24 to 31, wherein the hydrophilic group L₂ is selected from the group consisting of: 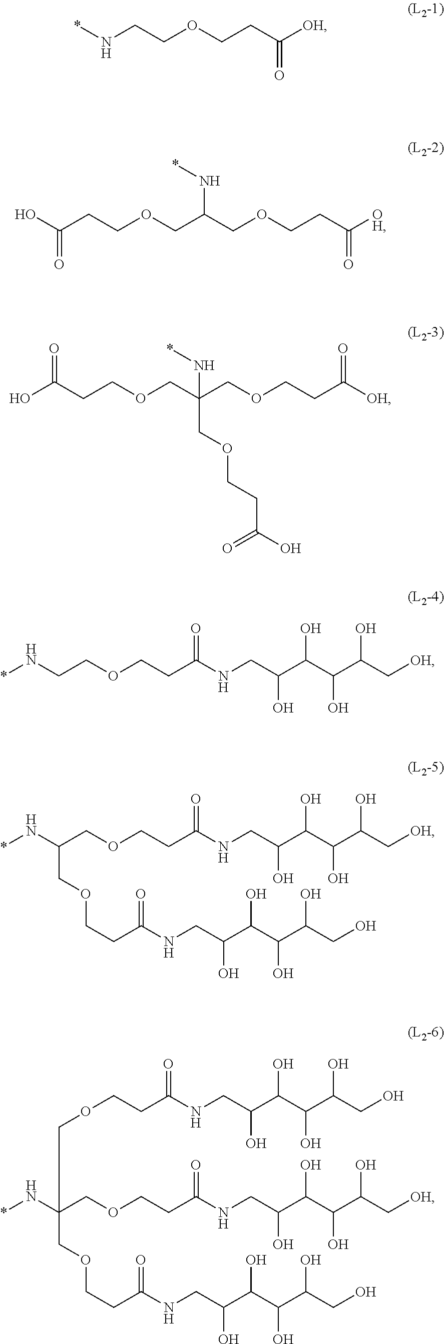 [SMILES: *NCCOCCC(=O)O ; *NC(COCCC(=O)O)COCCC(=O)O ; *NC(COCCC(=O)O)(COCCC(=O)O)COCCC(=O)O ; *NCCOCCC(=O)NCC(O)C(O)C(O)C(O)CO ; *NC(COCCC(=O)NCC(O)C(O)C(O)C(O)CO)COCCC(=O)NCC(O)C(O)C(O)C(O)CO ; *NC(COCCC(=O)NCC(O)C(O)C(O)C(O)CO)(COCCC(=O)NCC(O)C(O)C(O)C(O)CO)COCCC(=O)NCC(O)C(O)C(O)C(O)CO]
“*” denotes to the site covalently attached to polypeptide residue L′₁.

33. The ligand-drug conjugate or a pharmaceutically acceptable salt, solvate, stereoisomer, or isotopic variant thereof according to any of claims 24 to 32, wherein the auxiliary moiety Z is selected from the group consisting of mercapto, disulfide, amino, carboxyl, aldehyde, maleimide, haloacetyl, hydrazide and hydroxyl groups.

34. The ligand-drug conjugate or a pharmaceutically acceptable salt, solvate, stereoisomer, or isotopic variant thereof according to any of claims 24 to 34, wherein the ligand LG is an antibody or antigen-binding fragment thereof.

35. The ligand-drug conjugate or a pharmaceutically acceptable salt, solvate, stereoisomer, or isotopic variant thereof according to any of claims 24 to 34, wherein the therapeutic agent is a cytotoxic or cytostatic agent.

36. The ligand-drug conjugate or a pharmaceutically acceptable salt, solvate, stereoisomer, or isotopic variant thereof according to any of claims 24 to 35, wherein the therapeutic agent is a camptothecin compound, an analogue or a derivative thereof.

37. The ligand-drug conjugate or a pharmaceutically acceptable salt, solvate, stereoisomer, or isotopic variant thereof according to any of claims 24 to 36, wherein the ligand-drug conjugate is a compound of Formula (III′): 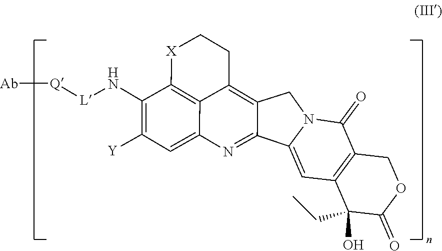
or a pharmaceutically acceptable salt, solvate, stereoisomer, or isotopic variant thereof, wherein
X is selected from the group consisting of —CH₂—, O, and S; Y is selected from the group consisting of H, D, and F;
Ab denotes to an antibody;
Q and L′ are as defined in the preceding claims; and
n is a number in the range from 1 to 8.

38. The ligand-drug conjugate or a pharmaceutically acceptable salt, solvate, stereoisomer, or isotopic variant thereof according to any of claims 24 to 37, wherein the ligand-drug conjugate is selected from the group consisting of: 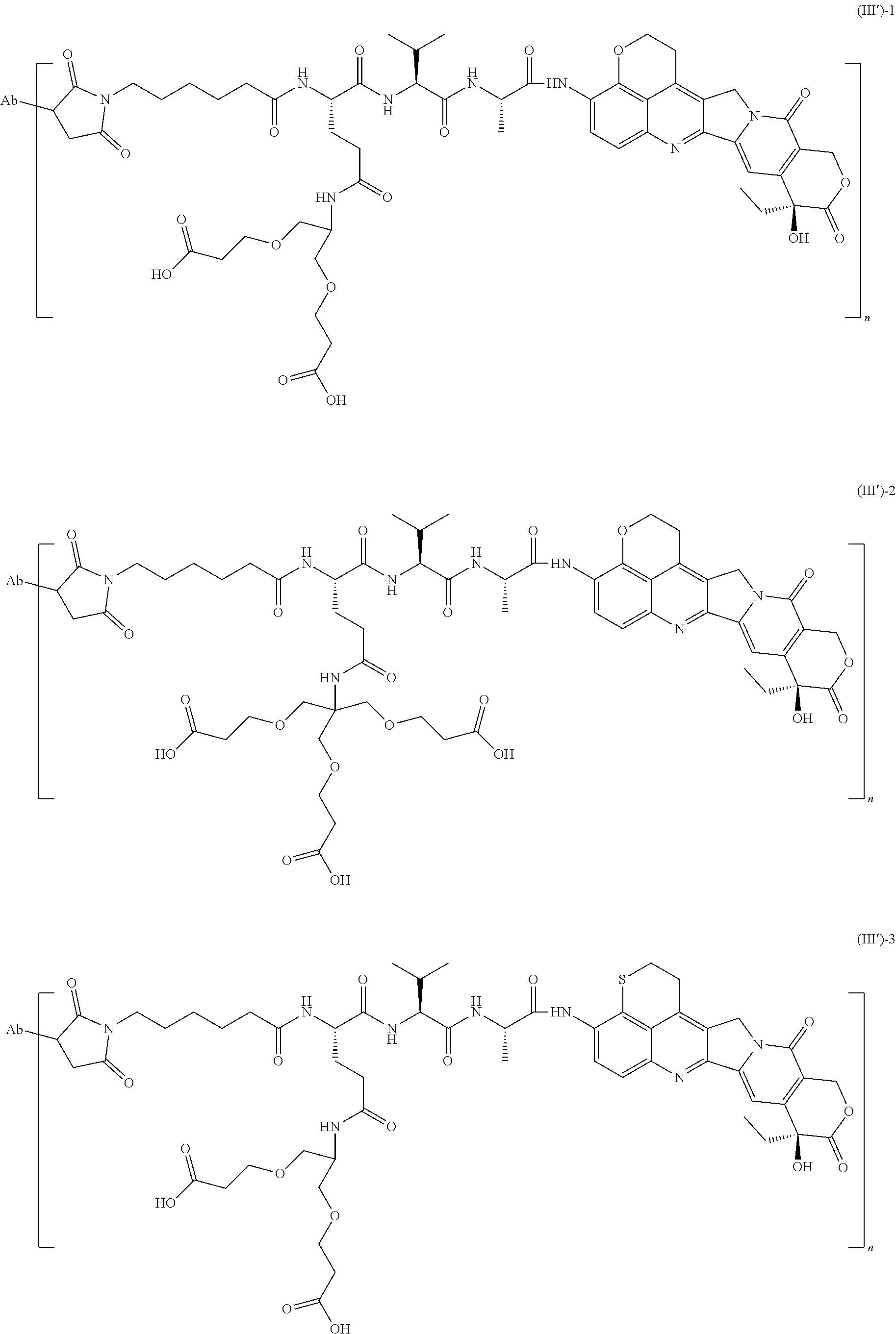 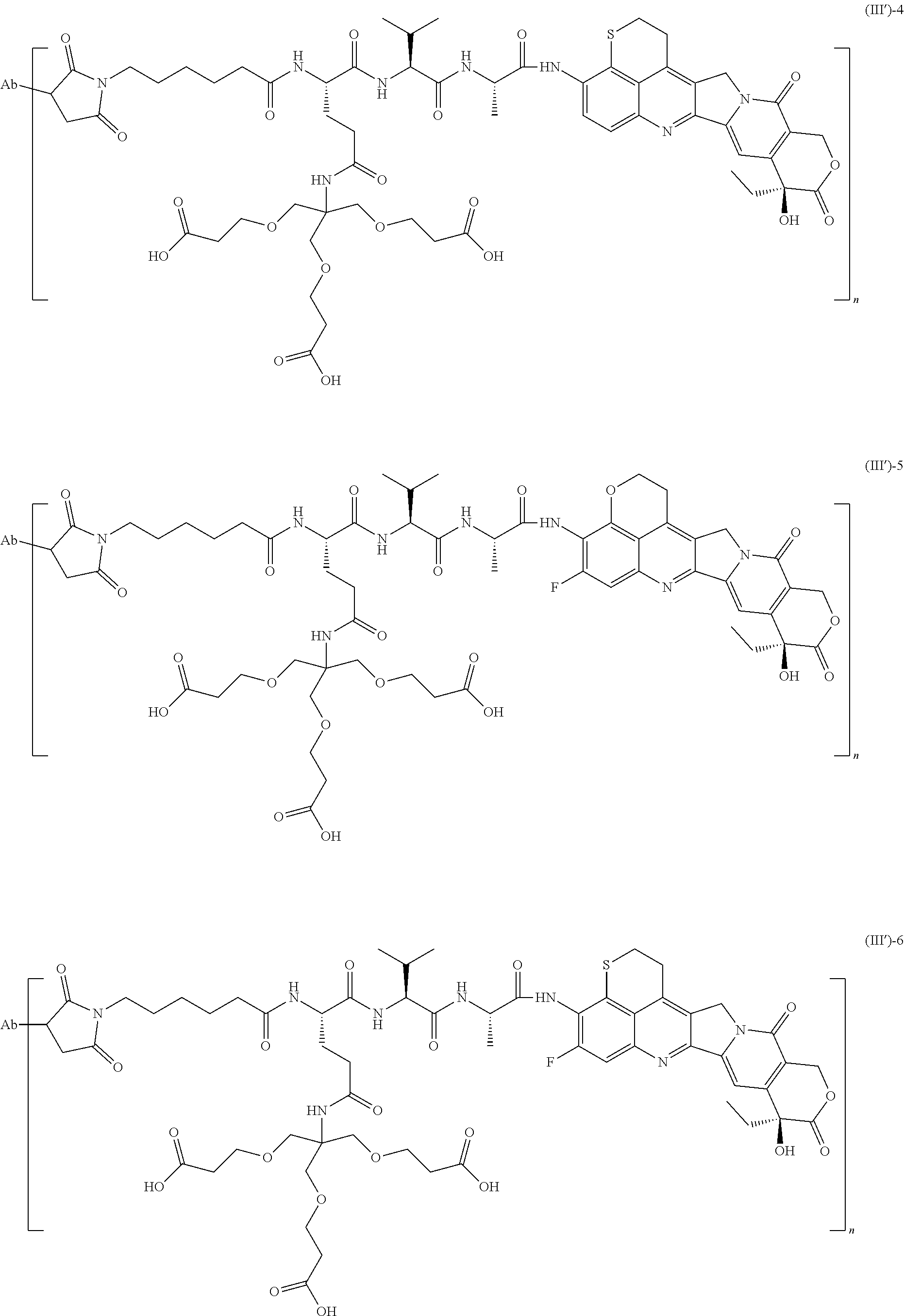 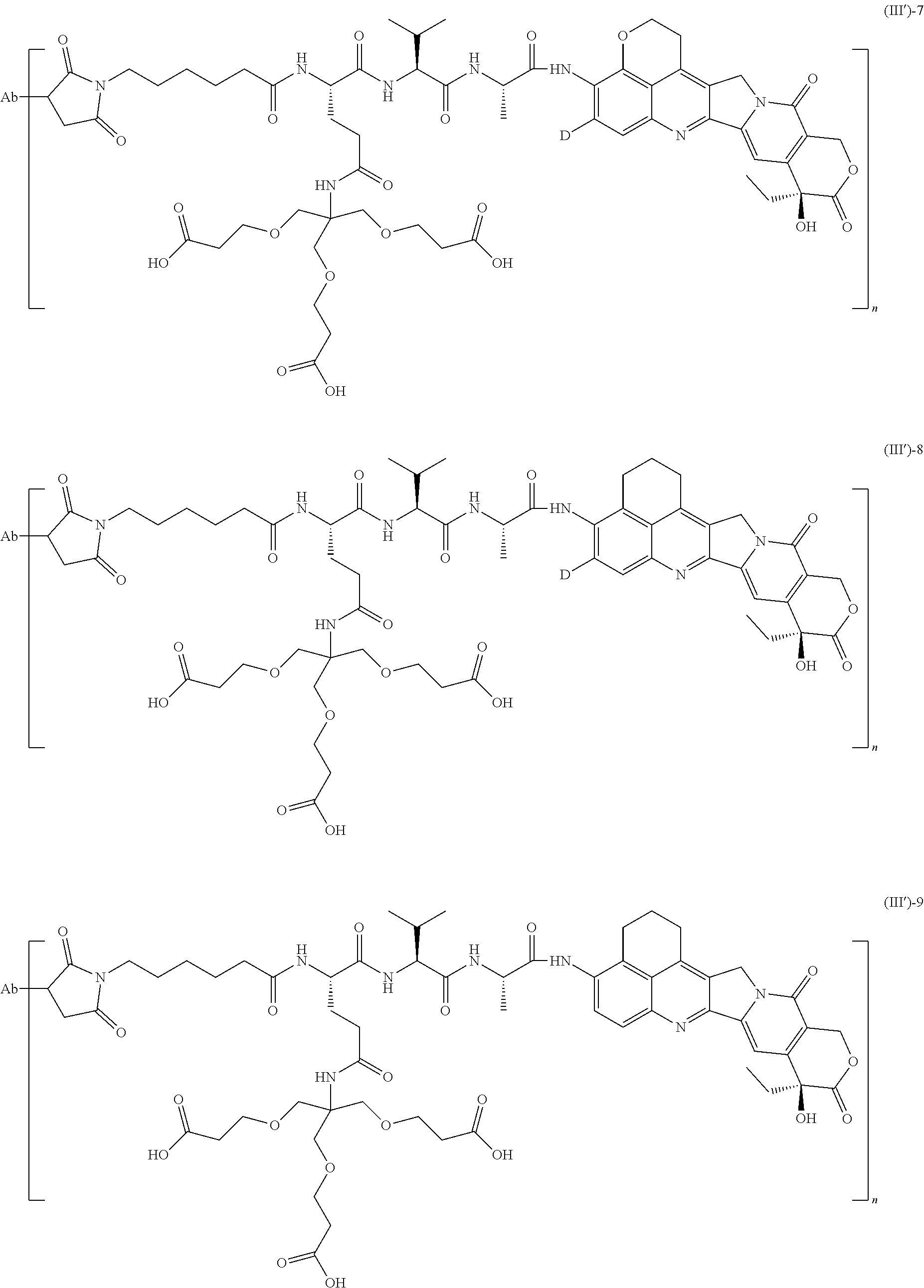 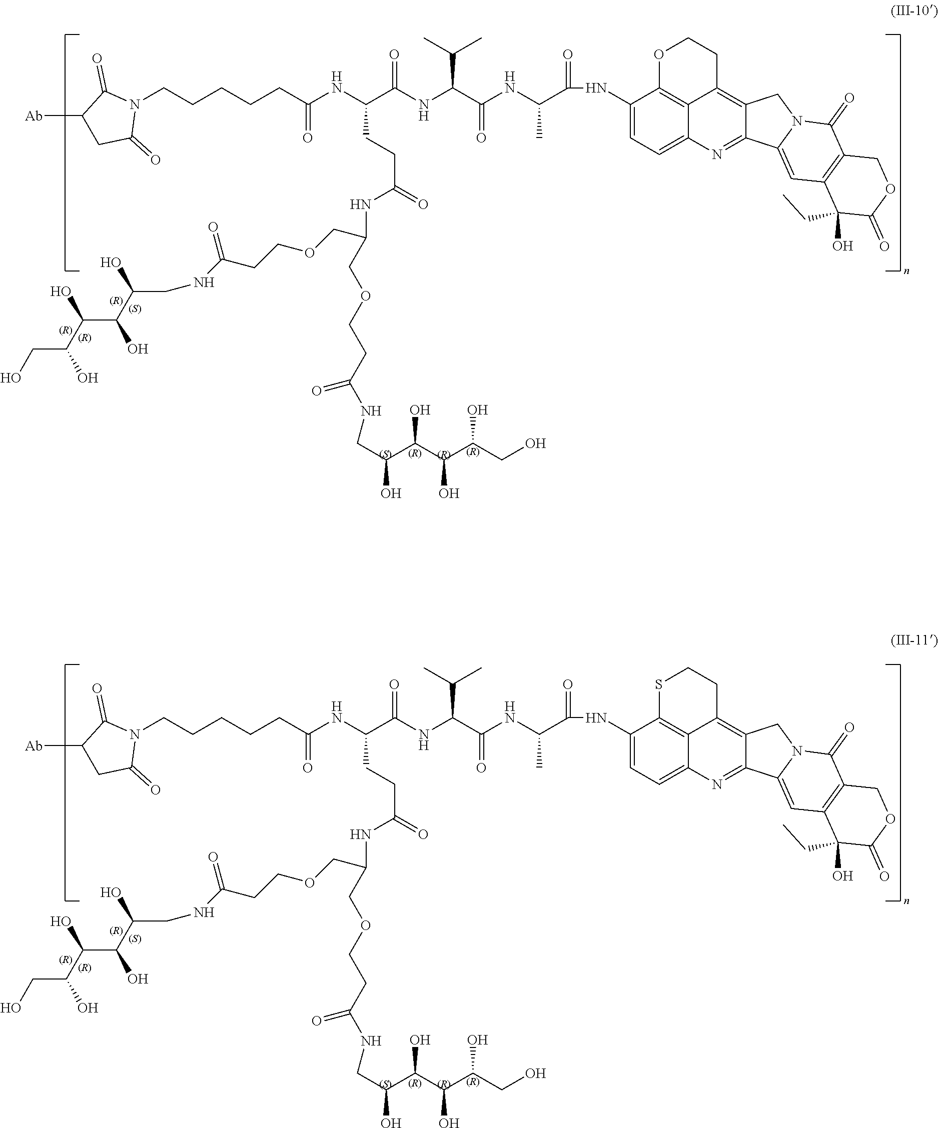 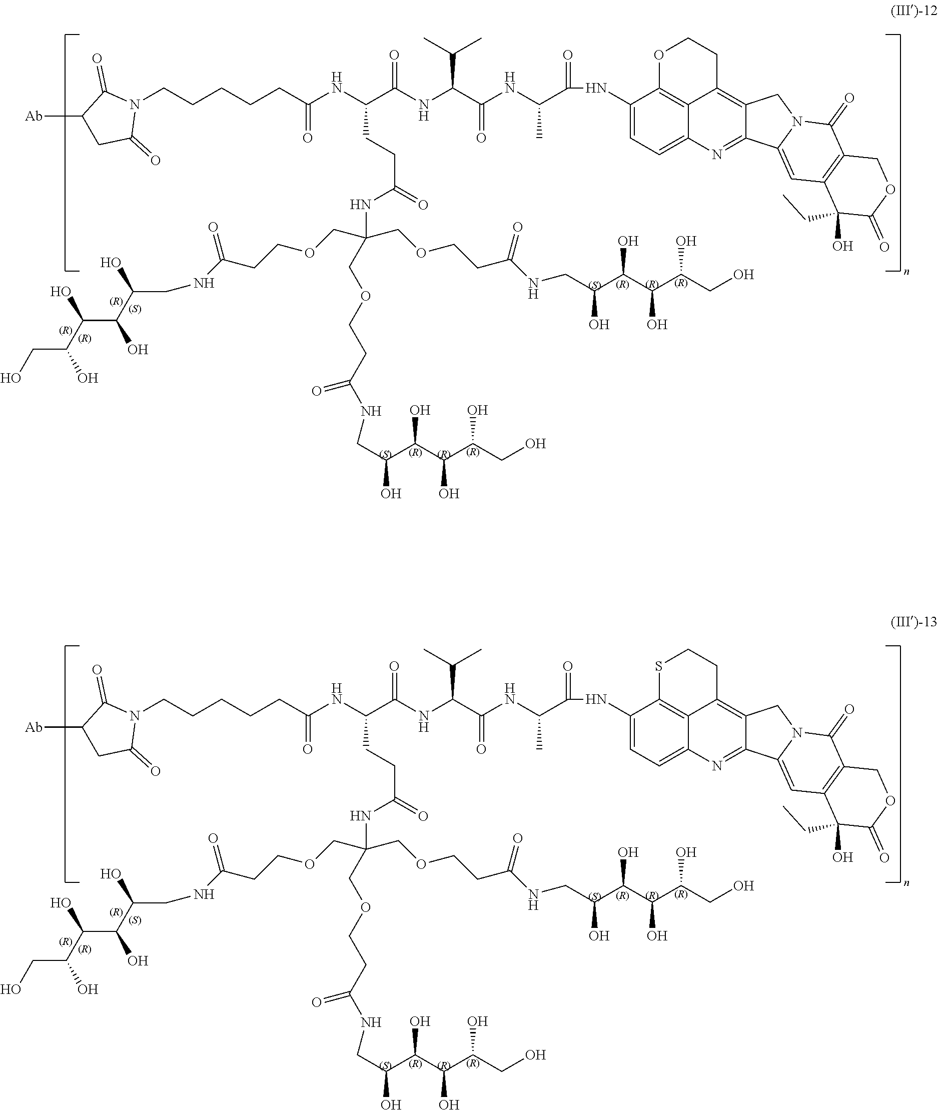 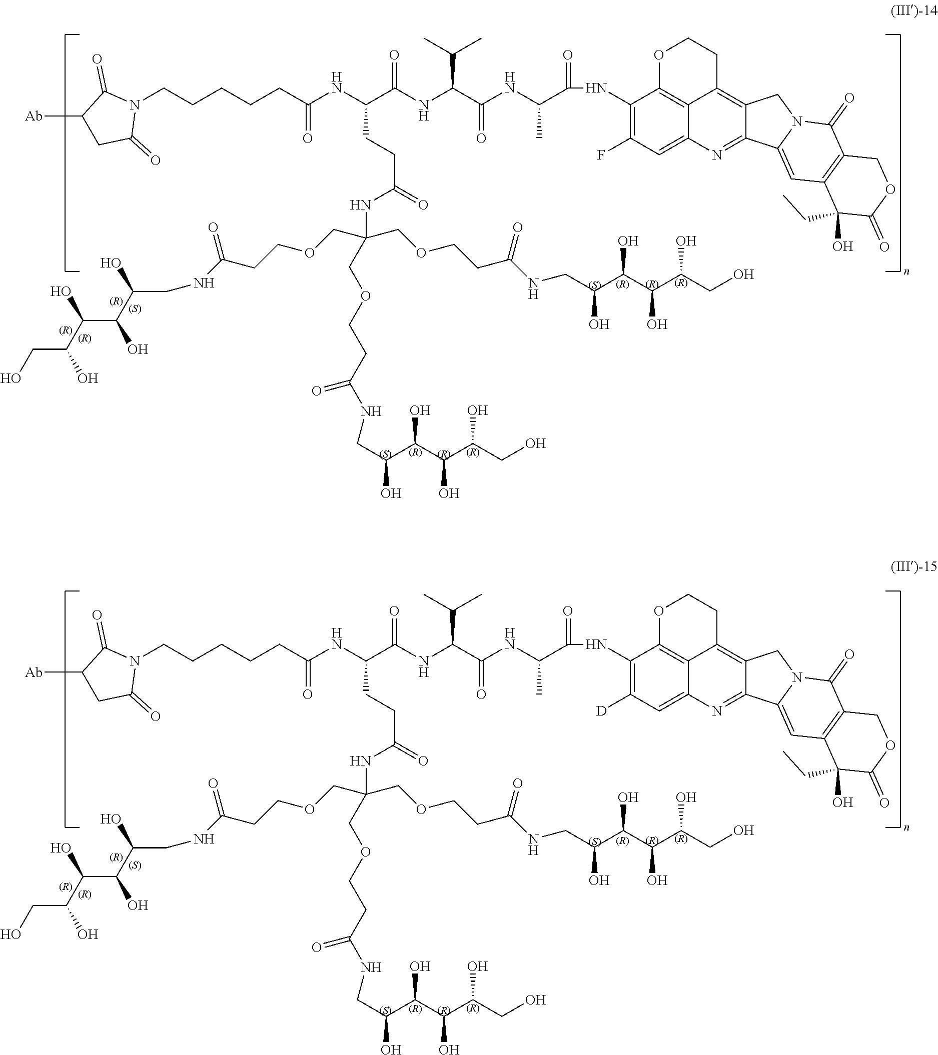 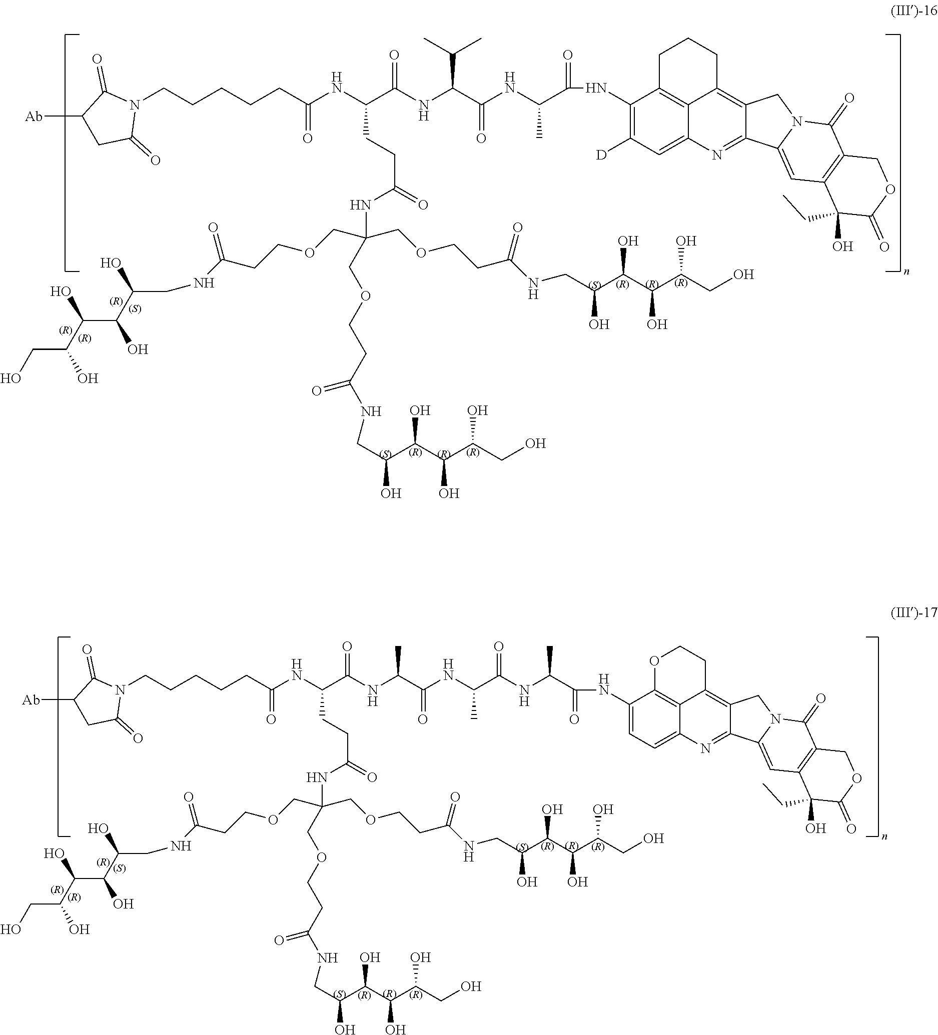 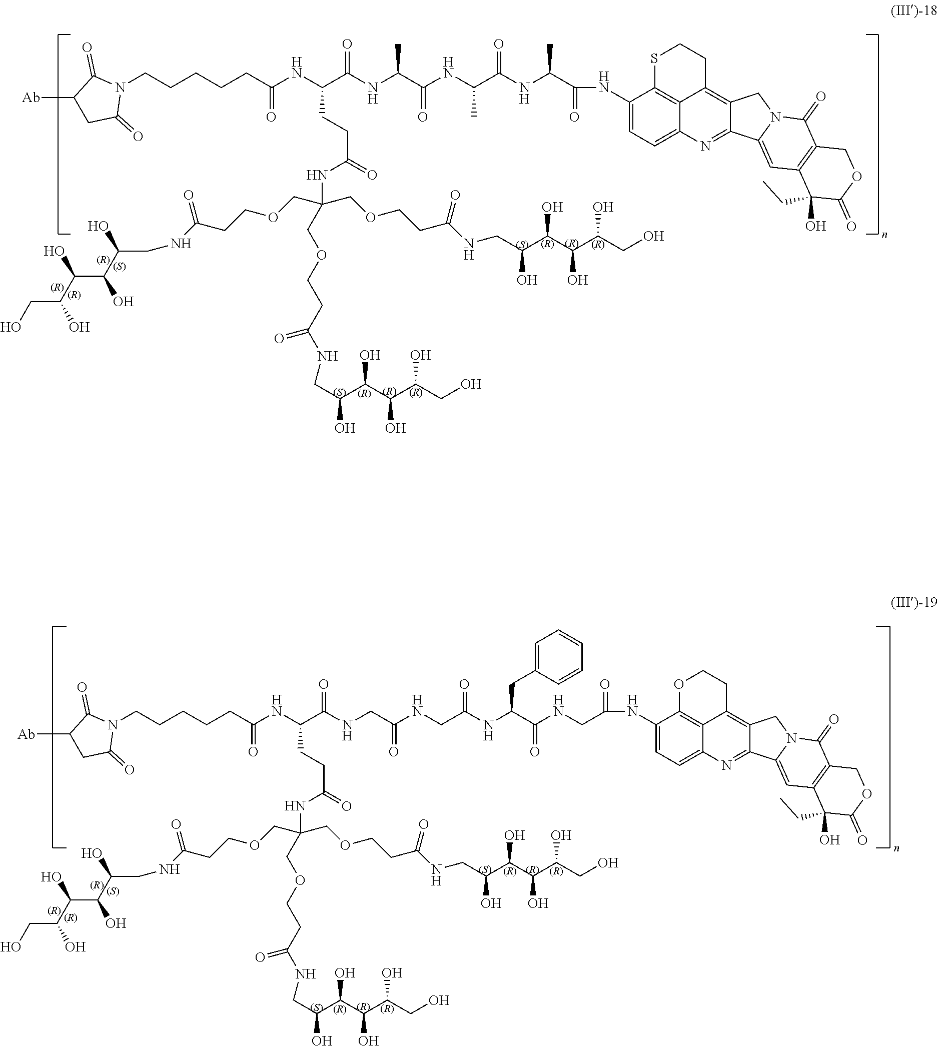 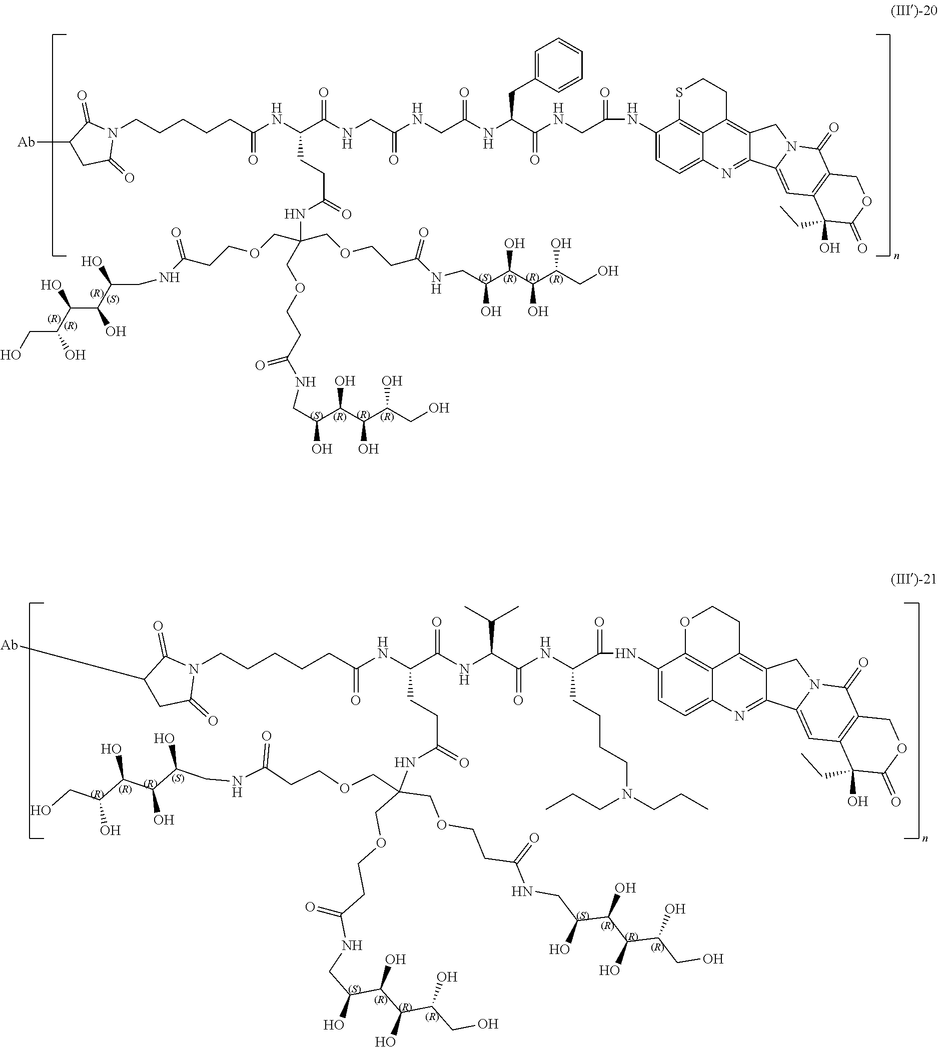 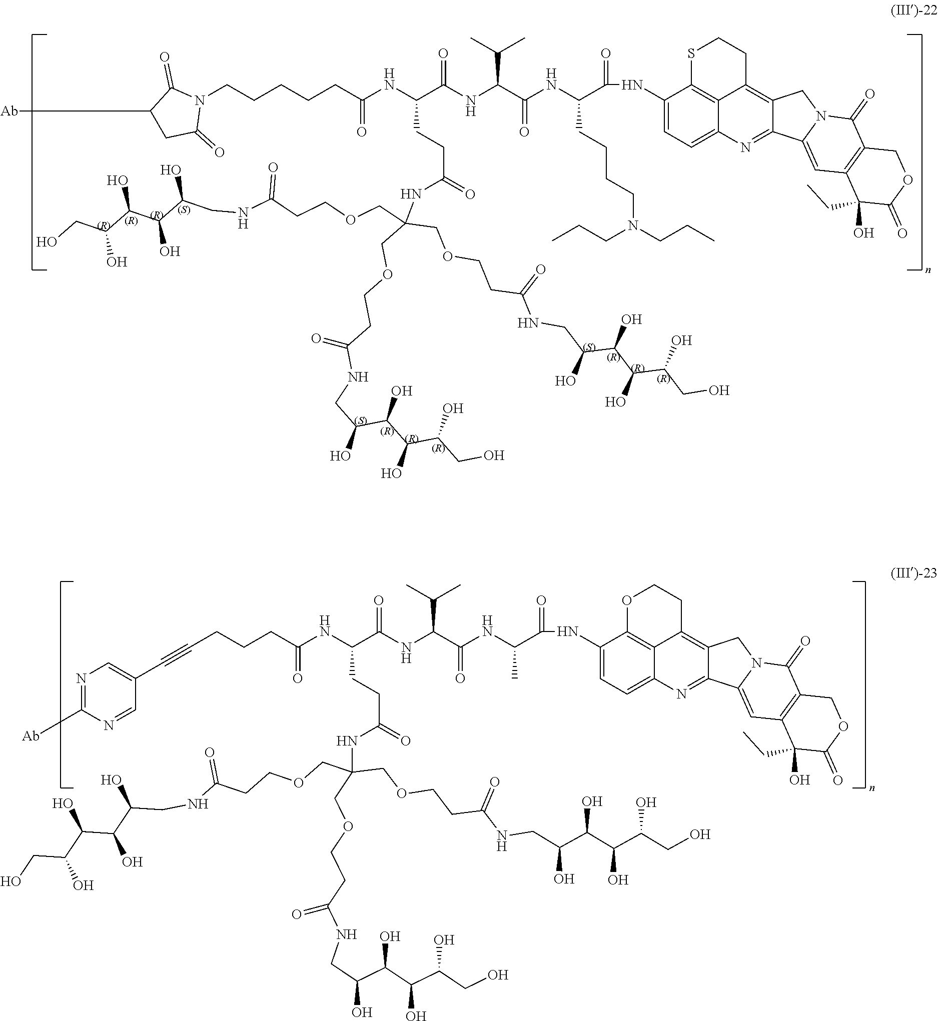 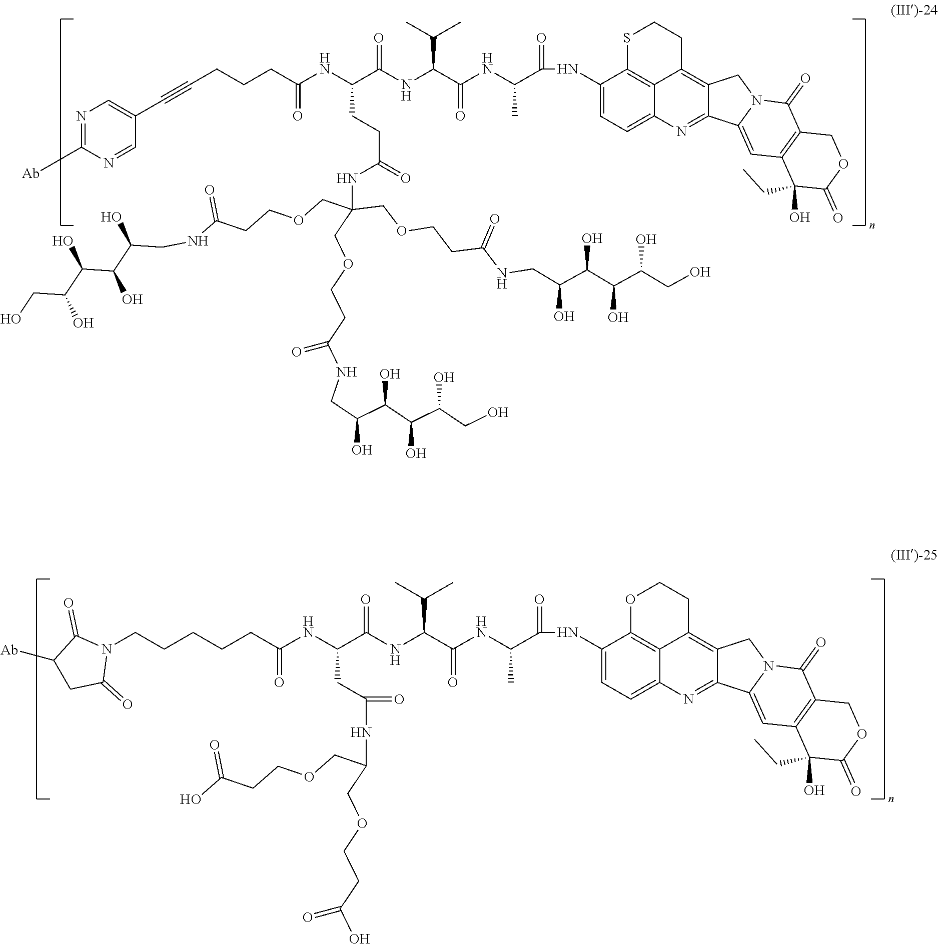 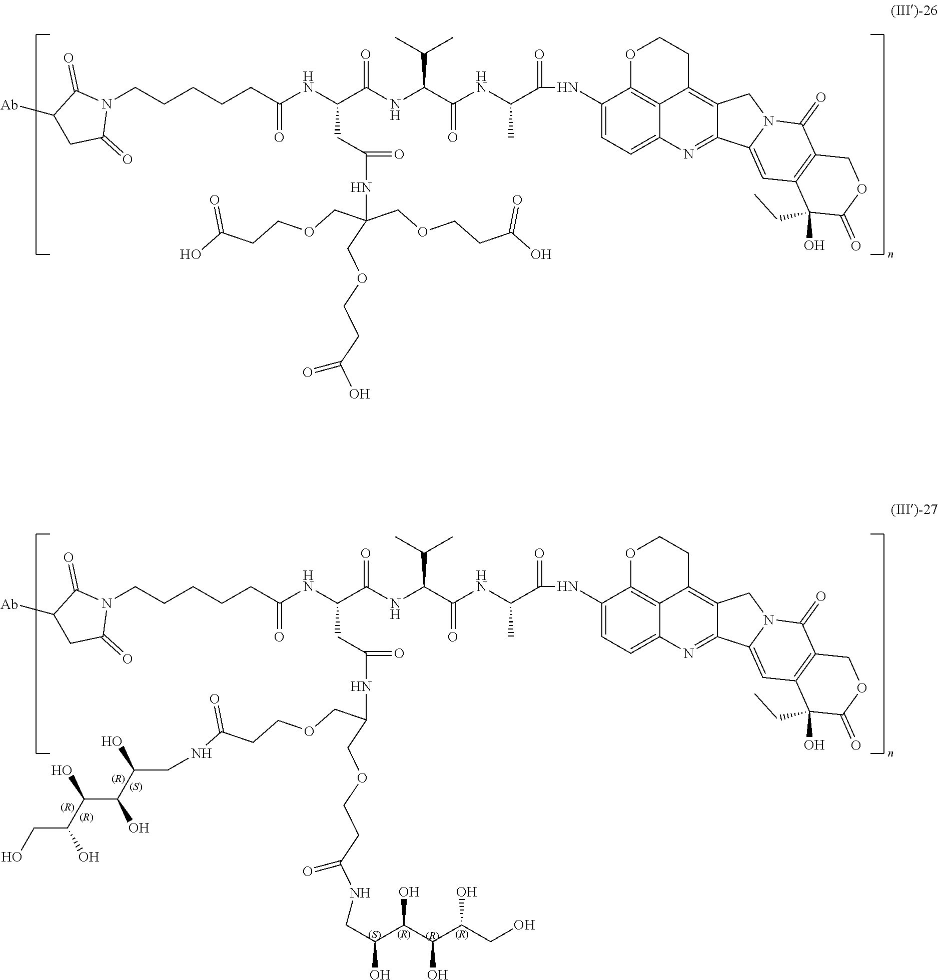 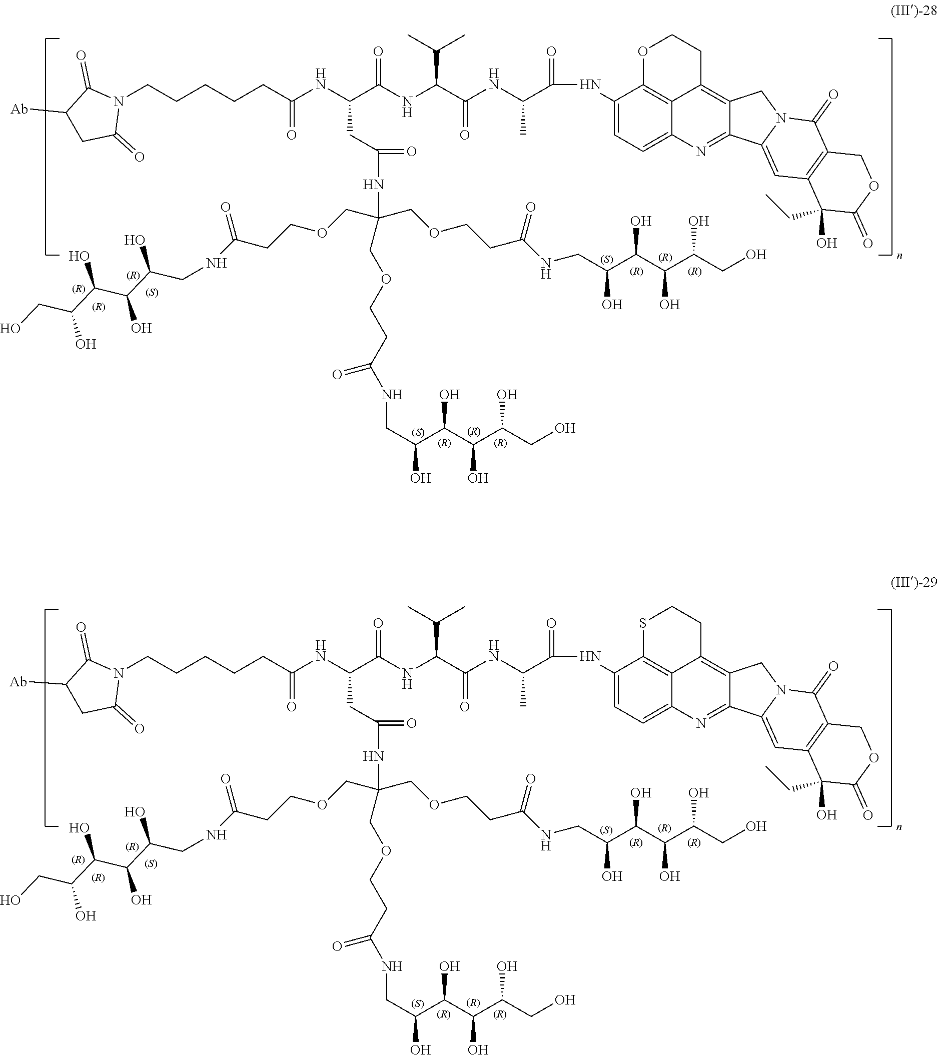 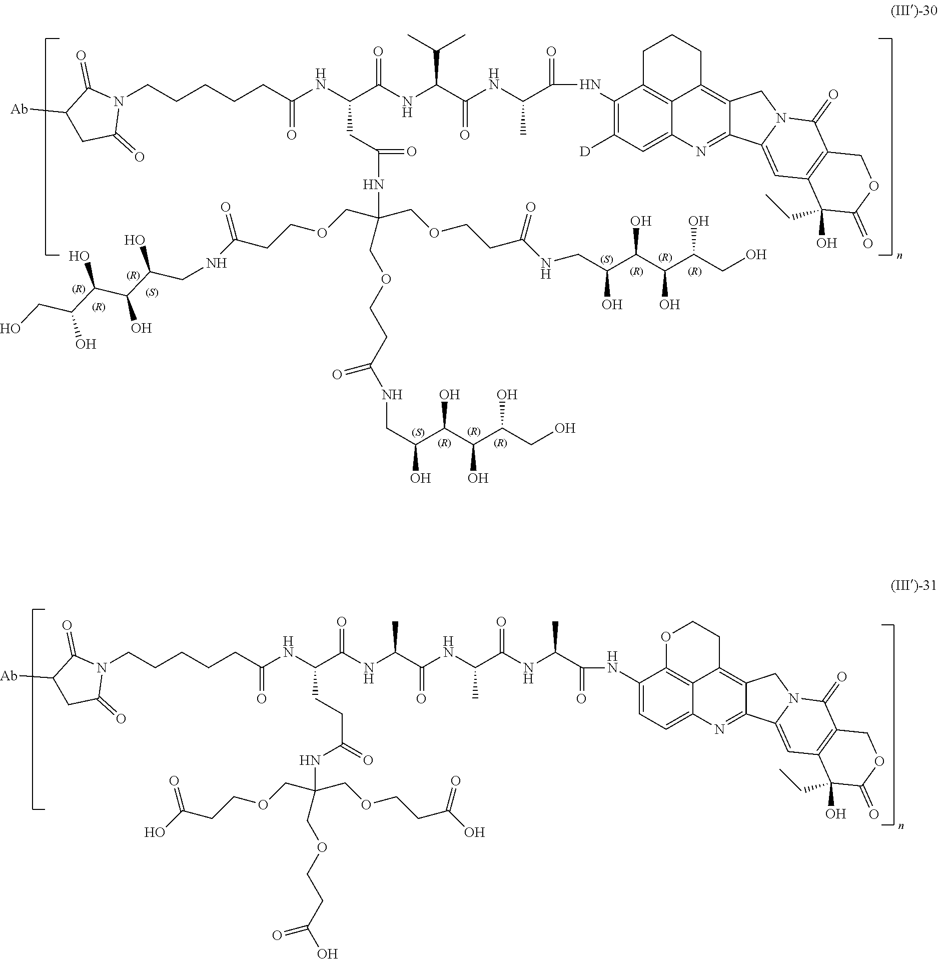 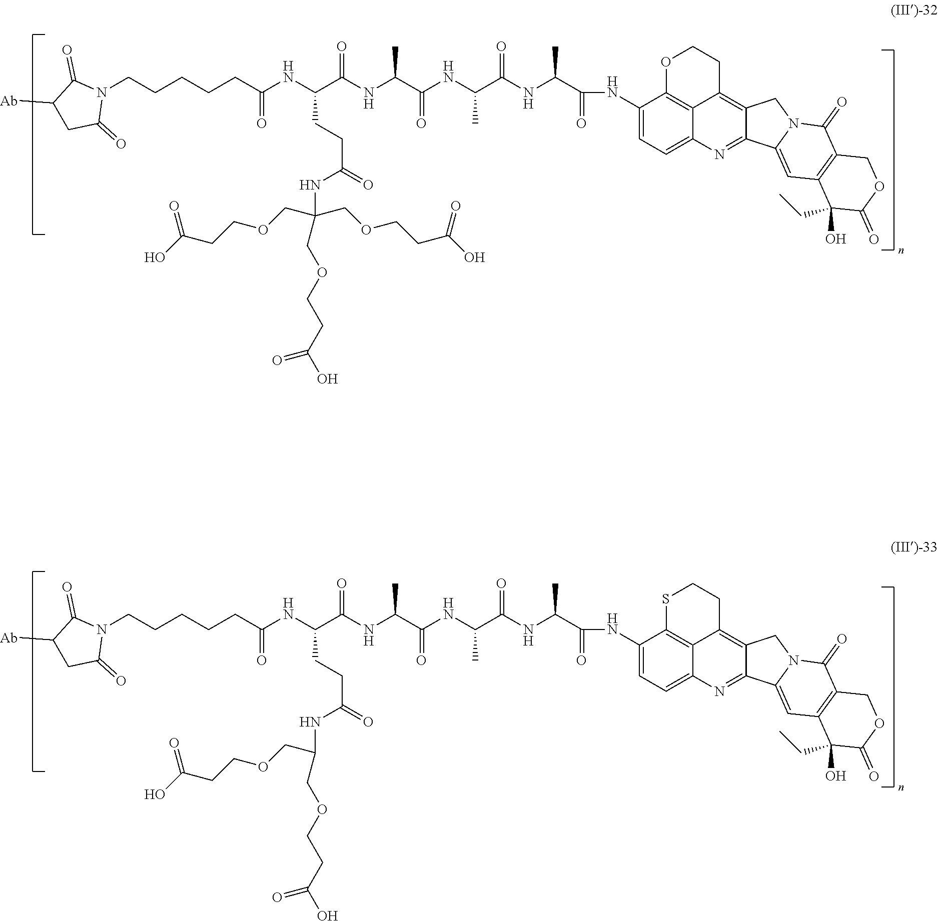 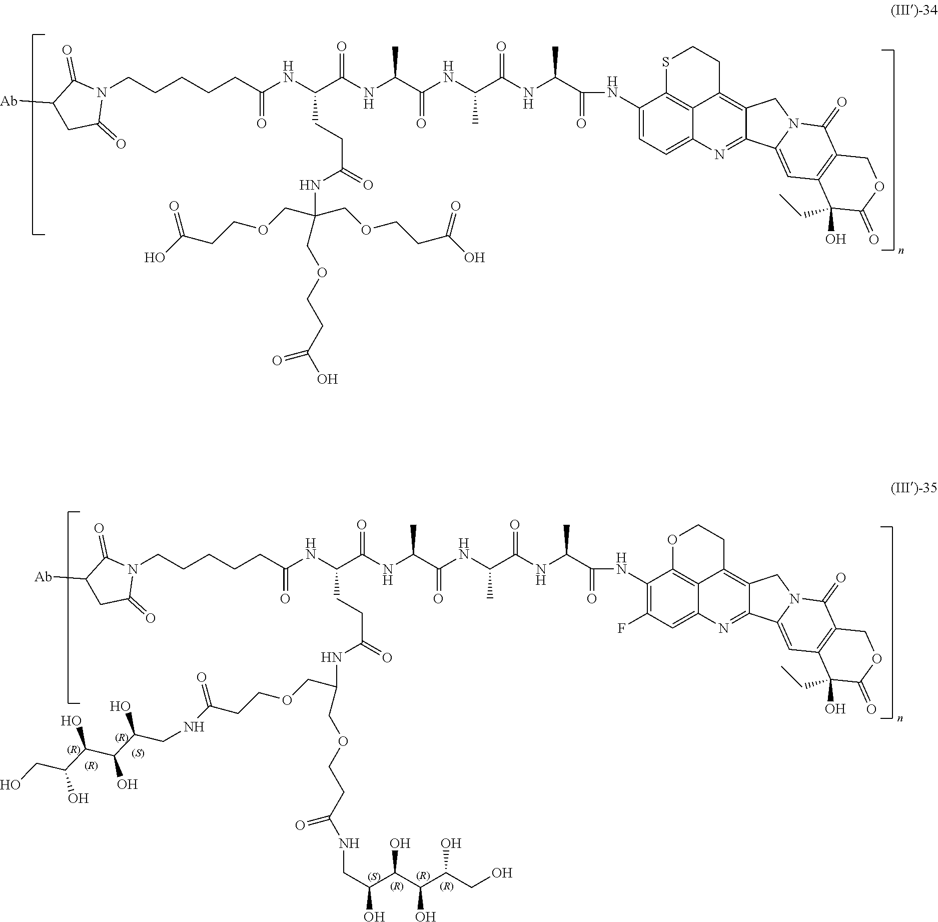 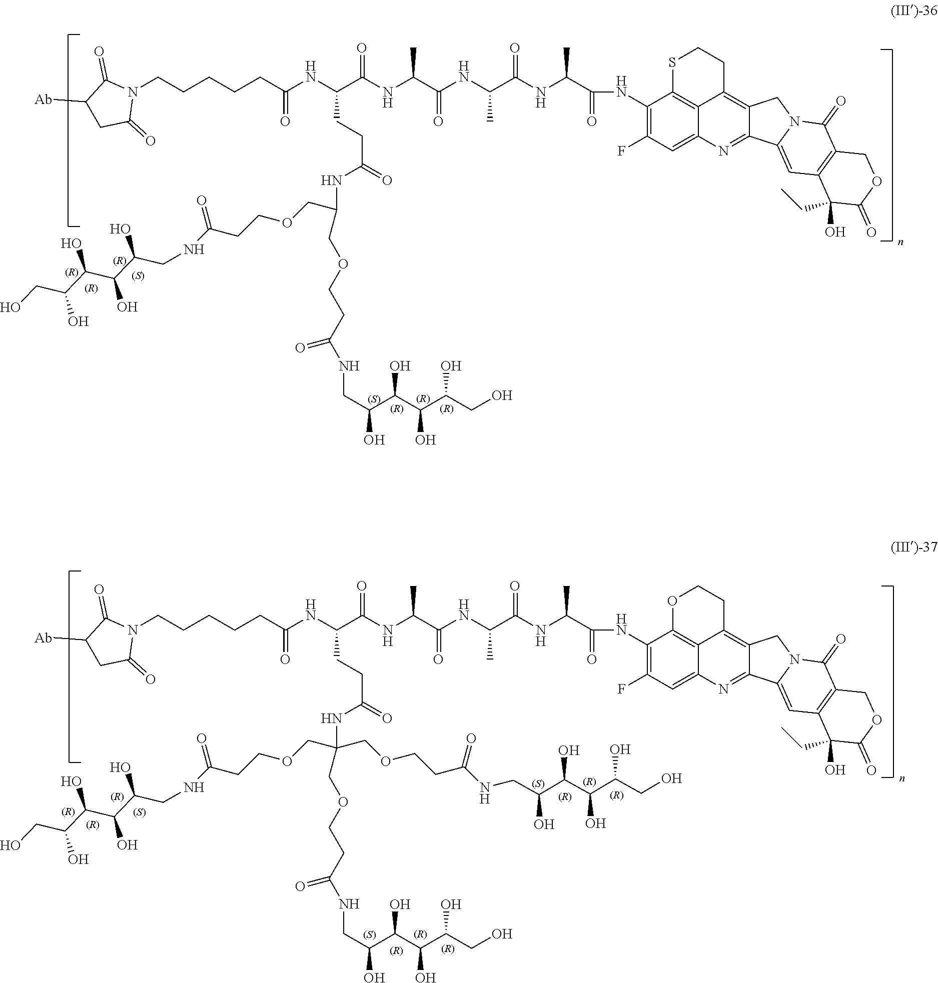 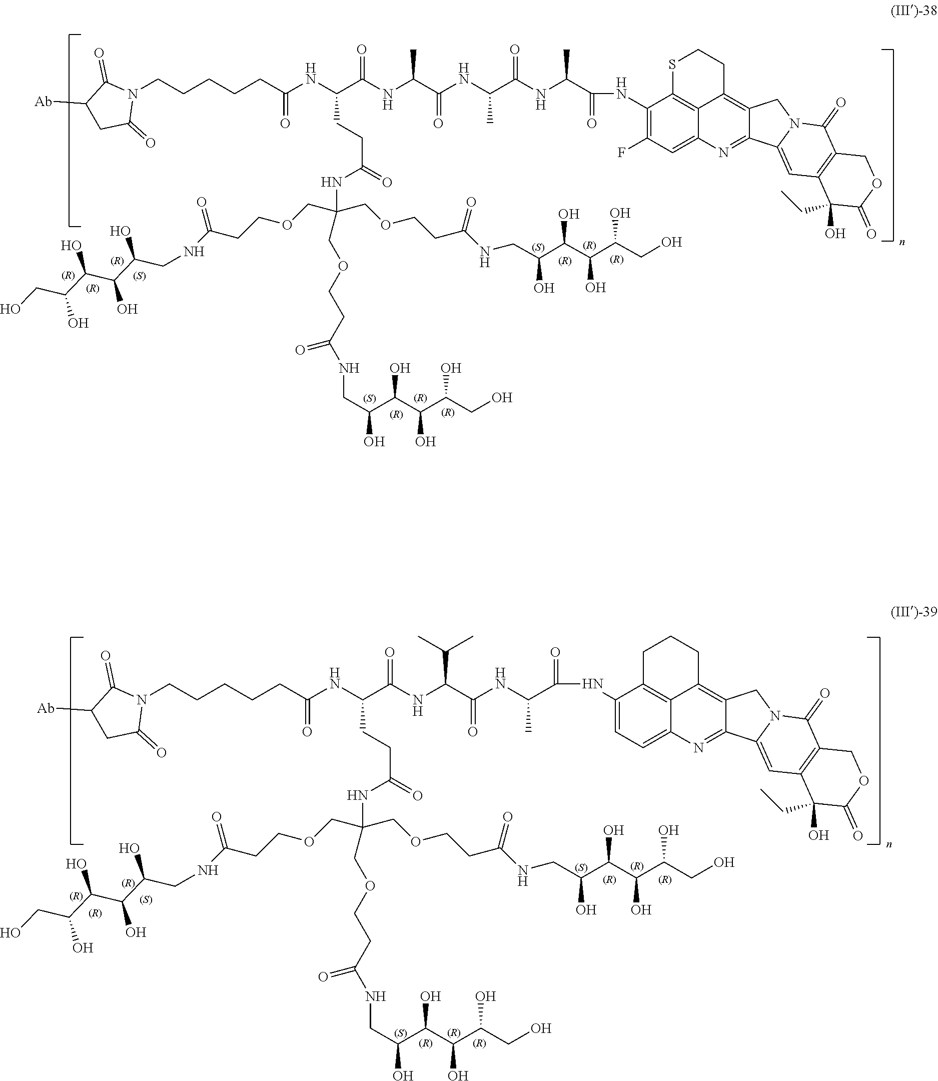 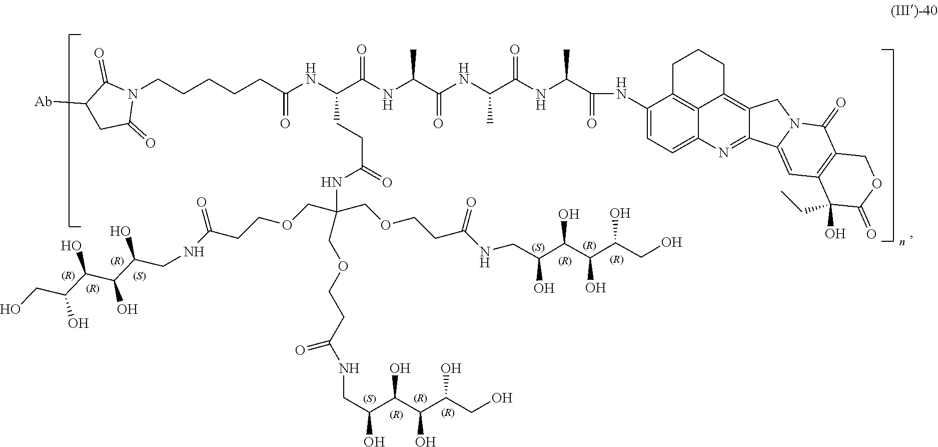
where n is a number in the range from 1 to 8.

39. The ligand-drug conjugate or a pharmaceutically acceptable salt, solvate, stereoisomer, or isotopic variant thereof according to any of claims 34 to 38, wherein the antibody is selected from the group consisting of mouse-derived antibodies, chimeric antibodies, humanized antibodies and fully humanized antibodies.

40. The ligand-drug conjugate or a pharmaceutically acceptable salt, solvate, stereoisomer, or isotopic variant thereof according to any of claims 34 to 39, wherein the antibody is mAb.

41. The ligand-drug conjugate or a pharmaceutically acceptable salt, solvate, stereoisomer, or isotopic variant thereof according to any of claims 34 to 40, wherein the antibody is bispecific antibody or multispecific antibody.

42. The ligand-drug conjugate or a pharmaceutically acceptable salt, solvate, stereoisomer, or isotopic variant thereof according to any of claims 34 to 41, wherein the antibody is antibody fragments, nanobody or fusion protein.

43. The ligand-drug conjugate or a pharmaceutically acceptable salt, solvate, stereoisomer, or isotopic variant thereof according to any of claims 34 to 42, wherein the antibody is an antibody capable of binding to CTLA4, CD137, OX40, HER2, HER3, CD19, CD20, CD22, CD30, CD33, CD37, CD45, CD56, CD66e, CD70, CD74, CD79b, CD137, CD138, CD147, CD223, EpCAM, Mucin 1, STEAPI, GPNMB, FGF2, FOLR1, EGFR, EGFRvIII, Tissue factor, c-MET, FGFR, Nectin 4, AGS-16, Guanylyl cyclase C, Mesothelin, SLC44A4, PSMA, EphA2, AGS-5, GPC-3, c-KIT, ROR1, PD-L1, CD27L, 5T4, Mucin 16, NaPi2b, STEAP, SLITRK6, ETBR, BCMA, Trop-2, CEACAM5, SC-16, SLC39A6, Delta-like protein3, or Claudin 18.2 tumor-associated antigen.

44. A pharmaceutical composition comprising a ligand-drug conjugate or a pharmaceutically acceptable salt, solvate, stereoisomer, or isotopic variant thereof according to any of claims 24 to 43; and a pharmaceutically acceptable diluent, carrier or excipient.

45. The pharmaceutical composition according to claim 44, wherein the ligand-drug conjugate has an ADC DAR value in the range from 1 to 8.

46. Use of a ligand-drug conjugate or a pharmaceutically acceptable salt, solvate, stereoisomer, or isotopic variant thereof according to any of claims 24 to 43 in preparation of a medicament for treating a tumor in a subject.

47. The use according to claim 46, wherein the tumor is selected from the group consisting of solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer pancreatic cancer, liver cancer, bladder cancer, stomach cancer or breast cancer.

48. A ligand-drug conjugate or a pharmaceutically acceptable salt, solvate, stereoisomer, or isotopic variant thereof according to any of claims 24 to 43 for use in treating a tumor in a subject.

49. The ligand-drug conjugate or a pharmaceutically acceptable salt, solvate, stereoisomer, or isotopic variant thereof according to any of claims 24 to 43 for use according to claim 48, wherein the tumor is selected from the group consisting of solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer pancreatic cancer, liver cancer, bladder cancer, stomach cancer or breast cancer.

50. A method for treating a tumor in a subject comprising administrating to the subject an effective amount of a ligand-drug conjugate or a pharmaceutically acceptable salt, solvate, stereoisomer, or isotopic variant thereof according to any of claims 24 to 43.

51. The method according to claim 50, wherein the tumor is selected from the group consisting of solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer pancreatic cancer, liver cancer, bladder cancer, stomach cancer or breast cancer.

52. A method for preparation of a ligand-drug conjugate or a pharmaceutically acceptable salt, solvate, stereoisomer, or isotopic variant thereof according to any of claims 24 to 43, comprising the following steps of:
a) reacting a ligand with a reducing agent in a buffer to obtain a reduced ligand; and
b) coupling a compound or a pharmaceutically acceptable salt, solvate, stereoisomer, or isotopic variant thereof according to any of claims 11 to 23 with the reduced ligand obtained in step a) in a mixture of a buffer and an organic solvent to obtain the ligand-drug conjugate;
or comprising the following step of:
coupling a compound or a pharmaceutically acceptable salt, solvate, stereoisomer, or isotopic variant thereof according to any of claims 11 to 23 with a ligand in a mixture of a buffer and an organic solvent to obtain the ligand-drug conjugate.

53. A compound of Formula (IV): 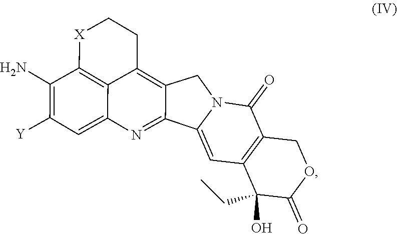
or a pharmaceutically acceptable salt, solvate, stereoisomer, or isotopic variant thereof, wherein
X is selected from the group consisting of —CH₂—, O, and S; and
Y is selected from the group consisting of H, D, and F.

54. A compound according to claim 53 for use in treating a tumor in a subject.
